SEQUENCE LISTING (129 sequences)

SEQ ID NO: 1 - protein, 123 aa, synthetic construct
  REGION 1..123: note = CD70 VHH (Binder 16)
  VARIANT 1: note = X is Q or pyroglutamate
XVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSS

SEQ ID NO: 2 - protein, 123 aa, synthetic construct
  REGION 1..123: note = CD70 VHH (Binder 16)
QVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSS

SEQ ID NO: 3 - protein, 123 aa, synthetic construct; identical to SEQ ID NO: 1
  REGION 1..123: note = CD70 VHH (Binder 16)
  VARIANT 1: note = X is pyroglutamate

SEQ ID NO: 4 - protein, 5 aa, synthetic construct
  REGION 1..5: note = CD70 CDR1 Kabat (Binder 16)
YSAIG

SEQ ID NO: 5 - protein, 17 aa, synthetic construct
  REGION 1..17: note = CD70 CDR2 Kabat (Binder 16)
CTSSSDGSIYYADSVKG

SEQ ID NO: 6 - protein, 14 aa, synthetic construct
  REGION 1..14: note = CD70 CDR3 Kabat (Binder 16)
GVFHGGSCRGALDT

SEQ ID NO: 7 - protein, 8 aa, synthetic construct
  REGION 1..8: note = CD70 CDR1 IMGT (Binder 16)
GVTLEYSA

SEQ ID NO: 8 - protein, 8 aa, synthetic construct
  REGION 1..8: note = CD70 CDR2 IMGT(Binder 16)
TSSSDGSI

SEQ ID NO: 9 - protein, 16 aa, synthetic construct
  REGION 1..16: note = CD70 CDR3 IMGT(Binder 16)
AIGVFHGGSCRGALDT

SEQ ID NO: 10 - protein, 39 aa, Homo sapiens
  REGION 1..39: note = Human CD28 Hinge
IEVMYPPPYLDNEKSNGTIIHVKGKHLCPSPLFPGPSKP

SEQ ID NO: 11 - protein, 27 aa, Homo sapiens
  REGION 1..27: note = Human CD28 Transmembrane domain
FWVLVVVGGVLACYSLLVTVAFIIFWV

SEQ ID NO: 12 - protein, 42 aa, synthetic construct
  REGION 1..42: note = 4-1BB-derived intracellular costimulatory domain (aa)
KRGRKKLLYIFKQPFMRPVQTTQEEDGCSCRFPEEEEGGCEL

SEQ ID NO: 13 - protein, 112 aa, synthetic construct
  REGION 1..112: note = CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 359 aa, synthetic construct
  REGION 1..359: note = CD70 CAR (Binder 16) with signal peptide (amino acid sequence)
MPLLLLLPLLWAGALAXVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 359 aa, synthetic construct
  REGION 1..359: note = CD70 CAR (Binder 16) with signal peptide (amino acid sequence)
MPLLLLLPLLWAGALAQVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 359 aa, synthetic construct; identical to SEQ ID NO: 14
  REGION 1..359: note = CD70 CAR (Binder 16) with signal peptide (amino acid sequence)

SEQ ID NO: 17 - DNA, 1077 bp, synthetic construct
  misc_feature 1..1077: note = CD70 CAR (Binder 16) (nucleic acid sequence)
  source 1..1077: mol_type = other DNA
atgcccctgctgctcctgctccctttgctgtgggcgggagccctcgcgcaagtgcaactggtagaaagcggcggcggcctggtgcagcctggcggctctctaagactgagctgcgccgcaagcggcgtgaccctggagtacagcgccatcggctggttcagacaagcccccggcaaggagcaagagggcgtgagctgcacaagcagcagcgacggcagcatctactacgccgacagcgtgaagggcagattcaccatcagcagagacaacagcaagaacaccctgtacctgcagatgaacagcctgagagccgaggacaccgccgtgtactactgcgccatcggcgttttccatggcggttcttgcagaggcgcccttgatacctggggccaaggcaccctggtgaccgtgagcagcattgaagttatgtatcctcctccttacctagacaatgagaagagcaatggaaccattatccatgtgaaagggaaacacctttgtccaagtcccctatttcccggaccttctaagcccttctgggtgctcgtggtcgttggcggagtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 18 - protein, 16 aa, synthetic construct
  REGION 1..16: note = CD33 signal peptide
MPLLLLLPLLWAGALA

SEQ ID NO: 19 - DNA, 209 bp, synthetic construct
  misc_feature 1..209: note = EF1alpha promoter
  source 1..209: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 20 - DNA, 544 bp, synthetic construct
  misc_feature 1..544: note = EF1alpha-HTLV1R promoter
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 21 - protein, 22 aa, synthetic construct
  REGION 1..22: note = F2A peptide
VKQTLNFDLLKLAGDVESNPGP

SEQ ID NO: 22 - protein, 20 aa, synthetic construct
  REGION 1..20: note = E2A peptide
QCTNYALLKLAGDVESNPGP

SEQ ID NO: 23 - protein, 24 aa, synthetic construct
  REGION 1..24: note = T2A peptide
LEGGGEGRGSLLTCGDVEENPGPR

SEQ ID NO: 24 - protein, 18 aa, synthetic construct
  REGION 1..18: note = T2A peptide
EGRGSLLTCGDVEENPGP

SEQ ID NO: 25 - protein, 21 aa, synthetic construct
  REGION 1..21: note = T2A peptide
GSGEGRGSLLTCGDVEENPGP

SEQ ID NO: 26 - protein, 22 aa, synthetic construct
  REGION 1..22: note = P2A peptide
GSGATNFSLLKQAGDVEENPGP

SEQ ID NO: 27 - protein, 123 aa, synthetic construct
  REGION 1..123: note = VHH (Binder 18)
  VARIANT 1: note = X is Q or pyroglutamate
XVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSS

SEQ ID NO: 28 - protein, 123 aa, synthetic construct
  REGION 1..123: note = VHH (Binder 18)
QVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSS

SEQ ID NO: 29 - protein, 123 aa, synthetic construct; identical to SEQ ID NO: 27
  REGION 1..123: note = VHH (Binder 18)
  VARIANT 1: note = X is pyroglutamate

SEQ ID NO: 30 - DNA, 600 bp, synthetic construct
  misc_feature 1..600: note = TRAC 5' homology arm (nt)
  source 1..600: mol_type = other DNA
ggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccag

SEQ ID NO: 31 - DNA, 600 bp, synthetic construct
  misc_feature 1..600: note = TRAC 3' homology arm (nt)
  source 1..600: mol_type = other DNA
ctctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaa

SEQ ID NO: 32 - DNA, 799 bp, synthetic construct
  misc_feature 1..799: note = B2M 5' homology arm (nt)
  source 1..799: mol_type = other DNA
agcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaa

SEQ ID NO: 33 - DNA, 800 bp, synthetic construct
  misc_feature 1..800: note = B2M 3' homology arm (nt)
  source 1..800: mol_type = other DNA
actgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgata

SEQ ID NO: 34 - protein, 500 aa, synthetic construct
  REGION 1..500: note = HLA-E single chain fusion (amino acid sequence)
MSRSVALAVLALLSLSGLEAVMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 35 - DNA, 1502 bp, synthetic construct
  misc_feature 1..1502: note = HLA-E single chain fusion (DNA)
  source 1..1502: mol_type = other DNA
atgtcaagatctgtcgctctggctgtcctggccctgctgtctctgtctggacttgaagcagtgatggcccctagaacactggtgcttggaggcggaggatctggcggaggtggaagtggcggaggcggatctatccaacggacacctaagatccaagtgtacagcagacaccccgccgagaacggcaagagcaacttcctgaactgctacgtgtccggcttccacccctctgacattgaggtggacctgctgaagaacggcgagcggatcgagaaggtggaacacagcgatctgagcttctccaaggattggtccttctacctgctctactacaccgagttcactcctaccgagaaggacgagtacgcatgtagagtgaaccacgtgacactgagccaacctaagatcgtgaaatgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggacggttcctgagaggctatgagcagttcgcctacgacggcaaggactacctgacactgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacatgtgacccaccatcctatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggctcctactataaggccgagtggagcgattctgcccagggctctgaaagccactctctgtg

SEQ ID NO: 36 - protein, 337 aa, synthetic construct
  REGION 1..337: note = mature HLA-E coding sequence without signal peptide
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 37 - protein, 99 aa, synthetic construct [sequence omitted]

SEQ ID NO: 38 - protein, 343 aa, synthetic construct
  REGION 1..343: note = CD70 CAR (Binder 16) without signal peptide (amino acid sequence)
XVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 343 aa, synthetic construct
  REGION 1..343: note = CD70 CAR (Binder 16) without signal peptide (amino acid sequence)
QVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 343 aa, synthetic construct; identical to SEQ ID NO: 38
  REGION 1..343: note = CD70 CAR without signal peptide (amino acid sequence)
  VARIANT 1: note = X is pyroglutamate

SEQ ID NO: 41 - protein, 342 aa, synthetic construct
  REGION 1..342: note = CD70 CAR (Binder 18) without signal peptide (amino acid sequence)
VQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 343 aa, synthetic construct
  REGION 1..343: note = CD70 CAR (Binder 18) without signal peptide (amino acid sequence)
QVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 343 aa, synthetic construct
  REGION 1..343: note = CD70 CAR (Binder 18) without signal peptide (amino acid sequence)
  VARIANT 1: note = X is pyroglutamate
XVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 359 aa, synthetic construct
  REGION 1..359: note = CD70 CAR (Binder 18) with signal peptide
  VARIANT 17: note = X is Q or pyroglutamate
MPLLLLLPLLWAGALAXVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 359 aa, synthetic construct
  REGION 1..359: note = CD70 CAR (Binder 18) with signal peptide
MPLLLLLPLLWAGALAQVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 359 aa, synthetic construct; identical to SEQ ID NO: 44
  REGION 1..359: note = CD70 CAR (Binder 18) with signal peptide

SEQ ID NO: 47 - protein, 20 aa, synthetic construct
  REGION 1..20: note = B2m leader
MSRSVALAVLALLSLSGLEA

SEQ ID NO: 48 - protein, 9 aa, synthetic construct
  REGION 1..9: note = HLA-E fusion protein signal peptide
VMAPRTLVL

SEQ ID NO: 49 - protein, 9 aa, synthetic construct
  REGION 1..9: note = HLA-E fusion protein peptide
VMAPRTLLL

SEQ ID NO: 50 - protein, 8 aa, synthetic construct
  REGION 1..8: note = HLA-E fusion protein peptide
VMAPRTVL

SEQ ID NO: 51 - protein, 9 aa, synthetic construct
  REGION 1..9: note = HLA-E fusion protein peptide
VMAPRTLFL

SEQ ID NO: 52 - protein, 9 aa, synthetic construct
  REGION 1..9: note = HLA-E fusion protein peptide
VMAPRTLIL

SEQ ID NO: 53 - protein, 15 aa, synthetic construct
  REGION 1..15: note = (G4S)x3 linker
GGGGSGGGGSGGGGS

SEQ ID NO: 54 - protein, 20 aa, synthetic construct
  REGION 1..20: note = (G4S)x4 linker
GGGGSGGGGSGGGGSGGGGS

SEQ ID NO: 55 - DNA, 66 bp, synthetic construct
  misc_feature 1..66: note = TRAC gRNA
  misc_feature 1..25: note = DNA
  misc_feature 26..66: note = RNA
  source 1..66: mol_type = other DNA
atgtgtttttgtcaaaagacctttttaatttctactcttgtagatgagtctctcagctggtacacg

SEQ ID NO: 56 - DNA, 25 bp, synthetic construct
  misc_feature 1..25: note = DNA extension
  source 1..25: mol_type = other DNA
atgtgtttttgtcaaaagacctttt

SEQ ID NO: 57 - RNA, 20 nt, synthetic construct
  misc_feature 1..20: note = Scaffold (RNA)
  source 1..20: mol_type = other RNA
taatttctactcttgtagat

SEQ ID NO: 58 - RNA, 21 nt, synthetic construct
  misc_feature 1..21: note = TRAC guide sequence (RNA)
  source 1..21: mol_type = other RNA
gagtctctcagctggtacacg

SEQ ID NO: 59 - DNA, 21 bp, synthetic construct
  misc_feature 1..21: note = TRAC target site
  source 1..21: mol_type = other DNA
gagtctctcagctggtacacg

SEQ ID NO: 60 - DNA, 987 bp, synthetic construct
  misc_feature 1..987: note = Homo sapiens beta-2-microglobulin (B2M), nucleic acid NCBI Reference Sequence: NM_004048.2
  source 1..987: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 61 - DNA, 66 bp, synthetic construct
  misc_feature 1..66: note = B2M gRNA
  misc_feature 1..25: note = DNA
  misc_feature 26..66: note = RNA
  source 1..66: mol_type = other DNA
atgtgtttttgtcaaaagacctttttaatttctactcttgtagatagtgggggtgaattcagtgta

SEQ ID NO: 62 - RNA, 21 nt, synthetic construct
  misc_feature 1..21: note = B2M guide sequence (RNA)
  source 1..21: mol_type = other RNA
agtgggggtgaattcagtgta

SEQ ID NO: 63 - DNA, 21 bp, synthetic construct
  misc_feature 1..21: note = B2M target site
  source 1..21: mol_type = other DNA
agtgggggtgaattcagtgta

SEQ ID NO: 64 - DNA, 66 bp, synthetic construct
  source 1..66: mol_type = other DNA
  misc_feature 1..66: note = CD70 gRNA
  misc_feature 1..25: note = DNA
  misc_feature 26..66: note = RNA
atgtgtttttgtcaaaagacctttttaatttctactcttgtagatttccagtgggacgtagctgag

SEQ ID NO: 65 - RNA, 21 nt, synthetic construct
  misc_feature 1..21: note = CD70 guide sequence (RNA)
  source 1..21: mol_type = other RNA
ttccagtgggacgtagctgag

SEQ ID NO: 66 - DNA, 21 bp, synthetic construct
  misc_feature 1..21: note = CD70 target site
  source 1..21: mol_type = other DNA
ttccagtgggacgtagctgag

SEQ ID NO: 67 - DNA, 2656 bp, synthetic construct
  misc_feature 1..2656: note = CD70 CAR TRAC KI template (DNA)
  source 1..2656: mol_type = other DNA
ggatccggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccgctagcgccaccatgcccctgctgctcctgctccctttgctgtgggcgggagccctcgcgcaagtgcaactggtagaaagcggcggcggcctggtgcagcctggcggctctctaagactgagctgcgccgcaagcggcgtgaccctggagtacagcgccatcggctggttcagacaagcccccggcaaggagcaagagggcgtgagctgcacaagcagcagcgacggcagcatctactacgccgacagcgtgaagggcagattcaccatcagcagagacaacagcaagaacaccctgtacctgcagatgaacagcctgagagccgaggacaccgccgtgtactactgcgccatcggcgttttccatggcggttcttgcagaggcgcccttgatacctggggccaaggcaccctggtgaccgtgagcagcattgaagttatgtatcctcctccttacctagacaatgagaagagcaatggaaccattatccatgtgaaagggaaacacctttgtccaagtcccctatttcccggaccttctaagcccttctgggtgctcgtggtcgttggcggagtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagatgagtcgactgctttatttgtgaaatttgtgatgctattgctttatttgtaaccattataagctgcaataaacaagttaacaacaacaattgcattcattttatgtttcaggttcagggggaggtgtgggaggttttttaaagggccc

SEQ ID NO: 68 - DNA, 305 bp, synthetic construct
  misc_feature 1..305: note = Non-coding element (full sequence)
  source 1..305: mol_type = other DNA
ctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactaccggaactatcttgaagagtagtagtggactagtgtgacgctgctgacccctttctttcccttctacagatccaagctgtgaccggcgcctacacctgcagcccaagcttacc

SEQ ID NO: 69 - DNA, 193 bp, synthetic construct
  misc_feature 1..193: note = 5' non-coding element (193 bp)
  source 1..193: mol_type = other DNA
ctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactac

SEQ ID NO: 70 - DNA, 112 bp, synthetic construct
  misc_feature 1..112: note = 3' non-coding element (112 bp)
  source 1..112: mol_type = other DNA
cggaactatcttgaagagtagtagtggactagtgtgacgctgctgacccctttctttcccttctacagatccaagctgtgaccggcgcctacacctgcagcccaagcttacc

SEQ ID NO: 71 - DNA, 899 bp, synthetic construct
  source 1..899: mol_type = other DNA
  misc_feature 1..899: note = shRNA cassette
ccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 72 - RNA, 22 nt, synthetic construct
  source 1..22: mol_type = other RNA
  misc_feature 1..22: note = FAS shRNA guide
ttaagaatcttttcaaacacta

SEQ ID NO: 73 - RNA, 22 nt, synthetic construct
  source 1..22: mol_type = other RNA
  misc_feature 1..22: note = PTPN2 shRNA guide
tctgacaagagcttcacactga

SEQ ID NO: 74 - RNA, 22 nt, synthetic construct
  source 1..22: mol_type = other RNA
  misc_feature 1..22: note = TGFBR2 shRNA 1 guide
taaaaatcaatctcatttcctg

SEQ ID NO: 75 - RNA, 22 nt, synthetic construct
  source 1..22: mol_type = other RNA
  misc_feature 1..22: note = TGFBR2 shRNA 2 guide
ttatgtaaaagacaaacaatgc

SEQ ID NO: 76 - DNA, 133 bp, synthetic construct
  source 1..133: mol_type = other DNA
  misc_feature 1..133: note = Sv40 pA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 77 - DNA, 3856 bp, synthetic construct
  source 1..3856: mol_type = other DNA
  misc_feature 1..3856: note = AAV6 vector #1 full insert
ggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagggatccggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccgctagcgccaccatgcccctgctgctcctgctccctttgctgtgggcgggagccctcgcgcaagtgcaactggtagaaagcggcggcggcctggtgcagcctggcggctctctaagactgagctgcgccgcaagcggcgtgaccctggagtacagcgccatcggctggttcagacaagcccccggcaaggagcaagagggcgtgagctgcacaagcagcagcgacggcagcatctactacgccgacagcgtgaagggcagattcaccatcagcagagacaacagcaagaacaccctgtacctgcagatgaacagcctgagagccgaggacaccgccgtgtactactgcgccatcggcgttttccatggcggttcttgcagaggcgcccttgatacctggggccaaggcaccctggtgaccgtgagcagcattgaagttatgtatcctcctccttacctagacaatgagaagagcaatggaaccattatccatgtgaaagggaaacacctttgtccaagtcccctatttcccggaccttctaagcccttctgggtgctcgtggtcgttggcggagtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagatgagtcgactgctttatttgtgaaatttgtgatgctattgctttatttgtaaccattataagctgcaataaacaagttaacaacaacaattgcattcattttatgtttcaggttcagggggaggtgtgggaggttttttaaagggcccctctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaa

SEQ ID NO: 78 - DNA, 6306 bp, synthetic construct
  source 1..6306: mol_type = other DNA
  misc_feature 1..6306: note = AAV6 vector #2 full insert
tgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatatcacgcgtagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaaggatccggcagcggcgagggcagaggcagcctgctgacctgcggcgacgtggaggagaaccccggccccatgtcaagatctgtcgctctggctgtcctggccctgctgtctctgtctggacttgaagcagtgatggcccctagaacactggtgcttggaggcggaggatctggcggaggtggaagtggcggaggcggatctatccaacggacacctaagatccaagtgtacagcagacaccccgccgagaacggcaagagcaacttcctgaactgctacgtgtccggcttccacccctctgacattgaggtggacctgctgaagaacggcgagcggatcgagaaggtggaacacagcgatctgagcttctccaaggattggtccttctacctgctctactacaccgagttcactcctaccgagaaggacgagtacgcatgtagagtgaaccacgtgacactgagccaacctaagatcgtgaaatgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggacggttcctgagaggctatgagcagttcgcctacgacggcaaggactacctgacactgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacatgtgacccaccatcctatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggctcctactataaggccgagtggagcgattctgcccagggctctgaaagccactctctgtgaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaagggcccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgatatcgcgactgcaggcgatctctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcatatctctcgatccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcttaaccctagaaagatagtctgcgtaaaattgacgca

SEQ ID NO: 79 - protein, 5 aa, synthetic construct
  REGION 1..5: note = CD70 CDR1 CAR (Binder 18) Kabat
YHNIG

SEQ ID NO: 80 - protein, 17 aa, synthetic construct
  REGION 1..17: note = CD70 CDR2 CAR (Binder 18) Kabat
CISSSDGSTYYADSVKG

SEQ ID NO: 81 - protein, 14 aa, synthetic construct
  REGION 1..14: note = CD70 CDR3 CAR (Binder 18) Kabat
GLFYPGSCRGALDA

SEQ ID NO: 82 - protein, 8 aa, synthetic construct
  REGION 1..8: note = CD70 CDR1 CAR (Binder 18) IMGT
GFTLDYHN

SEQ ID NO: 83 - protein, 8 aa, synthetic construct
  REGION 1..8: note = CD70 CDR2 CAR (Binder 18) IMGT
ISSSDGST

SEQ ID NO: 84 - protein, 16 aa, synthetic construct
  REGION 1..16: note = CD70 CDR3 CAR (Binder 18) IMGT
ATGLFYPGSCRGALDA

SEQ ID NO: 85 - RNA, 4530 nt, synthetic construct
  source 1..4530: mol_type = other RNA
  misc_feature 1..4530: note = TGFBR2 mRNA
actcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 86 - RNA, 3470 nt, synthetic construct
  source 1..3470: mol_type = other RNA
  misc_feature 1..3470: note = PTPN2 mRNA
gcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctcaaaaaaaaaaaaaaaaaaagtttgaaaaccattggtatagatagatattttgaattgatttgcatagtctccttgaatgtgttaaattatgttgaaagtatgaaagcaggatgtaggtggtactacatattaaataagatttatataaca

SEQ ID NO: 87 - RNA, 3696 nt, synthetic construct
  source 1..3696: mol_type = other RNA
  misc_feature 1..3696: note = FAS mRNA
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 88 - DNA, 684 bp, synthetic construct
  source 1..684: mol_type = other DNA
  misc_feature 1..684: note = IgG-Long hinge
gagtctaaatacggaccgccttgtcctccttgtcccgctcctcctgttgccggaccttccgtgttcctgtttcctccaaaacctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtattctcggctcaccgtggataaatcaagatggcaagagggcaacgtgttcagctgcagcgtgatgcacgaggccctgcacaaccactacacccagaaaagcctgagcctgtctctgggcaag

SEQ ID NO: 89 - DNA, 357 bp, synthetic construct
  source 1..357: mol_type = other DNA
  misc_feature 1..357: note = IgG-Medium hinge
gagtctaaatacggaccgccttgtccttcttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtaaactcaccgtggataaatcaagatggcaagagggcaacgtgttcagctgcagcgtgatgcacgaggccctgcacaaccactacacccagaaaagcctgagcctgtctctgggcaag

SEQ ID NO: 90 - DNA, 39 bp, synthetic construct
  source 1..39: mol_type = other DNA
  misc_feature 1..39: note = IgG-Short hinge
gagtctaaatacggaccgccttgtcctccttgtcccatg

SEQ ID NO: 91 - DNA, 141 bp, synthetic construct
  source 1..141: mol_type = other DNA
  misc_feature 1..141: note = CD8a hinge
accagcaccaccacaccagctcctagacctccaactcctgctcctacaatcgccagccagcctctgtctctgaggcccgaagcttgtagaccagctgctggcggagccgtgcatacaagaggactggatttcgcctgcgac

SEQ ID NO: 92 - DNA, 72 bp, synthetic construct
  source 1..72: mol_type = other DNA
  misc_feature 1..72: note = CD8a transmembrane domain
atctacatctgggccccactggctggaacatgtggcgttctgctgctgagcctggtcatcaccctgtactgc

SEQ ID NO: 93 - protein, 228 aa, synthetic construct
  REGION 1..228: note = IgG-Long hinge
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 119 aa, synthetic construct
  REGION 1..119: note = IgG-Medium hinge
ESKYGPPCPSCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 95 - protein, 13 aa, synthetic construct
  REGION 1..13: note = IgG-Short hinge
ESKYGPPCPPCPM

SEQ ID NO: 96 - protein, 47 aa, synthetic construct
  REGION 1..47: note = CD8a hinge
TSTTTPAPRPPTPAPTIASQPLSLRPEACRPAAGGAVHTRGLDFACD

SEQ ID NO: 97 - protein, 24 aa, synthetic construct
  REGION 1..24: note = CD8a transmembrane domain
IYIWAPLAGTCGVLLLSLVITLYC

SEQ ID NO: 98 - protein, 359 aa, synthetic construct
  REGION 1..359: note = anti-CD70 CAR-Fc (Binder 16)
QVQLVESGGGLVQPGGSLRLSCAASGVTLEYSAIGWFRQAPGKEQEGVSCTSSSDGSIYYADSVKGRFTISRDNSKNTLYLQMNSLRAEDTAVYYCAIGVFHGGSCRGALDTWGQGTLVTVSSGSAAAEPKSSDKTHTSPPSPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLRCHVKGFYPSDIAVEWESNGQPENNYKTTKPVLDSDGSFRLASTLTVDKSRWQQGNVFSCSVLHEALHNHYTQKSLSLSPG

SEQ ID NO: 99 - protein, 408 aa, synthetic construct
  REGION 1..408: note = Monomeric soluble CD27
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLSSSDFIRGSAAAEPKSSDKTHTSPPSPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLRCHVKGFYPSDIAVEWESNGQPENNYKTTKPVLDSDGSFRLASTLTVDKSRWQQGNVFSCSVLHEALHNHYTQKSLSLSPG

SEQ ID NO: 100 - protein, 210 aa, synthetic construct
  REGION 1..210: note = IgGL hinge region
GPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 101 - skipped

SEQ ID NO: 102 - protein, 45 aa, synthetic construct
  REGION 1..45: note = CD8alpha hinge domain
TTTPAPRPPTPAPTIASQPLSLRPEACRPAAGGAVHTRGLDFACD

SEQ ID NO: 103 - protein, 39 aa, synthetic construct; identical to SEQ ID NO: 10
  REGION 1..39: note = CD28 hinge domain

SEQ ID NO: 104 - protein, 12 aa, synthetic construct
  REGION 1..12: note = Short Spacer (IgG4 hinge) (aa)
ESKYGPPCPPCP

SEQ ID NO: 105 - protein, 119 aa, Homo sapiens
  REGION 1..119: note = Hinge-CH3 spacer Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 282 aa, Homo sapiens
  REGION 1..282: note = IgD-hinge-Fc Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 107 - protein, 15 aa, synthetic construct
  REGION 1..15: note = Hinge
EPKSCDKTHTCPPCP

SEQ ID NO: 108 - protein, 12 aa, synthetic construct
  REGION 1..12: note = Hinge
ERKCCVECPPCP

SEQ ID NO: 109 - protein, 61 aa, synthetic construct
  REGION 1..61: note = Hinge
ELKTPLGDTHTCPRCPEPKSCDTPPPCPRCPEPKSCDTPPPCPRCPEPKSCDTPPPCPRCP

SEQ ID NO: 110 - protein, 12 aa, synthetic construct
  REGION 1..12: note = Hinge
ESKYGPPCPSCP

SEQ ID NO: 111 - protein, 9 aa, synthetic construct
  REGION 1..9: note = Hinge
YGPPCPPCP

SEQ ID NO: 112 - protein, 10 aa, synthetic construct
  REGION 1..10: note = Hinge
KYGPPCPPCP

SEQ ID NO: 113 - protein, 14 aa, synthetic construct
  REGION 1..14: note = Hinge
EVVVKYGPPCPPCP

SEQ ID NO: 114 - protein, 326 aa, Homo sapiens
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 115 - protein, 327 aa, Homo sapiens
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 66 aa, Homo sapiens
  REGION 1..66: note = CD28 (amino acids 114-179 of Accession No. P10747) Homo sapiens
IEVMYPPPYLDNEKSNGTIIHVKGKHLCPSPLFPGPSKPFWVLVVVGGVLACYSLLVTVAFIIFWV

SEQ ID NO: 117 - protein, 41 aa, Homo sapiens
  REGION 1..41: note = CD28 (amino acids 180-220 of P10747) Homo sapiens
RSKRSRLLHSDYMNMTPRRPGPTRKHYQPYAPPRDFAAYRS

SEQ ID NO: 118 - protein, 41 aa, Homo sapiens
  REGION 1..41: note = CD28 (LL to GG) Homo sapiens
RSKRSRGGHSDYMNMTPRRPGPTRKHYQPYAPPRDFAAYRS

SEQ ID NO: 119 - protein, 20 aa, synthetic construct
  REGION 1..20: note = Linker, repeated 3-4 times
  VARIANT 16..20: note = may or may not be present
GGGGSGGGGSGGGGSGGGGS

SEQ ID NO: 120 - protein, 15 aa, synthetic construct
  REGION 1..15: note = Linker, repeated 2-3 times
  VARIANT 11..15: note = may or may not be present
GGGGSGGGGSGGGGS

SEQ ID NO: 121 - protein, 15 aa, synthetic construct
  REGION 1..15: note = Linker
GGGASGGGGSGGGGS

SEQ ID NO: 122 - protein, 15 aa, synthetic construct
  REGION 1..15: note = Linker
GCGASGGGGSGGGGS

SEQ ID NO: 123 - DNA, 347 bp, synthetic construct
  source 1..347: mol_type = other DNA
  misc_feature 1..347: note = MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 124 - DNA, 183 bp, synthetic construct
  source 1..183: mol_type = other DNA
  misc_feature 1..183: note = 5 prime non-coding element
tattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgttt

SEQ ID NO: 125 - DNA, 105 bp, synthetic construct
  source 1..105: mol_type = other DNA
  misc_feature 1..105: note = 3 prime non-coding element
aactgataggcacctattggtcttactgacatccactttgcctttctctccacaggtgtccactcccagttcaattacagctcttaaggctagagtactcaattc

SEQ ID NO: 126 - DNA, 2013 bp, synthetic construct
  source 1..2013: mol_type = other DNA
  misc_feature 1..2013: note = Masking CAR
atgcccctgctgctcctgctccctttgctgtgggcgggagccctcgcgcaaattcagctggtgcagtccggccccgaagttaaaaagcccggcgaaaccgtgaagattagttgcaaggctagcgggtatacctttacaaactacggcatgaactgggtcaagcaggctcctggaaaagggctgaagtggatgggttggatcaacacatacactggggaaccaacgtacgccgatgcctttaaggggcgattcgccttctctctcgaaacctcagcaagtacagcatacttgcagattaacaaccttaagaacgaagatactgctacttatttctgcgccagggactacggggattacggaatggattattggggacagggaactagtgtgactgtttcatccgggggcggcgggtccggtggtgggggatccggaggcgggggatcagacatcgtgctgacccagagccccgctagcctggccgtgagccttggacagcgcgccaccatcagttgtcgagcgtctaagtctgtatccactagcggatactcatttatgcactggtaccagcagaagcctggccagccgcccaagttgcttatctatttggcatccaacctggaatccggggttcctgcgcgcttctccggtagtggatccggcaccgattttaccctcaatatccacccggttgaggaggaggacgccgctacatactattgtcagcattcacgcgaggtgccatggacctttggaggcgggaccaaactcgagattaagagggagtctaaatacggaccgccttgtcctccttgtcccgctcctcctgttgccggaccttccgtgttcctgtttcctccaaaacctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtattctcggctcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttactccctgctggttaccgtggccttcatcatcttttgggtcaagcggggcagaaagaagctgctctacatcttcaagcagcccttcatgcggcccgtgcagaccacacaagaggaagatggctgctcctgccgattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 127 - DNA, 20 bp, synthetic construct
  source 1..20: mol_type = other DNA
  misc_feature 1..20: note = Cas9 TRAC gRNA target site
gagaatcaaaatcggtgaat

SEQ ID NO: 128 - RNA, 20 nt, synthetic construct
  source 1..20: mol_type = other RNA
  misc_feature 1..20: note = Cas9 TRAC gRNA spacer
gagaatcaaaatcggtgaat

SEQ ID NO: 129 - protein, 359 aa, synthetic construct
  REGION 1..359: note = anti-CD70 CAR-Fc (Binder 18)
QVQLVESGGGLVQPGGSLRLSCAASGFTLDYHNIGWFRQAPGKELEGVLCISSSDGSTYYADSVKGRFTISRDNSKNTLYLQMNSLRAEDTAVYYCATGLFYPGSCRGALDAWGQGTLVTVSSGSAAAEPKSSDKTHTSPPSPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLRCHVKGFYPSDIAVEWESNGQPENNYKTTKPVLDSDGSFRLASTLTVDKSRWQQGNVFSCSVLHEALHNHYTQKSLSLSPG

(19) United States
(12) Patent Application Publication
(10) Pub. No.: US 2026/0174853 A1
(43) Pub. Date: Jun. 25, 2026

(54) CD70-TARGETED CARS AND ENGINEERED CELLS COMPRISING SAME AND RELATED METHODS

(71) Applicant: Juno Therapeutics, Inc., Seattle, WA (US)

(72) Inventors: Geoffrey Patrick O’DONOGHUE, Seattle, WA (US); John-Michael Eric WILLIFORD, Seattle, WA (US); Nikhil HEBBAR, Renton, WA (US); Andres Adolfo ALVAREZ, Seattle, WA (US); Eric William JEFFERY, Seattle, WA (US); Douglas Scott JONES, Bellevue, WA (US); Ruth Amanda SALMON, Seattle, WA (US); Cédric CLEYRAT, Seattle, WA (US); Pallavur SIVAKUMAR, Seattle, WA (US); Erin Elizabeth NEVIUS, Seattle, WA (US); Kaya Brady PETERSON, Henrico, VA (US); Dean Y. TOY, Newcastle, WA (US); Brian BELMONT, Seattle, WA (US); Madeline WILLIAMS, Seattle, WA (US); Rebekah TURK, Seattle, WA (US); Felecia Dawn WAGENER, Maple Valley, WA (US)

(73) Assignee: Juno Therapeutics, Inc., Seattle, WA (US)

(21) Appl. No.: 19/427,047

(22) Filed: Dec. 19, 2025

Related U.S. Application Data
(60) Provisional application No. 63/737,537, filed on Dec. 20, 2024.
(60) Provisional application No. 63/794,739, filed on Apr. 25, 2025.
(60) Provisional application No. 63/802,526, filed on May 8, 2025.

(51) Int. Cl. A61K 40/11; A61K 9/00; A61K 31/47; A61K 31/675; A61K 31/7076; A61K 39/00; A61K 39/395; A61K 40/31; A61K 40/42; A61K 40/50; A61P 35/00; C07K 14/725; C07K 16/28

(52) U.S. Cl. CPC A61K 40/11; A61K 9/0019; A61K 31/47; A61K 31/675; A61K 31/7076; A61K 39/3955; A61K 40/31; A61K 40/4232; A61K 40/50; A61P 35/00; C07K 14/7051; C07K 16/2875; A61K 2039/505; A61K 2039/54; A61K 2039/545; C07K 2317/569

(57) ABSTRACT

Provided herein are CD70-targeted chimeric antigen receptors (CARs), genetically engineered cells such as T cells containing the same, and related methods and uses of the genetically engineered cells in allogeneic cell therapy. Also provided are T cells that are genetically engineered with a CAR, such as a CD70-targeted CAR, and are further genetically engineered by one or more strategies to reduce host immune recognition of the engineered T cells, such as by heterologous expression of one or more additional transgenes and by genetic disruption to reduce or eliminate expression or one or more endogenous protein. Also provided are methods of making and using the engineered T cells for cell therapy, including in connection with cancer immunotherapy comprising adoptive transfer of the engineered T cells.

### CROSS-REFERENCE TO RELATED APPLICATIONS

This application claims priority to U.S. Provisional Application No. 63/737,537, filed Dec. 20, 2024, entitled “CD70-TARGETED CARS AND ENGINEERED CELLS COMPRISING SAME AND RELATED METHODS,” U.S. Provisional Application No. 63/794,739, filed Apr. 25, 2025, entitled “CD70-TARGETED CARS AND ENGINEERED CELLS COMPRISING SAME AND RELATED METHODS,” and U.S. Provisional Application No. 63/802,526, filed May 8, 2025, entitled “CD70-TARGETED CARS AND ENGINEERED CELLS COMPRISING SAME AND RELATED METHODS,” the contents of which are incorporated by reference in their entireties.

### REFERENCE TO AN ELECTRONIC SEQUENCE LISTING

The present application is being filed with a Sequence Listing in electronic format. The Sequence Listing is provided as a file entitled 735042029700SeqList.xml, created on Dec. 18, 2025, which is 213,907 bytes in size. The information in electronic format of the Sequence Listing is incorporated by reference in its entirety.

### FIELD

The present disclosure relates in some aspects to CD70-targeted chimeric antigen receptors (CARs), genetically engineered cells such as T cells containing the same, and related methods and uses of the genetically engineered cells in allogeneic cell therapy. Also provided are T cells that are genetically engineered with a CAR, such as a CD70-targeted CAR, and are further genetically engineered by one or more strategies to reduce host immune recognition of the engineered T cells, such as by heterologous expression of one or more additional transgenes and by genetic disruption to reduce or eliminate expression or one or more endogenous protein. The present disclosure also provides methods of making and using the engineered T cells for cell therapy, including in connection with cancer immunotherapy comprising adoptive transfer of the engineered T cells.

### BACKGROUND

Various cell therapy methods are available for treating diseases and conditions. Among cell therapy methods are methods involving immune cells, such as T cells, genetically engineered with a recombinant receptor, such as a chimeric antigen receptor (CAR). In some contexts, CD70 is expressed by certain cancers and is an attractive therapeutic target for cell therapy. In some cases, current methods for generating CAR T cells are not ideal because they require patient-specific manufacturing for autologous delivery. Further, even for allogeneic cell therapies, there is in many cases a problem with the persistence of the cell therapy in the subject so that there can be a high rate of relapse. Improved CAR T cell therapies are needed, including in connection with allogeneic administration.

### SUMMARY

Provided herein are chimeric antigen receptors (CAR) directed against CD70, wherein the CAR comprises a CD70-binding domain that binds to CD70 comprising a heavy chain only variable (VHH) region, wherein: (i) the VHH region comprises a CDR-1, CDR-2, and CDR-3 each comprising a sequence that is contained within SEQ ID NO:1, wherein X is Q or pyroglutamate; or (ii) the VHH region comprises a CDR-1, CDR-2, and CDR-3 each comprising a sequence that is contained within SEQ ID NO:27, wherein X is Q or pyroglutamate. In some embodiments, the CAR further comprises a spacer domain, a transmembrane domain, and an intracellular signaling domain. In some embodiments, the VHH region of the CD70-binding domain comprises CDR-1, CDR-2, and CDR-3 sequences set forth in SEQ ID NOs: 4, 5, and 6, respectively. In some embodiments, the VHH region of the CD70-binding domain comprises CDR-1, CDR-2, and CDR-3 sequences set forth in SEQ ID NOs: 7, 8, and 9, respectively. In some embodiments, the VHH region of the CD70-binding domain comprises CDR-1, CDR-2, and CDR-3 sequences set forth in SEQ ID NOs: 79, 80, and 81, respectively. In some embodiments, the VHH region of the CD70-binding domain comprises CDR-1, CDR-2, and CDR-3 sequences set forth in SEQ ID NOs: 82, 83, and 84, respectively. In some embodiments, (i) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or (ii) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate. In some embodiments, the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 2. In some embodiments, the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 3, wherein X is pyroglutamate. In some embodiments, the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO:28. In some embodiments, the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 29, wherein X is pyroglutamate. In some embodiments, (i) the VHH region comprises a sequence set forth in SEQ ID NO: 1, wherein X is Q or pyroglutamate; or (ii) the VHH region comprises a sequence set forth in SEQ ID NO: 27, wherein X is Q or pyroglutamate. In some embodiments, the VHH region comprises a sequence set forth in SEQ ID NO: 2. In some embodiments, the VHH region comprises a sequence set forth in SEQ ID NO: 3, wherein X is pyroglutamate. In some embodiments, the VHH region comprises a sequence set forth in SEQ ID NO: 28. In some embodiments, the VHH region comprises a sequence set forth in SEQ ID NO: 29, wherein X is pyroglutamate. In some embodiments, the spacer domain comprises a hinge domain from human CD28.

In some embodiments, the spacer domain comprises an amino acid sequence having at least 85%, 86%, 87%, 88%, 89%, 90%, 91%, 92%, 93%, 94%, 95%, 96%, 97%, 98% or 99% identity to SEQ ID NO: 10. In some embodiments, the spacer domain comprises the sequence set forth in SEQ ID NO: 10. In some embodiments, the transmembrane domain comprises a transmembrane domain from human CD28. In some embodiments, the transmembrane domain comprises an amino acid sequence having at least at or about 85%, 86%, 87%, 88%, 89%, 90%, 91%, 92%, 93%, 94%, 95%, 96%, 97%, 98% or 99% identity to SEQ ID NO:11. In some embodiments, the transmembrane domain comprises the sequence set forth in SEQ ID NO: 11.

In some embodiments, the intracellular signaling domain comprises a cytoplasmic signaling domain of a human CD3-zeta (CD3ζ) chain. In some embodiments, the intracellular signaling domain comprises an amino acid sequence having at least at or about 90%, 91%, 92%, 93%, 94%, 95%, 96%, 97%, 98% or 99% sequence identity to SEQ ID NO: 13. In some embodiments, the intracellular signaling domain comprises the amino acid sequence set forth in SEQ ID NO: 13. In some embodiments, the intracellular signaling domain further comprises a costimulatory signaling region. In some embodiments, the costimulatory signaling region comprises an intracellular signaling domain of a T cell costimulatory molecule or a signaling portion thereof.

In some embodiments, the costimulatory signaling region comprises an intracellular signaling domain of human 4-1BB. In some embodiments, the costimulatory signaling region comprises an amino acid sequence having at least at or about 90%, 91%, 92%, 93%, 94%, 95%, 96%, 97%, 98% or 99% sequence identity to SEQ ID NO: 12. In some embodiments, the costimulatory signaling region comprises the amino acid sequence set forth in SEQ ID NO: 12. In some embodiments, the CAR comprises the amino acid sequence set forth in SEQ ID NO: 38 or SEQ ID NO: 41, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38 or SEQ ID NO: 41, wherein X is Q or pyroglutamate. In some embodiments, the CAR comprises the amino acid sequence set forth in SEQ ID NO: 38, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38, wherein X is Q or pyroglutamate. In some embodiments, the CAR comprises the amino acid sequence set forth in SEQ ID NO: 38, wherein X is Q or pyroglutamate. In some embodiments, the CAR comprises the amino acid sequence set forth in SEQ ID NO: 39, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 39. In some embodiments, the CAR comprises the amino acid sequence set forth in SEQ ID NO: 39. In some embodiments, the CAR comprises the amino acid sequence set forth in SEQ ID NO: 40, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 40, wherein X is pyroglutamate. In some embodiments, the CAR comprises the amino acid sequence set forth in SEQ ID NO: 40, wherein X is pyroglutamate. In some embodiments, the CAR comprises the amino acid sequence set forth in SEQ ID NO: 41, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 41, wherein X is Q or pyroglutamate. In some embodiments, the CAR comprises the amino acid sequence set forth in SEQ ID NO: 41, wherein X is Q or pyroglutamate. In some embodiments, the CAR comprises the amino acid sequence set forth in SEQ ID NO: 42, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 42. In some embodiments, the CAR comprises the amino acid sequence set forth in SEQ ID NO: 42. In some embodiments, the CAR comprises the amino acid sequence set forth in SEQ ID NO: 43, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 43, wherein X is pyroglutamate. In some embodiments, the CAR comprises the amino acid sequence set forth in SEQ ID NO: 43, wherein X is pyroglutamate. In some embodiments, the CAR comprises the amino acid sequence set forth in SEQ ID NO: 14 or SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14 or SEQ ID NO: 44, wherein X is Q or pyroglutamate. In some embodiments, the CAR comprises the amino acid sequence set forth in SEQ ID NO: 14, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14, wherein X is Q or pyroglutamate. In some embodiments, the CAR comprises the amino acid sequence set forth in SEQ ID NO: 14, wherein X is Q or pyroglutamate. In some embodiments, the CAR comprises the amino acid sequence set forth in SEQ ID NO: 15, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 15. In some embodiments, the CAR comprises the amino acid sequence set forth in SEQ ID NO: 15. In some embodiments, the CAR comprises the amino acid sequence set forth in SEQ ID NO: 16, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 16, wherein X is pyroglutamate. In some embodiments, the CAR comprises the amino acid sequence set forth in SEQ ID NO: 16, wherein X is pyroglutamate. In some embodiments, the CAR comprises the amino acid sequence set forth in SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 44, wherein X is Q or pyroglutamate. In some embodiments, the CAR comprises the amino acid sequence set forth in SEQ ID NO: 44, wherein X is Q or pyroglutamate. In some embodiments, the CAR comprises the amino acid sequence set forth in SEQ ID NO: 45, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 45. In some embodiments, the CAR comprises the amino acid sequence set forth in SEQ ID NO: 45. In some embodiments, the CAR comprises the amino acid sequence set forth in SEQ ID NO: 46, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 46, wherein X is pyroglutamate. In some embodiments, the CAR comprises the amino acid sequence set forth in SEQ ID NO: 46, wherein X is pyroglutamate.

Also provided here are genetically engineered human T cells comprising a CAR.

Also provided herein are genetically engineered human T cells comprising (a) a first nucleotide sequence encoding the CAR. In some embodiments, the first nucleotide sequence encoding the CAR comprises the nucleic acid sequence set forth in SEQ ID NO: 17. In some embodiments, the genetically engineered human T cell further comprises: (b) a first genetic disruption in an endogenous T Cell Receptor Alpha Constant (TRAC) gene; and (c) a second genetic disruption in an endogenous β2 microglobulin (B2M) gene.

Also provided herein are genetically engineered human T cells comprising: (a) a first nucleotide sequence encoding a chimeric antigen receptor (CAR); (b) a first genetic disruption in an endogenous TRAC gene; (c) a second genetic disruption in an endogenous B2 microglobulin (B2M) gene; (d) a second nucleotide sequence comprising a transgene encoding a single chain HLA-E fusion protein; and (e) a third nucleotide sequence comprising: (i) a first nucleic acid at least 15 nucleotides in length complementary to an mRNA encoding TGF-B Receptor 2 (TGFBR2); (ii) a second nucleic acid at least 15 nucleotides in length complementary to an mRNA encoding Protein Tyrosine Phosphatase Non-Receptor Type 2 (PTPN2); and (iii) a third nucleic acid at least 15 nucleotides in length complementary an mRNA encoding Fas Cell Surface Death Receptor (FAS). In some embodiments, the first nucleotide sequence encoding a chimeric antigen receptor (CAR) encodes the CAR of any of the embodiments described herein. In some embodiments, the first genetic disruption and second genetic disruption are by a gene editing technique comprising a CRISPR-Cas system. In some embodiments, the CRISPR-Cas system is a ribonucleoprotein (RNP) complex comprising a Cas protein and a guide RNA (gRNA), optionally wherein the Cas protein is a Cas12a protein. In some embodiments, the first genetic disruption in the endogenous TRAC gene is in a target site sequence in exon 1 of the endogenous TRAC gene. In some embodiments, target site sequence in exon 1 of the endogenous TRAC gene is located at hg38 genomic coordinates chr14:22,547,528-22,547,548. In some embodiments, the target site sequence in exon 1 of the endogenous TRAC gene has the sequence set forth in SEQ ID NO: 59, a contiguous portion thereof of at least 14 nucleotides (nt), or a complementary sequence of the foregoing. In some embodiments, the target site sequence in exon 1 of the endogenous TRAC gene has the sequence set forth in SEQ ID NO: 59. In some embodiments, the first genetic disruption is by a CRISPR-Cas system that comprises a Cas12a protein and a guide RNA (gRNA) comprising a spacer sequence comprising the nucleic acid sequence of SEQ ID NO:58, or a contiguous portion thereof of at least 14 nt. In some embodiments, the CRISPR-Cas system is a ribonucleoprotein (RNP) complex comprising the Cas12a protein and the gRNA. In some embodiments, the first genetic disruption disrupts one or more alleles of the endogenous TRAC gene.

In some embodiments, the first genetic disruption disrupts all alleles of the endogenous TRAC gene. In some embodiments, the first genetic disruption reduces protein expression of a TCR alpha chain encoded from the endogenous TRAC gene. In some embodiments, the first genetic disruption reduces protein expression of the TCR alpha chain on the surface of the genetically engineered human T cell. In some embodiments, there is no detectable expression of the TCR alpha chain in the genetically engineered human T cell. In some embodiments, the genetically engineered human T cell has reduced expression of CD3 on the cell surface of the genetically engineered human T cell. In some embodiments, the genetically engineered cell does not express detectable CD3 on the cell surface of the genetically engineered human T cell.

In some embodiments, the second genetic disruption in the endogenous B2M gene is in a target site sequence in exon 2 of the endogenous B2M gene. In some embodiments, the target site sequence in exon 2 of the endogenous B2M gene is located at hg38 genomic coordinates chr15:44,715,614-44,715,634. In some embodiments, the target site sequence in exon 2 of the endogenous B2M gene has the sequence set forth in SEQ ID NO: 63, a contiguous portion thereof of at least 14 nucleotides (nt), or a complementary sequence of the foregoing. In some embodiments, the target site sequence has the sequence set forth in SEQ ID NO: 63. In some embodiments, the second genetic disruption is by a CRISPR-Cas system that comprises a Cas12a protein and a guide RNA (gRNA) comprising a spacer sequence comprising the nucleic acid sequence SEQ ID NO:62, or a contiguous portion thereof of at least 14 nt. In some embodiments, the CRISPR-Cas system is a ribonucleoprotein (RNP) complex comprising the Cas12a protein and the gRNA. In some embodiments, the second genetic disruption disrupts one or more alleles of the endogenous B2M gene. In some embodiments, the second genetic disruption disrupts all alleles of the endogenous B2M gene. In some embodiments, the second genetic disruption reduces protein expression of B2M encoded from the endogenous B2M gene. In some embodiments, there is no detectable expression of endogenous B2M in the genetically engineered human T cell.

In some embodiments, the genetically engineered human T cell has reduced expression of one or more HLA class I molecules. In some embodiments, the genetically engineered human T cell has no detectable expression of one or more HLA class I molecules on the cell surface of the genetically engineered human T cell. In some embodiments, one or more HLA class I molecules is selected from HLA-A class I, HLA-B class I, and HLA-C class I.

In some embodiments, the genetically engineered human T cell further comprises: (d) a second nucleotide sequence comprising a transgene encoding a single chain HLA-E fusion protein. In some embodiments, the single chain HLA-E fusion protein comprises: (1) at least a portion of the B2M protein, (2) at least a portion of an HLA-E class I chain, and (3) a peptide that is a portion of a signal sequence from an MHC class I molecule that is presented by the single chain HLA-E fusion protein when expressed on the cell surface of the genetically engineered human T cell. In some embodiments, the portion of the B2M protein comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:47; (2) the portion of an HLA-E class I chain comprises the sequence set forth in, or a sequence that is at least 85%, 86%, 87%, 88%, 89%, 90%, 91%, 92%, 93%, 94%, 95%, 96%, 97%, 98% or 99% identical to SEQ ID NO: 36; and (3) the peptide is VMAPRTLVL (SEQ ID NO:48), VMAPRTLLL (SEQ ID NO:49), VMAPRTVLL (SEQ ID NO:50), VMAPRTLFL (SEQ ID NO:51), or VMAPRTLIL (SEQ ID NO:52). In some embodiments, the peptide is VMAPRTLVL (SEQ ID NO:48). In some embodiments, (1) the portion of the B2M protein comprises the sequence set forth in SEQ ID NO:47; (2) the portion of an HLA-E class I chain comprises the sequence set forth in SEQ ID NO: 36; and (3) the peptide is VMAPRTLVL (SEQ ID NO:48). In some embodiments, the single chain HLA-E fusion protein further comprises: (4) a peptide linker that links (1) and (2). In some embodiments, the peptide linker comprises a GS linker. In some embodiments, the GS linker is a (G4S)x3 linker set forth in SEQ ID NO:53. In some embodiments, the GS linker is a (G4S)x4 linker set forth in SEQ ID NO: 54. In some embodiments, the single chain HLA-E fusion protein comprises a sequence of amino acids that has at least about 85%, 86%, 87%, 88%, 89%, 90%, 91%, 92%, 93%, 94%, 95%, 96%, 97%, 98%, or 99% sequence identity to SEQ ID NO:34. In some embodiments, the single chain HLA-E fusion protein comprises the sequence of amino acids set forth in SEQ ID NO:34. In some embodiments, the single chain HLA-E fusion protein is capable of engaging an inhibitory receptor on the surface of an NK cell. In some embodiments, the inhibitory receptor on the surface of the NK cell is an NKG2A or NKG2B. In some embodiments, the second nucleotide sequence encoding the single chain HLA-E fusion protein is present in the disrupted endogenous B2M gene in the T cell under the operable control of a promoter. In some embodiments, the promoter is an endogenous promoter of the endogenous B2M gene. In some embodiments, the second nucleotide sequence encoding the single chain HLA-E fusion protein has been integrated in the disrupted endogenous B2M gene by homology directed repair (HDR).

In some embodiments, the genetically engineered human T cell further comprises (e) a third nucleotide sequence comprising: (i) a first nucleic acid sequence at least 15 nucleotides in length complementary to an mRNA encoding TGF-B Receptor 2 (TGFBR2); (ii) a second nucleic acid sequence at least 15 nucleotides in length complementary to an mRNA encoding Protein Tyrosine Phosphatase Non-Receptor Type 2 (PTPN2); and (iii) a third nucleic acid sequence at least 15 nucleotides in length complementary an mRNA encoding Fas Cell Surface Death Receptor (FAS). In some embodiments, the third nucleotide sequence further comprises: (iv) a fourth nucleic acid at least 15 nucleotides in length complementary to an mRNA encoding human TGF-B Receptor 2 (TGFBR2).

In some embodiments, the first and fourth nucleic acids are complementary to different nucleotides of the mRNA encoding human TGF-B Receptor 2 (TGFBR2). In some embodiments, each nucleic acid of the third nucleotide sequence of (e) is a short hairpin RNA (shRNA), a small interfering RNA (siRNA), double stranded RNA (dsRNA), or an antisense oligonucleotide. In some embodiments, each nucleic acid of the third nucleotide sequence of (e) is a short hairpin RNA (shRNA). In some embodiments, (i) the mRNA encoding TGFBR2 encodes human TGFBR2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85, (ii) the mRNA encoding PTPN2 encodes human PTPN2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:86, and (iii) the mRNA encoding FAS encodes human FAS and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:87. In some embodiments, the mRNA encoding TGFBR2 comprises a sequence set forth in SEQ ID NO:85. In some embodiments, the mRNA encoding PTPN2 comprises the sequence set forth in SEQ ID NO: 86. In some embodiments, the mRNA encoding FAS comprises the sequence set forth in SEQ ID NO:87. In some embodiments, (i) the first nucleic acid comprises a sequence complementary to nucleotides 2215 to 2236 of the mRNA encoding TGFBR2 comprising the sequence set forth in SEQ ID NO:85 (SEQ ID NO:74); the second nucleic acid comprises a sequence complementary to nucleotides 518 to 539 of the mRNA encoding PTPN2 comprising the sequence set forth in SEQ ID NO:86 (SEQ ID NO:73); and (iii) the third nucleic acid comprises a sequence complementary to nucleotides 1126 to 1147 of the mRNA encoding FAS comprising a sequence set forth in SEQ ID NO:87 (SEQ ID NO:72). In some embodiments, the fourth nucleic acid comprises a sequence complementary to nucleotides 4430 to 4451 of the mRNA encoding TGFBR2 comprising the sequence set forth in SEQ ID NO:85 (SEQ ID NO:75). In some embodiments, the third nucleotide sequence comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:71. In some embodiments, the third nucleotide sequence comprises the sequence set forth in SEQ ID NO:71.

In some embodiments, the T cell is characterized by: reduced expression of TGFBR2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid and/or the fourth nucleic acid, reduced expression of PTPN2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid; and/or reduced expression of FAS by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the third nucleic acid.

In some embodiments, the first nucleotide sequence of (a) and the third nucleotide sequence of (e) are on a same polynucleotide. In some embodiments, the polynucleotide is present in the disrupted endogenous TRAC gene in the genetically engineered human T cell, and the first and third nucleotide sequences are under the operable control of a promoter. In some embodiments, the promoter is a heterologous promoter of the endogenous TRAC gene. In some embodiments, the heterologous promoter is a constitutive promoter. In some embodiments, the heterologous promoter is or comprises a human elongation factor 1 alpha (EF1α) promoter or a variant thereof. In some embodiments, the promoter is a synthetic promoter. In some embodiments, the polynucleotide further comprises an intron between the first nucleotide of (a) and the second nucleotide of (c). In some embodiments, the polynucleotide comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:67. In some embodiments, the polynucleotide comprises the sequence set forth in SEQ ID NO:67. In some embodiments, the polynucleotide has been integrated in the disrupted endogenous TRAC gene by homology directed repair (HDR).

In some embodiments, the genetically engineered human T cell further comprises: (f) a third genetic disruption in an endogenous CD70 gene. In some embodiments, the third genetic disruption in the endogenous CD70 gene is in a target site sequence in exon 2 of the endogenous CD70 gene. In some embodiments, the target site sequence in exon 2 of the endogenous CD70 gene is located at hg38 genomic coordinates chr19:6,590,122-6,590,142. In some embodiments, the target site sequence in exon 2 of the endogenous CD70 gene has the sequence set forth in SEQ ID NO: 66, a contiguous portion thereof of at least 14 nucleotides (nt), or a complementary sequence of the foregoing. In some embodiments, the target site sequence in exon 2 of the endogenous CD70 gene has the sequence set forth in SEQ ID NO: 66. In some embodiments, the third genetic disruption is by a CRISPR-Cas system that comprises a Cas12a protein and a guide RNA (gRNA) comprising a spacer sequence comprising the nucleic acid sequence of SEQ ID NO: 65, or a contiguous portion thereof of at least 14 nt. In some embodiments, the CRISPR-Cas system is a ribonucleoprotein (RNP) complex comprising the Cas12a protein and the gRNA. In some embodiments, the third genetic disruption disrupts one or more alleles of the endogenous CD70 gene. In some embodiments, the third genetic disruption disrupts all alleles of the endogenous CD70 gene. In some embodiments, the third genetic disruption reduces protein expression of CD70 encoded from the endogenous CD70 gene. In some embodiments, the third genetic disruption reduces protein expression of CD70 on the surface of the genetically engineered human T cell. In some embodiments, there is no detectable expression of CD70 in the genetically engineered human T cell. In some embodiments, the genetically engineered human T cell has reduced fratricide as compared to a control cell that does not comprise the third genetic disruption. In some embodiments, the human T cell is a primary human T cell. In some embodiments, the primary human T cell is from a healthy human donor.

Also provided is a genetically engineered human T cell comprising: (a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising: (i) a nucleotide sequence comprising the sequence set forth in SEQ ID NO: 71 and (ii) a nucleotide sequence encoding a CAR comprising the amino acid sequence set forth in SEQ ID NO: 38 or SEQ ID NO: 41, wherein X is Q or pyroglutamate, is present in the disrupted endogenous TRAC gene in the genetically engineered human T cell, (b) a second genetic disruption in exon 2 of an endogenous B2M gene located at hg38 genomic coordinates chr15:44,715,614-44,715,634, wherein the second genetic disruption disrupts one or more alleles of the endogenous B2M gene, and wherein a nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and (c) a third genetic disruption in exon 2 of an endogenous CD70 gene located at hg38 genomic coordinates chr19:6,590,122-6,590,142, wherein the third genetic disruption disrupts all alleles of the endogenous CD70 gene. Also provided herein is a genetically engineered human T cell comprising: (a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising: (i) a nucleotide sequence comprising: (1) a first shRNA comprising the sequence set forth in SEQ ID NO:74, (2) a second shRNA comprising the sequence set forth in SEQ ID NO:73, (3) a third shRNA comprising the sequence set forth in SEQ ID NO:72, and (4) a fourth shRNA comprising the sequence set forth in SEQ ID NO:75; and (ii) a nucleotide sequence encoding a CAR targeting CD70 comprising the sequence set forth in SEQ ID NO: 38 or SEQ ID NO: 41, wherein X is Q or pyroglutamate, is present in the disrupted TRAC gene in the genetically engineered human T cell, (b) a second genetic disruption in exon 2 of the endogenous B2M gene located at hg38 genomic coordinates chr15:44,715,614-44,715,634, wherein the second genetic disruption disrupts one or more alleles of the endogenous B2M gene, and wherein the first nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and (c) a third genetic disruption in exon 2 of an endogenous CD70 gene located at hg38 genomic coordinates chr19:6,590,122-6,590,142, wherein the third genetic disruption disrupts all alleles of the endogenous CD70 gene.

Also provided herein are populations of genetically engineered human T cells comprising the genetically engineered human T cell. In some embodiments, at least or at about 50%, at least or at about 60%, at least or at about 70%, at least or at about 80%, or at least or at about 90% of the cells in the population have a genetic modification selected from: (a) the first genetic disruption in the endogenous TRAC gene; (b) the second genetic disruption in the endogenous B2M gene; (c) a knock-in of the first nucleotide sequence transgene encoding the single chain HLA-E fusion protein; (d) a knock-in of the second nucleotide sequence; (e) a knock-in of the third nucleotide sequence encoding the CAR; and/or (f) the third genetic disruption in the endogenous CD70 gene. In some embodiments, at least or at about 50%, at least or at about 60%, at least or at about 70%, at least or at about 80%, or at least or at about 90% of the cells in the population is characterized by: (1) one or more of (a)-(f); (2) two or more of (a)-(f); (3) three or more of (a)-(f); (4) four or more of (a)-(f); (5) five or more of (a)-(f); or (6) all of (a)-(f).

Also provided herein is a composition comprising the population of genetically engineered human T cells. In some embodiments, the composition is a pharmaceutical composition comprising a pharmaceutically acceptable excipient. In some embodiments, the composition comprises a cryoprotectant, optionally wherein the cryoprotectant is DMSO.

Also provided herein is method of treatment comprising administering the genetically engineered human T cell of any of the embodiments described herein, the population of genetically engineered human T cells of any of the embodiments described herein, or the composition of any of the embodiments described herein to a human subject having a disease or disorder. In some embodiments, the disease or disorder is associated with CD70. In some embodiments, the disease or disorder is a cancer. In some embodiments, the cancer is a solid tumor. In some embodiments, the solid tumor is a renal cell carcinoma. In some embodiments, the renal cell carcinoma is a clear cell renal cell carcinoma.

In some embodiments, the human subject is non-responsive or refractory to one or more standard therapies for cancer. In some embodiments, the human subject has developed drug resistance to one or more standard therapies for cancer. In some embodiments, the human subject has undergone surgery in an attempt to treat the disease or disorder. In some embodiments, the human subject has not been previously treated for cancer. In some embodiments, the human subject has not undergone surgery in an attempt to treat the disease or disorder.

In some embodiments, the method comprises allogeneic transfer, in which the cells are isolated and/or otherwise prepared from a donor other than the human subject. In some embodiments, the donor is a healthy donor. In some embodiments, the donor does not have the disease or disorder. In some embodiments, the donor and human subject are genetically identical or genetically similar. In some embodiments, the human subject expresses a HLA class or supertype that is the same as the donor. In some embodiments, the human subject does not express a HLA class or supertype that is the same as the donor.

In some embodiments, the method comprises administering a dose comprising a therapeutically effective amount of the genetically engineered human T cell, the population of genetically engineered T cells, or the composition. In some embodiments, the dose comprises about 25×10⁶, about 50×10⁶, 100×10⁶, about 150×10⁶, about 200×10⁶, about 250×10⁶, about 300×10⁶, about 350×10⁶, about 400×10⁶, about 450×10⁶, about 500×10⁶, about 550×10⁶, about 650×10⁶, about 700×10⁶, about 750×10⁶, about 800×10⁶, about 850×10⁶, about 900×10⁶, about 950×10⁶, about 1×10⁹, about 1.1×10⁹, about 1.2×10⁹, about 1.3×10⁹, about 1.4×10⁹, or about 1.5×10⁹ CAR+ engineered T cells. In some embodiments, the dose comprises about 50×10⁶ CAR+ engineered T cells. In some embodiments, the dose comprises about 100×10⁶ CAR+ engineered T cells. In some embodiments, the dose comprises about 300×10⁶ CAR+ engineered T cells. In some embodiments, the dose comprises about 900×10⁶ CAR+ engineered T cells.

In some embodiments, the dose is administered via an intravenous (IV) infusion. In some embodiments, the IV infusion is a single IV infusion. In some embodiments, the human subject receives more than one dose or more than one IV infusion. In some embodiments, the dose is provided as a suspension for administration via IV infusion.

In some embodiments, the human subject is pretreated with a lymphodepleting chemotherapy prior to administration of the genetically engineered human T cells. In some embodiments, the lymphodepleting chemotherapy comprises treating the human subject with fludarabine IV (30 mg/m²/day) and cyclophosphamide IV (500 mg/m²/day) for three days prior to administration of the genetically engineered human T cells. In some embodiments, the lymphodepleting chemotherapy comprises treating the human subject with fludarabine IV (30 mg/m²/day) and cyclophosphamide IV (500 mg/m²/day) for four days or five days prior to administration of the genetically engineered human T cells.

In some embodiments, the method further comprises at least one additional therapy, therapeutic agent, or modality. In some embodiments, the at least one additional therapeutic agent or modality comprises at least one PD-1 therapy. In some embodiments, the at least one PD-1 therapy is an antibody or antigen-binding fragment thereof that binds to PD-1. In some embodiments, the at least one PD-1 therapy is an antibody or antigen-binding fragment thereof that binds to PD-L1. In some embodiments, the at least one PD-1 therapy is selected from the group consisting of nivolumab, pembrolizumab, cemiplimab, dostarlimab, retifanlimab, toripalimab, atezolizumab, avelumab, durvalumab, cosibelimab, camrelizumab, sintilimab, and tislelizumab. In some embodiments, the at least one PD-1 therapy is nivolumab. In some embodiments, the nivolumab is administered to the human subject about once every two weeks at a dose of 240 mg. In some embodiments, the nivolumab is administered to the human subject about once every two weeks at a dose of 600 mg. In some embodiments, the nivolumab is administered to the human subject about once every two weeks at a dose of 720 mg. In some embodiments, the nivolumab is administered to the human subject about once every two weeks at a dose of 960 mg. In some embodiments, the nivolumab is administered to the human subject about once every two weeks at a dose of 1200 mg. In some embodiments, the nivolumab is administered to the human subject about once every three weeks at a dose of 360 mg. In some embodiments, the nivolumab is administered to the human subject about once every three weeks at a dose of 720 mg. In some embodiments, the nivolumab is administered to the human subject about once every three weeks at a dose of 900 mg. In some embodiments, the nivolumab is administered to the human subject about once every three weeks at a dose of 960 mg. In some embodiments, the nivolumab is administered to the human subject about once every three weeks at a dose of 1200 mg. In some embodiments, the nivolumab is administered to the human subject about once every four weeks at a dose of 480 mg. In some embodiments, the nivolumab is administered to the human subject about once every four weeks at a dose of 720 mg. In some embodiments, the nivolumab is administered to the human subject about once every four weeks at a dose of 960 mg. In some embodiments, the nivolumab is administered to the human subject about once every four weeks at a dose of 1200 mg. In some embodiments, the nivolumab is administered to the human subject about once every two weeks at a dose of between 3 mg/kg and 10 mg/kg.

In some embodiments, the nivolumab is administered intravenously.

In some embodiments, the nivolumab is administered subcutaneously. In some embodiments, the nivolumab is co-formulated with hyaluronidase. In some embodiments, the hyaluronidase is at a dose of about 10,000 units to about 20,000 units, inclusive. In some embodiments, the hyaluronidase is at a dose of about 10,000 units; about 12,000 units; 15,000 units; about 16,000 units; or about 20,000 units.

In some embodiments, the nivolumab co-formulated with hyaluronidase is administered to the human subject about once every two weeks at a dose of 600 mg nivolumab and a dose of about 10,000 units hyaluronidase. In some embodiments, the nivolumab co-formulated with hyaluronidase is administered to the human subject about once every three weeks at a dose of 900 mg nivolumab and a dose of about 15,000 units hyaluronidase. In some embodiments, the nivolumab co-formulated with hyaluronidase is administered to the human subject about once every four weeks at a dose of 1200 mg nivolumab and about 20,000 units hyaluronidase. In some embodiments, the nivolumab co-formulated with hyaluronidase is administered to the human subject about once every two to four weeks at a dose of 720 mg nivolumab and about 20,000 units hyaluronidase. In some embodiments, the nivolumab co-formulated with hyaluronidase is administered to the human subject about once every two to four weeks at a dose of 960 mg nivolumab and about 20,000 units hyaluronidase. In some embodiments, the nivolumab co-formulated with hyaluronidase is administered to the human subject about once every two to four weeks at a dose of 1200 mg nivolumab and about 20,000 units hyaluronidase. In some embodiments, the nivolumab co-formulated with hyaluronidase is administered to the human subject about once every two to four weeks at a dose of 720 mg nivolumab and about 12,000 units hyaluronidase. In some embodiments, the nivolumab co-formulated with hyaluronidase is administered to the human subject about once every two to four weeks at a dose of 960 mg nivolumab and about 16,000 units hyaluronidase. In some embodiments, the nivolumab co-formulated with hyaluronidase is administered to the human subject about once every two to four weeks at a dose of 1200 mg nivolumab and about 20,000 units hyaluronidase. In some embodiments, the once every two to four weeks is once every two weeks. In some embodiments, the once every two to four weeks is once every three weeks. In some embodiments, the once every two to four weeks is once every four weeks.

In some embodiments, the nivolumab is administered to the subject about once every two weeks at a dose of 3 mg/kg. In some embodiments, the at least one PD-1 therapy is administered to the human subject for 4, 6, 8, 12, 15, 18, 20, 24, 30, 36, 40, 48, or 60 months. In some embodiments, the at least one PD-1 therapy is discontinued after 4, 6, 8, 12, 15, 18, 20, 24, 30, 36, 40, 48, or 60 months. In some embodiments, the at least one PD-1 therapy is administered to the human subject for 12 months. In some embodiments, the at least one PD-1 therapy is administered to the human subject for 24 months. In some embodiments, the at least one PD-1 therapy is administered to the human subject until the disease or disorder progresses.

In some embodiments, the at least one additional therapeutic agent or modality comprises at least one tyrosine kinase inhibitor (TKI). In some embodiments, the at least one TKI is administered to the subject as an oral capsule or tablet. In some embodiments, the at least one TKI is a small-molecule inhibitor of at least one of c-Met (HGFR), VEGFR1, VEGFR2, VEGFR3, AXL, RET, and FLT3. In some embodiments, the at least one TKI is a small-molecule inhibitor of at least one of c-Met (HGFR), VEGFR2, AXL, RET, and FLT3. In some embodiments, the at least one TKI is or comprises cabozantinib, tivozanib, lenvatinib, axitinib, pazopanib or sunitinib. In some embodiments, the at least one TKI is or comprises cabozantinib. In some embodiments, the cabozantinib is administered to the subject once daily. In some embodiments, the cabozantinib is administered to the human subject until the disease or disorder progresses. In some embodiments, the cabozantinib is administered to the subject at a dose of 40 mg/day. In some embodiments, the cabozantinib is administered to the subject at a dose of 60 mg/day.

In some embodiments, the at least one TKI is a small-molecule inhibitor of at least one of VEGFR1, VEGFR2, and VEGFR3. In some embodiments, the at least one TKI is or comprises tivozanib. In some embodiments, the tivozanib is administered to the human subject once daily. In some embodiments, the tivozanib is administered to the human subject until the disease or disorder progresses. In some embodiments, the tivozanib is administered to the human subject in a dosing cycle. In some embodiments, the dosing cycle comprises a 28-day cycle comprising administering to the human subject one dose of tivozanib orally for 21 days followed by seven days without administration of tivozanib to the human subject. In some embodiments, the tivozanib is administered to the human subject at a dose of 1.34 mg/day. In some embodiments, the tivozanib is administered to the human subject at a dose of 0.89 mg/day.

Also provided herein is a guide RNA (gRNA), wherein the gRNA targets an endogenous TRAC gene and comprises the sequence set forth in SEQ ID NO:55. Also provided herein is a guide RNA (gRNA), wherein the gRNA targets an endogenous CD70 gene and comprises the sequence set forth in SEQ ID NO:64. Also provided herein is a polynucleotide encoding the CAR.

Also provided herein is a polynucleotide comprising the sequence set forth in, or a sequence that has at least about 85%, 86%, 87%, 88%, 89%, 90%, 91%, 92%, 93%, 94%, 95%, 96%, 97%, 98%, or 99% sequence identity to, SEQ ID NO:67. In some embodiments, the polynucleotide comprises the sequence set forth in SEQ ID NO:67.

Also provided herein is a polynucleotide, wherein the polynucleotide comprises the sequence set forth in, or a sequence of amino acids that has at least about 85%, 86%, 87%, 88%, 89%, 90%, 91%, 92%, 93%, 94%, 95%, 96%, 97%, 98%, or 99% sequence identity to, SEQ ID NO:77. In some embodiments, the polynucleotide comprises the sequence set forth in SEQ ID NO:77.

Also provided herein is a vector comprising the polynucleotide described above. In some embodiments, the vector is a viral vector. In some embodiments, the viral vector is an AAV.

Also provided herein is a cell comprising the CAR described above, the gRNA described above, the polynucleotide described above, or the vector described above. In some embodiments, the cell is a T cell. In some embodiments, the T cell is a primary T cell. In some embodiments, the primary T cell is from a human donor. In some embodiments, the T cell is a CD4+ T cell, a CD8+ T cell, or a CD4+ and CD8+ T cell.

Also provided herein is an antibody, or antigen-binding fragment thereof, comprising a CD70-binding domain that binds to CD70, wherein the CD70-binding domain comprises a heavy chain only variable region (VHH), wherein: (i) the VHH comprises a CDR-1, CDR-2, and CDR-3 each comprising a sequence that is contained within SEQ ID NO: 1, wherein X is Q or pyroglutamate; or (ii) the VHH comprises a CDR-1, CDR-2, and CDR-3 each comprising a sequence that is contained within SEQ ID NO:27, wherein X is Q or pyroglutamate. The antibody, or antigen-binding fragment thereof, wherein: (i) the VHH of the CD70-binding domain comprises CDR-1, CDR-2, and CDR-3 sequences set forth in SEQ ID NOs: 4, 5, and 6, respectively; (ii) the VHH of the CD70-binding domain comprises CDR-1, CDR-2, and CDR-3 sequences set forth in SEQ ID NOs: 7, 8, and 9, respectively; (iii) the VHH of the CD70-binding domain comprises CDR-1, CDR-2, and CDR-3 sequences set forth in SEQ ID NOs: 79, 80, and 81, respectively; or (iv) the VHH of the CD70-binding domain comprises CDR-1, CDR-2, and CDR-3 sequences set forth in SEQ ID NOs: 82, 83, and 84, respectively. In some embodiments, (i) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or (ii) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate. The antibody, or antigen-binding fragment thereof, wherein: (i) the VHH comprises a sequence set forth in SEQ ID NO: 1, wherein X is Q or pyroglutamate; or (ii) the VHH comprises a sequence set forth in SEQ ID NO: 27, wherein X is Q or pyroglutamate.

### BRIEF DESCRIPTION OF THE DRAWINGS

FIG. 1 depicts the target tumor cell killing ability of CAR T cells containing the indicated exemplary CD70 VHH binders and hinge domains in an in vivo mouse model containing a renal cell carcinoma cell line ACHN xenograft, as compared to mock T cells that contained a knock-out of the CD70 and TRAC loci but that did not contain the anti-CD70 CAR, shRNA cassette, or HLA-E(A) transgenes.

FIG. 2 depicts the level of cytokine IFN-γ production by T cells derived from one of three indicated donors expressing an exemplary CAR containing the anti-CD70 Binder 16 and CD28 hinge when co-cultured with either a CD70-containing target cell line A498 (top) or ACHN (bottom) and in the presence of soluble CAR-Fc, dimeric soluble CD27 extracellular domain, monomeric soluble CD27 extracellular domain, or no competitor.

FIG. 3 depicts a schematic of the gene-edited T cell product. The TRAC, B2M and CD70 loci are disrupted, thus preventing endogenous TCR, HLA-I and CD70 expression, respectively. A shRNA cassette containing shRNAs to knock down TGFBR2, PTPN2, and FAS and an anti-CD70 CAR transgene are delivered using rAAV6 vector for site-specific integration at the TRAC locus. In addition, an HLA-E fusion protein transgene is delivered using an additional rAAV6 vector for site-specific integration at the B2M locus.

FIG. 4A depicts a schematic of each component of AAV vector #1.

FIG. 4B depicts the percentage of engineered allogeneic T cells containing each of the indicated modifications, specifically the knock-in of an anti-CD70 CAR (CAR KI), knock-down of FAS (FAS KD), knock-down of PTPN2 (PTPN2 KD), knock-down of TGFBR2 (TGFBR2 KD), knock-out of the TRAC locus (TRAC KO), knock-in of the HLA-E fusion protein (HLA-E(A) KI), knock-out of the B2M locus (B2M KO), and knock-out of the CD70 locus CD70 KO).

FIG. 5 depicts the expression of the CAR transgene (far left, 80.8% positive), knock-down of FAS (middle left), TGFBR2 (middle right), or FAS (far right), and acute cytotoxicity of engineered T cells (bottom) containing the shRNA cassette and anti-CD70 CAR transgene, as compared to mock T cells containing a knock-out of the CD70 and TRAC loci but that did not contain the shRNA cassette, anti-CD70 CAR transgene, or HLA-E(A) transgene.

FIG. 6A depicts the expression of the CAR transgene in engineered T cells with TRAC and CD70 knocked out, the shRNA cassette optimized to contain one of the indicated promoter/non-coding element combinations, and an anti-CD70 CAR transgene knocked-in to either the TRAC or CD70 loci. Conditions with engineered T cells only containing an antiCD70 CAR transgene with no shRNA cassette (CAR alone) or mock T cells that contained a knock-out of the CD70 and TRAC loci but that did not contain a shRNA cassette, anti-CD70 CAR transgene, or HLA-E(A) transgene were also tested as controls.

FIG. 6B depicts the target tumor cell killing ability of engineered T cells containing the shRNA cassette optimized to contain either an HTLV or synthetic non-coding element and an anti-CD70 CAR transgene in an in vivo mouse model containing a renal cell carcinoma cell line xenograft, as compared to the use of no T cells (tumor alone).

FIG. 7 depicts a schematic of a transgene encoding: (a) an shRNA cassette to knockdown FAS, PTPN2, and TGFBR2 or a non-targeting control (NTC) shRNA cassette, and (b) an exemplary CD70 CAR.

FIGS. 8A-8D depicts the percentage of anti-CD70 CAR expression (FIG. 8A), gMFI of CAR-positive cells (FIG. 8B), percentage of cells containing FAS knock-down (FIG. 8C), and percentage of cells containing PTPN2 knock-down (FIG. 8D) in engineered T cells containing both a shRNA cassette and anti-CD70 CAR transgene (shRNA+) or an anti-CD70 CAR transgene and a non-targeting control shRNA cassette (shRNA−) in three separate donors.

FIG. 9A depicts the functional consequences of TGFBR2 knock-down in engineered T cells containing an shRNA cassette and anti-CD70 CAR transgene (+shRNA) as compared to engineered T cells containing an anti-CD70 CAR transgene and a non-targeting control shRNA cassette (−shRNA). The left panel shows representative flow cytometry plots depicting the expression of CD103, a TGFBR2 pharmacodynamic marker, in the +shRNA and −shRNA engineered T cells, while the right panel summarizes across the percentage of CD103+ CAR T cells in +shRNA and −shRNA engineered T cells across three replicates.

FIG. 9B depicts the functional knock-down of FAS in engineered T cells containing a shRNA cassette and anti-CD70 CAR transgene (+shRNA) as compared to engineered T cells containing an anti-CD70 CAR transgene and a non-targeting control shRNA cassette (−shRNA) following incubation with Fas agonist. The left panel shows representative flow cytometry plots depicting the percentage of live versus dead cells in the +shRNA and −shRNA engineered T cells, while the right panel summarizes across the percentage of apoptosis in +shRNA and −shRNA engineered T cells incubated with Fas agonist across three replicates.

FIG. 10A depicts the cytolytic killing of THP-1 cells expressing either a “masking” anti-CD70 CAR that masks the presence of CD70 or a control CAR directed to CD19 when cultured alone or co-cultured with T cells expressing the “masking” anti-CD70 CAR.

FIG. 10B and FIG. 10C depict the harvest yield (FIG. 10B) and harvest viability (FIG. 10C) of engineered T cells expressing a “masking” anti-CD70 CAR that contain either an unmodified wild-type CD70 locus (CD70 WT) or a CD70 knock-out (CD70 KO). Mock T cells that contained knock-outs of the TRAC and CD70 loci, but not anti-CD70 CAR or HLA-E(A) transgenes, were also tested as a control.

FIG. 10D and FIG. 10E depict the harvest yield (FIG. 10D) and harvest viability (FIG. 10E) of engineered T cells expressing an anti-CD70 CAR using Binder 16 that contain either an unmodified wild-type CD70 locus (CD70 WT) or a CD70 knock-out (CD70 KO). Mock T cells that contained a knock-out of the TRAC and CD70 loci but not anti-CD70 CAR or HLA-E(A) transgenes were also tested as a control.

FIG. 10F depicts the tumor cell killing ability of engineered T cells expressing a “masking” anti-CD70 CAR that contain either an unmodified wild-type CD70 locus (CD70 WT) or a CD70 knock-out (CD70 KO). Mock T cells that contained a knock-out of the TRAC locus, but not anti-CD70 CAR or HLA-E(A) transgenes or a knockout of the CD70 locus, were also tested as a control.

FIG. 11 depicts the protection of engineered allogeneic T cells from Donor 1 (left column) or Donor 2 (right column) containing all the modifications described in FIG. 4B (Allo CD70), engineered allogeneic T cells containing all the modifications described in FIG. 4B but not the knock-out of B2M and incorporation of an HLA-E fusion protein (CD70-CAR B2M (+) No HLA-E(A) transgene), and engineered allogeneic T cells containing all the modifications described in FIG. 4B but without the incorporation of an HLA-E fusion protein (CD70-CAR, B2M KO, No HLA-E(A) transgene). Cells were plated with primary NK cells from Donor 3 (top) or Donor 4 (bottom) to determine levels of NK-mediated killing of engineered allogeneic T cells. Protection from NK cells was measured as the percentage of live allogeneic T cells.

FIGS. 12A-C depict the functional ability of engineered allogeneic T cells containing a control shRNA cassette targeting genes not present in the human genome and anti-CD70 CAR transgene (CAR+cntrl shRNA) or a shRNA cassette and anti-CD70 CAR transgene (CAR+KD shRNA KD) in in vitro assays. FIG. 12A and FIG. 12B depict the cytolytic activity of engineered allogeneic T cells against A498 cells containing NucLight Red fluorescence whose integrated fluorescence intensity was measured using an Incucyte following an initial co-incubation (initial stimulation; FIG. 12A) and second incubation (re-stimulation; FIG. 12B). A control without engineered allogeneic T cells (target only) was also used. FIG. 12C depicts the level of cytokine IFN-γ production by engineered allogeneic T cells at day 16.

FIG. 12D depicts the cytolytic activity overtime of engineered T cells containing a shRNA cassette derived from one of three donors when co-cultured with REH tumor cells at one of four different effector to target (E:T) ratios: 1:2 (top left), 1:4 (top right), 1:8 (bottom left), and 1:16 (bottom right). As controls, the engineered T cells were replaced with engineered T cells containing a non-targeting control (NTC) shRNA cassette (CD70 CAR T) or mock T cells.

FIG. 12E depicts the percent target cell lysis at the indicated E:T ratios following co-culture of engineered T cells containing a shRNA cassette when co-cultured with REH tumor cells. As controls, the engineered T cells were replaced with engineered T cells containing a non-targeting control (NTC) shRNA cassette (CD70 CAR T) or mock T cells.

FIG. 12F depicts production of different cytokines IFNγ (left), IL-2 (middle), and TNFα (right) in co-cultures of REH tumor cells and engineered T cells. As controls, the engineered T cells were replaced with engineered T cells containing a non-targeting control (NTC) shRNA cassette (CD70 CAR T) or mock T cells.

FIG. 13 depicts the cytolytic activity of engineered CAR T cells derived from Donor 1 (left column) or Donor 2 (right column) containing the anti-CD70 CAR transgene and a non-targeting control (NTC) shRNA cassette targeting genes not present in the human genome (CAR+cntrl shRNA) or an anti-CD70 CAR transgene and an shRNA cassette knocking down FAS, PTPN2, and TGFBR2 (CAR+shRNA) in an in vivo model. Either 50,000 (left) or 150,000 (right) engineered allogeneic T cells were injected into mice containing 786-O xenografts and 786-O tumor volume was measured over time. As a control, mice were also injected with no engineered allogeneic T cells (tumor only).

FIG. 14 depicts the cytolytic activity of engineered allogeneic T cells containing an anti-CD70 CAR transgene (CAR alone) or an shRNA cassette knocking down FAS, PTPN2, and TGFBR2 and anti-CD70 CAR transgene (CAR+shRNA KD) in an in vivo model. Engineered allogeneic T cells were injected into mice containing A498 xenografts and A498 tumor volume was measured over time. As controls, mice were also injected with no T cells (tumor only) or mock T cells containing only knock-outs in the TRAC and CD70 loci (mock).

FIG. 15A depicts the cytolytic activity of engineered allogeneic T cells derived from Donor 1 (top row) or Donor 2 (bottom row) containing an anti-CD70 CAR transgene (CAR alone) or an shRNA cassette knocking down FAS, PTPN2, and TGFBR2 and anti-CD70 CAR transgene (CAR+shRNA) in an in vivo model. Engineered allogeneic T cells were injected into mice containing ACHN xenografts and ACHN tumor volume was measured over time. As a control, mice were also injected with no engineered allogeneic T cells (tumor only).

FIG. 15B depicts the functional TGFBR2 knock-down, as measured by percentage of CD103 (a pharmacodynamic marker for TGFBR2) in CAR+ T cells, of engineered allogeneic T cells containing an anti-CD70 CAR transgene (CAR alone) or an shRNA cassette knocking down FAS, PTPN2, and TGFBR2 and an anti-CD70 CAR transgene (CAR+shRNA) in an in vivo mouse model containing ACHN xenografts.

FIGS. 16A-16C depict production of different cytokines IFNγ (FIG. 16A), IL-2 (FIG. 16B), and TNFα (FIG. 16C) in co-cultures of K562 CD70 Tet-on cells—which express CD70 in a dose-dependent level in response to doxycycline—and engineered allogeneic T cells derived from Donor A (left), Donor B (middle), or Donor C (right), when incubated with indicated levels of doxycycline or with no doxycycline (see conditions marked “Media”). As controls, cytokine production was also measured when engineered allogeneic T cells were replaced with mock T cells and/or when K562 CD70 Tet-On cells were replaced with K562 parental cells that express no CD70.

### DETAILED DESCRIPTION

Provided herein are VHH domains directed against CD70 (also known as CD27 ligand, CD27LG, and TNFSF7, for example) and chimeric antigen receptors (CARs) incorporating a provided VHH single domain antibody as part of the extracellular antigen binding domain of the CAR. Also provided are cells, such as engineered or recombinant cells expressing such CD70-targeted CARs or containing nucleic acids encoding such CARs, and compositions and articles of manufacture and therapeutic doses containing such cells. Among the cells are T cells engineered with the CD70-targeted CAR.

CD70 is a member of the tumor necrosis factor superfamily, which has the ability to regulate the activation, proliferation and differentiation of T cells and B cells, and plays an important role in regulating the immune response. In normal tissues, CD70 is only expressed on activated T and B cells and mature DC cells, but CD70 is highly expressed in a variety of tumor tissues, making it an effective target molecule in tumor immunotherapy. A CD70-targeted CAR described herein, and T cell engineered products incorporating the same, can be used to treat CD70-positive tumors. Such CAR-engineered T cells can be used for targeting CD70-expressing cells, such as tumor cells associated with cancer. In addition to CD70 expression on T cell lymphoma, such as peripheral T cell lymphoma (PTCL), CD70 is expressed on many solid tumors, and cancers include renal, bladder, lung, breast, glioblastoma, pancreatic, and melanoma. It is only transiently found on activated T and B lymphocytes and dendritic cells. The embodiments provided herein relate to CAR T cells targeting CD70 for the treatment of cancer, including solid tumors, such as renal cell carcinomas.

In some embodiments, the genetically engineered T cell can further include genetic disruption by gene editing the endogenous CD70 gene loci that is bound or targeted by the CAR. In some embodiments, the gene editing reduces or prevents expression of CD70 on or by the T cells so that the engineered T cells are not targeted by the CD70-targeted CAR. In such aspects, fratricide of the T cells engineered with the CD70-targeted CAR is reduced. In some embodiments, the T cells include a genetic disruption to inactivate or delete by knock-out (KO) of the endogenous CD70 gene. In some embodiments, the genetic disruption of the endogenous CD70 gene is by CRISPR-Cas systems using gRNAs to create indels that result in disruption of the CD70 target gene, such as disruption of all alleles of the target gene, for example, resulting in reduction or elimination of gene expression and/or function.

Also among the provided T cells are allogeneic T cells that that are further genetically engineered to have reduced recognition by the host immune response. In some embodiments, provided T cells are genetically engineered with a CAR, such as a CD70-targeted CAR, and are genetically engineered by one or more strategies to mitigate graft versus host and host versus graft interaction as well as NK cell-mediated rejection, while preserving and in some cases enhancing T cell functions. In some embodiments, the provided engineered CAR T cell therapies are non-alloreactive so that they are not susceptible to, or exhibit reduced susceptibility compared to T cells without the genetic disruptions or modifications, to host immune system rejection. Also provided herein are methods for developing engineered non-alloreactive T-cells expressing the CAR, such as the CD70-targeted CAR, for immunotherapy and more specifically for methods for increasing the persistence and/or the engraftment of allogeneic T cells.

In some embodiments, the T cell is genetically engineered by altering or modulating by genetic disruption one or more endogenous gene in the T cell. In some embodiments, the endogenous gene can be a gene sequence associated with host versus graft response or a gene sequence associated with graft versus host response. In some embodiments, the endogenous gene can be a gene sequence associated with a host versus graft response that is selected from the group consisting of B2M, CIITA, and RFX5, and combinations thereof. B2M is a common (invariant) component of MHC I complexes. CIITA and RFX5 are components of a transcription regulatory complex that is required for the expression of MHC II genes. Disrupting gene expression of these genes to eliminate their expression by gene editing can prevent host versus graft (e.g. T cell therapy) leading to increased allogeneic T cell persistence. In some embodiments, the endogenous gene can be a gene sequence associated with a graft versus host response that is selected from the group consisting of TRAC, CD3-epsilon (CD3ε), and combinations thereof. TRAC and CD3ε are components of the T cell receptor (TCR). Disrupting them by gene editing can take away the ability of the T cells to cause graft versus host disease.

In some cases, cells with reduced or eliminated cell-surface expression of MHC may become susceptible to NK cell-mediated cytotoxicity. In some aspects, certain MHC class I molecules, such as the non-classical MHC molecules MHC-E (HLA-E in humans and Qa-1b in mice), are ligands of and can be recognized by Natural Killer (NK) inhibitory receptors expressed on the surface of NK cells to induce an inhibitory signal to “stop” or halt an NK cell killing response. For example, MHC-E can interact with an inhibitor receptor on the surface of an NK cell that comprises CD94 and/or NKG2A, such as heterodimer of NKG2A disulfide-linked with the CD94 molecule. In some cases, an NK cell response can be triggered to kill cells that they interact with, unless those cells express the MHC molecule recognized by an NK inhibitory cell receptor on the NK cell. In cells in which MHC-I has been downregulated (e.g. as occurs in tumor or virally-infected cells), the NK cells can provide an immune surveillance by detection of “missing self,” which then results in cell killing. In the context of the provided embodiments, while reduced or disrupted expression of certain regulatory molecules (e.g. B2M) in the provided cells can reduce or eliminate expression of classical MHC class I (MHC class Ia) by the cell or on the cell surface, such targeting of regulatory molecules also may reduce or eliminate expression of non-classical MHC class I molecules MHC-E by the cell or on the cell surface, which also may render the cell susceptible to NK cell killing.

In some embodiments, to overcome or reduce risks of or associated with exposure to NK cell-mediated cytotoxicity, e.g., due to the reduced MHC expression, the provided engineered cells include those that are reduced or prevented from being the subject of “missing self” recognition, e.g., by NK cells, to prevent NK cell-mediated immune surveillance that could kill a provided engineered cell lacking an MHC molecule (e.g. MHC-E, also known as HLA-E, respectively). Thus, in some embodiments, in addition to reducing or eliminating expression of an MHC molecule (e.g. MHC class I), the provided engineered cells also include a recombinant NK cell modulator that is or comprises an NK cell modulating (e.g. inhibiting) moiety on the surface of the engineered immune cell. In some embodiments, the NK cell modulating (e.g. inhibiting) moiety is capable of inducing an inhibitory signal in an NK cell. In some embodiments, the binding or modulating induces an inhibitory signal in the NK cell to reduce or prevent lack-of-self recognition and NK cell-mediated rejection. In some embodiments, the inhibitory signal is transduced by a CD94/NKG2A receptor. In some embodiments, the modulating (e.g. inhibiting) moiety is a recombinant HLA-E molecule or binding portion thereof, for example, one that is exogenously introduced for expression on the surface of the cell. In some embodiments, such features of the provided cells result in enhanced efficacy or longevity of adoptive cell therapy in the context of engineered immune cells susceptibility to natural killer (NK) cell-mediated cytotoxicity.

In some embodiments, the T cells are genetically engineered to have reduced or altered expression one or more endogenous genes in the T cell. In some embodiments, the one or more endogenous genes can be a gene sequence associated with chimeric antigen receptor (CAR) T cell potency or a gene sequence associated with T cell fitness and persistence. In some embodiments, the one or more endogenous genes can be a gene sequence associated with CAR T cell potency that is Transforming Growth Factor Beta Receptor 2 (TGFBR2). Lowering expression of TGFBR2 can increase CAR T cell potency. In some embodiments, the one or more endogenous genes can be a gene sequence associated with T cell fitness and persistence. In some embodiments, the one or more endogenous genes can be a gene sequence associated with T cell fitness and persistence that is Protein Tyrosine Phosphatase Non-Receptor Type 2 (PTPN2) and Fas Cell Surface Death Receptor (FAS). FAS is part of a known pathway leading to apoptosis. Lowering expression of these genes can lead to increased T cell fitness and persistence. In some embodiments, the T cells are engineered through molecule that affect RNA interference (RNAi) against the one or more endogenous genes. In some embodiments, the molecules that affect RNAi are encoded on an RNAi expression cassette.

In some embodiments, the engineered T cells are genetically engineered by expression of a CD70-targeted CAR transgene, a genetic disruption that reduces or eliminates the expression or activity of a regulatory molecule that regulates expression and/or surface expression of an endogenous major histocompatibility complex (MHC) class I, a genetic disruption that reduces or eliminates the expression of TRAC, expression of an NK cell inhibiting moiety transgene, and an inhibitory nucleic acid that reduces expression of TGFBR2, PTPN2 and FAS. In some embodiments, the engineered T cells include a CD70-targeted CAR transgene with an extracellular binding domain composed of a means for specifically binding CD70, a genetic disruption that reduces or eliminates the expression of B2M to reduce or eliminate surface expression of endogenous MHC class I, a genetic disruption that reduces or eliminates the expression of TRAC, a transgene for expression of a chimeric HLA-E transgene on the surface of the engineered T cell, and expression of an RNAi expression cassette that reduces expression of TGFBR2, PTPN2, and FAS.

In some embodiments, the provided engineering strategies can be carried out by gene editing methods, including those involving CRISPR-Cas systems. In addition to disrupting or deleting genes by nuclease-directed targeted gene editing, introduction of transgenes (such as the CAR or NK cell inhibiting moiety) can be carried out by insertion into genomic loci at the site of a double stranded break that is repaired by homology directed repair (HDR) using a delivered donor template (e.g. by AAV delivery) with homology around the target site. Gene editing using rare-cutting endonucleases, such as CRISPR-Cas systems using guide RNA/Cas, to disrupt by knock-out (KO) target genes as well as to introduce by knock-in (KI) transgenes to a defined genomic loci has the benefit to modulate gene activity while also providing precise genome modification as compared to alternative methods such as lentivirus delivery and integration.

In some embodiments, the provided engineered T cells include a genetic disruption to inactivate or delete one or more genes implicated in the self/non-self-recognition (e.g., the TRAC and/or B2M gene) by the use of specific rare-cutting endonuclease, followed by a step of knock-in (KI) of said engineered T cells with at least one non-endogenous polypeptide transgene (such as HLA-E fusion protein and/or a recombinant CAR). In some embodiments, provided herein is a genetically engineered T cell with a genetic disruption in the endogenous TRAC gene; a genetic disruption in the endogenous B-2 microglobulin (B2M) gene; a nucleotide sequence encoding a single chain HLA-E fusion protein; and a nucleotide sequence encoding a CD70-targeted CAR. In some embodiments, the genetic disruptions are by CRISPR-Cas systems using gRNAs useful for the creation of indels that result in disruption of the target gene, such as disruption of all alleles of the target gene, for example, reduction or elimination of gene expression and/or function. In some embodiments, the gRNAs are useful for the creation of double strand breaks (DSBs) that facilitate insertion of a donor template into the genome by HDR. In some embodiments, the CAR is integrated by KI into the disrupted TRAC gene by homology directed repair (HDR). In some embodiments, the single chain HLA-E fusion protein is integrated by KI into the disrupted B2M gene by homology directed repair (HDR).

Also provided herein are methods for engineering the T cells. Also provided herein are methods of administering the engineered T cells to a subject, such as for use in treating a disease or condition associated with expression of an antigen that is recognized by the recombinant receptor (e.g. CAR).

In some aspects, the provided engineered cells exhibit enhanced efficacy or longevity when used in adoptive cell therapy, for example, due to reduced or eliminated graft versus host rejection, host versus graft rejection and/or NK-cell mediated rejection. In some embodiments, the provided methods reduce or lessen or prevent an immune response in a subject administered with the genetically engineered T cells, compared to the immune response generated in the subject administered with T cells expressing the CAR in the absence of the genetic disruptions (e.g., KO of B2M and TRAC, and in some cases also CD70) and expression of an NK-cell inhibiting moiety transgene (e.g., HLA-E single chain fusion) in the engineered T cell. In some embodiments, the subject does not exhibit an immune response or a particular type or degree of immune response, against the genetically engineered T cells, such as following the administration of the cells to the subject. The type of immune response may be a detectable immune response, a humoral immune response, and/or a cell-mediated immune response. In some aspects, the provided genetically engineered T cells, compositions and methods result in an increased persistence and efficacy of cells used in adoptive cell therapy. In some aspects, the provided embodiments may reduce the number of T cells that need to be generated or delivered to each patient as the cells can be more efficacious and/or persist for longer. The provided embodiments may also reduce the number of sequential administrations of engineered cells required to treat a patient, or increase the amount of time needed between administrations as cells survive longer.

In some embodiments, the provided engineered cells, compositions and methods can be used regardless of the HLA type or subtype of a subject (e.g., a patient) to whom the cells may be administered, which can, in some aspects, permit “off-the-shelf” delivery to a wider variety of recipients. In some embodiments, the provided compositions and methods can be used to provide adoptive cell therapy using allogeneic cells engineered to treat a disease or disorder. In some cases, using allogeneic cells can provide certain advantages. In some embodiments, cells with known safety and efficacy profiles can be prepared for a wider variety of patients. For example, cells can be derived from a healthy donor and delivered to a subject that may be too sick to provide cells suitable for genetic engineering. In some cases, a subject may have a defect or disease in the cells or cell type typically used for a particular adoptive cell therapy regimen, such that cells from a healthy donor can be used that replace or supplement the diseased cells. In some cases, the ability to engineer or administer allogeneic cells permits the preparation of cells in advance, which can reduce the time needed before being delivered to a patient. In some cases, the engineered allogeneic cells may present lower risks of causing graft-versus-host disease or host-versus-graft disease.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The Section headings used herein are for organizational purposes only and are not to be construed as limiting the subject matter described.

### I. CHIMERIC ANTIGEN RECEPTORS

In some embodiments, provided herein are chimeric antigen receptors directed against CD70. The CAR generally includes an extracellular domain comprising an extracellular binding domain (also called “extracellular antigen binding domain”) directed against CD70, in which the extracellular domain is linked to one or more intracellular signaling components, in some aspects via linkers and/or transmembrane domain(s). In some embodiments, the extracellular binding domain provides a means for binding CD70.

In some embodiments, the CAR contains the extracellular binding domain, a transmembrane domain, and an intracellular signaling domain composed of one or more signaling domains. In some embodiments, the extracellular binding domain and transmembrane can be linked directly or indirectly. In some embodiments, the extracellular binding domain and transmembrane are linked by a spacer, such as any described herein. In some embodiments, the CAR also includes a spacer domain (e.g. hinge domain) separating the extracellular binding domain and the transmembrane domain. In some aspects, the spacer and/or transmembrane domain can link the extracellular portion containing the antigen binding domain and the intracellular signaling domain.

In some embodiments, the CAR includes in order from N- to C-terminus: the extracellular binding domain, a spacer, a transmembrane domain, and an intracellular signaling domain. In such embodiments, the spacer is interposed between the extracellular binding domain and the transmembrane domain.

In some embodiments, the chimeric antigen receptor contains an intracellular domain containing a CD3zeta intracellular signaling domain and a signaling domain of a T cell costimulatory molecule. In some embodiments, the costimulatory signaling domain is between the transmembrane domain and CD3zeta intracellular signaling domain. In some aspects, the T cell costimulatory molecule is 4-1BB.

Also provided herein are polynucleotides encoding any of the provided CARs.

In some embodiments, the CARs are encoded by polynucleotides. The provided polynucleotides can be incorporated into constructs, such as deoxyribonucleic acid (DNA) or RNA constructs, such as those that can be introduced into cells for expression of the encoded CAR. Hence, also provided herein are engineered cells containing any of the provided CARs. Exemplary engineered cells and methods of preparing same are described in Section III. Also provided herein are compositions and articles of manufacture and uses of any of the engineered cells. Also provided are cells expressing the CARs and uses thereof in adoptive cell therapy, such as treatment of diseases and disorders associated with expression of the antigen, such as CD70 expression.

### A. Extracellular Antigen-Binding Domains

The extracellular binding domain of a CAR provides a means for binding to CD70. In some embodiments, the CAR includes an extracellular binding domain that is a heavy chain only variable region (VHH) domain. In some embodiments, the VHH domain is a camelid-derived single domain antibody fragment. In some embodiments, the VHH domain contains a variable heavy chain with three CDRs, CDR1-3, that confer binding to CD70. Also provided herein are VHH domains that specifically bind CD70.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in the variable region (CDR-1, CDR-2, CDR-3).

The amino acid residues of a single domain antibody (e.g., VHH) are numbered according to the general numbering for VH domains given by Kabat et al. (“Sequence of proteins of immunological interest,” US Public Health Services, NIH Bethesda, Md., Publication No. 91), as applied to VHH domains from Camelids in the article of Riechmann and Muyldermans, J. Immunol. Methods 2000 Jun. 23; 240 (1-2): 185-195. According to this numbering, CDR1 of a VHH comprises the amino acid residues at positions 31-35, CDR2 of a VHH comprises the amino acid residues at positions 50-65, and CDR3 of a VHH comprises the amino acid residues at positions 95-102. In this respect, it should be noted that, as is well known in the art for VH domains and for VHH domains, the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). See, e.g., Deschacht et al., 2010. J Immunol 184: 5696-704 for an exemplary numbering for VHH domains according to Kabat.

IMGT (ImMunoGeneTics) numbering scheme is another standardized numbering system for protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)). For VHH camelid antibodies, the IMGT numbering scheme can be used to accurately identify the CDR regions by aligning the VHH sequences with the IMGT framework. According to this numbering, CDR1 of a VHH comprises the amino acid residues of positions 27-38, CDR2 of a VHH comprises amino acid residues 56-65 of a VHH, and CDR3 of a VHH comprises amino acid residues 105-117 of a VHH.

Correspondence between the numbering system, including, for example, the Kabat numbering and the IMGT unique numbering systems, is well known to one skilled in the art (see, e.g., Kabat, supra; Chothia and Lesk, supra; Martin, supra; Lefranc et al., supra).

Thus, unless otherwise specified, a “CDR” or “complementary determining region,” or individual specified CDRs (e.g., CDR-1, CDR-2, CDR-3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-3) contains the amino acid sequence of a corresponding CDR in a given VHH region amino acid sequence, it is understood that such a CDR has a sequence of the corresponding CDR (e.g., CDR-3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other known numbering schemes.

In the present disclosure, it is understood that reference to a sequence (such as sequences for a VHH region) by a SEQ ID NO includes the corresponding amino acid sequence or the corresponding amino acid sequence comprising a post-translational modification. Post-translational modifications can include, e.g., isomerization, ubiquitination, phosphorylation, acetylation, hydroxylation, methylation, glycyosylation, AMPylation, prenylation, deamidation, eliminylation, citrullination, and carbamoylation.

In some embodiments, the VHH of a CD70-targeted antibody, or any of such VHH antibody incorporated in a provided CAR, comprises a modification at the N-terminus. Recombinant monoclonal antibodies can undergo spontaneous or non-spontaneous (e.g., enzymatically catalyzed by glutaminyl cyclase) cyclization of glutamine (Q) or glutamate/glutamic acid (E) at the N-terminus, resulting in pyroglutamate (Liu et al. J Bio; Chem, volume 286:13 (2011)). In some embodiments, the N-terminal amino acid, 2 N-terminal amino acid and/or 3 N-terminal amino acid of any of the CD70-targeted antibodies provided herein are different from the N-terminal amino acid, 2 N-terminal amino acid, and/or 3 N-terminal amino acid of SEQ ID NOS: 2 and 28, e.g., due to a post-translational modification as described above. In some embodiments, the first N-terminal Q of any of SEQ ID NOS: 2 and 28 is a pyroglutamate. Provided herein are CD70-targeted VHH antibodies, or any of such VHH antibody incorporated in a provided CAR, wherein if the N-terminal amino acid of the amino acid sequence is Q or E, the first N-terminal amino acid residue may be pyroglutamate. Also provided are compositions comprising any of the provided CD70-targeted antibodies, or CARS incorporating the same, wherein if the N-terminal amino acid of the antibody sequence is Q or E, at least about 10%, 20%, 30%, 40%, 50%, 60%, 70%, 80%, 90%, 95% or 100% of the antibodies in the composition have a pyroglutamate at the first N-terminal amino acid residue of their sequence.

In some embodiments, the CD70-targeted antibody contains a heavy chain only variable (VHH) region sequence as described, or a sufficient antigen-binding portion thereof. In some embodiments, the CD70-directed antibody, or antigen-binding fragment thereof, contains a VHH region sequence or sufficient antigen-binding portion thereof that contains a CDR-1, CDR-2 and/or CDR-3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the VHH region of the CD70-targeted antibody, or of the extracellular antigen-binding domain of a provided CAR, has the amino acid sequence set forth in any one of SEQ ID NOs: 1-3 and 27-29, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in any one of SEQ ID NOs: 1-3 and 27-29, or contains a CDR-1, CDR-2, and/or CDR-3 present in such a VHH sequence.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO: 1. In some embodiments, the VHH region of the CD70-targeted antibody, or of the extracellular antigen-binding domain of a provided CAR, has the amino acid sequence set forth in SEQ ID NO: 1, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO: 1.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO:2. In some embodiments, the VHH region of the CD70-targeted antibody, or of the extracellular antigen-binding domain of a provided CAR, has the amino acid sequence set forth in SEQ ID NO:2, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO:2.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO:3. In some embodiments, the VHH region of the CD70-targeted antibody, or of the extracellular antigen-binding domain of a provided CAR, has the amino acid sequence set forth in SEQ ID NO:3, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO:3.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO:27. In some embodiments, the VHH region of the CD70-targeted antibody, or of the extracellular antigen-binding domain of a provided CAR, has the amino acid sequence set forth in SEQ ID NO: 27, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO: 27.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO:28. In some embodiments, the VHH region of the CD70-targeted antibody, or of the extracellular antigen-binding domain of a provided CAR, has the amino acid sequence set forth in SEQ ID NO:28, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO:28.

In some embodiments, the VHH region of the CD70-targeted antibody, or of the extracellular antigen-binding domain of a provided CAR, contains a CDR-1, CDR-2, and/or CDR-3 present in the amino acid sequence set forth in SEQ ID NO:29. In some embodiments, the VHH region of the CD70-targeted antibody, or of the extracellular antigen-binding domain of a provided CAR, has the amino acid sequence set forth in SEQ ID NO:29, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VHH region amino acid set forth in SEQ ID NO:29.

In some embodiments, the VHH region of a CD70-binding domain, such as an anti-CD70 antibody, or antigen-binding fragment thereof, comprises a CDR-1, CDR-2, and/or CDR-3 according to Kabat numbering. In some embodiments, the VHH region of a CD70-binding domain, such as an anti-CD70 antibody, or antigen-binding fragment thereof, comprises a CDR-1, CDR-2, and/or CDR-3 according to IMGT numbering.

In some embodiments, the VHH region of the CD70-targeted antibody, or of the extracellular antigen-binding domain of a provided CAR, comprises a CDR-1 comprising the amino acid sequence set forth in SEQ ID NO: 4, a CDR-2 comprising the amino acid sequence set forth in SEQ ID NO: 5, and a CDR-3 comprising the amino acid sequence set forth in SEQ ID NO: 6. In some embodiments, the VHH region of the CD70-targeted antibody, or of the extracellular antigen-binding domain of a provided CAR, comprises a VHH region comprising a CDR-1, a CDR-2, and a CDR-3 comprising the amino acid sequence set forth in SEQ ID NO: 4, 5, and 6, respectively. In some embodiments, the VHH region of the CD70-targeted antibody, or of the extracellular antigen-binding domain of a provided CAR, comprises a CDR-1 comprising the amino acid sequence set forth in SEQ ID NO: 7, a CDR-2 comprising the amino acid sequence set forth in SEQ ID NO: 8, and a CDR-3 comprising the amino acid sequence set forth in SEQ ID NO: 9. In some embodiments, the VHH region of the CD70-targeted antibody, or of the extracellular antigen-binding domain of a provided CAR, comprises a VHH region comprising a CDR-1, a CDR-2, and a CDR-3 comprising the amino acid sequence set forth in SEQ ID NO: 7, 8, and 9, respectively.

In some embodiments, the VHH region of the CD70-targeted antibody, or of the extracellular antigen-binding domain of a provided CAR, comprises a CDR-1 comprising the amino acid sequence set forth in SEQ ID NO: 79, a CDR-2 comprising the amino acid sequence set forth in SEQ ID NO: 80, and a CDR-3 comprising the amino acid sequence set forth in SEQ ID NO: 81. In some embodiments, the VHH region of the CD70-targeted antibody, or of the extracellular antigen-binding domain of a provided CAR, comprises a VHH region comprising a CDR-1, a CDR-2, and a CDR-3 comprising the amino acid sequence set forth in SEQ ID NO: 79, 80, and 81, respectively. In some embodiments, the VHH region of the CD70-targeted antibody, or of the extracellular antigen-binding domain of a provided CAR, comprises a CDR-1 comprising the amino acid sequence set forth in SEQ ID NO: 82, a CDR-2 comprising the amino acid sequence set forth in SEQ ID NO: 83, and a CDR-3 comprising the amino acid sequence set forth in SEQ ID NO: 84. In some embodiments, the VHH region of the CD70-targeted antibody, or of the extracellular antigen-binding domain of a provided CAR, comprises a VHH region comprising a CDR-1, a CDR-2, and a CDR-3 comprising the amino acid sequence set forth in SEQ ID NO: 82, 83, and 84, respectively.

Among the CD70-targeted antibodies provided herein is an antibody with a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in any one of SEQ ID NOs: 1-3 and 27-29. In some embodiments, the CD70-targeted antibody comprises a VHH region comprising the amino acid sequence set forth in any one of SEQ ID NOs: 1-3 and 27-29.

Among the CARs provided herein is a CAR in which the extracellular antigen-binding domain in the provided CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in any one of SEQ ID NOs: 1-3 and 27-29. In some embodiments, the CAR comprises a VHH region comprising the amino acid sequence set forth in any one of SEQ ID NOs: 1-3 and 27-29.

In some embodiments, the VHH region of the CD70-targeted antibody, or of the extracellular antigen-binding domain of a provided CAR, comprises a VHH region comprising the amino acid sequence set forth in SEQ ID NO: 1, or a sequence that has at least 85%, 86%, 87%, 88%, 89%, 90%, 91%, 92%, 93%, 94%, 95%, 96%, 97%, 98% or 99% sequence identity to SEQ ID NO: 1, wherein X is pyroglutamate or Q. In some embodiments, the VHH region of the CD70-targeted antibody, or of the extracellular antigen-binding domain of a provided CAR, comprises a VHH region comprising the amino acid sequence set forth in SEQ ID NO: 1, wherein X is pyroglutamate or Q.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 1, wherein X is pyroglutamate or Q. In some embodiments, the CAR comprises a VHH region comprising the amino acid sequence set forth in SEQ ID NO: 1, wherein X is pyroglutamate or Q.

In some embodiments, the VHH region of the CD70-targeted antibody, or of the extracellular antigen-binding domain of a provided CAR, comprises a VHH region comprising the amino acid sequence set forth in SEQ ID NO: 2, or a sequence that has at least 85%, 86%, 87%, 88%, 89%, 90%, 91%, 92%, 93%, 94%, 95%, 96%, 97%, 98% or 99% sequence identity to SEQ ID NO: 2. In some embodiments, the VHH region of the CD70-targeted antibody, or of the extracellular antigen-binding domain of a provided CAR, comprises a VHH region comprising the amino acid sequence set forth in SEQ ID NO: 2.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 2. In some embodiments, the CAR comprises a VHH region comprising the amino acid sequence set forth in SEQ ID NO: 2.

In some embodiments, the VHH region of the CD70-targeted antibody, or of the extracellular antigen-binding domain of a provided CAR, comprises a VHH region comprising the amino acid sequence set forth in SEQ ID NO: 3, or a sequence that has at least 85%, 86%, 87%, 88%, 89%, 90%, 91%, 92%, 93%, 94%, 95%, 96%, 97%, 98% or 99% sequence identity to SEQ ID NO: 3, wherein X is pyroglutamate. In some embodiments, the VHH region of the CD70-targeted antibody, or of the extracellular antigen-binding domain of a provided CAR, comprises a VHH region comprising the amino acid sequence set forth in SEQ ID NO: 3, wherein X is pyroglutamate.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 3, wherein X is pyroglutamate. In some embodiments, the CAR comprises a VHH region comprising the amino acid sequence set forth in SEQ ID NO: 3 wherein X is pyroglutamate.

In some embodiments, the VHH region of the CD70-targeted antibody, or of the extracellular antigen-binding domain of a provided CAR, comprises a VHH region comprising the amino acid sequence set forth in SEQ ID NO: 27, or a sequence that has at least 85%, 86%, 87%, 88%, 89%, 90%, 91%, 92%, 93%, 94%, 95%, 96%, 97%, 98% or 99% sequence identity to SEQ ID NO: 27, wherein X is pyroglutamate or Q. In some embodiments, the VHH region of the CD70-targeted antibody, or of the extracellular antigen-binding domain of a provided CAR, comprises a VHH region comprising the amino acid sequence set forth in SEQ ID NO: 27, wherein X is pyroglutamate or Q.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 27, wherein X is pyroglutamate or Q. In some embodiments, the CAR comprises a VHH region comprising the amino acid sequence set forth in SEQ ID NO: 27, wherein X is pyroglutamate or Q.

In some embodiments, the VHH region of the CD70-targeted antibody, or of the extracellular antigen-binding domain of a provided CAR, comprises a VHH region comprising the amino acid sequence set forth in SEQ ID NO: 28, or a sequence that has at least 85%, 86%, 87%, 88%, 89%, 90%, 91%, 92%, 93%, 94%, 95%, 96%, 97%, 98% or 99% sequence identity to SEQ ID NO: 28. In some embodiments, the VHH region of the CD70-targeted antibody, or of the extracellular antigen-binding domain of a provided CAR, comprises a VHH region comprising the amino acid sequence set forth in SEQ ID NO: 28.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 28. In some embodiments, the CAR comprises a VHH region comprising the amino acid sequence set forth in SEQ ID NO: 28.

In some embodiments, the VHH region of the CD70-targeted antibody, or of the extracellular antigen-binding domain of a provided CAR, comprises a VHH region comprising the amino acid sequence set forth in SEQ ID NO: 29, wherein X is pyroglutamate, or a sequence that has at least 85%, 86%, 87%, 88%, 89%, 90%, 91%, 92%, 93%, 94%, 95%, 96%, 97%, 98% or 99% sequence identity to SEQ ID NO: 29, wherein X is pyroglutamate. In some embodiments, the VHH region of the CD70-targeted antibody, or of the extracellular antigen-binding domain of a provided CAR, comprises a VHH region comprising the amino acid sequence set forth in SEQ ID NO: 29, wherein X is pyroglutamate.

In some embodiments, the CAR comprises a VHH region comprising the amino acid sequence having at least 85%, 86%, 87%, 88%, 89%, 90%, 91%, 92%, 93%, 94%, 95%, 96%, 97%, 98% or 99% sequence identity to the amino acid sequence set forth in SEQ ID NO: 29, wherein X is pyroglutamate. In some embodiments, the CAR comprises a VHH region comprising the amino acid sequence set forth in SEQ ID NO: 29, wherein X is pyroglutamate.

In certain embodiments, a CAR comprising the CD70-binding domain, such as an antibody or antigen-binding fragment thereof include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC).

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-3 is often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments, each CDR either is unaltered, or contains no more than one, two or three amino acid substitutions.

In some of or any of the provided embodiments, the CAR specifically binds to CD70, such as CD70 on the surface of a cancer cell. In some embodiments binding can be to a human CD70. In some embodiments, among provided CARs and/or CD70-binding domain are those that bind human CD70 protein. In some embodiments, the antibodies specifically bind to human CD70 protein, such as to an epitope or region of human CD70 protein.

In one embodiment, the extent of binding of an CD70 antibody or antigen-binding domain or CAR to an unrelated, non-CD70 protein, such as a non-human CD70 protein or other non-CD70 protein, is less than at or about 10% of the binding of the antibody or antigen-binding domain or CAR to human CD70 protein or human membrane-bound CD70 as measured, e.g., by a radioimmunoassay (RIA). In some embodiments, among the antibodies or antigen-binding domains in the provided CARs, are antibodies or antigen-binding domains or CARs in which binding to mouse CD70 protein or cynomolgus monkey protein is less than or at or about 10% of the binding of the antibody to human CD70 protein.

In some embodiments, the antibodies, in the provided CARs, are capable of binding CD70 protein, such as human CD70 protein, with at least a certain affinity, as measured by any of a number of known methods. In some embodiments, the affinity is represented by an equilibrium dissociation constant (KD); in some embodiments, the affinity is represented by EC₅₀. In some embodiments, the dissociation constant is determined using surface plasmon resonance (SPR) analysis such as by using a BIAcore® instrument. In some embodiments, EC₅₀ is determined using flow cytometry by detection of binding of antibodies to CD70-expressing cells.

In some embodiments, the binding molecule, e.g., antibody or fragment thereof or antigen-binding domain of a CAR, binds, such as specifically binds, to an antigen, e.g., a CD70 protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or kₐ] for this association reaction, assuming bimolecular interaction) equal to or greater than 10⁵ M⁻¹. In some embodiments, the antibody or fragment thereof or antigen-binding domain of a CAR exhibits a binding affinity for the peptide epitope with a KD (i.e., an equilibrium dissociation constant of a particular binding interaction with units of M; equal to the ratio of the off-rate [k_{off} or kₐ] to the on-rate [kₒₙ or kₐ] for this association reaction, assuming bimolecular interaction) of equal to or less than 10⁻⁵ M. For example, the equilibrium dissociation constant KD ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or kₐ; units of 1/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to CD70 protein, such as human CD70 protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a CD70 protein, such as human CD70 protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a CD70 protein, such as human CD70 protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC₅₀) and/or the equilibrium dissociation constant, KD, of the binding molecule, e.g., anti-CD70 antibody or fragment thereof or antigen-binding domain of a CAR, to a CD70 protein, is from or from about 0.01 nM to about 1 M, 0.1 nM to 1 M, 1 nM to 1 M, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, KD, of the binding molecule, e.g., anti-CD70 antibody or fragment thereof or antigen-binding domain of a CAR, to a CD70 protein, is at or about or less than at or about 1 M, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

### B. Spacer

In some embodiments, the CAR further include a spacer domain (in some cases also called a spacer) that is located between the extracellular binding domain and the transmembrane domain. In some embodiments, the spacer contains a hinge region sequence, which in some aspects is a sequence that promotes receptor dimerization. In some embodiments, the spacer is or includes at least a portion of an immunoglobulin constant region or variant or modified version thereof. In some embodiments, the spacer is or comprises a glycine-serine rich sequence or other flexible linker such as known flexible linkers.

In some embodiments, the spacer is a hinge region sequence. In some embodiments, the spacer is or includes at least a portion of human CD8 or CD28 proteins. In some embodiments, the spacer is or includes a hinge region from CD8, or CD28 extracellular domains. In some embodiments, the spacer is or includes a CD8 hinge domain. In some embodiments, the CD8 hinge domain comprises an amino acid sequence having at least 85%, 86%, 87%, 88%, 89%, 90%, 91%, 92%, 93%, 94%, 95%, 96%, 97%, 98% or 99% identity to SEQ ID NO:96. In some embodiments, the CD8 hinge domain comprises an amino acid sequence set forth in SEQ ID NO:96. In some embodiments, the CD8 hinge domain is encoded by a nucleotide sequence having at least 85%, 86%, 87%, 88%, 89%, 90%, 91%, 92%, 93%, 94%, 95%, 96%, 97%, 98% or 99% identity to SEQ ID NO:91. In some embodiments, the CD8 hinge domain is encoded by a nucleotide sequence set forth in SEQ ID NO:91.

In some embodiments, the spacer is or includes a CD28 hinge domain. In some embodiments the CD28 hinge domain comprises an amino acid sequence having at least 85%, 86%, 87%, 88%, 89%, 90%, 91%, 92%, 93%, 94%, 95%, 96%, 97%, 98% or 99% identity to SEQ ID NO: 10. In some embodiments the CD28 hinge domain comprises an amino acid sequence set forth in SEQ ID NO: 10.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises the formula X₁PPX₂P (SEQ ID NO: 101), where X₁ is glycine, cysteine or arginine and X₂ is cysteine or threonine.

In some embodiments, the spacer includes an IgG hinge linked to one or more of a CH2 and CH3 domain, such as an IgG hinge linked to the CH3 domain. In some embodiments, the spacer includes an IgG hinge alone. In some embodiments, the spacer can be a chimeric polypeptide containing one or more of a hinge, CH2 and/or CH3 sequence(s) derived from IgG4, IgG2, and/or IgG2 and IgG4.

In some embodiments, the spacer is or includes at least a portion of an immunoglobulin constant region. In some aspects, the portion of the constant region serves as a spacer region between the extracellular binding domain and transmembrane domain. Exemplary spacers include an IgG hinge and an IgGL hinge. In some embodiments, the spacer is or includes an IgG hinge. In some embodiments, the spacer comprises an amino acid sequence having at least 85%, 86%, 87%, 88%, 89%, 90%, 91%, 92%, 93%, 94%, 95%, 96%, 97%, 98% or 99% identity to SEQ ID NO: 93. In some embodiments, the spacer comprises an amino acid sequence set forth in SEQ ID NO: 93. In some embodiments, the spacer is encoded by a nucleic acid sequence having at least 85%, 86%, 87%, 88%, 89%, 90%, 91%, 92%, 93%, 94%, 95%, 96%, 97%, 98% or 99% identity to SEQ ID NO: 88. In some embodiments, the spacer is encoded by a nucleic acid sequence set forth in SEQ ID NO: 88. In some embodiments, the spacer comprises an amino acid sequence having at least 85%, 86%, 87%, 88%, 89%, 90%, 91%, 92%, 93%, 94%, 95%, 96%, 97%, 98% or 99% identity to SEQ ID NO: 94. In some embodiments, the spacer comprises an amino acid sequence set forth in SEQ ID NO: 94. In some embodiments, the spacer encoded by a nucleic acid sequence having at least 85%, 86%, 87%, 88%, 89%, 90%, 91%, 92%, 93%, 94%, 95%, 96%, 97%, 98% or 99% identity to SEQ ID NO: 89. In some embodiments, the spacer is encoded by a nucleic acid sequence set forth in SEQ ID NO: 89. In some embodiments, the spacer comprises an amino acid sequence having at least 85%, 86%, 87%, 88%, 89%, 90%, 91%, 92%, 93%, 94%, 95%, 96%, 97%, 98% or 99% identity to SEQ ID NO: 95. In some embodiments, the spacer comprises an amino acid sequence set forth in SEQ ID NO: 95. In some embodiments, the spacer is encoded by a nucleic acid sequence having at least 85%, 86%, 87%, 88%, 89%, 90%, 91%, 92%, 93%, 94%, 95%, 96%, 97%, 98% or 99% identity to SEQ ID NO: 90. In some embodiments, the spacer is encoded by a nucleic acid sequence set forth in SEQ ID NO: 90. In some embodiments, the spacer comprises an amino acid sequence having at least 85%, 86%, 87%, 88%, 89%, 90%, 91%, 92%, 93%, 94%, 95%, 96%, 97%, 98% or 99% identity to SEQ ID NO: 100. In some embodiments, the spacer comprises an amino acid sequence set forth in SEQ ID NO: 100.

In some embodiments, the spacer is or comprises a glycine-serine rich sequence or other flexible linker such as known flexible linkers.

In some embodiments, the spacer can be of a length that provides for increased responsiveness of the cell following antigen binding, as compared to in the absence of the spacer or as compared to an alternative spacer of a different length (e.g. longer in length). In some examples, the spacer is at or about 12 to 15 amino acids in length. In some examples, the spacer is about 110 to 130 amino acids in length. In some examples, the spacer is at or about 220 to 240 amino acids in length.

In some examples, the spacer is at or about 12 amino acids in length or is no more than at or about 12 amino acids in length. In some examples, the spacer is at or about 15 amino acids in length or is no more than at or about 15 amino acids in length.

In some embodiments, the spacer is a hinge region sequence set forth in any one of SEQ ID NOs: 10, 93-96, and 100-115. In some embodiments, the spacer is a hinge region sequence set forth in any one of SEQ ID NOs: 10, 93-96, and 100. In some embodiments, the spacer is a hinge region sequence set forth in SEQ ID NO: 10.

Exemplary spacers include, but are not limited to, those described in Hudecek et al. (2013) Clin. Cancer Res., 19:3153, Hudecek et al. (2015) Cancer Immunol. Res., 3(2):125-135, or WO2014031687.

### C. Transmembrane Domain

The CAR includes a transmembrane domain (also referred to as transmembrane region) linking the extracellular domain containing the antigen-binding domain (e.g., the CD70-binding domains) and the intracellular domain. In some embodiments, the VHH of the antigen-binding domain most proximal to the cellular membrane is linked to the transmembrane domain. In some embodiments, the transmembrane domain is fused to the extracellular domain.

In some embodiments, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. In some embodiments, the transmembrane domain can be a CD4 transmembrane. In some embodiments, the transmembrane domain is a transmembrane domain of human CD4 or variant thereof. In some embodiments, the transmembrane domain is a CD8 transmembrane domain. In some embodiments, the transmembrane domain is a transmembrane domain of human CD8 or variant thereof. In some embodiments, the transmembrane domain is a CD28 transmembrane domain. In some embodiments, the transmembrane domain is a transmembrane domain of human CD28 or variant thereof.

In some embodiments, the transmembrane domain is a CD28 transmembrane domain that comprises the sequence of amino acids set forth in SEQ ID NO: 11. In some embodiments, the transmembrane domain of the receptor is a transmembrane domain of human CD28 or variant thereof, e.g., a 2 transmembrane domain of a human CD28 (Accession No.: P10747.1). In some embodiments, the transmembrane domain is a transmembrane domain that comprises the sequence of amino acids set forth in SEQ ID NO: 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 11. In some embodiments, the transmembrane domain is a transmembrane domain that comprises the sequence of amino acids set forth in SEQ ID NO: 116 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 116.

In some embodiments, the transmembrane domain is or contains SEQ ID NO: 11 or an amino acid sequence having at least at or about 90%, 91%, 92%, 93%, 94%, 95%, 96%, 97%, 98% or 99% sequence identity to SEQ ID NO: 11. In some embodiments, the transmembrane domain is or contains the sequence set forth in SEQ ID NO: 11.

In some embodiments, the transmembrane domain of the is a transmembrane domain of a human CD8a. In some embodiments, the transmembrane domain is a transmembrane domain that comprises the sequence of amino acids set forth in SEQ ID NO: 97 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 97. In some embodiments, the transmembrane domain is encoded by the nucleotide sequence set forth in SEQ ID NO: 92 or a nucleotide sequence that exhibits at least or at least about 85%, 86%, 87%, 88%, 89%, 90%, 91%, 92%, 93%, 94%, 95%, 96%, 97%, 98%, 99% or more sequence identity to SEQ ID NO: 92.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

### D. Intracellular Signaling Domain

The receptor, e.g., the CAR, generally includes an intracellular signaling region comprising at least one intracellular signaling component or components. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

In some embodiments, upon ligation of the CAR, the cytoplasmic domain or intracellular signaling region of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some embodiments, the intracellular signaling domain is a stimulating or an activating intracellular domain portion, such as a T cell stimulating or activating domain, providing a primary activation signal or primary signal. In some embodiments, the intracellular signaling domain comprises an intracellular signaling domain capable of inducing a primary activation signal in a T cell. In some embodiments, the intracellular signaling domain is a domain from a T cell receptor (TCR) component and/or comprises an immunoreceptor tyrosine-based activation motif (ITAM). In some embodiments, the intracellular signaling domain is a cytoplasmic signaling domain of a CD3-zeta (CD3ζ) chain, for instance a human CD3ζ chain. In some embodiments, the intracellular signaling domain contains or additionally contains a costimulatory signaling domain to facilitate effector functions. In some embodiments, the intracellular signaling region further comprises a costimulatory signaling region, such as an intracellular signaling domain of a T cell costimulatory molecule or a signaling portion thereof. In some embodiments, the costimulatory signaling region is between the transmembrane region and the intracellular signaling domain. Upon specific binding to the molecule, e.g., antigen, the receptor generally delivers an immunostimulatory signal, such as an ITAM-transduced signal, into the cell, thereby promoting an immune response targeted to the disease or condition.

In some embodiments, the receptor includes an intracellular component or signaling domain of a TCR complex, such as a TCR CD3 chain that mediates T-cell activation and cytotoxicity, e.g., CD3 zeta (CD3-ζ) chain. Thus, in some aspects, the CD70-binding domain is linked to one or more cell signaling modules. In some embodiments, cell signaling modules include CD3 intracellular signaling domains and/or other CD transmembrane domains. In some embodiments, the receptor, e.g., CAR, further includes a portion of one or more additional molecules such as Fc receptor γ, CD8, CD4, CD25, or CD16.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO: 13.

In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling domain comprises the sequence of amino acids set forth in SEQ ID NO: 13 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13. In some embodiments, the CD3 zeta is or contains the sequence set forth in SEQ ID NO: 13.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some aspects, the same CAR includes both the primary (or activating) cytoplasmic signaling regions and costimulatory signaling components.

In some embodiments, the intracellular signaling regions, e.g., comprising intracellular domain or domains, include the cytoplasmic sequences of a region or domain that is involved in providing costimulatory signal. In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, CD2, CD40, CD7, CD27, GITR, and ICOS. In some embodiments, the CAR costimulatory domain is derived from immune-stimulatory receptors such as TACI, BAFF-R, or BCMA.

In some embodiments, the costimulatory molecule from 4-1BB comprises the amino acid sequence set forth in SEQ ID NO: 12. In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 117 or 118 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 117 or 118.

In some embodiments, the costimulatory molecule from 4-1BB is encoded by a polynucleotide that has been optionally optimized for codon usage and/or to reduce RNA heterogeneity, e.g., by removing cryptic splice sites.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta and 4-1BB.

### E. Exemplary CARs

In some embodiments, the CAR comprises, in order from N- to C-terminus: a CD70-binding domain, such as any CD70-binding domain described herein in Section I.A; a spacer, such as any spacer described herein in Section I.B; a transmembrane domain, such as any described herein in Section I.C; and an intracellular signaling domain, such as any described herein in Section I.D.

In some embodiments, the CAR contains an extracellular binding domain containing antibody variable chain sequences (e.g., VHH region sequences), a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an extracellular binding domain containing antibody variable chain sequences (e.g., VHH region sequences), a spacer (e.g., containing a hinge region, such as an Ig-hinge containing spacer), a transmembrane domain containing all or a portion of a CD28-derived transmembrane domain, a CD28-derived intracellular signaling domain, and a CD3 zeta signaling domain.

In some embodiments, the CAR contains an extracellular binding domain containing antibody variable chain sequences (e.g., VHH region sequences), a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. For example, in some embodiments, the CAR includes an extracellular binding domain containing antibody variable chain sequences (e.g., variable heavy chain and variable light chain sequences), a spacer (e.g., containing a hinge region, such as an Ig-hinge containing spacer), a CD28-derived transmembrane domain, a 4-1BB-derived intracellular signaling domain, and a CD3 zeta-derived signaling domain.

In some embodiments, the CAR comprises, in order from N- to C-terminus, a CD70-binding domain comprising a VHH region comprising a sequence set forth in any one of SEQ ID NO: 1-3 and 27-29; a spacer comprising a human CD28 hinge domain set forth in SEQ ID NO: 10; a transmembrane membrane comprising a human CD28 transmembrane domain set forth in SEQ ID NO: 11, and an intracellular signaling domain comprising a cytoplasmic signaling domain of a human CD3-zeta (CD3ζ) chain set forth in SEQ ID NO:13 and a costimulatory signaling region comprising 4-1BB set forth in SEQ ID NO: 12.

In some embodiments, the CAR comprises the amino acid sequence set forth in any one of SEQ ID NOs: 38-43, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in any one of SEQ ID NOs: 38-43. In some embodiments, the CAR comprises the amino acid sequence set forth in any one of SEQ ID NOs: 38-43.

In some embodiments, the CAR comprises the amino acid sequence set forth in SEQ ID NO: 38, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38, wherein X is pyroglutamate or Q. In some embodiments, the CAR comprises the amino acid sequence set forth in SEQ ID NO: 38, wherein X is pyroglutamate or Q.

In some embodiments, the CAR comprises the amino acid sequence set forth in SEQ ID NO: 39, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 39. In some embodiments, the CAR comprises the amino acid sequence set forth in SEQ ID NO: 39.

In some embodiments, the CAR comprises the amino acid sequence set forth in SEQ ID NO: 40, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 40, wherein X is pyroglutamate. In some embodiments, the CAR comprises the amino acid sequence set forth in SEQ ID NO: 40, wherein X is pyroglutamate.

In some embodiments, the CAR comprises the amino acid sequence set forth in SEQ ID NO: 41, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 41, wherein X is pyroglutamate or Q. In some embodiments, the CAR comprises the amino acid sequence set forth in SEQ ID NO: 41, wherein X is pyroglutamate or Q.

In some embodiments, the CAR comprises the amino acid sequence set forth in SEQ ID NO: 42, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 42. In some embodiments, the CAR comprises the amino acid sequence set forth in SEQ ID NO: 42.

In some embodiments, the CAR comprises the amino acid sequence set forth in SEQ ID NO: 43, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 43, wherein X is pyroglutamate. In some embodiments, the CAR comprises the amino acid sequence set forth in SEQ ID NO: 43, wherein X is pyroglutamate.

In some embodiments, the CAR further comprises a signal peptide. In some embodiments, the signal peptide is upstream of the CD70-binding domain. In some embodiments, the signal peptide comprises the amino acid sequence set forth in SEQ ID NO: 18.

In some embodiments, the CAR comprises the amino acid sequence set forth in any one of SEQ ID NOs: 14-16 and SEQ ID NO: 44-46, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in any one of SEQ ID NOs: 14-16 and SEQ ID NO: 44-46. In some embodiments, the CAR comprises the amino acid sequence set forth in any one of SEQ ID NOs: 14-16 and SEQ ID NO: 44-46.

In some embodiments, the CAR comprises the amino acid sequence set forth in SEQ ID NO: 14, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14, wherein X is pyroglutamate or Q. In some embodiments, the CAR comprises the amino acid sequence set forth in SEQ ID NO: 14, wherein X is pyroglutamate or Q.

In some embodiments, the CAR comprises the amino acid sequence set forth in SEQ ID NO: 15, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 15. In some embodiments, the CAR comprises the amino acid sequence set forth in SEQ ID NO: 15.

In some embodiments, the CAR comprises the amino acid sequence set forth in SEQ ID NO: 16, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 16, wherein X is pyroglutamate. In some embodiments, the CAR comprises the amino acid sequence set forth in SEQ ID NO: 16, wherein X is pyroglutamate.

In some embodiments, the CAR comprises the amino acid sequence set forth in SEQ ID NO: 44, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 44, wherein X is pyroglutamate or Q. In some embodiments, the CAR comprises the amino acid sequence set forth in SEQ ID NO: 44, wherein X is pyroglutamate or Q.

In some embodiments, the CAR comprises the amino acid sequence set forth in SEQ ID NO: 45, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 45. In some embodiments, the CAR comprises the amino acid sequence set forth in SEQ ID NO: 45.

In some embodiments, the CAR comprises the amino acid sequence set forth in SEQ ID NO: 46, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 46, wherein X is pyroglutamate. In some embodiments, the CAR comprises the amino acid sequence set forth in SEQ ID NO: 46, wherein X is pyroglutamate.

In some embodiments, the CAR is encoded by a nucleic acid sequence set forth in SEQ ID NO: 17, or a nucleic acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleic acid sequence set forth in SEQ ID NO: 17. In some embodiments, the CAR is encoded by a nucleic acid sequence set forth in SEQ ID NO: 17.

### F. Features of Provided CARs

Among the provided CARs are CARs that exhibit antigen-dependent activity or signaling, against CD70 target cells. In some embodiments, the provided CAR-expressing cells exhibit biological activity or function, including cytotoxic activity, cytokine production, and ability to proliferate when contacted with CD70-expressing target cells. In some embodiments, the CAR activity is measurably absent or at background levels in the absence of antigen, e.g. CD70. Thus, in some aspects, provided CARs do not exhibit, or exhibit no more than background or a tolerable or low level of, tonic signaling or antigen-independent activity or signaling in the absence of antigen, e.g. CD70, being present.

In some cases, the CD70-targeted extracellular-antigen binding domain of the CAR can be blocked or inhibited by the presence of soluble CD27, which can reduce the ability of the anti-CD70 CAR to bind CD70. In some embodiments, the binding of the CD70-extracellular-antigen binding domain of the CAR to CD27 on target cells is reduced in the presence of soluble CD27. In some embodiments, the binding of the CD70-extracellular antigen-binding domain of the CAR to CD27 on target cells in the presence of soluble CD27 is reduced no more than 40% relative to binding in the absence of soluble CD27. In some embodiments, the binding of the CD70-targeted extracellular antigen binding domain of the CAR to CD27 on target cells in the presence of soluble CD27 is reduced no more than 30% relative to binding in the absence of soluble CD27. In some embodiments, the binding of the CD70-targeted extracellular antigen binding domain of the CAR to CD27 on target cells in the presence of soluble CD27 is reduced no more than 20% relative to binding in the absence of soluble CD27. In some embodiments, the binding of the CD70-targeted extracellular antigen binding domain of the CAR to CD27 on target cells in the presence of soluble CD27 is reduced no more than 10% relative to binding in the absence of soluble CD27. In some embodiments, the binding of the CD70-targeted extracellular antigen binding domain of the CAR is not reduced or blocked or is not substantially reduced or blocked in the presence of soluble CD27.

### G. Polynucleotides Encoding the Chimeric Antigen Receptor

Also provided are polynucleotides encoding the CD70-targeted antibodies provided here. Also provided are polynucleotides encoding the chimeric antigen receptor provided herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some embodiments, the polynucleotide contains a signal sequence that encodes a signal peptide, in some cases encoded upstream of the nucleic acid sequences encoding the CD70-targeted VHH antibody, or joined at the 5′ terminus of the nucleic acid sequences encoding the CD70-targeted VHH antibody or CARs incorporating the same. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of a CD33 signal peptide set forth in SEQ ID NO: 18.

In some cases, the polynucleotide encoding the CD70-targeted VHH antibody or a CAR incorporating the same can contain nucleic acid sequence encoding additional molecules, such as a surrogate marker or other markers, or can contain additional components, such as promoters, regulatory elements and/or multicistronic elements. As used herein, the term “intron” and “non-coding element” may be used interchangeably.

In some embodiments, the polynucleotide comprises a promoter. Exemplary promoters include an EF1α promoter and a MND promoter.

In some embodiments, the promoter is a EF1α promoter. In some embodiments, the promoter comprises the nucleic acid sequence set forth in SEQ ID NO: 19, or a nucleic acid sequence having at least 85%, 86%, 87%, 88%, 89%, 90%, 91%, 92%, 93%, 94%, 95%, 96%, 97%, 98% or 99% identity to SEQ ID NO: 19. In some embodiments, the promoter comprises the nucleic acid sequence set forth in SEQ ID NO: 19.

In some embodiments, the promoter is a MND promoter. In some embodiments, the promoter comprises the nucleic acid sequence set forth in SEQ ID NO: 123, or a nucleic acid sequence having at least 85%, 86%, 87%, 88%, 89%, 90%, 91%, 92%, 93%, 94%, 95%, 96%, 97%, 98% or 99% identity to SEQ ID NO: 123. In some embodiments, the promoter comprises the nucleic acid sequence set forth in SEQ ID NO: 123.

In some embodiments, the polynucleotide further comprises anon-coding element, such as a HTLV or synthetic non-coding element.

In some embodiments, the polynucleotide further comprises a HTLV non-coding element. An exemplary HTLV non-coding element comprises a 5′ non-coding element set forth in SEQ ID NO: 124 and a 3′ non-coding element set forth in SEQ ID NO: 125. In some embodiments, the HTLV non-coding element comprises a 5′ non-coding element comprising the nucleic acid sequence set forth in SEQ ID NO: 124, or a nucleic acid sequence having at least 85%, 86%, 87%, 88%, 89%, 90%, 91%, 92%, 93%, 94%, 95%, 96%, 97%, 98% or 99% identity to SEQ ID NO: 124, and a 3′ non-coding element comprising a nucleic acid sequence set forth in SEQ ID NO: 125, or a nucleic acid sequence having at least 85%, 86%, 87%, 88%, 89%, 90%, 91%, 92%, 93%, 94%, 95%, 96%, 97%, 98% or 99% identity to SEQ ID NO: 125. In some embodiments, the polynucleotide comprises the nucleic sequence set forth in SEQ ID NO: 124, or a nucleic acid sequence having at least 85%, 86%, 87%, 88%, 89%, 90%, 91%, 92%, 93%, 94%, 95%, 96%, 97%, 98% or 99% identity to SEQ ID NO: 124. In some embodiments, the polynucleotide comprises the nucleic sequence set forth in SEQ ID NO: 124. In some embodiments, the polynucleotide comprises the sequence set forth in SEQ ID NO: 125, or a nucleic acid sequence having at least 85%, 86%, 87%, 88%, 89%, 90%, 91%, 92%, 93%, 94%, 95%, 96%, 97%, 98% or 99% identity to SEQ ID NO: 125. In some embodiments, the polynucleotide comprises the nucleic sequence set forth in SEQ ID NO: 125. In some embodiments, the polynucleotide comprises the nucleic sequence set forth in SEQ ID NO: 124 and SEQ ID NO: 125.

In some particular embodiments, the polynucleotide comprises a EF1α promoter-HTLV non-coding element comprising a nucleic acid sequence set forth in SEQ ID NO:20, or a nucleic acid sequence having at least 85%, 86%, 87%, 88%, 89%, 90%, 91%, 92%, 93%, 94%, 95%, 96%, 97%, 98% or 99% identity to SEQ ID NO: 20. In some embodiments, the polynucleotide comprises a EF1α promoter-HTLV non-coding element comprising the nucleic acid sequence set forth in SEQ ID NO: 20.

In some embodiments, the polynucleotide further comprises a synthetic non-coding element. An exemplary synthetic non-coding element is set forth in SEQ ID NO: 68. In some embodiments, the synthetic non-coding element comprises a 5′ non-coding element comprising the nucleic acid sequence set forth in SEQ ID NO: 69, or a nucleic acid sequence having at least 85%, 86%, 87%, 88%, 89%, 90%, 91%, 92%, 93%, 94%, 95%, 96%, 97%, 98% or 99% identity to SEQ ID NO: 69, and a 3′ non-coding element comprising a nucleic acid sequence set forth in SEQ ID NO: 70, or a nucleic acid sequence having at least 85%, 86%, 87%, 88%, 89%, 90%, 91%, 92%, 93%, 94%, 95%, 96%, 97%, 98% or 99% identity to SEQ ID NO: 70. In some embodiments, the polynucleotide comprises the nucleic sequence set forth in SEQ ID NO: 69, or a nucleic acid sequence having at least 85%, 86%, 87%, 88%, 89%, 90%, 91%, 92%, 93%, 94%, 95%, 96%, 97%, 98% or 99% identity to SEQ ID NO: 69. In some embodiments, the polynucleotide comprises the nucleic sequence set forth in SEQ ID NO: 69. In some embodiments, the polynucleotide comprises the sequence set forth in SEQ ID NO: 70, or a nucleic acid sequence having at least 85%, 86%, 87%, 88%, 89%, 90%, 91%, 92%, 93%, 94%, 95%, 96%, 97%, 98% or 99% identity to SEQ ID NO: 70. In some embodiments, the polynucleotide comprises the nucleic acid sequence set forth in SEQ ID NO: 70. In some embodiments, the polynucleotide comprises the nucleic acid sequence set forth in SEQ ID NO: 69 and SEQ ID NO:70.

In some embodiments, the polynucleotide comprises a synthetic promoter. In some embodiments, the synthetic promoter comprises a EF1α promoter and a synthetic non-coding element. In some embodiments, the synthetic promoter comprises a nucleic acid sequence set forth in SEQ ID NO: 19, or a nucleic acid sequence having at least 85%, 86%, 87%, 88%, 89%, 90%, 91%, 92%, 93%, 94%, 95%, 96%, 97%, 98% or 99% identity to SEQ ID NO: 19; and a nucleic acid sequence set forth in SEQ ID NO: 69, or a nucleic acid sequence having at least 85%, 86%, 87%, 88%, 89%, 90%, 91%, 92%, 93%, 94%, 95%, 96%, 97%, 98% or 99% identity to SEQ ID NO: 69. In some embodiments, the synthetic promoter comprises a nucleic acid sequence set forth in SEQ ID NO: 19; and a nucleic acid sequence set forth in SEQ ID NO: 69. In some embodiments, the polynucleotide comprising the synthetic promoter further comprises a nucleic acid sequence set forth in SEQ ID NO: 70, or a nucleic acid sequence having at least 85%, 86%, 87%, 88%, 89%, 90%, 91%, 92%, 93%, 94%, 95%, 96%, 97%, 98% or 99% identity to SEQ ID NO: 70. In some embodiments, the polynucleotide comprising the synthetic promoter further comprises a nucleic acid sequence set forth in SEQ ID NO: 70.

In some embodiments, the nucleic acid sequence encoding the CARs can be operably linked to any of the additional components.

Also provided are cells expressing the CAR encoded by the polynucleotides provided herein and uses thereof in adoptive cell therapy, such as treatment of diseases and disorders associated with CD70 expression.

Among the provided cells are T cells engineered to express a CAR encoded by a provided polynucleotide, and compositions containing such cells. In some embodiments, the cells are human cells. In some embodiments, the polynucleotide constructs are codon optimized for expression in a human cell.

### II. CAR-ENGINEERED T CELLS AND METHODS OF MAKING THE SAME

Provided herein are genetically engineered T cells that express a chimeric antigen receptor (CAR), and that also are genetically engineered to have reduced or eliminated expression an endogenous major histocompatibility complex (MHC), e.g. MHC class I or MHC class II by genetic disruption of B2M; reduced or eliminated expression of TRAC; and introduction of a transgene sequence to express a NK cell inhibiting moiety, such as an HLA-E. In some embodiments, the HLA-E is a single chain fusion protein with at least a portion of B2M to promote expression on the cell surface. In any of such embodiments, the CAR is a CD70-targeted CAR provided herein.

In some embodiments, the provided engineered T cells comprise a genetic disruption at a target site at an endogenous T cell receptor alpha constant (TRAC) locus, for example, to knock-out (KO) or reduce or eliminate the expression of the gene product of the TRAC locus. In some embodiments, the provided engineered T cells comprise a genetic disruption at a target site at Beta-2 microglobulin (B2M) locus, for example, to knock-out (KO) or reduce or eliminate the expression of the gene product of the B2M locus.

In some aspects, the provided engineered T cells comprise a genetic disruption at a target site at an endogenous cluster of differentiation 70 (CD70) locus, for example, to knock-out (KO) or reduce or eliminate the expression of the gene product of the CD70 locus.

In some embodiments, in addition to being engineered to express the CAR, the provided T cells are engineered to express one or more heterologous transgene sequences (e.g., sequences that are exogenous or heterologous to the T cell). In some embodiments, the provided T cells are engineered to express a single chain HLA-E fusion transgene. In some embodiments, the T cells are engineered with an inhibitory molecule that affects RNA interference (RNAi) against one or more of PTPN2, FAS and TGFBR2. In some embodiments, the T cells are engineered with an inhibitory molecule that affects RNA interference (RNAi) against each of PTPN2, FAS and TGFBR2.

In some aspects, the provided cells are engineered by CRISPR/Cas mediated gene editing to introduce the genetic disruption at a target site of the locus. In some aspects, the provided cells are engineered by CRISPR/Cas mediated gene editing to introduce a genetic disruption at a target site and targeted integration (targeted knock-in, KI) of transgene sequences, for example encoding the recombinant CAR, HLA-E fusion protein, or RNA inhibitory molecule, at or near one of the target sites with the genetic disruption. In some aspects, a genetic disruption is introduced at a target site at a TRAC or B2M locus, and the transgene sequences encoding a recombinant CAR, HLA-E fusion protein, and/or or RNA inhibitory molecule, are integrated into a location at or near one of the target sites with the genetic disruption, for example, by homology-directed repair (HDR). In some embodiments, the provided engineered T cells comprise a modified T cell receptor alpha constant (TRAC) locus comprising a transgene encoding the recombinant CAR. In some aspects, the transgene encoding the recombinant CAR is integrated at the TRAC locus of the T cell, by targeted knock-in (KI), and the expression of the endogenous TRAC gene product, the TCRα constant region, is reduced or eliminated. In some aspects, the transgene encoding the RNA inhibitory molecule is integrated at the TRAC locus of the T cell, by targeted knock-in (KI), and the expression of the endogenous TRAC gene product, the TCRα constant region, is reduced or eliminated. In some aspects, the transgene sequences encoding the CAR and the RNA inhibitory molecule are integrated at the TRAC locus of the T cell, by targeted knock-in (KI), and the expression of the endogenous TRAC gene product, the TCRα constant region, is reduced or eliminated. In some aspects, the provided engineered T cells also comprise a modified Beta-2 microglobulin (B2M) locus comprising a transgene encoding the recombinant HLA-E fusion protein. In some aspects, the transgene encoding the recombinant HLA-E fusion protein is integrated at the B2M locus of the T cell, by targeted knock-in (KI), and the expression of the endogenous B2M gene product is reduced or eliminated.

Exemplary methods for carrying out genetic disruptions at the endogenous loci and/or for carrying out HDR for targeted integration of the transgenes are described in this disclosure. Further, the engineered T cells can be generated using other methods, for example, as described in WO2015/161276, WO2015/070083, WO2019/070541, WO2019/195491, WO2019/195492, WO2019/089884, and WO2020/223535, the contents of which are incorporated by reference.

### A. Source T Cells

Any source of human T cells can be used for genetically engineering T cells in accord with the provided embodiments. In some embodiments, the cells are derived from the blood, bone marrow, lymph, or lymphoid organs. The cells typically are primary cells, such as those isolated directly from a subject and/or isolated from a subject and frozen. In some embodiments, the T cells are from a healthy donor.

In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, provided herein is a T cell comprising a provided CAR. In some embodiments the T cell is a CD4+ T cell. In some embodiments, the T cell is a CD8+ T cell.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

With reference to the subject to be treated, the cells may be allogeneic and/or autologous. Typically, the cells are allogeneic to the subject being treated. Among the methods include off-the-shelf methods.

In some embodiments, the methods include isolating cells from the subject, preparing, processing, culturing, and/or engineering them, as described herein, and re-introducing them into the same patient, before or after cryopreservation.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid comprising a nucleic acid encoding a recombinant receptor described herein.

In some embodiments, the donor subject is a subject that does not have a particular disease or condition or is not in need of a cell therapy or to which cell therapy will be administered. In some embodiments, the donor subject is a healthy subject or is believed to be a healthy subject (i.e. has not been diagnosed with a disease or condition).

In some embodiments, the T cells are primary T cells, such as primary human T cells. In some embodiments, the donor sample is a sample from an individual donor. In some embodiments, samples from a plurality of different individual donors are combined into a donor sample. In some aspects, the donor sample is from samples from a plurality of different individual donors. In some aspects, the donor sample is from a plurality of different donors. In some embodiments, the sample (e.g. donor sample) comprises primary human T cells from an individual donor. In some embodiments, each of the samples (e.g. donor samples) from a plurality of different individual donors are combined. In some embodiments, the sample (e.g. donor sample) comprises primary human T cells from a plurality of different donors. In some embodiments, the human donor is a healthy human donor.

In some embodiments, the samples include tissue, fluid, and other samples taken directly from the donor. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In some embodiments, the samples are from allogeneic sources (e.g. allogeneic donors). In some embodiments, the samples are from autologous sources (e.g. autologous donors). In some embodiments, the sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some examples, cells from the circulating blood of a donor are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the sample containing cells (e.g., donor sample, such as an apheresis product or a leukapheresis product) is cryopreserved and/or cryoprotected (e.g., frozen) and then thawed and optionally washed prior to any steps for isolating or selecting T cells or genetically engineering the cells.

In certain embodiments, subsets of T cells, e.g., CD3+, CD4+ or CD8+ T cells, are selected, isolated, or enriched from the donor sample or pooled donor samples. In some embodiments, CD4+ and CD8+ T cells are selected, isolated, or enriched the donor sample or pooled donor samples. In particular embodiments, CD3+ T cells are selected, isolated, or enriched the donor sample or pooled donor samples.

In some embodiments, selection, isolation, or enrichment includes one or more selection steps. The selection can be a negative selection to deplete or remove unwanted cells or can be a positive selection of desired cells. In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In certain embodiments, such surface proteins may include CD3, CD4, or CD8. In some embodiments, the selection reagent or reagents result in a separation that is affinity- or immunoaffinity-based separation.

In some embodiments, selected or isolated T cells are further enriched for naive, central memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In certain embodiments, central memory T cells may include cells in various differentiation states and may be characterized by positive or high expression (e.g., surface expression) of certain cell markers and/or negative or low expression (e.g., surface expression) of other cell markers. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571. In some aspects, central memory T cells are characterized by positive or high expression of CD45RO, CD62L, CCR7, CD28, CD3, and/or CD127. In some aspects, central memory T cells are characterized by negative or low expression of CD45RA and/or granzyme B. In certain embodiments, central memory T cells or the T cells that are surface positive for a marker expressed on central memory T cells are CCR7+CD45RA−.

In some embodiments, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. In general, the term enriching does not require complete removal of other cells, cell type, or populations from the composition and does not require that the cells so enriched be present at or even near 100% in the enriched composition.

Hence, it is understood that the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some embodiments, the selected CD4+ cell population and the selected CD8+ cell population may be combined subsequent to the selecting. In some embodiments, the T cell population has a ratio of CD4+ to CD8+ T cells of between at or about 1:5 and at or about 5:1. In some embodiments, the T cell population has a ratio of CD4+ to CD8+ T cells of between at or about 1:3 and at or about 3:1. In some embodiments, the T cell population has a ratio of CD4+ to CD8+ T cells of between at or about 1:2 and at or about 2:1.

### B. Allogeneic Cells and Engineering

In some embodiments, a population of T cells, such as T cells selected from a healthy donor or a pool of healthy donors, are genetically engineered to modify the cells to be hypoimmune to have reduced recognition by the host immune response. In some embodiments, the modifications involve genetic engineering by one or more strategies to mitigate graft versus host and host versus graft interaction as well as NK cell-mediated rejection, while preserving and in some cases enhancing T cell functions. In some embodiments, the provided engineered CAR T cell therapies are non-alloreactive so that they are not susceptible to, or exhibit reduced susceptibility compared to T cells without the genetic disruptions or modifications, to host immune system rejection. In provided embodiments, the engineered T cells, including by engineering the T cells to be non-alloreactive, permits them to be used as a cell source for an allogeneic CAR-T cell product.

### 1. Genetic Disruption

In some embodiments, one or more genetic disruption is induced at one or more target sites in the T cell. In some aspects, during the engineering of the T cell, one or more genetic disruption is induced at one or more target sites in the T cell. In some aspects, at least two genetic disruptions are induced, one at a target site at the endogenous TRAC locus and another at a target site at the endogenous B2M locus. In some aspects, at least three genetic disruptions are induced, one at a target site at the endogenous CD70 locus, another at a target site at the endogenous TRAC locus, and another at a target site at the endogenous B2M locus. In some aspects, the genetic disruptions at target sites at the endogenous TRAC or the B2M locus can be used for targeted integration of transgene sequences at or near that target site.

In some embodiments, a genetic disruption is induced at a target site of the endogenous TRAC locus. In some embodiments, the genetic disruption is induced in an exon of the endogenous TRAC locus. In some embodiments, the genetic disruption is induced in an intron of the endogenous TRAC locus. In some aspects, the presence of the genetic disruption and a polynucleotide, e.g., a template polynucleotide that contains transgene sequences encoding a recombinant CAR and/or a RNAi expression cassette, can result in targeted integration of the transgene sequences at or near the genetic disruption at the endogenous TRAC locus. In some aspects, such targeted integration produces a modified TRAC locus comprising a transgene encoding the recombinant CAR and/or an RNAi expression cassette.

In some embodiments, a genetic disruption is induced at a target site of the endogenous B2M locus. In some embodiments, the genetic disruption is induced in an exon of the endogenous B2M locus. In some embodiments, the genetic disruption is induced in an intron of the endogenous B2M locus. In some aspects, the presence of the genetic disruption and a polynucleotide, e.g., a template polynucleotide that contains transgene sequences encoding a recombinant HLA-E fusion protein, can result in targeted integration of the transgene sequences at or near the genetic disruption at the endogenous B2M locus. In some aspects, such targeted integration produces a modified B2M locus comprising a transgene encoding the recombinant HLA-E fusion protein.

In some embodiments, a genetic disruption is induced at a target site of the endogenous CD70 locus. In some embodiments, the genetic disruption is induced in an exon of the endogenous CD70 locus. In some embodiments, the genetic disruption is induced in an intron of the endogenous CD70 locus.

In some embodiments, a genetic disruption results in a DNA break or a nick. In some embodiments, at the site of the DNA break, action of cellular DNA repair mechanisms can result in a knock-out (KO), an indel, an insertion, a missense or a frameshift mutation, such as a biallelic frameshift mutation, and/or a deletion of all or part of the gene. In some embodiments, the genetic disruption can be targeted to one or more exon of a gene or portion thereof, such as within the first or second exon. In some embodiments, a DNA binding protein or DNA-binding nucleic acid, which specifically binds to or hybridizes to the sequences at a region near one of the at least one target site(s), is used for targeted disruption. In some aspects, in the absence of exogenous template polynucleotides for HDR the disruption, the targeted genetic disruption results in an indel, a deletion, a mutation and/or an insertion within an exon of the gene.

In some embodiments, polynucleotides, e.g., template polynucleotides that include a transgene encoding a recombinant CAR, RNAi expression cassette, HLA-E fusion protein, and homology sequences, can be introduced for targeted integration of the recombinant CAR-encoding transgene, RNAi expression cassette, and/or the recombinant HLA-E fusion protein-encoding transgene at or near the sites of the genetic disruptions, by HDR.

In some embodiments, the genetic disruption is carried out by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target sites, e.g., a first target site at a CD70 locus and/or a second target site at a TRAC locus and/or a third target site at a B2M locus.

In some embodiments, the genetic disruption occurs at a target site of the locus (also referred to and/or known as “target position,” “target DNA sequence,” or “target location”). In some embodiments, target site is or includes a site on a target DNA (e.g., genomic DNA) that is modified by the one or more agent(s) capable of inducing a genetic disruption, e.g., a Cas molecule complexed with a gRNA that specifies the target site. For example, in some embodiments, the target site may include locations in the DNA, e.g., at an endogenous CD70, TRAC and/or B2M loci, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

In some embodiments, genetic disruption results in a DNA break, such as a double-strand break (DSB) or a cleavage, or a nick, such as a single-strand break (SSB), at one or more target site in the genome. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., a template polynucleotide, can alter the DNA sequence based on the repair template, such as integration or insertion of the nucleic acid sequences, such as a transgene encoding all or a portion of a recombinant CAR and/or a recombinant HLA-E fusion protein and/or a RNAi expression cassette, contained in the template. In some embodiments, the genetic disruption can be targeted to one or more exon of a gene or portion thereof. In some embodiments, the genetic disruption can be targeted near a desired site of targeted integration of exogenous sequences, e.g., transgene sequences encoding a recombinant CAR and/or a recombinant HLA-E fusion protein and/or a RNAi expression cassette.

a. Target Site at an Endogenous TRAC Locus

In some aspects, the provided engineered T cells comprise a genetic disruption at the endogenous genes that encode one or more domains, regions and/or chains of the endogenous T cell receptor (TCR). In some embodiments, the genetic disruption is targeted at the endogenous gene locus that encodes TCRα. In some embodiments, the genetic disruption is targeted at the endogenous gene encoding TCRα constant domain (TRAC in humans).

In some aspects, a genetic disruption at the TRAC locus reduces expression of the gene product of the TRAC locus (e.g., endogenous TCR alpha chain constant region (Cα)) in the T cells. In some embodiments, the reduced expression of TRAC includes reduced expression of an endogenous TRAC mRNA. In some embodiments, the genetic disruption that reduces expression of TRAC includes reduced expression of an endogenous TCR alpha chain constant region (Cα) protein, the protein encoded by the TRAC mRNA. In some embodiments, the genetic disruption eliminates TRAC gene activity. In some embodiments, the genetic disruption includes inactivation or disruption of both alleles of the TRAC locus. In some embodiments, the genetic disruption includes inactivation or disruption of all alleles of the TRAC locus. In some embodiments, the genetic disruption comprises inactivation or disruption of all TRAC coding sequences in the cell. In some embodiments, the genetic disruption comprises an insertion of the transgene at the TRAC locus. In some embodiments, the genetic disruption comprises an indel at the TRAC locus. In some embodiments, the genetic disruption comprises an indel and results in a knock-out (KO) at the TRAC locus. In some embodiments, the genetically engineered T cell has reduced expression of CD3 on the cell surface. In some embodiments, the genetically engineered T cell does not express detectable CD3 on the cell surface.

In some embodiments, the endogenous TCR Cα is encoded by the TRAC gene (IMGT nomenclature). In certain embodiments, a genetic disruption is targeted at, near, or within a TRAC locus. In certain embodiments, the genetic disruption is targeted at, near, or within an open reading frame that encodes a TCRα constant domain.

In humans, an exemplary genomic locus of TRAC comprises an open reading frame that contains 4 exons and 3 introns. An exemplary mRNA transcript of TRAC can span the sequence corresponding to coordinates Chromosome 14: 22,547,506-22,552,154, on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript of an exemplary human TRAC locus.

TABLE 1
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In some embodiments, the genetic disruption is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption is targeted at early coding region of a gene of interest, e.g., TRAC, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In certain embodiments, the genetic disruption is within the first exon, second exon, third exon, or fourth exon of the TRAC gene, open reading frame, or locus. In particular embodiments, the genetic disruption is within the first exon of the TRAC gene, open reading frame, or locus. In some embodiments, the genetic disruption is within 500 base pairs (bp) downstream from the 5′ end of the first exon in the TRAC gene, open reading frame, or locus. In particular embodiments, the genetic disruption is between the most 5′ nucleotide of exon 1 and upstream of the most 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the TRAC gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRAC gene, open reading frame, or locus, each inclusive. In certain embodiments, the genetic disruption is between 100 bp and 150 bp downstream from the 5′ end of the first exon in the TRAC gene, open reading frame, or locus, inclusive.

In some aspects, the target site is within an exon, such as exons corresponding to early coding regions. In some embodiments, the target site is within or in close proximity to exons corresponding to early coding region, e.g., exon 1, 2, 3, 4 or 5 of the open reading frame of the endogenous TRAC locus (such as described in Table 1 herein), or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some aspects, the target site is at or near exon 1 of the endogenous TRAC locus, e.g., within less than 500, 450, 400, 350, 300, 250, 200, 150, 100 or 50 bp of exon 1. In some embodiments, the target site is at or near exon 2 of the endogenous TRAC locus, or within less than 500, 450, 400, 350, 300, 250, 200, 150, 100 or 50 bp of exon 2. In some aspects, the target site is at or near exon 3 of the endogenous TRAC locus, e.g., within less than 500, 450, 400, 350, 300, 250, 200, 150, 100 or 50 bp of exon 3. In some aspects, the target site is at or near exon 4 of the endogenous TRAC locus, e.g., within less than 500, 450, 400, 350, 300, 250, 200, 150, 100 or 50 bp of exon 4. In some aspects, the target site is at or near exon 5 of the endogenous TRAC locus, e.g., within less than 500, 450, 400, 350, 300, 250, 200, 150, 100 or 50 bp of exon 5. In some aspects, the target site is within a regulatory or control element, e.g., a promoter, of the TRAC locus.

In some embodiments, the genetic disruption is in a target site sequence in exon 1 of the TRAC gene. In some embodiments, the target site sequence in exon 1 of the endogenous TRAC gene is located within a TRAC genome region at contiguous positions within the hg38 genomic region chr14:22,547,506-22,547,778. In some embodiments, the target site sequence in exon 1 of the endogenous TRAC gene is located at hg38 genomic coordinates chr14:22,547,528-22,547,548. In some embodiments, the target site sequence in exon 1 of the endogenous TRAC gene has the sequence set forth in SEQ ID NO: 59, a contiguous portion thereof of at least 14 nucleotides (nt), or a complementary sequence of the foregoing. In some embodiments, the target site sequence is 14, 15, 16, 17, 18 or 20 contiguous nucleotides of SEQ ID NO:59.

In some embodiments, a target site at the TRAC locus comprises SEQ ID NO:59 (GAGTCTCTCAGCTGGTACACG).

In some embodiments, the genetic disruption is introduced by editing a genomic locus, e.g., a TRAC locus, with an RNA-guided nuclease. In some embodiments, the RNA-guided nuclease is a CRISPR/Cas nuclease. In some embodiments, the RNA-guided nuclease is Cas12a (Cpf1). In some embodiments, the CRISPR-Cas system is a ribonucleoprotein (RNP) complex comprising the Cas12a protein and the gRNA.

In some embodiments, the nuclease is an Acidaminococcus sp. Cpf1 variant (AsCpf1 variant). Any of the targeting domains can be used with a Acidaminococcus sp. Cpf1 molecule that generates a double stranded break (Cas12a nuclease) or a single-stranded break (Cas12a nickase). Cas12a molecules of, derived from, or based on the Cas12a proteins of other species listed herein can be used as well. In other words, while much of the description herein uses Acidaminococcus sp. Cas12a, Cas12a molecules from the other species can replace them, such as Lachnospiraceae bacterium or Francisella novicida.

In some embodiments, a gRNA sequence comprises CRISPR (cr)RNA and trans-activating (tra) CRISPR (cr) RNA.

In some embodiments, for genetic disruption using a CRISPR/Cas based gene editing, a gRNA sequence that is or comprises a targeting domain sequence (in some cases also referred to as a spacer sequence) that can bind to and/or target a target site in the genome, e.g., a target site at a TRAC locus. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, a guide RNA (gRNA) sequence comprises CRISPR (cr)RNA. In some embodiments, the crRNA comprises a DNA extension to improve efficacy of the Cas12a activity. In some embodiments, the DNA extension is set forth in SEQ ID NO:56.

In some embodiments, the spacer sequence is SEQ ID NO:58, or a sequence having at or at least 70%, 75%, 80%, 85%, 90%, 95%, 97%, 98%, 99%, 99.5%, or 99.9% sequence identity to SEQ ID NO:58. In some embodiments, the spacer sequence comprises a nucleic acid sequence set forth in SEQ ID NO:58, or a contiguous portion thereof of at least 5 nucleotides (nt), 6, 7, 8, 9, 10, 11, 12, 13, 14, 15, 16, 17, 18, 19, 20, 21, 22, 23, 24, or 25 nt. In some embodiments, the spacer sequence comprises a nucleic acid sequence set forth in SEQ ID NO:58, or a contiguous portion thereof of at least 14 nt. In some embodiments, the spacer sequence is SEQ ID NO:58.

In some embodiments, the target site that the targeting domain of the gRNA binds to or targets is located at an early coding region of a gene of interest, such as TRAC. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target site or a complement of the target site, such as the target nucleic acid in the TRAC locus, or the targeting domain of the gRNA can bind to or hybridize to the target site or a complement of the target site.

In some embodiments, the gRNA can target a site at the TRAC locus near a desired site of targeted integration of transgene sequences, e.g., encoding a recombinant receptor. In some aspects, the gRNA can target a site based on the amount of sequences encoding the TRAC that is desired for expression in the cell expressing the recombinant receptor. In some aspects, the gRNA can target a site within an exon of the open reading frame of the endogenous TRAC locus. In some aspects, the gRNA can target a site within an intron of the open reading frame of the TRAC locus. In some aspects, the gRNA can target a site within a regulatory or control element, e.g., a promoter, of the TRAC locus. In some aspects, the target site at the TRAC locus that is targeted by the gRNA can be any target sites described herein. In some embodiments, the gRNA can target a site within or in close proximity to exons corresponding to early coding region, e.g., exon 1, 2, 3, 4 or 5 of the open reading frame of the endogenous TRAC locus, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the endogenous TRAC locus, or within less than 500, 450, 400, 350, 300, 250, 200, 150, 100 or 50 bp of exon 2.

In some embodiments, for genetic disruption using a CRISPR/Cas based gene editing, a gRNA sequence further comprises a scaffold sequence that is responsible for Cas12a binding. In some embodiments, the scaffold sequence is SEQ ID NO:57, or a sequence having at or at least 70%, 75%, 80%, 85%, 90%, 95%, 97%, 98%, 99%, 99.5%, or 99.9% sequence identity to SEQ ID NO:57. In some embodiments, the scaffold sequence is SEQ ID NO:57.

A Cas12a molecule can interact with a gRNA molecule and, in concert with the gRNA molecule, homes or localizes to a site which comprises a target domain and PAM sequence.

In some embodiments, the Cas12a molecule interacts with a gRNA molecule. In some embodiments, the gRNA targets for disruption the TRAC target site sequence GAGTCTCTCAGCTGGTACACG (SEQ ID NO:59) at the TRAC locus in which the gRNA includes the DNA extension is set forth in SEQ ID NO:56, the spacer sequence set forth in SEQ ID NO:58, and the scaffold sequence set forth in SEQ ID NO:57 for Cas12a. In some embodiments, the gRNA includes base modifications. In some embodiments, the gRNA is modified by one or more modified nucleotides, wherein the one or more modified nucleotides are for increased stability of the gRNA. In some embodiments, the gRNA sequence is SEQ ID NO:55, or a sequence having at or at least 70%, 75%, 80%, 85%, 90%, 95%, 97%, 98%, 99%, 99.5%, or 99.9% sequence identity to SEQ ID NO:55. In some embodiments, the gRNA sequence is SEQ ID NO:55.

In some aspects, a genetic disruption at the TRAC locus is introduced using a first agent comprising a zinc finger nuclease (ZFN), a TAL-effector nuclease (TALEN), or a CRISPR-Cas combination.

b. Target Site at an Endogenous B2M Locus

In some aspects, the provided engineered T cells comprise a genetic disruption at the endogenous genes that encode one or more domains, regions and/or chains of the endogenous Beta-2 microglobulin (B2M), for example, to knock-out (KO) or reduce or eliminate the expression of the gene product of the B2M locus.

B2M is a component of the Major Histocompatibility Complex (MIHC) class I molecule, which would not assemble on the cell surface without B2M. Thus, knockout of B2M is a method of eliminating MHC class I molecules, which reduces GVHD when CAR T cells are administered to allogeneic patients.

In some aspects, a genetic disruption at the B2M locus reduces expression of the gene product of the B2M locus in the T cells. In some embodiments, the reduced expression of B2M includes reduced expression of an endogenous B2M mRNA. In some embodiments, the genetic disruption eliminates B2M gene activity. In some embodiments, the genetic disruption includes inactivation or disruption of both alleles of the B2M locus. In some embodiments, the genetic disruption includes inactivation or disruption of all alleles of the B2M locus. In some embodiments, the genetic disruption comprises inactivation or disruption of all B2M coding sequences in the cell. In some embodiments, the genetic disruption comprises an insertion of the transgene at the B2M locus. In some embodiments, the genetic disruption comprises an indel at the B2M locus. In some embodiments, the genetic disruption comprises an indel and results in a knock-out (KO) at the B2M locus. In some embodiments, the B2M indels can be detected or quantitated, among a population of engineered T cells, by PCR-based methods such as ddPCR. In some embodiments, the genetic disruption is a frameshift mutation or a deletion of a contiguous stretch of genomic DNA of the B2M gene. In some embodiments, the B2M gene is knocked out. In some embodiments, the genetically engineered T cell has reduced expression of one or more HLA class I molecules (e.g., HLA-A class I, HLA-B class I and/or HLA-C class I) on the cell surface, optionally wherein the genetically engineered cell has no detectable expression of one or more HLA class I molecules (e.g., HLA-A class I, HLA-B class I and/or HLA-C class I) on the cell surface. In some embodiments, the genetically engineered T cell has no detectable expression of HLA-A class I, HLA-B class I and HLA-C class I on the cell surface.

In some aspects, the genetically engineered T cell does not encode a functional endogenous B2M polypeptide. In some aspects, the genetically engineered T cell does not encode an endogenous B2M polypeptide. In some aspects, the genetically engineered T cell does not encode a full length endogenous B2M polypeptide. In some aspects, the expression of an endogenous B2M polypeptide is reduced or eliminated in the genetically engineered T cell.

In some embodiments, the endogenous Beta-2 microglobulin is encoded by the B2M gene (IMGT nomenclature). The sequence of an exemplary B2M gene is set forth in SEQ ID NO: 60. In certain embodiments, a genetic disruption is targeted at, near, or within a B2M locus. In certain embodiments, a genetic disruption is targeted at, near, or within a B2M locus. In particular embodiments, the genetic disruption is targeted at, near, or within an open reading frame of the B2M locus.

An exemplary mRNA transcript of B2M can span the sequence corresponding to coordinates Chromosome 15: 44,711,517-44,718,145, on the forward strand, with reference to human genome version GRCh38 (UCSC Genome Browser on Human December 2013 (GRCh38/hg38) Assembly).

In some embodiments, the genetic disruption is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption is targeted at early coding region of a gene of interest, e.g., B2M, including sequence immediately following a transcription start site, within a second exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the genetic disruption is within 500 base pairs (bp) downstream from the 5′ end of the second exon in the B2M gene, open reading frame, or locus. In particular embodiments, the genetic disruption is between the most 5′ nucleotide of exon 2 and upstream of the most 3′ nucleotide of exon 2. In certain embodiments, the genetic disruption is within 400 bp, 350 bp, 300 bp, 250 bp, 200 bp, 150 bp, 100 bp, or 50 bp downstream from the 5′ end of the second exon in the B2M gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the second exon in the B2M gene, open reading frame, or locus, each inclusive. In certain embodiments, the genetic disruption is between 100 bp and 150 bp downstream from the 5′ end of the second exon in the B2M gene, open reading frame, or locus, inclusive.

In particular embodiments, a genetic disruption is targeted at or within an intron. In certain embodiments, a genetic disruption is targeted at or within an exon. In some embodiments, a genetic disruption is targeted at or within an exon of a gene of interest, e.g., B2M locus.

In some embodiments, the genetic disruption in the endogenous B2M gene is in a target site sequence in exon 2 of the B2M gene. In some embodiments, the target site sequence in exon 2 of the endogenous B2M gene is located within a B2M genome region at contiguous positions within hg38 the genomic region 44,715,423-44,715,701. In some embodiments, the target site sequence in exon 2 of the endogenous B2M gene is located at hg38 genomic coordinates chr15:44,715,614-44,715,634.

In some embodiments, the target site sequence in exon 2 of the endogenous B2M gene has the sequence set forth in SEQ ID NO:63, a contiguous portion thereof of at least 14 nucleotides (nt), or a complementary sequence of the foregoing. In some embodiments, the target site sequence includes 14, 15, 16, 17, 18, 19, 20 or 21 contiguous nucleotides of SEQ ID NO:63.

In some embodiments, a target site at the B2M locus comprises SEQ ID NO:63 (AGTGGGGGTGAATTCAGTGTA).

In some embodiments, the genetic disruption is by editing a genomic locus, e.g., a B2M locus, with an RNA-guided nuclease. In some embodiments, the RNA-guided nuclease is a CRISPR/Cas nuclease. In some embodiments, the RNA-guided nuclease is Cas12a (Cpf1). In some embodiments, the CRISPR-Cas system is a ribonucleoprotein (RNP) complex comprising the Cas12a protein and the gRNA.

In some embodiments, the nuclease is an Acidaminococcus sp. Cpf1 variant (AsCpf1 variant). Any of the targeting domains can be used with a Acidaminococcus sp. Cpf1 molecule that generates a double stranded break (Cas12a nuclease) or a single-stranded break (Cas12a nickase). Cas12a molecules of, derived from, or based on the Cas12a proteins of other species listed herein can be used as well. In other words, while much of the description herein uses Acidaminococcus sp. Cas12a, Cas12a molecules from the other species can replace them, such as Lachnospiraceae bacterium or Francisella novicida.

In some embodiments, a guide RNA (gRNA) sequence comprises CRISPR (cr)RNA. In some embodiments, the crRNA comprises a DNA extension to improve efficacy of the Cas12a activity. In some embodiments, the DNA extension is set forth in SEQ ID NO:56.

In some embodiments, for genetic disruption using a CRISPR/Cas based gene editing, a gRNA sequence that is or comprises a targeting domain sequence (in some cases also referred to as a spacer sequence) that can bind to and/or target a target site in the genome, e.g., a target site at a B2M locus. A genome-wide gRNA database for CRISPR genome editing is publicly available, which contains exemplary guide RNA sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the spacer sequence is SEQ ID NO:62, or a sequence having at or at least 70%, 75%, 80%, 85%, 90%, 95%, 97%, 98%, 99%, 99.5%, or 99.9% sequence identity to SEQ ID NO:62. In some embodiments, the spacer sequence comprises a nucleic acid sequence set forth in SEQ ID NO:62, or a contiguous portion thereof of at least 5 nucleotides (nt) 6, 7, 8, 9, 10, 11, 12, 13, 14, 15, 16, 17, 18, 19, 20, 21, 22, 23, 24, or 25 nt. In some embodiments, the spacer sequence comprises a nucleic acid sequence set forth in SEQ ID NO:62, or a contiguous portion thereof of at least 14 nt. In some embodiments, the spacer sequence is SEQ ID NO:62.

In some embodiments, for genetic disruption using a CRISPR/Cas based gene editing, a gRNA sequence further comprises a scaffold sequence that is responsible for Cas12a binding. In some embodiments, the scaffold sequence is SEQ ID NO:57, or a sequence having at or at least 70%, 75%, 80%, 85%, 90%, 95%, 97%, 98%, 99%, 99.5%, or 99.9% sequence identity to SEQ ID NO:57. In some embodiments, the scaffold sequence is SEQ ID NO:57.

A Cas12a molecule can interact with a gRNA molecule and, in concert with the gRNA molecule, homes or localizes to a site which comprises a target domain and PAM sequence.

In some embodiments, the Cas12a molecule interacts with a gRNA molecule. In some embodiments, the gRNA targets for disruption the B2M target site sequence AGTGGGGGTGAATTCAGTGTA (SEQ ID NO:63) at the B2M locus in which the gRNA includes the DNA extension is set forth in SEQ ID NO:56, the spacer sequence set forth in SEQ ID NO:62, and the scaffold sequence set forth in SEQ ID NO:57 for Cas12a. In some embodiments, the gRNA includes base modifications. In some embodiments, the gRNA is modified by one or more modified nucleotides, wherein the one or more modified nucleotides are for increased stability of the gRNA. In some embodiments, the gRNA is SEQ ID NO:61, or a sequence having at or at least 70%, 75%, 80%, 85%, 90%, 95%, 97%, 98%, 99%, 99.5%, or 99.9% sequence identity to SEQ ID NO:61. In some embodiments, the gRNA sequence is SEQ ID NO:61.

In some aspects, a genetic disruption at the B2M locus is introduced using a first agent comprising a zinc finger nuclease (ZFN), a TAL-effector nuclease (TALEN), or a CRISPR-Cas combination.

c. Target Site at an Endogenous CD70 Locus

In some aspects, the provided engineered T cells comprise a further genetic disruption at the endogenous genes that encode one or more domains, regions and/or chains of the endogenous Cluster of Differentiation 70 (CD70).

Any of the known methods can be used to target and generate a genetic disruption of the endogenous CD70 locus can be used in the embodiments provided herein.

In some aspects, the provided engineered T cells comprise a genetic disruption at a target site at an endogenous CD70 locus, for example, to knock-out (KO) or reduce or eliminate the expression of the gene product of the CD70 locus.

In some embodiments, the genetic disruption is targeted at the CD70 locus. In some aspects, the genetic disruption is targeted at a target site within the CD70 locus containing an open reading frame encoding CD70, such that the genetic disruption occurs at or near a first target site at the CD70 locus. In some aspects, the genetic disruption is targeted at or near an exon of the open reading frame encoding CD70. In some aspects, the genetic disruption is targeted at or near an intron of the open reading frame encoding CD70.

In some aspects, a genetic disruption of the CD70 locus reduces expression of the gene product of the CD70 locus in the T cells. In some embodiments, the reduced expression of CD70 includes reduced expression of a CD70 mRNA. In some embodiments, the genetic disruption that reduces expression of CD70 includes reduced expression of CD70 protein, the protein encoded by the CD70 mRNA. In some embodiments, the genetic disruption eliminates CD70 gene activity. In some embodiments, the genetic disruption includes inactivation or disruption of both alleles of the CD70 locus. In some embodiments, the genetic disruption includes inactivation or disruption of all alleles of the CD70 locus. In some embodiments, the genetic disruption comprises inactivation or disruption of all CD70coding sequences in the cell. In some embodiments, the genetic disruption comprises insertions or deletions (indel) at the CD70 locus. In some embodiments, the genetic disruption comprises an indel and results in a knock-out (KO) at the CD70 locus. In some embodiments, the indel can be detected or quantitated, among a population of engineered T cells, by PCR-based methods such as ddPCR. In some embodiments, the genetic disruption is a frameshift mutation or a deletion of a contiguous stretch of genomic DNA of the CD70 gene. In some embodiments, the CD70 gene is knocked out. In some aspects, the genetically engineered T cell does not encode a functional CD70 polypeptide. In some aspects, the genetically engineered T cell does not encode a CD70 polypeptide. In some aspects, the genetically engineered T cell does not encode a full length CD70 polypeptide. In some aspects, the expression of CD70 polypeptide is reduced or eliminated in the genetically engineered T cell.

In some embodiments, the endogenous Cluster of Differentiation 70 is encoded by the CD70 gene (IMGT nomenclature). In certain embodiments, a genetic disruption is targeted at, near, or within a CD70 locus. In certain embodiments, a genetic disruption is targeted at, near, or within a CD70 locus. In particular embodiments, the genetic disruption is targeted at, near, or within an open reading frame of the CD70 locus.

An exemplary mRNA transcript of CD70 can span the sequence corresponding to coordinates Chromosome 16: 6,581,648-6,591,150 on the forward strand, with reference to human genome version GRCh38 (UCSC Genome Browser on Human December 2013 (GRCh38/hg38) Assembly).

CD70 is an antigen for CD70-targeting CARs. Thus, deletion of CD70 can reduce fratricide or self-targeting of CD70-targeting CARs.

In some embodiments, the genetic disruption is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption is targeted at early coding region of a gene of interest, e.g., CD70, including sequence immediately following a transcription start site, within a sixth exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the genetic disruption is within 500 base pairs (bp) downstream from the 5′ end of the second exon in the CD70 gene, open reading frame, or locus. In certain embodiments, the genetic disruption is within 400 bp, 350 bp, 300 bp, 250 bp, 200 bp, 150 bp, 100 bp, or 50 bp downstream from the 5′ end of the second exon in the PTPN2 gene, open reading frame, or locus. In particular embodiments, the genetic disruption is between 1 bp and 400 bp, between 1 and 100 bp, between 1 bp and 50 bp, or between 1 bp and 25 bp downstream from the 5′ end of the second exon in the CD70 gene, open reading frame, or locus, each inclusive. In certain embodiments, the genetic disruption is between 100 bp and 150 bp downstream from the 5′ end of the second exon in the CD70 gene, open reading frame, or locus, inclusive.

In particular embodiments, a genetic disruption is targeted at or within an intron. In certain embodiments, a genetic disruption is targeted at or within an exon. In some embodiments, a genetic disruption is targeted at or within an exon of a gene of interest, e.g., CD70 locus.

In some embodiments, the genetic disruption in the endogenous CD70 gene is in a target site sequence in exon 2 of the CD70 gene. In some embodiments, the target site sequence in exon 2 of the endogenous CD70 gene is located within a CD70 genome region at contiguous positions within hg38 genomic region chr19:6,590,103-6,590,143. In some embodiments, the target site sequence in exon 2 of the endogenous CD70 gene is located at hg38 genomic coordinates chr19:6,590,122-6,590,142.

In some embodiments, the target site sequence in exon 2 of the endogenous CD70 gene has the sequence set forth in SEQ ID NO:66, a contiguous portion thereof of at least 14 nucleotides (nt), or a complementary sequence of the foregoing. In some embodiments, the target site sequence includes 14, 15, 16, 17, 18, 19, 20 or 21 contiguous nucleotides of SEQ ID NO:66.

In some embodiments, a target site at the CD70 locus comprises SEQ ID NO:66 (TTCCAGTGGGACGTAGCTGAG).

In some embodiments, the genetic disruption is by editing a genomic locus, e.g., a CD70 locus, with an RNA-guided nuclease. In some embodiments, the RNA-guided nuclease is a CRISPR/Cas nuclease. In some embodiments, the RNA-guided nuclease is Cas12a (Cpf1). In some embodiments, the CRISPR-Cas system is a ribonucleoprotein (RNP) complex comprising the Cas12a protein and the gRNA.

In some embodiments, the nuclease is an Acidaminococcus sp. Cpf1 variant (AsCpf1 variant). Any of the targeting domains can be used with a Acidaminococcus sp. Cpf1 molecule that generates a double stranded break (Cas12a nuclease) or a single-stranded break (Cas12a nickase). Cas12a molecules of, derived from, or based on the Cas12a proteins of other species listed herein can be used as well. In other words, while much of the description herein uses Acidaminococcus sp. Cas12a, Cas12a molecules from the other species can replace them, such as Lachnospiraceae bacterium or Francisella novicida.

In some embodiments, a guide RNA (gRNA) sequence comprises CRISPR (cr)RNA. In some embodiments, the crRNA comprises a DNA extension to improve efficacy of the Cas12a activity. In some embodiments, the DNA extension is set forth in SEQ ID NO:56.

In some embodiments, for genetic disruption using a CRISPR/Cas based gene editing, a gRNA sequence that is or comprises a targeting domain sequence (in some cases also referred to as a spacer sequence) that can bind to and/or target a target site in the genome, e.g., a target site at a CD70 locus. A genome-wide gRNA database for CRISPR genome editing is publicly available, which contains exemplary guide RNA sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the spacer sequence is SEQ ID NO:65, or a sequence having at or at least 70%, 75%, 80%, 85%, 90%, 95%, 97%, 98%, 99%, 99.5%, or 99.9% sequence identity to SEQ ID NO:65. In some embodiments, the spacer sequence comprises a nucleic acid sequence set forth in SEQ ID NO:65, or a contiguous portion thereof of at least 5 nucleotides (nt), 6, 7, 8, 9, 10, 11, 12, 13, 14, 15, 16, 17, 18, 19, 20, 21, 22, 23, 24, or 25 nt. In some embodiments, the spacer sequence comprises a nucleic acid sequence set forth in SEQ ID NO:65, or a contiguous portion thereof of at least 14 nt. In some embodiments, the spacer sequence is SEQ ID NO:65.

In some embodiments, for genetic disruption using a CRISPR/Cas based gene editing, a gRNA sequence further comprises a scaffold sequence that is responsible for Cas12a binding. In some embodiments, the scaffold sequence is SEQ ID NO:57, or a sequence having at or at least 70%, 75%, 80%, 85%, 90%, 95%, 97%, 98%, 99%, 99.5%, or 99.9% sequence identity to SEQ ID NO:57. In some embodiments, the scaffold sequence is SEQ ID NO:57.

A Cas12a molecule can interact with a gRNA molecule and, in concert with the gRNA molecule, homes or localizes to a site which comprises a target domain and PAM sequence.

In some embodiments, the Cas12a molecule interacts with a gRNA molecule. In some embodiments, the gRNA targets for disruption the CD70 target site sequence at the CD70 locus in which the gRNA includes DNA extension is set forth in SEQ ID NO:56, the spacer sequence set forth in SEQ ID NO:65, and the scaffold sequence set forth in SEQ ID NO:57 for Cas12a. In some embodiments, the gRNA includes base modifications. In some embodiments, the gRNA is modified by one or more modified nucleotides, wherein the one or more modified nucleotides are for increased stability of the gRNA. In some embodiments, the gRNA is SEQ ID NO:64, or a sequence having at or at least 70%, 75%, 80%, 85%, 90%, 95%, 97%, 98%, 99%, 99.5%, or 99.9% sequence identity to SEQ ID NO:64. In some embodiments, the gRNA sequence is SEQ ID NO:64.

In some aspects, a genetic disruption at the CD70 locus is introduced using a first agent comprising a zinc finger nuclease (ZFN), a TAL-effector nuclease (TALEN), or a CRISPR-Cas combination.

d. Methods for Genetic Disruption

In some aspects, the methods for generating the genetically engineered cells involve introducing a genetic disruption at one or more target site(s), e.g., one or more target sites at a TRAC, B2M, and/or CD70 locus.

Methods for generating a genetic disruption, including those described herein, can involve the use of one or more agent(s) capable of inducing a genetic disruption, such as engineered systems to induce a genetic disruption, a cleavage and/or a double strand break (DSB) or a nick in a target site or target position in the endogenous DNA such that repair of the break by an error born process such as non-homologous end joining (NHEJ) or repair using a repair template HDR can result in the knock out of a gene and/or the insertion of a sequence of interest (e.g., exogenous nucleic acid sequences or transgene encoding a portion of a chimeric receptor) at or near the target site or position. Also provided are one or more agent(s) capable of inducing a genetic disruption, for example at one or more target sites described herein, for use in the methods provided herein. In some aspects, the one or more agent(s) can be used in combination with the template nucleotides provided herein, for homology directed repair (HDR) mediated targeted integration of the transgene sequences. Also provided are polynucleotides (e.g., nucleic acid molecules) encoding one or more components of the one or more agent(s) capable of inducing a genetic disruption.

In some aspects, the methods for generating the genetically engineered cells involve introducing a genetic disruption at a target site at a TRAC locus, a further target site at a B2M locus and/or a further target site at a CD70 locus.

In some aspects, the genetic disruptions are introduced using one or more agents comprising a zinc finger nuclease (ZFN), a TAL-effector nuclease (TALEN), or a CRISPR-Cas combination. In some aspects, the one or more agents comprise a CRISPR-Cas combination comprising a guide RNA (gRNA) comprising a targeting domain that binds to the target site, and a Cas protein. In some aspects, the one or more agents comprise a first ribonucleoprotein (RNP) complex comprising the gRNA and the Cas protein.

In some embodiments, the one or more agent(s) specifically targets the at least one target site(s), e.g., a target site at a TRAC locus, a target site at a B2M locus, and/or a target site at a B2M locus. In some embodiments, the agent comprises a ZFN, TALEN or a CRISPR/Cas combination that specifically binds to, recognizes, or hybridizes to the target site(s). In some embodiments, the CRISPR/Cas system includes an engineered crRNA/tracr RNA (“single guide RNA”) to guide specific cleavage. In some embodiments, the CRISPR/Cas system does not include an engineered tracr RNA to guide specific cleavage. In some embodiments, the agent comprises nucleases based on the Argonaute system (e.g., from T. thermophilus, known as ‘TtAgo’, (Swarts et al. (2014) Nature 507(7491): 258-261). Targeted cleavage using any of the nuclease systems described herein can be exploited to insert the sequences of a transgene, e.g., nucleic acid sequences encoding a recombinant CAR or HLA-E fusion protein or RNAi expression cassette, into a specific target location, e.g., at a TRAC or at a B2M locus, using either HDR or NHEJ-mediated processes.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the targeted genetic disruption of the endogenous genes such as TRAC, B2M and/or CD70 in humans is carried out using clustered regularly interspaced short palindromic repeats (CRISPR) and CRISPR-associated (Cas) proteins. See Sander and Joung, (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a targeting domain sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9 or Cas12a), with nuclease functionality.

In some embodiments, the one or more agent(s) comprises a guide RNA (gRNA), having a targeting domain that binds to and/or is complementary with a target site at a TRAC gene or a complement thereof. In some embodiments, the one or more agent(s) comprises a further guide RNA (gRNA) having a targeting domain that binds to and/or is complementary with a target site at a B2M gene or a complement thereof. In some embodiments, the one or more agent(s) comprises a further guide RNA (gRNA) having a targeting domain that binds to and/or is complementary with a target site at a CD70 gene or a complement thereof.

In some aspects, a “gRNA molecule” is to a nucleic acid that promotes the specific targeting or homing of a gRNA molecule/Cas molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the TRAC, B2M and/or CD70 genes in humans, to hybridize with the target sequence at the target site and direct sequence-specific binding of the CRISPR complex to the target sequence. In some embodiments, in the context of formation of a CRISPR complex, “target sequence” generally refers to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus is used to direct RNA-guided nucleases, e.g., Cas, to induce a DNA break at the target site or target position. Methods for designing gRNAs and exemplary targeting domains can include those described in, e.g., WO2015/161276, WO2017/193107, WO2017/093969, US2016/272999 and US2015/056705, the contents of which are incorporated by reference. Methods for introducing a genetic disruption at one or more target sites and gRNAs that target the target sites include those described in, e.g., WO2015/161276, WO2015/070083, WO2019/070541, WO2019/195491, WO2019/195492, WO2019/089884, and WO2020/223535, the contents of which are incorporated by reference.

Several exemplary gRNA structures, with domains indicated thereon, are described in WO2015/161276.

In some cases, the gRNA is a unimolecular or chimeric gRNA comprising, from 5′ to 3′: a targeting domain which targets a target site (e.g., at the TRAC locus, the B2M locus, and/or the PTPN2 locus); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which targets a target site e.g., at the TRAC locus, the B2M locus, and/or the CD70 locus); and a first complementarity domain. The second strand generally includes, from 5′ to 3′: optionally, a 5′ extension domain; a second complementarity domain; a proximal domain; and optionally, a tail domain.

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

### 2. Targeted Integration Via Homology-Directed Repair (HDR)

In some of the embodiments provided herein, homology-directed repair (HDR) can be utilized for targeted integration of a specific portion of the template polynucleotide containing a transgene, e.g., nucleic acid sequence encoding a recombinant CAR, a RNAi expression cassette, and/or a NK cell inhibitory moiety (e.g., recombinant HLA-E fusion protein). In some embodiments, the targeted integration is at a particular location in the genome, e.g., the TRAC locus or the B2M locus.

In some embodiments, homology-directed repair (HDR) can be utilized for targeted integration or insertion of one or more nucleic acid sequences, e.g., transgene sequences or expression cassettes, at one or more target site(s) in the genome. In some embodiments, the nuclease-induced HDR can be used to alter a target sequence, integrate a transgene at a particular target location, and/or to edit or repair a mutation in a particular target gene.

Alteration of nucleic acid sequences at the target site can occur by HDR with an exogenously provided polynucleotide (also referred to as donor polynucleotide or template sequence). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Single stranded template polynucleotides can be provided by the recombinant genomes of engineered single-stranded DNA viruses, including AAV. Template polynucleotide-effected alteration of a target sequence depends on cleavage by a nuclease, e.g., a targeted nuclease such as CRISPR/Cas. Cleavage by the nuclease can comprise a double strand break or two single strand breaks.

In some embodiments, double strand cleavage is affected by a nuclease, e.g., a Cas molecule having cleavage activity associated with an HNH-like domain and cleavage activity associated with a RuvC-like domain, e.g., an N-terminal RuvC-like domain, e.g., a wild type Cas nuclease.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

In some embodiments, DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

In some embodiments, one or more different template polynucleotides are used for targeting integration of the transgene at one or more different target sites. For targeting integration at different target sites, one or more genetic disruptions (e.g., DNA break) are generated at one or more of the target sites; and one or more different homology sequences are used for targeting integration of the transgene into the respective target site. In some embodiments, the transgene inserted at each site is the same or substantially the same. In some embodiments, transgene inserted at each site are different. In some embodiments, two or more different transgenes, encoding two or more different domains or chains of a protein, is inserted at one or more target sites.

The sequence of interest in the template polynucleotide may comprise one or more sequences encoding a functional polypeptide (e.g., a cDNA), with or without a promoter.

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Publication Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Publication No. 20130326645.

Also provided are polynucleotides (in some aspects, referred to as “template polynucleotides”, e.g., comprising transgene sequences encoding a recombinant CAR, RNAi expression cassette, and/or NK cell inhibitory moiety), as described herein. In some embodiments, the provided polynucleotides can be employed in the methods described herein, e.g., involving HDR, to target transgene sequences.

In some embodiments, the template polynucleotide is or comprises a polynucleotide containing a transgene (exogenous or heterologous nucleic acids sequences) encoding a recombinant CAR, RNAi expression cassette, recombinant CAR and RNAi expression cassette, or an NK cell inhibitory moiety (e.g., a recombinant HLA-E fusion protein or a portion thereof), and homology sequences (e.g., homology arms) that are homologous to sequences at or near the endogenous genomic site, e.g., at the endogenous TRAC locus or at the endogenous B2M locus. In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes.

In some embodiments, the template polynucleotide contains the transgene flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which resection may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

In some embodiments, the template polynucleotide is encoded on the same vector backbone, e.g. AAV genome, plasmid DNA, as the Cas12a and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas12a and gRNA. In some embodiments, the Cas12a and the gRNA are introduced in the form of a ribonucleoprotein (RNP) complex, and the template polynucleotide is introduced as a polynucleotide molecule, e.g., in a vector.

In some embodiments, a template polynucleotide comprises the following components: [5′ homology arm]-[transgene]-[3′ homology arm]. The homology arms provide for recombination into the chromosome, thus insertion of the transgene into the DNA at or near the cleavage site, e.g., target site(s). In some embodiments, the homology arms flank the most distal target site(s).

It is contemplated herein that template polynucleotides for targeted insertion may be designed for use as a single-stranded oligonucleotide, e.g., a single-stranded oligodeoxynucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

In some instances, the template polynucleotide comprises a promoter, e.g., a promoter that is exogenous and/or not present at or near the target locus, such as any promoter disclosed herein in Section I.G. In some embodiments in which the functional polypeptide encoding sequences are promoterless, expression of the integrated transgene is then ensured by transcription driven by an endogenous promoter or other control element in the region of interest.

The transgene can be inserted so that its expression is driven by the endogenous promoter at the integration site, namely the promoter that drives expression of the endogenous gene into which the transgene is inserted. For example, the coding sequences in the transgene can be inserted without a promoter, but in-frame with the coding sequence of the endogenous target gene, such that expression of the integrated transgene is controlled by the transcription of the endogenous promoter at the integration site.

In some embodiments, the polynucleotide further comprises anon-coding element, such as any non-coding element disclosed herein in Section I.G. In some embodiments, the polynucleotide further comprises a synthetic non-coding element. In some embodiments, the polynucleotide comprises the nucleic acid sequence set forth in SEQ ID NO:69 and SEQ ID NO:70.

The transgene may be inserted into an endogenous gene such that all, some or none of the endogenous gene is expressed. In some embodiments, the transgene (e.g., with or without peptide-encoding sequences) is integrated into any endogenous locus.

In some embodiments, exogenous sequences may also include transcriptional or translational regulatory sequences, for example, promoters, enhancers, insulators, internal ribosome entry sites, sequences encoding 2A peptides, introns, and/or polyadenylation signals. Further, the control elements of the genes of interest can be operably linked to reporter genes to create chimeric genes (e.g., reporter expression cassettes).

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into an endogenous TRAC locus or B2M locus.

In some embodies, the template polynucleotide comprises other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences.

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In some embodiments, the template polynucleotide is delivered to the cell via an adeno associated virus (AAV), such as any described herein.

In some embodiments, the template polynucleotide is an adenovirus vector, e.g., an adeno-associated virus (AAV) vector, e.g., a ssDNA molecule of a length and sequence that allows it to be packaged in an AAV capsid. In some embodiments, the AAV vector in an adeno-associated virus 1 (AAV6) vector. The vector may be, e.g., less than 5 kb and may contain an ITR sequence that promotes packaging into the capsid. The vector may be integration-deficient.

In some embodiments, the template polynucleotide is a lentiviral vector, e.g., an IDLV (integration deficiency lentivirus).

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

The polynucleotide may comprise any transgene of interest (exogenous sequence). Exemplary exogenous sequences include, but are not limited to any polypeptide coding sequence (e.g., cDNAs or fragments thereof), promoter sequences, enhancer sequences, epitope tags, marker genes, cleavage enzyme recognition sites and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide. In some embodiments, the transduction marker can indicate or confirm modification of a cell. In some embodiments, the surrogate marker is a protein that is made to be co-expressed on the cell surface. In particular embodiments, such a surrogate marker is a surface protein that has been modified to have little or no activity. In certain embodiments, the surrogate marker is encoded on the same polynucleotide that encodes the transgene. In some embodiments, the nucleic acid sequence encoding the transgene is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

a. CAR and RNAi Expression Cassette Transgene

In some embodiments, the T cell is engineered with a CAR and/or RNAi expression cassette. In some embodiments, the T cell is engineered with a CAR and RNAi expression cassette. In some embodiments, transgene sequences encoding the CAR and RNAi expression site are integrated into the same locus of the engineered T cells, such as by knock-in of a template polynucleotide containing both transgene sequences using HDR. In some embodiments, transgene sequences encoding the CAR and RNAi expression site are integrated into different loci of the engineered T cells, such as by separate knock-in of a template polynucleotide encoding one of the transgene sequences using HDR.

In provided embodiments, any of a variety of CARs can be engineered into a provided T cells. Exemplary antigen receptors, including CARs, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PloS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In provided embodiments, the CAR is a CD70-targeted CAR. In particular, the CAR that is engineered into the T cell include any described in Section I.

In provided embodiments, any of a variety of RNAi expression cassettes can be engineered into the T cell, such any described in Section V.

In some embodiments, the CAR or RNAi expression cassette is introduced into the T cell by targeted insertion into a genomic locus in the T cell. In some embodiments, the targeted insertion is by HDR. In some embodiments, the targeted insertion is by CRISPR/Cas-mediated HDR of a donor template comprising a polynucleotide sequence encoding the CAR, RNAi expression cassette, or CAR and RNAi expression cassette. In some embodiments, the endogenous gene loci are any of the disrupted loci as described herein. In some embodiments, the endogenous gene locus is the endogenous TRAC gene.

In some aspects, in the presence of a genetic disruption at a target site at a TRAC locus (e.g., as described in Section II.B.1), and a polynucleotide, such as the template polynucleotide having homology with sequences at or near the target site in an endogenous TRAC locus, can be used to modify the DNA in the T cell by targeted insertion (for example, a knock-in (KI)) of a transgene (e.g., a recombinant CAR and/or RNAi expression cassette). In some embodiment, the transgene is targeted at or around the TRAC locus, for example by homology-dependent repair (HDR). In some embodiments, the homology sequences of the template polynucleotide target the transgene at a TRAC locus.

In some embodiments, a polynucleotide, such as a template polynucleotide having homology with sequences at or near one or more target site(s) in the endogenous DNA can be used to alter the structure of a target DNA, e.g., targeted insertion of the transgene encoding a recombinant CAR or a portion thereof and/or a RNAi expression cassette. In some embodiments, the template polynucleotide contains homology sequences (e.g., homology arms) flanking the transgene, e.g., nucleic acid sequences encoding a recombinant CAR or a portion thereof, and/or a RNAi expression cassette for targeted insertion. In some embodiments, the homology sequences target the transgene at a TRAC locus. A person of ordinary skill in the art will recognize that methods of and compositions for inserting transgenes into the B2M locus, including aspects of the homology arms and insertion sites, are similar to and can be adapted from the disclosure for methods of and compositions for inserting transgenes into endogenous loci above in Section II.B.2.

In some embodiments, the transgene contained in the polynucleotide, e.g., template polynucleotide, comprises a sequence encoding a RNAi expression cassette. In some embodiments, the transgene can encode any of the RNAi expression cassettes described herein or any nucleic acids, e.g., shRNAs, thereof. In some embodiments, the transgene encodes a first nucleic acid complementary to an mRNA encoding TGFBR2, a second nucleic acid complementary to an mRNA encoding PTPN2, and a third nucleic acid complementary to an mRNA encoding FAS. In some embodiments, the RNAi expression cassette further encodes a fourth nucleic acid complementary to an mRNA encoding TGFBR2. In some aspects, the polynucleotide, e.g., template polynucleotide, comprises any transgene sequences provided herein or a nucleic acid sequence encoding any RNAi expression cassette described herein, e.g., in Section V.

In some embodiments, the transgene contained in the polynucleotide, e.g., template polynucleotide, comprises: (a) a sequence encoding a CAR, and (b) a sequence encoding a RNAi expression cassette. In some aspects, the transgene encodes: (a) a CAR comprising a CD70-targeting domain, and (b) a RNAi expression cassette encoding a first nucleic acid complementary to an mRNA encoding TGFBR2, a second nucleic acid complementary to an mRNA encoding PTPN2, and a third nucleic acid complementary to an mRNA encoding FAS. In some embodiments, the RNAi expression cassette further encodes a fourth nucleic acid complementary to an mRNA encoding TGFBR2. In some aspects, the polynucleotide, e.g., template polynucleotide, comprises any transgene sequences provided herein or a nucleic acid sequence encoding any CAR described herein, e.g., in Section I and a nucleic acid sequence encoding any RNAi expression cassette described herein, e.g., in Section V.

In some embodiments, the transgene encoding the CAR comprises the nucleic acid sequence set forth in SEQ ID NO: 17 or any of the CARs described in Section I.

In particular embodiments, the transgene, e.g., encoding a CAR, an RNAi expression cassette, or a CAR and a RNAi expression cassette, is targeted at a target site(s) of an endogenous locus of the T cell by homology directed repair. In certain embodiments, the polynucleotide is a template polynucleotide that includes a transgene, e.g., encoding a CAR and an RNAi expression cassette, and homology arms that are complementary to sequences of the endogenous locus. In particular embodiments, the endogenous locus of the T cell is the TRAC locus.

In some embodiments, the template polynucleotide contains homology arms for targeting the endogenous TRAC locus. In some embodiments, the genetic disruption of the TRAC locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section I.B.1 herein. In some embodiments, the template polynucleotide comprises about 500 to 1000, e.g., 600 to 900 or 700 to 800, nucleotides of homology on either side of the genetic disruption introduced by the targeted nucleases and/or gRNAs. In some embodiments, the template polynucleotide comprises about 500, 600, 700, 800, 900 or 1000 nucleotides of 5′ homology arm sequences, which is homologous to 500, 600, 700, 800, 900 or 1000 nucleotides of sequences 5′ of the genetic disruption (e.g., at TRAC locus), the transgene, and about 500, 600, 700, 800, 900 or 1000 nucleotides of 3′ homology arm sequences, which is homologous to 500, 600, 700, 800, 900 or 1000 nucleotides of sequences 3′ of the genetic disruption (e.g., at TRAC locus). In some embodiments, exemplary 5′ and 3′ homology arms for targeted integration at the TRAC locus are set forth in SEQ ID NO:30 and SEQ ID NO:31, respectively.

In some embodiments, the transgene encoding the recombinant CAR or a portion thereof and/or the RNAi expression cassette independently comprises one or more multicistronic element(s). In some embodiments, the one or more multicistronic element(s) are upstream of the transgene encoding the recombinant CAR or a portion thereof and/or the RNAi expression cassette. In some embodiments, the multicistronic element(s) is positioned between the transgene encoding the recombinant CAR or a portion thereof and the RNAi expression cassette. In some embodiments, the multicistronic element(s) is positioned between the RNAi expression cassette. In some embodiments, the nucleic acid sequence set forth in SEQ ID NO: 69 is upstream of the RNAi cassette and the nucleic acid sequence set forth in SEQ ID NO: 70 is downstream of the RNAi cassette.

In an exemplary embodiment, the template polynucleotide includes homology arms for targeting at the TRAC locus, regulatory sequences, e.g., promoter, nucleic acid sequences encoding a recombinant CAR, and nucleic acid sequences encoding a RNAi expression cassette.

In some cases, non-coding elements can lead to separation between specific nucleic regions, e.g., exons. Non-coding elements are recognized by spliceosomal RNA molecules when splicing reactions are initiated, leading to the separation of multiple RNAs, such as a RNAi expression cassette and a nucleic acid encoding a CAR. This allows multiple sequences in the inserted transgene to be controlled by the transcription under the operable control of single promoter, e.g., EF1α promoter or synthetic promoter.

In some embodiments, transgene may comprise a promoter and/or enhancer, for example a constitutive promoter or an inducible or tissue-specific promoter. In some embodiments, the promoter is or comprises a constitutive promoter. Exemplary constitutive promoters include, any described herein, such as a human elongation factor 1α promoter (EF1α). In some embodiments, the constitutive promoter is a synthetic or modified promoter. In some embodiments, the constitutive promoter is a synthetic promoter. In some embodiments, the synthetic promoter comprises a synthetic non-coding element, such as the non-coding element comprising the nucleic acid sequence set forth in SEQ ID NO: 69. In some embodiments, the transgene comprising the synthetic promoter may further comprise the sequence set forth in SEQ ID NO: 70. In some particular embodiments, the synthetic promoter comprises a EF1α promoter set forth in SEQ ID NO: 19 and a 5′ non-coding element set forth in SEQ ID NO: 69. In some particular embodiments, the transgene comprising the synthetic promoter further comprises a 3′ non-coding element set forth in SEQ ID NO: 70. In some embodiments, the promoter is a tissue-specific promoter or a viral promoter. In some embodiments, the promoter is a non-viral promoter. In some embodiments, the promoter is a EF1α promoter, for example set forth in SEQ ID NO: 19. In some embodiments, the transgene does not include a regulatory element, e.g. promoter.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different.

In some embodiments, exemplary template polynucleotides contain transgene encoding a CAR (sequence set forth in SEQ ID NO: 17), 5′ homology arm sequence of approximately 600 bp (e.g., set forth in SEQ ID NO:30), 3′ homology arm sequence of approximately 600 bp (e.g., set forth in SEQ ID NO:31) that are homologous to sequences surrounding the target integration site in exon 1 of the human TCRα constant domain (TRAC) gene. In some embodiments, the template polynucleotide further contains a transgene encoding a RNAi expression cassette (sequence set forth in SEQ ID NO: 71). In some embodiments, exemplary template polynucleotides contain transgene encoding a CAR (sequence set forth in SEQ ID NO: 17) and a RNAi expression cassette (sequence set forth in SEQ ID NO: 71), 5′ homology arm sequence of approximately 600 bp (e.g., set forth in SEQ ID NO:30), 3′ homology arm sequence of approximately 600 bp (e.g., set forth in SEQ ID NO:31) that are homologous to sequences surrounding the target integration site in exon 1 of the human TCRα constant domain (TRAC) gene.

In some embodiments, exemplary template polynucleotides contain transgene encoding a CAR (sequence set forth in SEQ ID NO: 17) and a RNAi expression cassette (sequence set forth in SEQ ID NO: 71) under the operable control of the human elongation factor 1 alpha (EF1α) promoter (sequence forth in SEQ ID NO: 17), 5′ homology arm sequence of approximately 600 bp (e.g., set forth in SEQ ID NO:30), 3′ homology arm sequence of approximately 600 bp (e.g., set forth in SEQ ID NO:31) that are homologous to sequences surrounding the target integration site in exon 1 of the human TCRα constant domain (TRAC) gene.

In some embodiments, exemplary template polynucleotides contain a transgene encoding a CAR (sequence set forth in SEQ ID NO: 17) and a RNAi expression cassette (sequence set forth in SEQ ID NO:71) under the operable control of a synthetic promoter comprising (a) an EF1α promoter (sequence forth in SEQ ID NO:17), (b) a synthetic 5′ non-coding element (sequence set forth in SEQ ID NO: 69, and (c) a synthetic 3′ non-coding element (sequence set forth in SEQ ID NO: 70), 5′ homology arm sequence of approximately 600 bp (e.g., set forth in SEQ ID NO:30), 3′ homology arm sequence of approximately 600 bp (e.g., set forth in SEQ ID NO:31) that are homologous to sequences surrounding the target integration site in exon 1 of the human TCRα constant domain (TRAC) gene.

In some embodiments, the template polynucleotide comprises the sequence set forth in SEQ ID NO:77, or a sequence that has at least at or about 90%, 91%, 92%, 93%, 94%, 95%, 96%, 97%, 98%, or 99% sequence identity to SEQ ID NO:77. In some embodiments, the nucleotide sequence of the template polynucleotide comprises the sequence set forth in SEQ ID NO:77.

In some embodiments, the RNAi expression cassette is upstream of the transgene encoding the CAR. In some embodiments, the transgene encoding the CAR is upstream of the RNAi expression cassette.

In some embodiments, the polynucleotide contains the structure: [5′ homology arm]-[transgene sequence]-[3′ homology arm]. In some embodiments, the polynucleotide contains the structure: [5′ homology arm]-[RNAi expression cassette transgene sequence]-[CAR transgene sequence]-[3′ homology arm]. In some embodiments, the polynucleotide contains the structure: [5′ homology arm]-[promoter]-[CAR transgene sequence]-[RNAi expression cassette transgene sequence]-[3′ homology arm].

Construction of such expression cassettes, following the teachings of the present specification, utilizes methodologies well known in molecular biology (see, for example, Ausubel or Maniatis).

b. NK Cell Inhibitory Moiety

In some embodiments, the T cell is genetically engineered with an NK cell inhibitory moiety that is a recombinant ligand of an NK inhibitory receptor. In some aspects, the provided engineered cells lack endogenous expression of, or have reduced expression of, a ligand for an NK inhibitory receptor, which may otherwise render the cell susceptible to NK cell-mediated cytotoxicity. For instance, as a result of complete elimination of B2M in accord with provided methods, T cells can become more vulnerable to attack by Natural Killer (NK) cells, which treat them as non-self.

In some embodiments, the NK cell inhibitory moiety is introduced into the T cell by targeted insertion into a genomic locus in the T cell. In some embodiments, the targeted insertion is by HDR. In some embodiments, the targeted insertion is by CRISPR/Cas-mediated HDR of a donor template comprising a polynucleotide sequence encoding the NK cell inhibitory moiety. In some embodiments, the endogenous gene loci are any of the disrupted loci as described herein. In some embodiments, the endogenous gene locus is the endogenous B2M gene.

In some aspects, in the presence of a genetic disruption at a target site at a B2M locus (e.g., as described in Section II.B.2), and a polynucleotide, such as the template polynucleotide having homology with sequences at or near the target site in an endogenous B2M locus, can be used to modify the DNA in the T cell by targeted insertion (for example, a knock-in (KI)) of a transgene (e.g., encoding a recombinant HLA-E fusion protein). In some embodiments, the targeted insertion is at or around the B2M locus, for example by homology-dependent repair (HDR). In some embodiments, the homology sequences of the template polynucleotide target the transgene at a B2M locus.

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences (e.g., encoding a recombinant HLA-E fusion protein or a portion thereof). In some aspects, the target site is within an exon of the open reading frame of the B2M locus. In some aspects, the target site is within an intron of the open reading frame of the B2M locus.

In some embodiments, the recombinant NK cell modulator includes a ligand or binding portion of a ligand capable of binding to an NK cell inhibitory receptor CD94/NKG2A, LIR-1/ILT2, KIR2DL4, LIR-2/ILT4 or SIRPα.

In some embodiments, the NK cell inhibitory moiety is an MHC-E (or HLA-E).

In some embodiments, the NK cell inhibitory moiety includes an HLA-E. The sequence of an exemplary HLA-E is set forth in SEQ ID NO:34 (the nucleic acid sequence encoding the exemplary HLA-E is set forth in SEQ ID NO: 35, which corresponds to nucleotides 1020-2521 of SEQ ID NO:78). Expression of HLA-E on the surface of cells can be recognized by inhibitory receptor on NK cells to modulate NK cell activation. In some cases, the binding of peptides (e.g. nonameric peptides), typically derived from signal peptides of classical MHC class I molecules, can stabilize expression of HLA-E on the surface of cells. Typically, like the classical MHC molecules, HLA-E are expressed as a heterodimer containing an α heavy chain and a light chain (also called β2 microglobulin). Thus, stable expression of HLA-E NK cell inhibitory receptors typically also require expression of B2M. In some cases, the complex of NKG2A and CD94 is involved in the recognition of HLA-E and its peptide (e.g. derived from a leader sequence of another peptide), which can mediate an inhibitory signal by the NK cell.

In some embodiments, an HLA-E chain can be introduced into the cells, such as by using an expression vector. In some embodiments, the cell also expresses a β2 microglobulin (β2M) or a component or functional fragment thereof. In some cases, at least a portion of the β2M enhances proper MHC folding and expression on the cell surface, including proper folding and expression of HLA-E. An exemplary sequence of β2M is set forth in SEQ ID NO: 37. In some embodiments, the β2M is covalently associated with the HLA-E. In some embodiments, the β2M is expressed as a hybrid or fusion molecule with HLA-E. In some embodiments, a single HLA-E chain and β2-microglobulin can be introduced into cell as a fusion protein. In some embodiments, expression of a recombinant HLA-E molecule on the surface of the cell can be stabilized by the addition of a binding peptide. In some embodiments, the binding peptide comprises a nonameric peptide.

Single chain fusion molecules of MHC proteins are known and described in the art (see e.g. published U.S. Pat. Appl. No. US20050196404. In some embodiments, the single chain fusion HLA-E protein comprises β2M or a functional portion thereof covalently linked to the mature α chain HLA-E or functional portion thereof. In some embodiments, the α chain HLA-E can include a transmembrane domain for cell surface expression of the fusion molecule. In some embodiments, the transmembrane domain is the native transmembrane domain of the α chain of the HLA-E. In some embodiments, the single chain fusion can further include an HLA-E binding peptide sufficient to stabilize expression of the HLA-E molecule on the surface. For example, in some embodiment, for stable expression of HLA-E, the binding peptide is a leader sequence of another MHC class I molecule, such as a classical MHC class I molecule, as described or known in the art.

In some embodiments, the binding peptide is a portion of a signal sequence from an MHC class I molecule. In some embodiments, the binding peptide is VMAPRTLVL (SEQ ID NO:48), VMAPRTLLL (SEQ ID NO:49), VMAPRTVLL (SEQ ID NO:50), VMAPRTLFL (SEQ ID NO:51), or VMAPRTLIL (SEQ ID NO:52). In some embodiments, the binding peptide is VMAPRTLVL (SEQ ID NO:48).

In some embodiments, the single chain MHC fusion molecule includes one or more linkers joining the components of the fusion molecule. In some embodiments, the fusion comprises one or more linkers between the binding peptide and B2M, the B2M and class I (e.g. HLA-E) α chain and/or between the binding peptide and class I α chain. In some embodiments, the fusion molecule is constructed to contain in order: HLA-E binding peptide, linker 1, B2M, linker 2 and HLA-E α chain. The linker typically is a peptide linker, e.g., a flexible and/or soluble peptide linker. Among the linkers are those rich in glycine and serine and/or in some cases threonine. In some embodiments, the linker comprises 10 to 20 residues, such as at least or about 10, 15, or 20 residues. In some embodiments, one or more linkers is (G₄S)₃₋₄ (SEQ ID NO: 119). In some embodiments, the linker is (G₄S)₂₋₃ (SEQ ID NO: 120) or GGGAS(G₄S)₂ (SEQ ID NO: 121). In some embodiments, the encoding nucleic acid molecule of the HLA-E fusion protein can include an N-terminal signal sequence for entry into the ER is required. In some embodiments, the signal sequence of B2M is normally used. In some embodiments, the N-terminal signal sequence for entry is the amino acid sequence set forth in SEQ ID NO: 47.

In some embodiments, the MHC molecule is a single chain trimer (SCT). In some embodiments, the SCT comprises a single polypeptide comprising an antigenic peptide followed by a first flexible linker that connects the C terminus of the peptide to the N terminus of a B2M, and a second flexible linker that connects the C terminus of the B2M with the N terminus of a heavy chain of an HLA-E molecule. In some embodiments, the linker comprises a cysteine, which can form a disulfide bond with a cysteine on the HLA-E heavy chain, including a disulfide trap SCT (dtSCT). For example, in some embodiments the linker between the peptide and the B2M comprises the sequence GCGASGGGGSGGGGS (SEQ ID NO: 122). Examples of SCT molecules are known in the art, including, for example, as described in US20050196404.

In some embodiments, the covalently linked peptide epitope is cleaved via a built-in protease cleavage site, and the cleaved peptide epitope can bind to the peptide binding site of the single chain protein for stabilization of the molecule.

In some embodiments, the transgene encoding the HLA-E fusion protein comprises a nucleotide sequence recited in SEQ ID NO:35 or a sequence that has at least at or about 90%, 91%, 92%, 93%, 94%, 95%, 96%, 97%, 98%, or 99% sequence identity to SEQ ID NO:35. In some embodiments, the nucleotide sequence of the transgene is recited in SEQ ID NO:35.

In some embodiments, a polynucleotide, such as a template polynucleotide having homology with sequences at or near one or more target site(s) in the endogenous DNA can be used to alter the structure of a target DNA, e.g., targeted insertion of the transgene encoding an NK cell inhibitory moiety, such as a recombinant HLA-E fusion protein or a portion thereof. In some embodiments, the template polynucleotide contains homology sequences (e.g., homology arms) flanking the transgene, e.g., nucleic acid sequences encoding a recombinant HLA-E fusion protein or a portion thereof, for targeted insertion. In some embodiments, the homology sequences target the transgene at a B2M locus. A person of ordinary skill in the art will recognize that methods of and compositions for inserting transgenes into the B2M locus, including aspects of the homology arms and insertion sites, are similar to and can be adapted from the disclosure for methods of and compositions for inserting transgenes into endogenous loci above in Section II.B.2.

In some embodiments, the transgene contained in the polynucleotide, e.g., template polynucleotide, comprises a sequence encoding a recombinant HLA-E fusion protein or a portion thereof. In some embodiments, the transgene can encode any of the recombinant HLA-E molecules described herein. In some aspects, the polynucleotide, e.g., template polynucleotide, comprises any transgene sequences provided herein or a nucleic acid sequence encoding any recombinant HLA-E described herein.

In certain embodiments, the polynucleotide, e.g., template polynucleotide contains and/or includes a transgene encoding all or a portion of a recombinant HLA-E fusion protein. In particular embodiments, the transgene is targeted at a target site(s) that is within a gene, locus, or open reading frame that encodes an endogenous receptor, e.g., an endogenous gene encoding one or more regions of a HLA-E fusion protein.

In some embodiments, the template polynucleotide contains homology arms for targeting the endogenous B2M locus. In some embodiments, the genetic disruption of the B2M locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section II.B herein. In some embodiments, the template polynucleotide comprises about 500 to 1000, e.g., 600 to 900 or 700 to 800, nucleotides of homology on either side of the genetic disruption introduced by the targeted nucleases and/or gRNAs. In some embodiments, the template polynucleotide comprises about 500, 600, 700, 800, 900 or 1000 nucleotides of 5′ homology arm sequences, which is homologous to 500, 600, 700, 800, 900 or 1000 nucleotides of sequences 5′ of the genetic disruption (e.g., at B2M locus), the transgene, and about 500, 600, 700, 800, 900 or 1000 nucleotides of 3′ homology arm sequences, which is homologous to 500, 600, 700, 800, 900 or 1000 nucleotides of sequences 3′ of the genetic disruption (e.g., at B2M locus). In some embodiments, exemplary 5′ and 3′ homology arms for targeted integration at the B2M locus are set forth in SEQ ID NO:32 and SEQ ID NO:33, respectively.

The transgene, including the transgene encoding the recombinant HLA-E fusion protein or a portion thereof, can be inserted so that its expression is driven by the endogenous promoter at the integration site, namely the promoter that drives expression of the endogenous gene into which the transgene is inserted (e.g., B2M). For example, the coding sequences in the transgene can be inserted without a promoter, but in-frame with the coding sequence of the endogenous target gene, such that expression of the integrated transgene is controlled by the transcription of the endogenous promoter at the integration site. In some embodiments, the transgene encoding the recombinant HLA-E fusion protein or a portion thereof and/or the one or more further transgene independently is operably linked to the endogenous promoter of the gene at the target site. In some embodiments, a ribosome skipping element/self-cleavage element, such as a 2A element, is placed upstream of the transgene coding sequence, such that the ribosome skipping element/self-cleavage element is placed in-frame with the endogenous gene, such that the expression of the transgene encoding the recombinant or a portion thereof and/or the one or more further transgene is operably linked to the endogenous promoter. In some embodiments, the ribosome skipping element/self-cleavage element (e.g., 2A element) comprises the amino acid sequence set forth in any one of SEQ ID NOs: 21-26. In some embodiments, the ribosome skipping element/self-cleavage element (e.g., 2A element) comprises the amino acid sequence set forth in SEQ ID NO: 25.

In some embodiments, exemplary template polynucleotides contain a transgene encoding a HLA-E fusion protein (sequence set forth in SEQ ID NO:34), 5′ homology arm sequence of approximately 800 bp (e.g., set forth in SEQ ID NO:32), 3′ homology arm sequence of approximately 800 bp (e.g., set forth in SEQ ID NO:33) that are homologous to sequences surrounding the target integration site of the human B2M gene. In some embodiments, the template polynucleotide further contains other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences.

In some embodiments, the template polynucleotide comprises the sequence set forth in SEQ ID NO:78, or a sequence that has at least at or about 90%, 91%, 92%, 93%, 94%, 95%, 96%, 97%, 98%, or 99% sequence identity to SEQ ID NO:78. In some embodiments, the nucleotide sequence of the template polynucleotide comprises the sequence set forth in SEQ ID NO:78.

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In some embodiments, the template polynucleotide is delivered to the cell via an adeno associated virus (AAV), such as any described herein.

In some embodiments, the template polynucleotide is comprised in a viral vector.

In some embodiments, the template polynucleotide is an adenovirus vector, e.g., an AAV vector. In some embodiments, the AAV vector is an AAV6 vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient.

In some embodiments, the template polynucleotide is a lentiviral vector, e.g., an IDLV (integration deficiency lentivirus).

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant HLA-E fusion protein. In some embodiments, the transduction marker can indicate or confirm modification of a cell. In some embodiments, the surrogate marker is a protein that is made to be co-expressed on the cell surface with the recombinant HLA-E fusion protein. In particular embodiments, such a surrogate marker is a surface protein that has been modified to have little or no activity.

In some embodiments, the polynucleotide contains the structure: [5′ homology arm]-[transgene sequence]-[3′ homology arm]. In some embodiments, the polynucleotide contains the structure: [5′ homology arm]-[multicistronic element]-[transgene sequence]-[3′ homology arm]. In some embodiments, the polynucleotide contains the structure: [5′ homology arm]-[promoter]-[transgene sequence]-[3′ homology arm].

Construction of such expression cassettes, following the teachings of the present specification, utilizes methodologies well known in molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

### 3. Delivery of Agents for Genetic Disruption and Template Polynucleotides

In some embodiments, the genetic disruption, such as a genetic disruption at an endogenous TRAC, B2M, and/or CD70 locus is carried out by delivering or introducing one or more agent(s) capable of inducing a genetic disruption, e.g., Cas12a and/or gRNA components, to a cell, using any of a number of known delivery method or vehicle for introduction or transfer to cells. Such delivery methods include, for example, using viral delivery vectors, or any of the known methods or vehicles for delivering Cas molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas enzyme can be delivered into the cell.

In some embodiments, the one or more agent(s) capable of inducing a genetic disruption, e.g., one or more agent(s) that is a Cas12a/gRNA, is introduced into the cell as a ribonucleoprotein (RNP) complex. RNP complexes include a sequence of ribonucleotides, such as an RNA or a gRNA molecule, and a protein, such as a Cas12a protein, or variant thereof. For example, the Cas protein is delivered as RNP complex that comprises a Cas protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells. In some embodiments, the one or more transgenes are delivered by AAV and the RNPs are delivered by electroporation.

Agent(s) and components capable of inducing a genetic disruption, e.g., a Cas12a molecule and gRNA molecule, can be introduced into target cells in a variety of forms using a variety of delivery methods and formulations, as set forth in Table 2 and Table 3, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas protein or gRNA component is encoded as DNA for delivery, the DNA may typically but not necessarily include a control region, e.g., comprising a promoter, to effect expression. Exemplary promoters for Cas12a molecule sequences include, e.g., CMV, EF1α, EFS, MSCV, PGK, or CAG promoters. Useful promoters for gRNAs include, e.g., H1, EF-1α, tRNA or U6 promoters. Promoters with similar or dissimilar strengths can be selected to tune the expression of components. Sequences encoding a Cas molecule may comprise a nuclear localization signal (NLS), e.g., an SV40 NLS. In some embodiments a promoter for a Cas molecule or a gRNA molecule may be, independently, inducible, tissue specific, or cell specific. In some embodiments, an agent capable of inducing a genetic disruption is introduced RNP complexes.

TABLE 2
Exemplary Delivery Methods

| Elements | | |
|---|---|---|
| Cas | gRNA | |
| Molecule(s) | molecule(s) | Comments |
| DNA | DNA | In this embodiment, a Cas molecule and a gRNA are |
| | | transcribed from DNA. In this embodiment, they are encoded on |
| | | separate molecules. |
| DNA | | In this embodiment, a Cas molecule and a gRNA are |
| | | transcribed from DNA, here from a single molecule. |
| DNA | RNA | In this embodiment, a Cas molecule is transcribed from DNA, |
| | | and a gRNA is provided as in vitro transcribed or synthesized |
| | | RNA |
| mRNA | RNA | In this embodiment, a Cas molecule is translated from in vitro |
| | | transcribed mRNA, and a gRNA is provided as in vitro |
| | | transcribed or synthesized RNA. |
| mRNA | DNA | In this embodiment, a Cas molecule is translated from in vitro |
| | | transcribed mRNA, and a gRNA is transcribed from DNA. |
| Protein | DNA | In this embodiment, a Cas molecule is provided as a protein, |
| | | and a gRNA is transcribed from DNA. |
| Protein | RNA | In this embodiment, a Cas molecule is provided as a protein, |
| | | and a gRNA is provided as transcribed or synthesized RNA. |

TABLE 3
Comparison of Exemplary Delivery Methods

| | | Delivery | | | |
|---|---|---|---|---|---|
| | | into Non- | Duration | | Type of |
| | | Dividing | of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non- | Cationic | YES | Transient | Depends | Nucleic |
| Viral | Liposomes | | | on what is | Acids and |
| | | | | delivered | Proteins |
| | Polymeric | YES | Transient | Depends | Nucleic |
| | Nanoparticles | | | on what is | Acids and |
| | | | | delivered | Proteins |
| | Engineered | YES | Transient | NO | Nucleic |
| | Bacteriophages | | | | Acids |

In some embodiments, DNA encoding Cas molecules and/or gRNA molecules, or RNP complexes comprising a Cas molecule and/or gRNA molecules, can be delivered into cells by known methods or as described herein. For example, Cas12a-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (such as described in Lee, et al. (2012) Nano Lett 12: 6322-27, Kollmannsperger et al (2016) Nat Comm 7, 10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas- and/or gRNA-encoding DNA or RNP complex, or RNA encoding Cas molecules and/or gRNA molecules in a cartridge, chamber or cuvette and applying one or more electrical impulses of defined duration and amplitude. In some embodiments, delivery via electroporation is performed using a system in which cells are mixed with the Cas- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., lipid nanoparticles (LNP), SNALP liposomes that contain cationic lipids together with neutral helper lipids which are coated with polyethylene glycol (PEG), and protamine-nucleic acid complexes coated with lipid. Exemplary lipids and polymers for gene transfer include those described in, for example, WO 2019/195492 and WO 2020/223535.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides. In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, RNA encoding Cas molecules and/or gRNA molecules, can be delivered into cells, e.g., target cells described herein, by known methods or as described herein. For example, Cas-encoding and/or gRNA-encoding RNA can be delivered, e.g., by microinjection, electroporation, transient cell compression or squeezing (such as described in Lee, et al. (2012) Nano Lett 12: 6322-27), lipid-mediated transfection, peptide-mediated delivery, e.g., cell-penetrating peptides, or a combination thereof.

In some embodiments, Cas molecules can be delivered into cells by known methods or as described herein. For example, Cas protein molecules can be delivered, e.g., by microinjection, electroporation, transient cell compression or squeezing (such as described in Lee, et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, the one or more agent(s) capable of introducing a cleavage, e.g., a Cas/gRNA system, is introduced into the cell as a ribonucleoprotein (RNP) complex. RNP complexes include a sequence of ribonucleotides, such as an RNA or a gRNA molecule, and a protein, such as a Cas protein or variant thereof. For example, the Cas protein is delivered as RNP complex that comprises a Cas protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing.

In some embodiments, delivery via electroporation comprises mixing the cells with the Cas molecules with or without gRNA molecules in a cartridge, chamber or cuvette and applying one or more electrical impulses of defined duration and amplitude. In some embodiments, delivery via electroporation is performed using a system in which cells are mixed with the Cas molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, delivery via electroporation comprises mixing the cells with the Cas molecules with or without gRNA molecules in a cartridge, chamber or cuvette and applying one or more electrical impulses of defined duration and amplitude. In some embodiments, delivery via electroporation is performed using a system in which cells are mixed with the Cas molecules.

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of three or more locations in the genome, e.g., a target site at a TRAC locus, a target site at a B2M locus, and a target site at a CD70 locus are delivered to the cell. In some embodiments, agent(s) and components thereof are delivered using one method. For example, in some embodiments, one or more agents, for example, for inducing a genetic disruption at a target site at a TRAC locus, a further genetic disruption at a target site at a B2M locus, and a further genetic disruption at a target site at a CD70 locus are delivered as a first agent, e.g., a first RNP, and a second agent, e.g., a second RNP, and a third agent, e.g., a third RNP, respectively. In some aspects, the three or more different RNP complexes, such as an RNP targeting a target site at a TRAC locus, a further RNP targeting a target site at a B2M locus, and a further RNP targeting a target site at a CD70 locus are delivered together, such as electroporated together, for example, in one electroporation reaction.

In some embodiments, one or more polynucleotides other than the one or more agent(s) capable of inducing a genetic disruption and/or component thereof, e.g., one or more CRISPR-Cas combinations, such as a template polynucleotide for HDR-directed integration (such as any template polynucleotide described herein, e.g., in Section II.B.2), are delivered. In some embodiments, the polynucleotide, e.g., template polynucleotide, is delivered at the same time as one or more of the components of the Cas system. In some embodiments, the polynucleotide is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the polynucleotide, e.g., template polynucleotide, is delivered by a different means from one or more of the components of the Cas system, e.g., the Cas12a molecule component and/or the gRNA molecule component. The polynucleotide, e.g., template polynucleotide, can be delivered by any of the delivery methods described herein. For example, the polynucleotide, e.g., template polynucleotide, can be delivered by a viral vector, e.g., any described herein such as an AAV vector, and the Cas component and/or the gRNA molecule component can be delivered by electroporation. In some embodiments, the polynucleotide, e.g., template polynucleotide, includes one or more exogenous sequences, e.g., transgene sequences that encode a recombinant CAR, a RNAi expression cassette, a recombinant HLA-E fusion protein and/or a portion thereof and/or other exogenous gene nucleic acid sequences.

In some embodiments, the polynucleotide, e.g., a polynucleotide such as a template polynucleotide encoding the recombinant CAR and/or RNAi expression cassette, are introduced into the cells in nucleotide form, e.g., as a polynucleotide or a vector. In particular embodiments, the polynucleotide contains a transgene that encodes the recombinant CAR and the RNAi expression cassette.

In some embodiments, the polynucleotide, e.g., a polynucleotide such as a template polynucleotide encoding the recombinant HLA-E fusion protein, are introduced into the cells in nucleotide form, e.g., as a polynucleotide or a vector. In particular embodiments, the polynucleotide contains a transgene that encodes the recombinant HLA-E fusion protein or a portion thereof.

In some embodiments, the polynucleotide, e.g., template polynucleotide, is introduced into the cell for engineering, in addition to the agent(s) capable of inducing a targeted genetic disruption, e.g., nuclease and/or gRNAs. In some embodiments, the polynucleotide(s) may be delivered prior to, simultaneously or after the agent(s) capable of inducing a targeted genetic disruption is introduced into a cell. In some embodiments, the polynucleotide(s) are delivered simultaneously with the agents. In some embodiments, the polynucleotides are delivered prior to the agents, for example, seconds to hours to days before the agents, including, but not limited to, 1 to 60 minutes (or any time therebetween) before the agents, 1 to 24 hours (or any time therebetween) before the agents or more than 24 hours before the agents. In some embodiments, the polynucleotides are delivered after the agents, seconds to hours to days after the agents, including immediately after delivery of the agent, e.g., between or between about between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the polynucleotide is delivered more than 4 hours after delivery of the agents. In some embodiments, the polynucleotides are delivered after the agents, for example, including, but not limited to, within 1 second to 60 minutes (or any time therebetween) after the agents, 1 to 4 hours (or any time therebetween) after the agents or more than 4 hours after the agents.

In some embodiments, the polynucleotide sequence can be comprised in a vector molecule containing sequences that are not homologous to the region of interest in the genomic DNA.

In some embodiments, the polynucleotides and sequences encoding the one or more agents may be on the same vector, for example an AAV vector. In some embodiments, the polynucleotides are delivered using an AAV vector and the one or more agents for inducing a genetic disruption, e.g., one or more CRISPR-Cas combination, are delivered as a different form, e.g., as mRNAs encoding the nucleases and/or gRNAs. In some embodiments, the polynucleotides and nucleases are delivered using the same type of method, e.g., a viral vector, but on separate vectors. In some embodiments, the polynucleotides are delivered in a different delivery system as the agents capable of inducing a genetic disruption, e.g., nucleases and/or gRNAs. In some embodiments, the polynucleotide is excised from a vector backbone in vivo, e.g., it is flanked by gRNA recognition sequences. In some embodiments, the polynucleotide is on a separate polynucleotide molecule as the Cas and gRNA. In some embodiments, the Cas and the gRNA are introduced in the form of a ribonucleoprotein (RNP) complex, and the polynucleotide is introduced as a polynucleotide molecule, e.g., in a vector or a linear polynucleotide, e.g., linear DNA.

The vector or construct can contain any of the polynucleotides described herein, including those described in Section I.G or Section II.B.2.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

### 4. Incubation, Cultivation and Harvesting

In some embodiments, the provided methods involve incubating the immune cells, e.g., T cells. In some embodiments, the incubating is carried out after the introducing of the one or more gene-editing agents.

In some embodiments, the incubating can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to stimulate the immune cells.

In some embodiments, the incubation is carried out until a threshold number of population doublings has occurred. In some embodiments, the incubation under conditions for cultivating the cells is carried out until 2, 3, 4, 5, 6, 7, 8, 9, 10 or more population doublings have occurred. In some embodiments, the incubation under conditions for cultivating the cells is carried out until 2 to 8 population doublings has occurred. In some embodiments, the incubation under conditions for cultivating the cells is carried out until 4 to 8 population doublings has occurred. In some embodiments, the incubation under conditions for cultivating the cells is carried out until 5, 6, or 7 population doublings has occurred.

In some embodiments, the incubation is carried out until a threshold number of cells are produced. In some embodiments, the incubation under conditions for cultivating the cells is carried out until 1×10¹⁰ to 4.0×10¹¹ cells are produced. In some embodiments, the incubation under conditions for cultivating the cells is carried out until 9.6×10¹⁰ to 3.2×10¹¹ cells are produced. In some embodiments, the incubation under conditions for cultivating the cells is carried out until 1×10¹¹ to 3.5×10¹¹ cells are produced. In some embodiments, the incubation under conditions for cultivating the cells is carried out until 2.0×10¹¹ to 3.0×10¹¹ cells are produced.

In some embodiments, the provided methods involve harvesting the genetically engineered immune cells, e.g., T cells, expressing the recombinant protein. In some embodiments, the harvesting is carried out following the engineering. In some embodiments, the harvesting is performed carried out following the cultivating.

In some embodiments, one or more polishing step of the harvested cells can be carried out. In some embodiments, the polishing step is carried out to enrich for successfully engineered cells by removing or depleting cells that have not been engineered. In some embodiments, polishing of the engineered cells is carried out by depleting CD3+ T cells to remove T cells that have not been edited by disruption of the TRAC locus. In some embodiments, a selection step (e.g., polishing step) is useful for increasing product control and/or decreasing between engineering process variance. In some embodiments, the engineered T cells are depleted for CD3+ T cells, such as by immunoaffinity-based selection for CD3+ T cells. In some embodiments, the immunoaffinity-based selection involves contacting the cells with an antibody or antigen-binding fragment thereof directed against the target antigen (e.g., CD3). In certain embodiments, the immunoaffinity reagents are immobilized on the outside surface of a bead. In particular embodiments, the bead is a magnetic bead. In some embodiments, the depleting cells bound to the immunoaffinity reagent involves exposing the cells to a magnetic field, in which the cells not bound to the magnet are recovered as CD3+ depleted cells and harvested. In some embodiments, the affinity-based selection is via magnetic-activated cell sorting (MACS) (Miltenyi Biotech, Auburn, CA). Magnetic Activated Cell Sorting (MACS), e.g., CliniMACS systems are capable of high-purity selection of cells having magnetized particles attached thereto.

In some embodiments, the provided methods involve formulating the harvested genetically engineered immune cells, e.g., T cells. In some embodiments, the harvested genetically engineered immune cells, e.g., T cells, are formulated in a container, such as a bag or vial.

In some embodiments, the harvested genetically engineered immune cells, e.g., T cells, are formulated for administration to a subject. In some embodiments, the harvested genetically engineered immune cells, e.g., T cells, are formulated in a pharmaceutically acceptable buffer, which may, in some aspects, include a pharmaceutically acceptable carrier or excipient. In some embodiments, the harvested genetically engineered immune cells, e.g., T cells, are formulated in the presence of a pharmaceutically acceptable excipient.

In some embodiments, the harvested genetically engineered immune cells, e.g., T cells, are formulated for cryopreservation. In some embodiments, the harvested genetically engineered immune cells, e.g., T cells, are formulated in the presence of a cryoprotectant. In some embodiments, the harvested genetically engineered immune cells, e.g., T cells, are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the harvested genetically engineered immune cells, e.g., T cells, are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the harvested genetically engineered immune cells, e.g., T cells, are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

### III. ENGINEERED CELLS, POPULATIONS OF ENGINEERED CELLS AND PHARMACEUTICAL COMPOSITIONS THEREOF

Provided herein are plurality or populations of the engineered T cells, such as containing features or generated by methods described herein, and compositions containing such cells. In some aspects, the provided engineered cells and/or composition of engineered cells include any described herein, e.g., comprising a genetic disruption at a CD70 locus, a genetic disruption of the TRAC locus and modification thereof by insertion of a transgene sequence encoding a recombinant CAR and a RNAi expression cassette, and/or a genetic disruption of the B2M locus and modification thereof by insertion of a transgene sequence encoding a recombinant NK cell inhibitor moiety (e.g., HLA-E fusion protein). In some aspects, the plurality or population of engineered cells contain any of the engineered cells described herein, e.g., in Section II herein. In some aspects, the provided cells and cell composition can be engineered using any of the methods described herein, e.g., using agent(s) or methods for introducing genetic disruption, for example, as described in Section II.B herein, and/or using polynucleotides, such as template polynucleotide descried herein, for example in Section I.G, via homology-directed repair (HDR). In some aspects, such cell population is formulated as a pharmaceutical composition or a composition for therapeutic uses or methods, for example, as described in Section VI herein.

In some embodiments, the provided compositions contain cells in which cells expressing the recombinant CAR make up at least 30%, 40%, 50%, 60%, 70%, 75%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75% or more of the total cells in the composition, or cells of a certain type, such as T cells or CD8+ or CD4+ cells, express the recombinant CAR.

In some embodiments, at least or greater than 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition comprise a genetic disruption at a target site within a gene encoding a domain or region of B2M gene and/or does not express a gene product of an endogenous B2M locus. In some embodiments, at least or greater than 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition does not express a gene product of an endogenous B2M locus. In some embodiments, at least or greater than 95% of the cells in the composition does not express a gene product of an endogenous B2M locus.

In some embodiments, at least or greater than 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition comprise a genetic disruption at a target site within a gene encoding a domain or region of T cell receptor alpha constant (TRAC) gene and/or does not express a gene product of an endogenous TRAC locus. In some embodiments, at least or greater than 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition does not express a gene product of an endogenous TRAC locus. In some embodiments, at least or greater than 95% of the cells in the composition does not express a gene product of an endogenous TRAC locus.

In some embodiments, the provided compositions containing cells in which cells expressing the recombinant HLA-E fusion protein make up at least 30%, 40%, 50%, 60%, 70%, 75%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75% or more of the total cells in the composition, or cells of a certain type, such as T cells or CD8+ or CD4+ cells, express the recombinant HLA-E fusion protein.

In some embodiments, the provided compositions containing cells in which cells comprising a genetic disruption at the CD70 locus make up at least 30%, 40%, 50%, 60%, 70%, 80%, 90%, 91%, 92%, 93%, 94%, 95%, 96%, 97%, 98%, 99%, or more of the total cells in the composition, or cells of a certain type, such as T cells or CD8+ or CD4+ cells. In some aspects, at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells do not encode a functional CD70 polypeptide, does not encode a functional PTPN2 polypeptide, do not encode a CD70 polypeptide, do not encode a full length CD70 polypeptide, or the expression of CD70 polypeptide is reduced or eliminated in the cell. In some embodiments, at least 80%, or more of the total cells in the composition, or cells of a certain type, such as T cells or CD8+ or CD4+ cells, comprise a genetic disruption at the CD70 locus.

In some embodiments, the provided compositions containing cells in which cells express reduced TGFBR2 make up at least 30%, 40%, 50%, 60%, 70%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75%, or more of the total cells in the composition, or cells of a certain type, such as T cells or CD8+ or CD4+ cells, have reduced expression of TGFBR2 by at least 10%, 15%, 20%, 25%, 30%, 35%, 40%, 45%, 50%, 55%, 60%, 65%, 75%, 80%, 85%, 90%, 95%, or 99% as compared to a control cell.

In some embodiments, the provided compositions containing cells in which cells express reduced PTPN2 make up at least 30%, 40%, 50%, 60%, 70%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75%, or more of the total cells in the composition, or cells of a certain type, such as T cells or CD8+ or CD4+ cells, have reduced expression of PTPN2 by at least 10%, 15%, 20%, 25%, 30%, 35%, 40%, 45%, 50%, 55%, 60%, 65%, 75%, 80%, 85%, 90%, 95%, or 99% as compared to a control cell.

In some embodiments, the provided compositions containing cells in which cells express reduced FAS make up at least 30%, 40%, 50%, 60%, 70%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 60%, or more of the total cells in the composition, or cells of a certain type, such as T cells or CD8+ or CD4+ cells, have reduced expression of FAS by at least 10%, 15%, 20%, 25%, 30%, 35%, 40%, 45%, 50%, 55%, 60%, 65%, 75%, 80%, 85%, 90%, 95%, or 99% as compared to a control cell.

In some embodiments, at least at or about 70%, 75%, 80%, 85%, 90%, or 95% of the engineered T cells, or of the total cells or total T cells, in a composition containing a plurality of engineered T cells comprise a genetic disruption at a target site within an endogenous CD70 locus. In some embodiments, at least at or about 50%, 55%, 60%, 65%, 70%, 75%, 80%, 85%, 90%, or 95% of the engineered T cells, or of the total cells or total T cells, in a composition containing a plurality of engineered T cells express the recombinant CAR and/or exhibits binding to the antigen recognized by the recombinant CAR. In some embodiments, at least at or about 50%, 55%, 60%, 65%, 70%, 75%, 80%, 85%, 90%, or 95% of the engineered T cells, or of the total cells or total T cells, in a composition containing a plurality of engineered T cells express the recombinant HLA-E fusion protein. In some embodiments, at least at or about 70%, 75%, 80%, 85%, 90%, or 95% of the engineered T cells, or of the total cells or total T cells, in a composition containing a plurality of engineered T cells do not express a gene product of an endogenous TRAC and/or B2M locus.

In some embodiments, provided are cell population and/or compositions that include a plurality of engineered T cells expressing a recombinant CAR, wherein the nucleic acid sequence encoding the recombinant CAR is present at the TRAC locus, e.g., by integration of a transgene encoding recombinant CAR or a portion thereof at the TRAC locus via homology directed repair (HDR). In some embodiments, at least or greater than 30%, 35%, 40%, 45%, 50%, 55%, 60%, 65%, 70%, 75%, 80%, 85%, 90%, or 95% of the cells in the composition and/or cells in the composition that contains a genetic disruption at the CD70 locus comprise integration of the transgene encoding recombinant CAR or a portion thereof at the TRAC locus. In some embodiments, the provided compositions containing cells such as in which cells expressing the recombinant CAR make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the CD70 locus.

In some embodiments, provided are cell population and/or compositions that include a plurality of engineered T cells expressing a recombinant HLA-E fusion protein, wherein the nucleic acid sequence encoding the recombinant HLA-E fusion protein is present at the B2M locus, e.g., by integration of a transgene encoding recombinant HLA-E fusion protein or a portion thereof at the B2M locus via homology directed repair (HDR). In some embodiments, at least or greater than 30%, 35%, 40%, 45%, 50%, 55%, 60%, 65%, 70%, 75%, 80%, 85%, 90%, or 95% of the cells in the composition and/or cells in the composition that contains a genetic disruption at the CD70 locus comprise integration of the transgene encoding recombinant HLA-E fusion protein or a portion thereof at the B2M locus. In some embodiments, the provided compositions containing cells such as in which cells expressing the recombinant HLA-E fusion protein make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the CD70 locus.

In some embodiments, provided are cell populations and/or compositions that include a plurality of engineered T cells expressing a recombinant CAR and/or HLA-E fusion protein, wherein the nucleic acid sequence encoding the recombinant CAR and/or HLA-E fusion protein is present at the TRAC and/or B2M locus, respectively, e.g., by integration of a transgene encoding recombinant CAR and/or HLA-E fusion protein or a portion thereof at the TRAC and/or B2M locus, respectively, via homology directed repair (HDR). In some embodiments, at least or greater than 30%, 35%, 40%, 45%, 50%, 55%, 60%, 65%, 70%, 75%, 80%, 85%, 90%, or 95% of the cells in the composition and/or cells in the composition that contains a genetic disruption at the CD70 locus comprise integration of the transgene encoding recombinant CAR and/or HLA-E fusion protein or a portion thereof at the TRAC and/or B2M locus. In some embodiments, the provided compositions containing cells such as in which cells expressing the recombinant CAR and/or HLA-E fusion protein make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the CD70 locus.

In some embodiments, provided are cell populations and/or compositions that include a plurality of engineered T cells expressing a RNAi expression cassette and/or HLA-E fusion protein, wherein the nucleic acid sequence encoding the RNAi expression cassette and/or HLA-E fusion protein is present at the TRAC and/or B2M locus, respectively, e.g., by integration of a transgene encoding RNAi expression cassette and/or HLA-E fusion protein or a portion thereof at the TRAC and/or B2M locus, respectively, via homology directed repair (HDR). In some embodiments, at least or greater than 30%, 35%, 40%, 45%, 50%, 55%, 60%, 65%, 70%, 75%, 80%, 85%, 90%, or 95% of the cells in the composition and/or cells in the composition that contains a genetic disruption at the CD70 locus comprise integration of the transgene encoding RNAi expression cassette and/or HLA-E fusion protein or a portion thereof at the TRAC and/or B2M locus. In some embodiments, the provided compositions containing cells such as in which cells expressing the RNAi expression cassette and/or HLA-E fusion protein make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the CD70 locus.

In some embodiments, provided are cell populations and/or compositions that include a plurality of engineered T cells expressing a recombinant CAR and RNAi expression cassette and/or HLA-E fusion protein, wherein the nucleic acid sequence encoding the recombinant CAR and RNAi expression cassette and/or HLA-E fusion protein is present at the TRAC and/or B2M locus, respectively, e.g., by integration of a transgene encoding recombinant CAR and RNAi expression cassette and/or HLA-E fusion protein or a portion thereof at the TRAC and/or B2M locus, respectively, via homology directed repair (HDR). In some embodiments, at least or greater than 30%, 35%, 40%, 45%, 50%, 55%, 60%, 65%, 70%, 75%, 80%, 85%, 90%, or 95% of the cells in the composition and/or cells in the composition that contains a genetic disruption at the CD70 locus comprise integration of the transgene encoding recombinant CAR and RNAi expression cassette and/or HLA-E fusion protein or a portion thereof at the TRAC and/or B2M locus. In some embodiments, the provided compositions containing cells such as in which cells expressing the recombinant CAR and RNAi expression cassette and/or HLA-E fusion protein make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the CD70 locus.

In some of any embodiments, the genetic disruption is a genetic disruption of the CD70 locus that is a knockout of the CD70 gene, a further genetic disruption of the TRAC locus that is a knockout of the TRAC gene, and the recombinant CAR is knocked in to the TRAC locus by homology directed repair, and/or a further genetic disruption of the B2M locus that is a knockout of the B2M gene, and the recombinant HLA-E fusion protein is knocked in to the B2M locus by homology directed repair. In some of any embodiments, at least at or about 80% of the engineered T cells, or of the total cells or total T cells, in a population containing a plurality of engineered T cells generated by any of the provided methods comprise a genetic disruption at a target site within an endogenous CD70 locus; at least at or about 50% of the engineered T cells, or of the total cells or total T cells, in a population containing a plurality of engineered T cells generated by any of the provided methods express the recombinant CAR or exhibits binding to the antigen recognized by the recombinant CAR; and at least at or about 90% of the engineered T cells, or of the total cells or total T cells, in a population containing a plurality of engineered T cells generated by any of the provided methods do not express a gene product of an endogenous TRAC locus; at least at or about 75% of the engineered T cells, or of the total cells or total T cells, in a population containing a plurality of engineered T cells generated by any of the provided methods express the recombinant HLA-E fusion protein; and at least at or about 95% of the engineered T cells, or of the total cells or total T cells, in a population containing a plurality of engineered T cells generated by any of the provided methods do not express a gene product of an endogenous B2M locus.

In some of any embodiments, among a population of cells containing a plurality of engineered T cells generated by any of the provided methods greater than 70%, 80%, 90%, 91%, 92%, 93%, 94%, 95%, 96%, 97%, 98%, or 99% are knocked out for TRAC (TRAC KO) and/or for B2M (B2M KO), greater than 70%, 80%, 90%, 91%, 92%, 93%, 94%, 95%, 96%, 97%, 98%, or 99% are knocked out for CD70 (CD70 KO) and greater than 70%, 80%, 90%, 91%, 92%, 93%, 94%, 95%, 96%, 97%, 98%, or 99% have knock-in of the recombinant CAR and/or greater than 70%, 80%, 90%, 91%, 92%, 93%, 94%, 95%, 96%, 97%, 98%, or 99% have knock in of the recombinant HLA-E fusion protein and/or greater than 70%, 80%, 90%, 91%, 92%, 93%, 94%, 95%, 96%, 97%, 98%, or 99% have knock in of the recombinant CAR and RNAi expression cassette and/or greater than 70%, 80%, 90%, 91%, 92%, 93%, 94%, 95%, 96%, 97%, 98%, or 99% have knock in of the RNAi expression cassette.

In some of any embodiments, among a population of cells containing a plurality of engineered T cells generated by any of the provided methods greater than 85% are knocked out for TRAC (TRAC KO) and/or for B2M (B2M KO), greater than 80% are knocked out for CD70 (CD70 KO) and greater than 50% have knock-in of the recombinant CAR, RNAi expression cassette, and/or HLA-E fusion protein. In some of any embodiments, among a population of cells containing a plurality of engineered T cells generated by any of the provided methods greater than 90% are knocked out for TRAC (TRAC KO) and/or for B2M (B2M KO), greater than 85% are knocked out for CD70 (CD70 KO) and greater than 70% have knock-in of the recombinant CAR, RNAi expression cassette, and/or HLA-E fusion protein. In some of any embodiments, among a population of cells containing a plurality of engineered T cells generated by any of the provided methods greater than 95% are knocked out for TRAC (TRAC KO) and/or for B2M (B2M KO), greater than 90% are knocked out for PTPN2 (PTPN2 KO) and greater than 75% have knock-in of the recombinant CAR, RNAi expression cassette, and/or HLA-E fusion protein.

In some embodiments, knock-out of the TRAC, B2M, or CD70 loci is determined by a PCR-based method, such as ddPCR. In some embodiments, knock-out of the TRAC, B2M, or CD70 loci is determined by flow cytometry for expression of TCR alpha chain constant region (Cα) protein and/or B2M and/or CD70. In some embodiments, knock-out of the TRAC, B2M, or CD70 loci is determined by a PCR-based method (e.g., ddPCR) or next genome sequencing (NGS).

In some embodiments, knock-in of the recombinant CAR, RNAi expression cassette, and/or HLA-E fusion protein is determined by a PCR-based method, such as ddPCR. In some embodiments, knock-in of the recombinant CAR, RNAi expression cassette, and/or HLA-E fusion protein is determined by flow cytometry for expression of the recombinant CAR and/or HLA-E fusion protein and/or target genes complementary to the nucleic acids encoded by the RNAi expression cassette. In some embodiments, knock-in of the recombinant CAR, RNAi expression cassette, and/or HLA-E fusion protein locus is determined molecularly, such as by PCR-based methods (e.g. ddPCR) or next genome sequencing (NGS).

In some embodiments, at least at or about 50%, 60%, 70%, 80%, 90%, 91%, 92%, 93%, 94%, 95%, 96%, 97%, 98%, or 99% of the engineered T cells in a composition or a plurality of T cells comprise a genetic disruption at a first target site within an endogenous CD70 locus; at least at or about 50%, 60%, 70%, 80%, 90%, 91%, 92%, 93%, 94%, 95%, 96%, 97%, 98%, or 99% of the engineered T cells in a composition or a plurality of T cells express the recombinant CAR, RNAi expression cassette, and/or HLA-E fusion; and at least at or about 50%, 60%, 70%, 80%, 90%, 91%, 92%, 93%, 94%, 95%, 96%, 97%, 98%, or 99% of the engineered T cells in a composition or a plurality of T cells do not express a gene product of an endogenous TRAC and/or B2M locus.

In some embodiments, at least at or about 70% of the engineered T cells in a composition or a plurality of T cells comprise a genetic disruption at a first target site within an endogenous CD70 locus; at least at or about 50% of the engineered T cells in a composition or a plurality of T cells express the recombinant CAR, RNAi expression cassette, and/or HLA-E fusion; and at least at or about 70% of the engineered T cells in a composition or a plurality of T cells do not express a gene product of an endogenous TRAC and/or B2M locus.

In some embodiments, at least at or about 80% of the engineered T cells, or of the total cells or total T cells, in a composition containing a plurality of engineered T cells comprise a genetic disruption at a first target site within an endogenous CD70 locus; at least at or about 75% of the engineered T cells, or of the total cells or total T cells, in a composition containing a plurality of engineered T cells express the recombinant CAR, RNAi expression cassette, and/or HLA-E fusion; and at least at or about 95% of the engineered T cells, or of the total cells or total T cells, in a composition containing a plurality of engineered T cells do not express a gene product of an endogenous TRAC and/or B2M locus.

In some embodiments, at least at or about 80% of the total cells or total T cells, in a composition containing a plurality of engineered T cells comprise a genetic disruption at a first target site within an endogenous CD70 locus; at least at or about 75% of the total cells or total T cells, in a composition containing a plurality of engineered T cells express the recombinant CAR, RNAi expression cassette, and/or HLA-E fusion; and at least at or about 95% of the total cells or total T cells, in a composition containing a plurality of engineered T cells do not express a gene product of an endogenous TRAC and/or B2M locus.

In some embodiments, the provided cell population and/or compositions containing engineered cells include a cell population that exhibits more improved, uniform, homogeneous and/or stable expression by the recombinant CAR, RNAi expression cassette, and/or HLA-E fusion, e.g., exhibit reduced coefficient of variation, compared to the expression and/or antigen binding of cell populations and/or compositions generated using other methods. In some embodiments, the cell population and/or compositions exhibit at least 100%, 95%, 90%, 80%, 70%, 60%, 50%, 40%, 30%, 20% or 10% lower coefficient of variation of expression of the recombinant CAR, RNAi expression cassette, and/or HLA-E fusion protein to a respective population generated using other methods, e.g., random integration of sequences encoding the recombinant CAR, RNAi expression cassette, and/or HLA-E fusion protein. The coefficient of variation is defined as standard deviation of expression of the nucleic acid of interest (e.g., transgene sequences encoding a recombinant CAR and/or HLA-E fusion or portion thereof) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, the provided cell population and/or compositions containing engineered cells include a cell population that exhibits minimal or reduced random integration of the transgene encoding a recombinant CAR, RNAi expression cassette, and/or HLA-E fusion protein or a portion thereof. In some aspects, random integration of transgene into the genome of the cell can result in adverse effects or cell death due to integration of the transgene into undesired location in the genome, e.g., into an essential gene or a gene critical in regulating the activity of the cell, and/or unregulated or uncontrolled expression of the receptor. In some aspects, random integration of the transgene is reduced by at least or greater than 50%, 60%, 70%, 80%, 90%, 91%, 92%, 93%, 94%, 95%, 96%, 97%, 98%, 99%, or more compared to cell populations generated using other methods.

In some of any provided embodiments, a population of cells containing a plurality of engineered T cells contains greater than 75% T cells, such as greater than 80% T cells, greater than 85% T cells, greater than 90% T cells or greater than 95% T cells. In some embodiments, the T cells are CD3+. In some embodiments, the T cells include CD4+ and CD8+ T cells. In some embodiments, the ratio of CD4+ and CD8+ T cells in the population, or among cells expressing the CAR, is between at about 1:5 and 5:1, such as 1:3 and 3:1 or 1:2 and 2:1, such as at or about 1:1.

In some aspects, the composition of cells comprises CD4+ T cells and/or CD8+ T cells. In some aspects, the composition of cells comprises CD4+ T cells and CD8+ T cells. In some aspects, the percentage of CD4+ T cells in the composition is between at or about 20% and at or about 80%, or at or about 20%, 30%, 40%, 50%, 60%, 70%, or 80% of the total cells in the composition. In some aspects, the percentage of CD8+ T cells in the composition is between at or about 20% and at or about 80%, or at or about 20%, 30%, 40%, 50%, 60%, 70%, or 80% of the total cells in the composition. In some embodiments, the percentage of CD4+ T cells in the composition is between at or about 20% and at or about 80%, or at or about 20%, 30%, 40%, 50%, 60%, 70%, or 80% of the total cells in the composition; and the percentage of CD8+ T cells in the composition is between at or about 20% and at or about 80%, or at or about 20%, 30%, 40%, 50%, 60%, 70%, or 80% of the total cells in the composition. In some aspects, the composition comprises CD4+ T cells and CD8+ T cells, and the ratio of CD4+ T cells to CD8+ T cells is from at or about 1:3 to at or about 3:1. In some aspects, the composition comprises CD4+ T cells and CD8+ T cells, and the ratio of CD4+ T cells to CD8+ T cells is at or about 1:1.

Also provided are compositions, such as pharmaceutical compositions and formulations for administration, containing any of the engineered cells. Such compositions can be used in accord with the provided methods, and/or with the provided articles of manufacture or compositions, such as in the prevention or treatment of diseases, conditions, and disorders, or in detection, diagnostic, and prognostic methods.

### IV. EXEMPLARY FEATURES OF GENETICALLY ENGINEERED CELLS

In some embodiments, cells and compositions according to the provided disclosure (e.g. cells comprising a genetic disruption at a CD70 locus, a modified TRAC locus comprising a transgene sequence encoding a recombinant CAR or a portion thereof and/or a RNAi expression cassette; and/or a modified B2M locus comprising a transgene sequence encoding a recombinant HLA-E fusion protein or a portion thereof) exhibit an altered immunogenic profile compared to a corresponding or reference cell or composition.

Graft-versus-host disease (GVHD), also known as graft versus host disease, is a common complication following an allogeneic tissue or cell transplant. It is commonly associated with stem cell or bone marrow transplant but the term also applies to other forms of tissue or cell therapy graft. Immune cells (white blood cells) in the tissue (the graft) recognize the recipient (the host) as “foreign”. The transplanted immune cells then attack the host's body cells. GVHD can also occur after a blood transfusion if the blood products used have not been irradiated or treated with an approved pathogen reduction system.

In some aspects, the provided embodiments are based on observations that the efficacy of adoptive cell therapy may be limited by the development of an immune response in the subject to the cells and/or construct administered. In some embodiments, exogenous engineered cells when administered to a subject can be recognized by a host's immune response as foreign. In some embodiments, such recognition can occur when cells are allogeneic to the host subject to which they are administered. In some embodiments, such recognition can occur even when the cells are autologous to the host subject to which they are administered, such as when the cells are engineered with a recombinant molecule or receptor (e.g. CAR) that is not native to or normally expressed by the cells. In some embodiments, development of a host immune response to administered cells, either allogeneic or autologous cells, can result in host-versus-graft responses that can lead to rejection of adoptively transferred cells.

In some embodiments, the provided engineered cells, compositions and methods can be used regardless of the HLA type or subtype of the subject, which can, in some aspects, permit “off-the-shelf” delivery to a wider variety of recipients. In some embodiments, the provided compositions and methods can be used to provide adoptive cell therapy using allogeneic cells engineered to treat a disease or disorder. In some cases, using allogeneic cells can provide certain advantages. In some embodiments, cells with known safety and efficacy profiles can be prepared for a wider variety of patients. For example, cells can be derived from a healthy donor and delivered to a subject that may be too sick to provide cells suitable for genetic engineering. In some cases, a subject may have a defect or disease in the cells or cell type typically used for a particular adoptive cell therapy regimen, such that cells from a healthy donor can be used that replace or supplement the diseased cells. In some cases, the ability to engineer or administer allogeneic cells permits the preparation of cells in advance, which can reduce the time needed before being delivered to a patient. In some cases, the engineered allogeneic cells may present lower risks of causing graft-versus-host disease or host-versus-graft disease.

In some embodiments, the provided cells and compositions are less immunogenic and/or result in a reduced degree of recognition of the engineered cell by the host immune system upon administration of the cells. In some embodiments, the provided cells and compositions result in a reduced risk of development of graft-vs-host and/or host-vs-graft disease. In some embodiments, the provided cells and compositions are more cytotoxic and/or result in increased killing of tumor cells upon administration of the cells. In some embodiments, the provided cells and compositions exhibit increased efficiency and/or result in longer utility in the killing of tumor cells upon administration of the cells. In some cases, reference to a “reference cell” or “reference composition” (and/or a “corresponding cell” or a “corresponding composition”), may refer to a cell or composition (such as a T cell or composition of T cells) that are obtained, isolated, generated, produced and/or incubated under the same or substantially the same conditions, except that the such cells do not have a reduction, deletion, elimination, knockout or disruption in expression of the TRAC, B2M, and/or CD70 gene and/or do not express a recombinant CAR, RNAi expression cassette, and/or HLA-E fusion protein. In some aspects, except for not containing introduction of the CAR, RNAi expression cassette, and/or the recombinant HLA-E fusion protein and/or genetic disruption of the TRAC, B2M, and/or CD70 genes, such cells or T cells are treated identically or substantially identically as T cells or cells that have been introduced with CAR, RNAi expression cassette, and/or the recombinant HLA-E fusion protein and/or genetic disruption of the TRAC, B2M, and/or CD70 genes, such that any one or more conditions that can influence the activity or properties of the cell is not varied or not substantially varied between the cells other than the introduction of the agent.

In some embodiments, the immune response is a humoral immune response. In some embodiments, the immune response is a cell-mediated immune response. In some embodiments, the immune response is reduced greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the reduced immunogenicity comprises a reduced CD4+ T cell immune response, a reduced CD8+ T cell immune response, and/or a reduced NK cell immune response. In some embodiments, immunogenicity is reduced greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the cytotoxicity against tumor cells is increased greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the cytotoxicity against tumor cells is increased greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the CART cell efficacy is increased greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the CART cell efficacy is increased greater than 1.5-fold, 2-fold, 3-fold, 4-fold, 5-fold, 6-fold, 7-fold, 8-fold, 9-fold, 10-fold, 50-fold, 100-fold or more compared to a reference cell or composition.

In some embodiments, the reduction, disruption, deletion or elimination of expression of the TRAC, B2M, and/or CD70 genes in the provided engineered cells does not impair or alter the function or activity of the recombinant receptor compared to a reference cell or composition. In some embodiments, the recombinant receptor retains specific binding to the antigen. In some embodiments, the recombinant receptor retains activating or stimulating activity, upon antigen binding, to induce cytotoxicity, proliferation, survival or cytokine secretion in cells. In some embodiments, the engineered cells of the provided compositions retain a functional property or activity compared to a corresponding or reference composition when assessed under the same conditions. In some embodiments, the cells retain cytotoxicity, proliferation, survival or cytokine secretion compared to such a corresponding or reference composition.

### V. RNAI EXPRESSION CASSETTES

In some embodiments, the provided genetically engineered T cells contain or are engineered to contain an RNA interference (RNAi) expression cassette. The RNAi expression cassette generally includes a nucleic acid that is complementary to a target gene. In some embodiments, the expression of the target gene (e.g., the target gene that is complementary to the nucleic acid sequence) in a cell may be specifically and effectively modulated using the nucleic acid sequence present in the RNAi expression cassette. In some embodiments, the target gene may be implicated in the growth (proliferation), maintenance (survival), and/or immune behavior of an individual's immune cells.

In some embodiments, the RNA interference (RNAi) expression cassette is a nucleic acid. Exemplary RNAi expression cassettes (e.g., antisense RNA, siRNA, microRNA, shRNA, etc.), and methods for engineering and introducing such RNAi expression cassette into cells, include those described, for example, in International Publication Nos. WO2018/232356, WO2019/084552, WO2019/226998, WO2020/014235, WO2020/123871, WO2020/186219, and WO2024/059618, each of which is incorporated by reference in its entirety for all purposes. RNAi expression cassettes may include nucleic acids that are short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species.

Expression of shRNA from a plasmid or viral vector is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a viral vector can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference.

siRNA molecules and methods of use and production are described in U.S. Pat. No. 7,361,752 and US Patent Publication No. US20050048647, both of which are hereby incorporated by reference.

Additional methods and compositions for RNAi such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO001/29058; and International PCT Publication No. WO00/44914; and International PCT Publication No. WO04/030634; each of which are hereby incorporated by reference.

In some embodiments, more than one target gene is modulated using a nucleic acid that is complementary to a target gene. In some embodiments, at least two target genes are modulated using the nucleic acid e.g., nucleic acid of the RNAi expression cassette) described herein. In some embodiments, the nucleic acid is at least one, two, three, or four nucleic acids. In some embodiments, the nucleic acid is an shRNA. In some embodiments, the at least two target genes are at least TGFBR2, FAS and PTPN2.

In some embodiments, the target gene is Transforming Growth Factor Beta Receptor 2 (TGFBR2). In some embodiments, one nucleic acid targets the TFGBR2 gene. In some embodiments, two or more nucleic acids target the TFGBR2 gene. Transforming Growth Factor Beta Receptor 2 (TGFBR2; HGNC: 11773, NCBI Entrez Gene: 7048, UniProtKB/Swiss-Prot: P37173) is a transmembrane serine/threonine protein kinase and forms a heterodimeric complex with TGF-beta receptor type-1 (TGFBR1) when bound to TGF-beta, resulting in transduction of the TGF-beta signal from the cell surface to the cytoplasm.

In some embodiments, the nucleic acid comprises a nucleic acid sequence at least 15 nucleotides in length complementary to an mRNA encoding human Transforming Growth Factor Beta Receptor 2 (TGFBR2). In some embodiments, the nucleic acid comprises a nucleic acid sequence at least 15 nucleotides in length complementary to an mRNA encoding human Transforming Growth Factor Beta Receptor 2 (TGFBR2). In some embodiments, the mRNA encoding human TGFBR2 comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85. In some embodiments, the mRNA encoding human TGFBR2 comprises a sequence set forth in SEQ ID NO:85.

In some embodiments, the nucleic acid comprises a nucleic acid sequence at least 15 nucleotides in length complementary to the nucleotides 2215-2236, 4430-4451, or 3761-3782 of an mRNA encoding human Transforming Growth Factor Beta Receptor 2 (TGFBR2) comprising the sequence set forth in SEQ ID NO:85. In some embodiments, the nucleic acid comprises a nucleic acid sequence at least 15 nucleotides in length complementary to the nucleotides 2215-2236 or 4430-4451 of an mRNA encoding human Transforming Growth Factor Beta Receptor 2 (TGFBR2) comprising the sequence set forth in SEQ ID NO:85. In some embodiments, the nucleic acid comprises a nucleic acid sequence at least 15 nucleotides in length complementary to the nucleotides 2215-2236 of an mRNA encoding human Transforming Growth Factor Beta Receptor 2 (TGFBR2) comprising the sequence set forth in SEQ ID NO:85. In some embodiments, the nucleic acid comprises a nucleic acid sequence at least 15 nucleotides in length complementary to the nucleotides 4430-4451 of an mRNA encoding human Transforming Growth Factor Beta Receptor 2 (TGFBR2) comprising the sequence set forth in SEQ ID NO:85.

In some embodiments, the nucleic acid comprises a sequence selected from the group consisting of the sequences set forth in SEQ ID NOs: 74 and 75. In some embodiments, the nucleic acid comprises a sequence selected from the group consisting of the sequences set forth in SEQ ID NOs: 74 and 75. In some embodiments, the nucleic acid comprises the sequence set forth in SEQ ID NO: 74. In some embodiments, the nucleic acid comprises the sequence set forth in SEQ ID NO: 75.

In some embodiments, the nucleic acid comprises two nucleic acids. In some embodiments, the first and second of the two nucleic acids each comprises a sequence selected from the group consisting of the sequences set forth in SEQ ID NOs: 74 and 75. In some embodiments, the nucleic acid or the first of the two nucleic acids comprise the sequence set forth in SEQ ID NO: 74. In some embodiments, the nucleic acid or first of the two nucleic acids comprise the sequence set forth in SEQ ID NO: 75. In some embodiments, the first of the two nucleic acids comprises the sequence set forth in SEQ ID NO: 74. In some embodiments, the second of the two nucleic acids comprises the sequence set forth in SEQ ID NO: 75. In some embodiments, the first of the two nucleic acids comprises the sequence set forth in SEQ ID NO: 74 and the second of the two nucleic acids comprises the sequence set forth in SEQ ID NO: 75. In some embodiments, the first of the two nucleic acids and the second of the two nucleic acids comprise the sequence set forth in SEQ ID NOs: 74 and 75, respectively.

In some embodiments, the nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the target gene is Protein Tyrosine Phosphatase Non-Receptor Type 2 (PTPN2). In some embodiments, one nucleic acid targets the PTPN2 gene. PTPN2 is a phosphatase that regulates interferon and many other signaling pathways.

In some embodiments, the nucleic acid comprises a nucleic acid sequence at least 15 nucleotides in length complementary to an mRNA encoding human Protein Tyrosine Phosphatase Non-Receptor Type 2 (PTPN2). In some embodiments, the mRNA encoding human Protein Tyrosine Phosphatase Non-Receptor Type 2 (PTPN2) comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:86. In some embodiments, the mRNA encoding human Protein Tyrosine Phosphatase Non-Receptor Type 2 (PTPN2) comprises the sequence set forth in SEQ ID NO:86.

In some embodiments, the nucleic acid comprises a nucleic acid sequence at least 15 nucleotides in length complementary nucleotides 518 to 559 of an mRNA encoding human Protein Tyrosine Phosphatase Non-Receptor Type 2 (PTPN2) comprising the sequence set forth in SEQ ID NO:86. In some embodiments, the nucleic acid comprises a nucleic acid sequence at least 15 nucleotides in length complementary nucleotides 518 to 539 of an mRNA encoding human Protein Tyrosine Phosphatase Non-Receptor Type 2 (PTPN2) comprising the sequence set forth in SEQ ID NO: 86.

In some embodiments, the nucleic acid comprises a sequence set forth in SEQ ID NO: 73.

In some embodiments, the nucleic acid reduces expression of PTPN2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the target gene is Fas Cell Surface Death Receptor (FAS). In some embodiments, one nucleic acid targets the FAS gene. FAS is an apoptosis-inducing TNF receptor superfamily member.

In some embodiments, the nucleic acid comprises a nucleic acid sequence at least 15 nucleotides in length complementary to an mRNA encoding human Fas Cell Surface Death Receptor (FAS). In some embodiments, the mRNA encoding human Fas Cell Surface Death Receptor (FAS) comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:87. In some embodiments, the mRNA encoding human Fas Cell Surface Death Receptor (FAS) comprises the sequence set forth in SEQ ID NO:87.

In some embodiments, the nucleic acid comprises a nucleic acid sequence at least 15 nucleotides in length complementary to nucleotides 1126 to 1364 of an mRNA encoding human Fas Cell Surface Death Receptor (FAS) comprising the sequence set forth in SEQ ID NO:87. In some embodiments, the nucleic acid comprises a nucleic acid sequence at least 15 nucleotides in length complementary to nucleotides 1126 to 1147 of an mRNA encoding human Fas Cell Surface Death Receptor (FAS) comprising the sequence set forth in SEQ ID NO:87.

In some embodiments, the nucleic acid comprises a sequence set forth in SEQ ID NO: 72.

In some embodiments, the nucleic acid reduces expression of FAS in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid sequence is at least 16, 17, 18, 19, 20, 21, or 22 nucleotides in length.

In some embodiments, the nucleic acid comprises: (i) a nucleic acid, such as a first nucleic acid, comprising a nucleic acid sequence at least 15 nucleotide in length complementary to an mRNA encoding human Transforming Growth Factor Beta Receptor 2 (TGFBR2), such any nucleic acids described herein, (ii) a nucleic acid, such as a second nucleic acid, comprising a nucleic acid sequence at least 15 nucleotides in length complementary to an mRNA encoding human Protein Tyrosine Phosphatase Non-Receptor Type 2 (PTPN2), such any nucleic acids described herein, and (iii) a nucleic acid, such as a third nucleic acid, comprising a nucleic acid sequence at least 15 nucleotides in length complementary to an mRNA encoding human Fas Cell Surface Death Receptor (FAS), such any nucleic acids described herein. In some embodiments, the nucleic acid further comprises: (iv) a nucleic acid, such as a fourth nucleic acid, comprising a nucleic acid sequence at least 15 nucleotide in length complementary to an mRNA encoding human Transforming Growth Factor Beta Receptor 2 (TGFBR2), such any nucleic acids described herein.

In some embodiments, the nucleic acid comprises: (i) a nucleic acid, such as a first nucleic acid, comprising a nucleic acid sequence at least 15 nucleotide in length complementary to an mRNA encoding human Transforming Growth Factor Beta Receptor 2 (TGFBR2), wherein the mRNA encoding human TGFBR2 comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85, (ii) a nucleic acid, such as a second nucleic acid, comprising a nucleic acid sequence at least 15 nucleotides in length complementary to an mRNA encoding human Protein Tyrosine Phosphatase Non-Receptor Type 2 (PTPN2), wherein the mRNA encoding human PTPN2 comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:86, and (iii) a nucleic acid, such as a third nucleic acid, comprising a nucleic acid sequence at least 15 nucleotides in length complementary to an mRNA encoding human Fas Cell Surface Death Receptor (FAS), wherein the mRNA encoding human FAS comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:87. In some embodiments, the nucleic acid further comprises: (iv) a nucleic acid, such as a fourth nucleic acid, comprising a nucleic acid sequence at least 15 nucleotide in length complementary to an mRNA encoding human Transforming Growth Factor Beta Receptor 2 (TGFBR2), wherein the mRNA encoding human TGFBR2 comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85.

In some embodiments, the nucleic acid comprises: (i) a nucleic acid, such as a first nucleic acid, comprising a nucleic acid sequence at least 15 nucleotide in length complementary to an mRNA encoding human Transforming Growth Factor Beta Receptor 2 (TGFBR2), wherein the mRNA encoding human TGFBR2 comprises a sequence set forth in SEQ ID NO:85, (ii) a nucleic acid, such as a second nucleic acid, comprising a nucleic acid sequence at least 15 nucleotides in length complementary to an mRNA encoding human Protein Tyrosine Phosphatase Non-Receptor Type 2 (PTPN2), wherein the mRNA encoding human PTPN2 comprises a sequence set forth in SEQ ID NO: 86, and (iii) a nucleic acid, such as a third nucleic acid, comprising a nucleic acid sequence at least 15 nucleotides in length complementary to an mRNA encoding human Fas Cell Surface Death Receptor (FAS), wherein the mRNA encoding human FAS comprises a sequence set forth in SEQ ID NO:87. In some embodiments, the nucleic acid further comprises: (iv) a nucleic acid, such as a fourth nucleic acid, comprising a nucleic acid sequence at least 15 nucleotide in length complementary to an mRNA encoding human Transforming Growth Factor Beta Receptor 2 (TGFBR2), wherein the mRNA encoding human TGFBR2 comprises a sequence set forth in SEQ ID NO:85.

In some embodiments, the nucleic acid comprises: (i) a nucleic acid, such as a first nucleic acid, comprising a nucleic acid sequence at least 15 nucleotide in length complementary to nucleotides 2215 to 2236 of the mRNA encoding human Transforming Growth Factor Beta Receptor 2 (TGFBR2), wherein the mRNA encoding human TGFBR2 comprises a sequence set forth in SEQ ID NO:85, (ii) a nucleic acid, such as a second nucleic acid, comprising a nucleic acid sequence at least 15 nucleotides in length complementary to nucleotides 2215 to 2236 of the mRNA encoding human Protein Tyrosine Phosphatase Non-Receptor Type 2 (PTPN2), wherein the mRNA encoding human PTPN2 comprises a sequence set forth in SEQ ID NO:86, and (iii) a nucleic acid, such as a third nucleic acid, comprising a nucleic acid sequence at least 15 nucleotides in length complementary to nucleotides 1126 to 1127 of the mRNA encoding human Fas Cell Surface Death Receptor (FAS), wherein the mRNA encoding human FAS comprises a sequence set forth in SEQ ID NO:87. In some embodiments, the nucleic acid further comprises: (iv) a nucleic acid, such as a fourth nucleic acid, comprising a nucleic acid sequence at least 15 nucleotide in length complementary to nucleotides 4430 to 4451 of the mRNA encoding human Transforming Growth Factor Beta Receptor 2 (TGFBR2), wherein the mRNA encoding human TGFBR2 comprises a sequence set forth in SEQ ID NO:85.

In some embodiments, the nucleic acid comprises: (i) a nucleic acid, such as a first nucleic acid, comprising the sequence set forth in SEQ ID NO:74, (ii) a nucleic acid, such as a second nucleic acid, comprising a nucleic acid sequence comprising the sequence set forth in SEQ ID NO:73, and (iii) a nucleic acid, such as a third nucleic acid, comprising the sequence set forth in SEQ ID NO:72. In some embodiments, the nucleic acid further comprises: (iv) a nucleic acid, such as a fourth nucleic acid comprising the sequence set forth in SEQ ID NO:75.

In some embodiments, the RNAi expression cassette comprises a nucleic acid, wherein the nucleic acid comprises an shRNA module. In some embodiments, the shRNA module comprises a first strand and a second strand of one or more shRNAs. In some embodiments, the shRNA module comprises two shRNAs. In some embodiments, the shRNA module comprises three shRNA sequences. In some embodiments, the shRNA module comprises four shRNA sequences. In some embodiments, the shRNA module further comprises one or more backbones of one or more miRs. In some embodiments, the one or more backbones comprises a 5′ backbone, a loop, and a 3′ backbone. In some embodiments the shRNA module comprises the first and second strands of the one or more shRNAs within the backbone of the one or more miRs. In some embodiments the first strand of an shRNA is located between the 5′ backbone and the loop of a miR and the second strand of the shRNA is located between the loop and the 3′ backbone of the miR.

In some embodiments the shRNA module is a triple shRNA module. In some embodiments the shRNA module comprises, from 5′ to 3′, (1) a 5′ backbone of a first miR (e.g., a miR-3G 5′ backbone), (2) a first strand of a first shRNA, (3) a loop of the first miR (e.g., a miR-3G loop), (4) a second strand of the first shRNA, (5) a 3′ backbone of the first miR (e.g., a miR-3G 3′ backbone), (6) a first spacer, (7) a 5′ backbone of a second miR (e.g., a miR-E 5′ backbone), (8) a first strand of a second shRNA, (9) a loop of the second miR (e.g., a miR-E loop), (10) a second strand of the second shRNA, (11) a 3′ backbone of the second miR (e.g., a miR-E 3′ backbone), (12) a second spacer, (13) a 5′ backbone of a third miR (e.g., a miR-3G 5′ backbone), (14) a first strand of a third shRNA, (15) a loop of the third miR (e.g., a miR-3G loop), (16) a second strand of the third shRNA, (17) a 3′ backbone of the third miR (e.g., a miR-3G 3′ backbone), and (18) a PPT sequence. In some embodiments, the first, second, and third miRs are identical. In some embodiments, the first, second, and third miRs are all distinct. In some embodiments, the first and second miRs are identical and the third miR is distinct. In some embodiments, the first and third miRs are identical and the second miR is distinct. In some embodiments, the second and third miRs are identical and the first miR is distinct. In some embodiments, the first and third miRs are miR-3G and the second miR is miR-E (“3G-E-3G format”). In some embodiments, the first, second, and third miRs are all miR-3G (“3G-3G-3G format”).

In some embodiments the shRNA module is a quadruple shRNA module. In some embodiments the shRNA module comprises, from 5′ to 3′, (1) a 5′ backbone of a first miR (e.g., a miR-3G 5′ backbone), (2) a first strand of a first shRNA, (3) a loop of the first miR (e.g., a miR-3G loop), (4) a second strand of the first shRNA, (5) a 3′ backbone of the first miR (e.g., a miR-3G 3′ backbone), (6) a first spacer, (7) a 5′ backbone of a second miR (e.g., a miR-E 5′ backbone), (8) a first strand of a second shRNA, (9) a loop of the second miR (e.g., a miR-E loop), (10) a second strand of the second shRNA, (11) a 3′ backbone of the second miR (e.g., a miR-E 3′ backbone), (12) a second spacer, (13) a 5′ backbone of a third miR (e.g., a miR-3G 5′ backbone), (14) a first strand of a third shRNA, (15) a loop of the third miR (e.g., a miR-3G loop), (16) a second strand of the third shRNA, (17) a 3′ backbone of the third miR (e.g., a miR-3G 3′ backbone), (18) a third spacer, (19) a 5′ backbone of a fourth miR (e.g., a miR-3G 5′ backbone), (20) a first strand of a fourth shRNA, (21) a loop of the fourth miR (e.g., a miR-3G loop), (22) a second strand of the fourth shRNA, (23) a 3′ backbone of the fourth miR (e.g., a miR-3G 3′ backbone), (24) a PPT sequence. In some embodiments, the first, second, third, and fourth miRs are identical. In some embodiments, the first, second, third, and fourth miRs are all distinct. In some embodiments, a first group of two miRs are identical and a second group of two miRs are identical but distinct from the first group. In some embodiments, a first group of two miRs are identical and the remaining two miRs are each distinct from the first group and each other. In some embodiments, three of the miRs are identical and the last miR is distinct. In some embodiments, the first, third, and fourth miRs are miR-3G and the second miR is miR-E (“3G-E-3G-3G format”).

In some embodiments, the nucleic acid comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:71. In some embodiments, the nucleic acid comprises the sequence set forth in SEQ ID NO:71.

Also provided herein are polynucleotides encoding any of the provided RNAi expression cassettes.

In some embodiments, the RNAi expression cassettes are encoded by polynucleotides. The polynucleotides that may be used to encode the RNAi expression cassette, such as an shRNA described herein, may comprise a promoter, which is operably linked (or connected), directly or indirectly, to a sequence encoding the RNAi expression cassettes. Such promoters may be selected based on the host cell and the effect sought. Non-limiting examples of suitable promoters include constitutive and inducible promoters, such as inducible RNA polymerase II (pol II)-based promoters. Non-limiting examples of suitable promoters further include the tetracycline inducible or repressible promoter, EF1α, RNA polymerase I or III-based promoters, the pol II dependent viral promoters, such as the CMV-IE promoter, and the pol III U6 and H1 promoters. In some embodiments, the promoter is any of the promoters disclosed herein in Section I.G. In some embodiments, the promoter comprises an EF1α promoter. In some embodiments, the promoter comprises the nucleic acid sequence set forth in SEQ ID NO: 19. In some embodiments, the promoter is a synthetic promoter further comprising a non-coding element, such as any non-coding element described herein in Section I.G. In some embodiments, the synthetic promoter comprises the nucleic acid sequences set forth in SEQ ID NO: 19 and SEQ ID NO: 69. In some embodiments, the polynucleotide encoding the synthetic promoter further comprises the nucleic acid sequence set forth in SEQ ID NO: 70.

In some embodiments, the polynucleotide (e.g., the polynucleotide used to encode the RNAi expression cassette) comprising a synthetic promoter, comprises, from 5′ to 3′, the EF1α promoter set forth in SEQ ID NO: 19, the synthetic 5′ non-coding element set forth in SEQ ID NO: 69, any shRNA cassette disclosed herein, and the synthetic 3′ non-coding element set forth in SEQ ID NO: 70. In some embodiments, the polynucleotide (e.g., the polynucleotide used to encode the RNAi expression cassette) comprising a synthetic promoter, comprises, from 5′ to 3′, the EF1α promoter set forth in SEQ ID NO: 19, the synthetic 5′ non-coding element set forth in SEQ ID NO: 69, the shRNA cassette set forth in SEQ ID NO: 71, and the synthetic 3′ non-coding element set forth in SEQ ID NO: 70.

The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

Introduction of such constructs into host cells may be effected under conditions whereby the two or more nucleic acids that are contained within the recombinant nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature nucleic acids (e.g., shRNAs) may then induce degradation, and/or translation repression, of target gene mRNA transcripts produced in the cell. Alternatively, each of the precursor stemloop structures may be produced as part of a separate transcript, in which case each nucleic acid sequence will preferably include its own promoter and transcription terminator sequences. Additionally, the multiple nucleic acid precursor transcripts may reside within a single primary transcript.

In some aspects, the expression level of multiple target genes may be modulated using the methods and nucleic acids described herein. For example, the provided RNAi expression cassette contains a first set of nucleic acids that may be designed to include a sequence (a guide strand) that is designed to reduce the expression level of a first target gene, whereas a second set of nucleic acids may be designed to include a sequence (a guide strand) that is designed to reduce the expression level of a second target gene. The different sets of nucleic acids may be expressed and reside within the same, or separate, preliminary transcripts. In certain embodiments, such multiplex approach, i.e., the use of the nucleic acids described herein to modulate the expression level of two or more target genes, may have an enhanced therapeutic effect on a patient. For example, if a patient is provided with cells expressing the nucleic acid molecules described herein to treat, prevent, or ameliorate the effects of cancer, it may be desirable to provide the patient with two or more types of nucleic acids, which are designed to reduce the expression level of multiple genes that are implicated in activation or repression of immune cells.

The RNAi expression cassette (e.g., the one or more nucleic acid(s)) described herein may be capable of reducing target gene expression in a cell by at least more than about 50% as compared to a control cell that does not comprise the RNAi expression cassette (e.g., nucleic acid molecule(s)). For example, the RNAi expression cassette, such as the nucleic acid(s) (e.g., shRNA), can be capable of reducing expression of a target gene selected from the group consisting of TGFBR2, FAS, and/or PTPN2 in the cell by at least 10%, 15%, 20%, 25%, 30%, 35%, 40%, 45%, 50%, 55%, 60%, 65%, 75%, 80%, 85%, 90%, 95%, 97%, 98%, 99%, or more as compared to a control cell that does not comprise the respective RNAi expression cassette, such as the nucleic acid(s). The RNAi expression cassette (e.g., one or more nucleic acid molecule(s)) can be capable of reducing expression of a target gene selected from the group consisting of TGFBR2, FAS, and/or PTPN2 in the cell by at least between about 10-50%, 10-20%, 10-30%, 10-40%, 20-50%, 30-50%, 40-50%, 10-100%, 50-100%, 50-99%, 50-95%, 50-90%, 50-85%, 50-80%, 50-75%, 50-70%, 50-65%, 50-60%, 50-55%, or as compared to a control cell that does not comprise the respective RNAi expression cassette (e.g., nucleic acid(s)). In some embodiments, RNAi expression cassette (e.g., one or more nucleic acid(s)) reduces expression of TGFBR2 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the RNAi expression cassette (e.g., one or more nucleic acid(s)). In some embodiments, the RNAi expression cassette (e.g., one or more nucleic acid(s)) reduces expression of FAS in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the RNAi expression cassette (e.g., one or more nucleic acid(s)). In some embodiments, the RNAi expression cassette (e.g., one or more nucleic acid(s)) reduces expression of PTPN2 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the RNAi expression cassette (e.g., one or more nucleic acid(s)).

The nucleic acid(s) may be chemically synthesized, or in vitro transcribed, and may further include one or more modifications to phosphate-sugar backbone or nucleosides residues.

In some embodiments, the RNAi expression cassette is a triple shRNA expression cassette. In some embodiments, the triple shRNA expression cassette comprises, from 5′ to 3′, (1) a synthetic promoter (e.g., an EF1α promoter set forth in SEQ ID NO: 19 and a synthetic 5′ non-coding element set forth in SEQ ID NO: 69), (2) a 5′ backbone of a first miR (e.g., a miR-3G 5′ backbone), (3) a first strand of a first shRNA, (4) a loop of the first miR (e.g., a miR-3G loop), (5) a second strand of the first shRNA, (6) a 3′ backbone of the first miR (e.g., a miR-3G 3′ backbone), (7) a first spacer, (8) a 5′ backbone of a second miR (e.g., a miR-E 5′ backbone), (9) a first strand of a second shRNA, (10) a loop of the second miR (e.g., a miR-E loop), (11) a second strand of the second shRNA, (12) a 3′ backbone of the second miR (e.g., a miR-E 3′ backbone), (13) a second spacer, (14) a 5′ backbone of a third miR (e.g., a miR-3G 5′ backbone), (15) a first strand of a third shRNA, (16) a loop of the third miR (e.g., a miR-3G loop), (17) a second strand of the third shRNA, (18) a 3′ backbone of the third miR (e.g., a miR-3G 3′ backbone), (19) a PPT sequence, (20) a 5′ untranslated region (UTR), (21) an optional transgene (e.g., encoding a chimeric antigen receptor), and (22) a poly adenylation (poly A) sequence (e.g., a human growth hormone (GH1) poly A sequence). In some embodiments, the first, second, and third miRs are identical. In some embodiments, the first, second, and third miRs are all distinct. In some embodiments, the first and second miRs are identical and the third miR is distinct. In some embodiments, the first and third miRs are identical and the second miR is distinct. In some embodiments, the second and third miRs are identical and the first miR is distinct. In some embodiments, the first and third miRs are miR-3G and the second miR is miR-E (“3G-E-3G format”). In some embodiments, the first, second, and third miRs are all miR-3G (“3G-3G-3G format”).

In some embodiments, the expression cassette is a quadruple shRNA expression cassette. In some embodiments, the quadruple expression cassette comprises, from 5′ to 3′, (1) a synthetic promoter (e.g., an EF1α promoter set forth in SEQ ID NO: 19 and a synthetic 5′ non-coding element set forth in SEQ ID NO: 69), (2) a 5′ backbone of a first miR (e.g., a miR-3G 5′ backbone), (3) a first strand of a first shRNA, (4) a loop of the first miR (e.g., a miR-3G loop), (5) a second strand of the first shRNA, (6) a 3′ backbone of the first miR (e.g., a miR-3G 3′ backbone), (7) a first spacer, (8) a 5′ backbone of a second miR (e.g., a miR-E 5′ backbone), (9) a first strand of a second shRNA, (10) a loop of the second miR (e.g., a miR-E loop), (11) a second strand of the second shRNA, (12) a 3′ backbone of the second miR (e.g., a miR-E 3′ backbone), (13) a second spacer, (14) a 5′ backbone of a third miR (e.g., a miR-3G 5′ backbone), (15) a first strand of a third shRNA, (16) a loop of the third miR (e.g., a miR-3G loop), (17) a second strand of the third shRNA, (18) a 3′ backbone of the third miR (e.g., a miR-3G 3′ backbone), (19) a third spacer, (20) a 5′ backbone of a fourth miR (e.g., a miR-3G 5′ backbone), (21) a first strand of a fourth shRNA, (22) a loop of the fourth miR (e.g., a miR-3G loop), (23) a second strand of the fourth shRNA, (24) a 3′ backbone of the fourth miR (e.g., a miR-3G 3′ backbone), (25) a PPT sequence, (26) a 5′ untranslated region (UTR), (27) an optional transgene (e.g., a chimeric antigen receptor), and (28) a polyadenylation (poly A) sequence (e.g., a human growth hormone (GH1) polyA sequence). In some embodiments, the first, second, third, and fourth miRs are identical. In some embodiments, the first, second, third, and fourth miRs are all distinct. In some embodiments, a first group of two miRs are identical and a second group of two miRs are identical but distinct from the first group. In some embodiments, a first group of two miRs are identical and the remaining two miRs are each distinct from the first group and each other. In some embodiments, three of the miRs are identical and the last miR is distinct. In some embodiments, the first, third, and fourth miRs are miR-3G and the second miR is miR-E (“3G-E-3G-3G format”). In some embodiments, the quadruple expression cassette comprises a synthetic promoter comprising the nucleic acid sequences set forth in SEQ ID NO: 19 and SEQ ID NO: 69 followed by the nucleic acid sequence set forth in SEQ ID NO: 71, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto.

### VI. METHODS AND USES

Also provided are methods of using and uses of the provided genetically engineered T cells, and pharmaceutical compositions and formulations thereof, such as in the treatment of diseases, conditions, and disorders. Such methods and uses include therapeutic methods and uses, for example, involving administration of the genetically engineered T cells, or compositions containing the same, to a subject having a disease, condition, or disorder.

In some embodiments, the CAR of the genetically engineered T cell is directed against CD70, and the diseases, conditions and disorders include any in which CD70 is expressed. Such methods and uses include therapeutic methods and uses, for example, involving administration of the genetically engineered T cells to a subject having a disease, condition, or disorder associated with CD70 such as a disease, condition, or disorder associated with CD70 expression, and/or in which cells or tissues express, e.g., specifically express, CD70. Such methods and uses include therapeutic methods and uses, for example, involving administration of the genetically engineered T cells to a subject having a disease, condition, or disorder associated with CD70 such as a disease, condition, or disorder associated with CD70 expression, and/or in which cells or tissues express, e.g., specifically express, CD70. Such methods and uses include therapeutic methods and uses, for example, involving administration of the genetically engineered T cells, or compositions containing the same, to a subject having a disease, condition, or disorder associated with CD70 such as a disease, condition, or disorder associated with CD70 expression, and/or in which cells or tissues express, e.g., specifically express, CD70.

In some embodiments, the genetically engineered T cells are administered in an effective amount to effect treatment of the disease or disorder. Provided herein are uses of the recombinant receptors (e.g., CARs), and cells (e.g., engineered cells) in such methods and treatments, and in the preparation of a medicament to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with CD70, such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, a “subject” or an “individual” is a human.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor (CAR) or transgenic TCR specifically binds to an antigen associated with the disease or condition.

Among the diseases, conditions, and disorders are tumors, including solid tumors, and melanomas, and including localized and metastatic tumors.

Provided herein are methods of treatment that involve administering engineered cells or compositions containing engineered cells, such as engineered T cells, including methods for the treatment of subjects with systemic autoimmune diseases. In some embodiments, provided herein are methods and use of genetically engineered T cells (e.g., CD70-targeted CAR engineered T cells) and/or compositions thereof, including methods for the treatment of subjects with cancer. In particular embodiments, the method includes administering to the subject a dose of T cells that includes CD4+ and CD8+ T cells, wherein the T cells comprises a chimeric antigen receptor (CAR) that specifically binds to an antigen associated with cells of the condition. In some embodiments, the CAR specifically binds to CD70. In particular embodiments, the method includes administering to the subject a dose of T cells that includes CD4+ and CD8+ T cells, wherein the T cells comprises a chimeric antigen receptor (CAR) that specifically binds to CD70.

In some embodiment, the methods provided herein are used to treat cancer caused by, associated with and/or specific to cells expressing an antigen targeted by the CAR, such as CD70. In some embodiment, the methods provided herein are used to treat cancer caused by, associated with and/or specific to cells expressing CD70.

In some embodiments, the cancer is or includes, but is not limited to, T cell lymphoma, peripheral T cell lymphoma, solid tumors, renal cell carcinoma, clear cell renal cell carcinoma, renal cancer, bladder cancer, lung cancer, breast cancer, glioblastoma, pancreatic cancer, and melanoma.

In some embodiment, the compositions and methods provided herein are used to treat cancer such as solid tumors. In some embodiments, the compositions and methods provided herein are used to treat renal cell carcinoma. In some embodiments, the compositions and methods provided herein are used to treat clear cell renal cell carincoma. In some embodiments, the compositions and methods provided herein are used to treat sarcomatoid, papillary, chromophobe, and oncotytoma renal cell carcinoma.

In some embodiments, the cancer is a solid tumor, such as renal cell carcinoma. Among provided methods are methods of treatment that involve administering engineered cells or compositions containing engineered cells, such as engineered T cells to subjects with solid tumors. Also provided are methods and uses of provided CD70-targeted CAR engineered cells (e.g., T cells) and/or compositions thereof, including methods for the treatment of subjects having a solid tumor that involves administration of the engineered cells and/or compositions thereof. In some embodiments, the methods and use of provided genetically engineered T cells, and compositions thereof, include methods for the treatment of subjects with solid tumors that have failed at least two or more prior therapies. In particular embodiments, the method includes administering to the subject a dose of T cells that includes CD4+ and CD8+ T cells, wherein the T cells comprises a chimeric antigen receptor (CAR) that specifically binds to an antigen associated with the solid tumors, such as CD70.

Also provided herein are methods of treating subjects who have been previously treated for cancer, but who are non-responsive or insufficiently responsive to standard therapies, as well as those who have never been previously treated. In some embodiments, the subjects to be treated with one of the methods provided herein have developed drug resistance to one or more standard therapies. Further encompassed are methods of treating subjects who have undergone surgery in an attempt to treat the disease or condition at issue, as well as those who have not.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the methods include adoptive cell therapy, whereby genetically engineered cells expressing the provided recombinant receptors comprising a CD70-binding domain (e.g., CARs comprising anti-CD70 antibody or antigen-binding fragment thereof) are administered to subjects. Such administration can promote activation of the cells (e.g., T cell activation) in a CD70-targeted manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a CD70-expressing cancer.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a donor other than a subject who is to receive or who ultimately receives the cell therapy, e.g., a first subject. In some embodiments, the donor is a healthy donor. In some embodiments, the donor does not have the disease or disorder. In some embodiments, the donor and subject are genetically identical. In some embodiments, the donor and subject are genetically similar. In some embodiments, the subject expresses the same HLA class or supertype as the donor. In some embodiments, the subject does not express the same HLA class or supertype as the donor.

In some embodiments the cells administered are immune cells engineered to express the CD70-binding recombinant receptor, e.g., CAR. In some embodiments the immune cells are T cells. In some embodiments, the administered cells are CD4+ T cells. In some embodiments the administered cells are CD8+ T cells. In some embodiments, the administered cells are a combination of CD4+ and CD8+ T cells, such as CAR T cells. In some examples the ratio of CD4+ cells to CD8+ cells (CD4:CD8) is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

### A. Doses and Regimens

In certain embodiments, a dose of a composition comprising a plurality (e.g., a population) of engineered T cells described herein comprises a therapeutically effective amount of the engineered T cells. In some of any embodiments, the dose of engineered T cells comprises viable engineered T cells.

In some embodiments, the engineered T cells of the dose comprise CD4+ and CD8+ T cells.

In some embodiments, the dose comprises from about 25×10⁶ engineered T cells to about 1.5×10⁹ of the engineered T cells, from about 50×10⁶ engineered T cells to about 1.5×10⁹ of the engineered T cells, from about 100×10⁶ engineered T cells to about 1.5×10⁹ of the engineered T cells, from about 300×10⁶ engineered T cells to about 1.5×10⁹ of the engineered T cells, from about 900×10⁶ engineered T cells to about 1.5×10⁹ of the engineered T cells, from about 25×10⁶ engineered T cells to about 1×10⁹ of the engineered T cells, from about 50×10⁶ engineered T cells to about 1×10⁹ of the engineered T cells, from about 100×10⁶ engineered T cells to about 1×10⁹ of the engineered T cells, from about 300×10⁶ engineered T cells to about 1×10⁹ of the engineered T cells, from about 900×10⁶ engineered T cells to about 1×10⁹ of the engineered T cells, from about 25×10⁶ engineered T cells to about 900×10⁶ of the engineered T cells, from about 50×10⁶ engineered T cells to about 900×10⁶ of the engineered T cells, from about 100×10⁶ engineered T cells to about 900×10⁶ of the engineered T cells, from about 300×10⁶ engineered T cells to about 900×10⁶ of the engineered T cells, from about 25×10⁶ engineered T cells to about 300×10⁶ of the engineered T cells, from about 50×10⁶ engineered T cells to about 300×10⁶ of the engineered T cells, from about 100×10⁶ engineered T cells to about 300×10⁶ of the engineered T cells, from about 25×10⁶ engineered T cells to about 150×10⁶ of the engineered T cells, from about 50×10⁶ engineered T cells to about 100×10⁶ of the engineered T cells, or from about 25×10⁶ engineered T cells to about 50×10⁶ of the engineered T cells. In some embodiments, the dose comprises from about 50×10⁶ engineered T cells to about 900×10⁶ of the engineered T cells. In some embodiments, the engineered T cells are viable engineered T cells. In some embodiments, the engineered T cells are CAR+ engineered T cells.

In some embodiments, the dose comprises about 25×10⁶ engineered T cells. In some embodiments, the dose comprises about 50×10⁶ engineered T cells. In some embodiments, the dose comprises about 100×10⁶ engineered T cells. In some embodiments, the dose comprises about 150×10⁶ engineered T cells. In some embodiments, the dose comprises about 200×10⁶ engineered T cells. In some embodiments, the dose comprises about 250×10⁶ of the engineered T cells. In some embodiments, the dose comprises about 300×10⁶ of the engineered T cells. In some embodiments, the dose comprises about 350×10⁶ of the engineered T cells. In some embodiments, the dose comprises about 400×10⁶ of the engineered T cells. In some embodiments, the dose comprises about 450×10⁶ of the engineered T cells. In some embodiments, the dose comprises about 500×10⁶ of the engineered T cells. In some embodiments, the dose comprises about 550×10⁶ of the engineered T cells. In some embodiments, the dose comprises about 650×10⁶ of the engineered T cells. In some embodiments, the dose comprises about 700×10⁶ of the engineered T cells. In some embodiments, the dose comprises about 750×10⁶ of the engineered T cells. In some embodiments, the dose comprises about 800×10⁶ of the engineered T cells. In some embodiments, the dose comprises about 850×10⁶ of the engineered T cells. In some embodiments, the dose comprises about 900×10⁶ of the engineered T cells. In some embodiments, the dose comprises about 950×10⁶ of the engineered T cells. In some embodiments, the dose comprises about 1×10⁹ of the engineered T cells. In some embodiments, the dose comprises about 1.1×10⁹ of the engineered T cells. In some embodiments, the dose comprises about 1.2×10⁹ of the engineered T cells. In some embodiments, the dose comprises about 1.3×10⁹ of the engineered T cells. In some embodiments, the dose comprises about 1.4×10⁹ of the engineered T cells. In some embodiments, the dose comprises about 1.5×10⁹ of the engineered T cells. In some embodiments, the engineered T cells are viable engineered T cells. In some embodiments, the engineered T cells are CAR+ engineered T cells. In certain embodiments, the dose is administered via intravenous (IV) infusion. In some embodiments, the IV infusion is a single IV infusion. In certain embodiments, the dose is provided as a suspension for administration via IV infusion.

In some embodiments, the subject receives one dose or a single IV infusion. In some embodiments, the subject receives more than one dose (e.g., two, three, four, five, six, seven, eight, nine, ten or more doses) or more than one IV infusion (e.g., two, three, four, five, six, seven, eight, nine, ten or more IV infusions).

In some embodiments, the subject or patient is pretreated with a lymphodepleting therapy, such as a lymphodepleting chemotherapy, prior to administration of the engineered T cells. In some embodiments, the lymphodepleting chemotherapy comprises administering fludarabine. In some embodiments, the fludarabine is administered at a dose of 5 mg/m²/day to 50 mg/m²/day via IV infusion. In some embodiments, the fludarabine is administered at a dose of 20 mg/m²/day to 40 mg/m²/day via IV infusion. In some embodiments, the fludarabine is administered at a dose of 30 mg/m²/day via IV infusion. In some embodiments, the lymphodepleting chemotherapy comprises administering cyclophosphamide. In some embodiments, the cyclophosphamide is administered at a dose of about 250 mg/m²/day to about 1500 mg/m²/day. In some embodiments, the cyclophosphamide is administered at a dose of about 500 mg/m²/day to about 1200 mg/m²/day. In some embodiments, the cyclophosphamide is administered at a dose of about 300 mg/m²/day to about 700 mg/m²/day. In some embodiments, the cyclophosphamide is administered at a dose of about 400 mg/m²/day to about 600 mg/m²/day. In some embodiments, the cyclophosphamide is administered at a dose of 250 mg/m²/day, 300 mg/m²/day, 500 mg/m²/day, 700 mg/m²/day, or 1500 mg/m²/day. In some embodiments, the cyclophosphamide is administered at a dose of 750 mg/m²/day. In some embodiments, the cyclophosphamide is administered at a dose of 1000 mg/m²/day. In some embodiments, the cyclophosphamide is administered at a dose of 60 mg/kg/day. The lymphodepleting chemotherapy can be administered for 3, 4, or 5 days prior to administration of the engineered T cells. In some embodiments, the lymphodepleting chemotherapy comprises treating the patient with fludarabine IV and cyclophosphamide IV for three, four, or five days prior to administration of the engineered T cells. In some embodiments, the lymphodepleting chemotherapy comprises treating the patient with fludarabine IV and cyclophosphamide IV for three days prior to administration of the engineered T cells. In some embodiments, the lymphodepleting chemotherapy comprises treating the patient with fludarabine IV (30 mg/m²/day) and cyclophosphamide IV (500 mg/m²/day) for three days prior to administration of the engineered T cells. In some embodiments, the lymphodepleting chemotherapy comprises treating the patient with fludarabine IV and cyclophosphamide IV for four days or five days prior to administration of the engineered T cells. In some embodiments, the lymphodepleting chemotherapy comprises treating the patient with fludarabine IV (30 mg/m²/day) and cyclophosphamide IV (500 mg/m²/day) for four days or five days prior to administration of the engineered T cells. In some embodiments, the lymphodepleting chemotherapy comprises treating the patient with fludarabine IV (30 mg/m²/day) and cyclophosphamide IV (750 mg/m²/day) for three days prior to administration of the engineered T cells. In some embodiments, the lymphodepleting chemotherapy comprises treating the patient with fludarabine IV (30 mg/m²/day) and cyclophosphamide IV (1000 mg/m²/day) for three days prior to administration of the engineered T cells. In some embodiments, the lymphodepleting chemotherapy comprises treating the patient with fludarabine IV (30 mg/m²/day) and cyclophosphamide IV (750 mg/m²/day) for four days or five days prior to administration of the engineered T cells. In some embodiments, the lymphodepleting chemotherapy comprises treating the patient with fludarabine IV (30 mg/m²/day) and cyclophosphamide IV (1000 mg/m²/day) for four or five days prior to administration of the engineered T cells.

### B. Combination Therapies

The method provided herein, in some instances, further comprises administering the engineered T cells in combination with at least one additional therapy, therapeutic agent, or modality. The engineered T cells can be administered at any of the doses described above and either simultaneously or sequentially with the at least one additional therapy, therapeutic agent, or modality. In some embodiments, the at least one additional therapy, therapeutic agent, or modality is administered prior to administration of the engineered T cells. Alternatively or in addition, the at least one additional therapy, therapeutic agent, or modality can be administered after the administration of the engineered T cells. In some embodiments, the subject or patient is pretreated with a lymphodepleting therapy, such as a lymphodepleting chemotherapy, prior to administration of the engineered T cells and initiation of the treatment with the at least one additional therapy, therapeutic agent, or modality.

In some embodiments, the method provided herein does not comprise administering the engineered T cells in combination with certain medications or treatments, the use of which may be restricted or prohibited. In some embodiments, the engineered T cells are not administered simultaneously or sequentially, such as after administration of the engineered T cells or during pretreatment with a lymphodepleting therapy with potentially restricted or prohibited medications or treatments. In some embodiments, the medications or treatments comprise steroids, corticosteroids, plasmapheresis, immunosuppressive and/or immunomodulatory therapies, antineoplastic agents, live vaccines, anti-epidermal growth factor receptor-targeted treatments (e.g., cetuximab), experimental agents, G-CSF, curative radiation, or strong inducers or inhibitors of CYP3A4.

In some embodiments, steroids are not administered for a period of time prior to, during, or after administration of engineered T cells. In some embodiments, is tapered≤10 mg/day prednisone or equivalent 14 days prior to leukapheresis. In some embodiments, steroids are not administered after administration of engineered T cells.

In some embodiments, plasmapheresis is not administered prior to or during administration of engineered T cells.

In some embodiments, immunosuppressive and/or immunomodulatory therapies and antineoplastic agents are not administered during or after administration of engineered T cells. In some embodiments, immunosuppressive and/or immunomodulatory therapies and antineoplastic agents are not administered during or after administration of engineered T cells during the treatment period. However, in some embodiments, immunosuppressive and/or immunomodulatory therapies and antineoplastic agents are administered as part of a lymphodepletion chemotherapy or for treatment of uncontrolled proliferation of the engineered T cells, cytokine release syndrome, or immune effector cell-associated neurotoxicity syndrome.

In some embodiments, live vaccines are not administered within 6 weeks prior to, during, or after administration of engineered T cells.

In some embodiments, short-acting G-CSF (e.g., filgrastim) is not administered from about 7 days prior to about 7 days after administration of engineered T cells. In some embodiments, long-acting G-CSF is not administered from about 14 days prior to about 7 days after administration of engineered T cells.

### 1. PD-1 Therapies

In some embodiments, the at one additional therapeutic agent or modality comprises at least one PD-1 therapy. In some embodiments, the PD-1 therapy is an antibody or antigen-binding fragment thereof that binds to PD-1, or an antibody or antigen-binding fragment thereof that binds to PD-L1. In some embodiments, the PD-1 therapy is a small molecule targeting PD-1 or PD-L1.

In some embodiments, the engineered T cells and the PD-1 therapy are administered separately. In some embodiments, the engineered T cells and the PD-1 therapy are administered sequentially. In some embodiments, the engineered T cells are administered after the PD-1 therapy. In some embodiments, the PD-1 therapy is administered after the engineered T cells. In some embodiments, the engineered T cells and the PD-1 therapy are co-administered, wherein the engineered T cells are in a first bag and the PD-1 therapy is in a second bag, and wherein the engineered T cells and the PD-1 therapy are administered simultaneously.

In some embodiments, the PD-1 therapy is administered to the subject by intravenous administration. In some embodiments, the PD-1 therapy is administered to the subject by subcutaneous administration. In some embodiments, the PD-1 therapy is administered to the subject by any other standard means known in the art.

The PD-1 therapy, in some instances, is selected from nivolumab, pembrolizumab, cemiplimab, dostarlimab, retifanlimab, toripalimab, atezolizumab, avelumab, durvalumab, cosibelimab, camrelizumab, sintilimab, and tislelizumab.

In some embodiments, the PD-1 therapy is nivolumab. In some embodiments, the PD-1 therapy is pembrolizumab. In some embodiments, the PD-1 therapy is cemiplimab. In some embodiments, the PD-1 therapy is dostarlimab. In some embodiments, the PD-1 therapy is retifanlimab. In some embodiments, the PD-1 therapy is toripalimab. In some embodiments, the PD-1 therapy is atezolizumab. In some embodiments, the PD-1 therapy is avelumab. In some embodiments, the PD-1 therapy is durvalumab. In some embodiments, the PD-1 therapy is avelumab. In some embodiments, the PD-1 therapy is cosibelimab. In some embodiments, the PD-1 therapy is avelumab. In some embodiments, the PD-1 therapy is tislelizumab. In some embodiments, the PD-1 therapy is avelumab. In some embodiments, the PD-1 therapy is camrelizumab. In some embodiments, the PD-1 therapy is avelumab. In some embodiments, the PD-1 therapy is sintilimab.

In some embodiments, the nivolumab is administered to the subject about once every two weeks at a dose of or about 240 mg, about once every three weeks at a dose of or about 360 mg, or about once every four weeks at a dose of or about 480 mg. In some embodiments, nivolumab is administered to the subject once every two weeks at a dose of or about 320 mg to a dose to or to about 600 mg, inclusive. In some embodiments, nivolumab is administered to the subject about once every two weeks at a dose of or about 240 mg. In some embodiments, nivolumab is administered to the subject about once every two weeks at a dose of or about 600 mg. In some embodiments, nivolumab is administered to the subject once every three weeks at a dose of or about 360 mg to a dose to or to about 1200 mg, inclusive. In some embodiments, nivolumab is administered to the subject about once every three weeks at a dose of or about 360 mg. In some embodiments, nivolumab is administered to the subject about once every three weeks at a dose of or about 720 mg. In some embodiments, nivolumab is administered to the subject about once every three weeks at a dose of or about 900 mg. In some embodiments, nivolumab is administered to the subject about once every three weeks at a dose of or about 960 mg. In some embodiments, nivolumab is administered to the subject about once every three weeks at a dose of or about 1200 mg. In some embodiments, nivolumab is administered to the subject about once every four weeks at a dose of or about 480 mg to or to about 1200 mg, inclusive. In some embodiments, nivolumab is administered to the subject about once every four weeks at a dose of or about 480 mg. In some embodiments, nivolumab is administered to the subject about once every four weeks at a dose of or about 720 mg. In some embodiments, nivolumab is administered to the subject about once every four weeks at a dose of or about 960 mg. In some embodiments, nivolumab is administered to the subject about once every four weeks at a dose of or about 1200 mg. In some embodiments, nivolumab is administered about once every two weeks at a dose of between at or about 3 mg/kg and at or about 10 mg/kg. In some embodiments, nivolumab is administered about once every two weeks at a dose of or about 3 mg/kg.

In some embodiments, nivolumab is administered intravenously to the subject about once every four weeks at a dose or of about 480 mg.

In some embodiments, nivolumab is administered subcutaneously to the subject once every two to four weeks at a dose of or about 600 mg to or to about 1200 mg, inclusive. In some embodiments, nivolumab is administered subcutaneously to the subject about once every two weeks (Q2W) at a dose of or about 600 mg. In some embodiments, nivolumab is administered subcutaneously to the subject about once every three weeks (Q3W) at a dose of or about 720 mg, of or about 900 mg, of or about 960 mg, or of or about 1200 mg. In some embodiments, nivolumab is administered subcutaneously to the subject about once every four weeks (Q4W) at a dose of or about 720 mg, of or about 960 mg, or of or about 1200 mg. In some embodiments, nivolumab is administered subcutaneously to the subject about once every four weeks (Q4W) at a dose of or about 1200 mg.

In some of any such embodiments, the nivolumab administered subcutaneously is co-formulated with hyaluronidase. Co-formulation of nivolumab with hyaluronidase allows administration of larger volumes with fewer injections subcutaneously than without hyaluronidase, as described in Albiges L et al., Ann Oncol. 2025; 36(1):99-107, which is incorporated by reference in its entirety. In some embodiments, the hyaluronidase is recombinant human hyaluronidase (rHuPH20; Halozyme Therapeutics, Inc.), which is a recombinant form of human PH20 that is a 447 amino acid sequence devoid of the GPI anchor attachment motif. See e.g., Bookbinder et al. (2006) Journal of Controlled Release, 114:230-241; described in Locke et al. (2019) Drug Delivery, 26:98-106, which are incorporated by reference in their entirety. In some embodiments, nivolumab is co-formulated with hyaluronidase at a dose of or about 10,000 units to 20,000 units, inclusive. In some embodiments, nivolumab is co-formulated with hyaluronidase at a dose of or about 10,000 units; of or about 12,000 units; of or about 15,000 units; of or about 16,000 units; or of or about 20,000 units.

In some embodiments, nivolumab co-formulated with hyaluronidase is administered subcutaneously to the subject once every two to four weeks at a dose of or about 600 mg to or to about 1200 mg nivolumab, inclusive, and of or about 10,000 units to of or about 20,000 units hyaluronidase, inclusive.

In some embodiments, nivolumab co-formulated with hyaluronidase is administered subcutaneously to the subject once every two weeks at a dose of or about 720 mg nivolumab and of or about 20,000 units hyaluronidase. In some embodiments, nivolumab co-formulated with hyaluronidase is administered subcutaneously to the subject once every two weeks at a dose of or about 960 mg nivolumab and of or about 20,000 units hyaluronidase. In some embodiments, nivolumab co-formulated with hyaluronidase is administered subcutaneously to the subject once every two weeks at a dose of or about 1200 mg nivolumab and of or about 20,000 units hyaluronidase. In some embodiments, nivolumab co-formulated with hyaluronidase is administered subcutaneously to the subject once every two weeks at a dose of or about 720 mg nivolumab and of or about 12,000 units hyaluronidase. In some embodiments, nivolumab co-formulated with hyaluronidase is administered subcutaneously to the subject once every two weeks at a dose of or about 960 mg nivolumab and of or about 12,000 units hyaluronidase. In some embodiments, nivolumab co-formulated with hyaluronidase is administered subcutaneously to the subject once every two weeks at a dose of or about 1200 mg nivolumab and of or about 12,000 units hyaluronidase.

In some embodiments, nivolumab co-formulated with hyaluronidase is administered subcutaneously to the subject once every two weeks at a dose of or about 600 mg nivolumab and of or about 10,000 units hyaluronidase.

In some embodiments, nivolumab co-formulated with hyaluronidase is administered subcutaneously to the subject once every three weeks at a dose of or about 720 mg nivolumab and of or about 20,000 units hyaluronidase. In some embodiments, nivolumab co-formulated with hyaluronidase is administered subcutaneously to the subject once every three weeks at a dose of or about 960 mg nivolumab and of or about 20,000 units hyaluronidase. In some embodiments, nivolumab co-formulated with hyaluronidase is administered subcutaneously to the subject once every three weeks at a dose of or about 1200 mg nivolumab and of or about 20,000 units hyaluronidase. In some embodiments, nivolumab co-formulated with hyaluronidase is administered subcutaneously to the subject once every three weeks at a dose of or about 720 mg nivolumab and of or about 12,000 units hyaluronidase. In some embodiments, nivolumab co-formulated with hyaluronidase is administered subcutaneously to the subject once every three weeks at a dose of or about 960 mg nivolumab and of or about 12,000 units hyaluronidase. In some embodiments, nivolumab co-formulated with hyaluronidase is administered subcutaneously to the subject once every three weeks at a dose of or about 1200 mg nivolumab and of or about 12,000 units hyaluronidase.

In some embodiments, nivolumab co-formulated with hyaluronidase is administered subcutaneously to the subject once every three weeks at a dose of or about 900 mg nivolumab and of or about 15,000 units hyaluronidase.

In some embodiments, nivolumab co-formulated with hyaluronidase is administered subcutaneously to the subject once every four weeks at a dose of or about 720 mg nivolumab and of or about 20,000 units hyaluronidase. In some embodiments, nivolumab co-formulated with hyaluronidase is administered subcutaneously to the subject once every four weeks at a dose of or about 960 mg nivolumab and of or about 20,000 units hyaluronidase. In some embodiments, nivolumab co-formulated with hyaluronidase is administered subcutaneously to the subject once every four weeks at a dose of or about 1200 mg nivolumab and of or about 20,000 units hyaluronidase. In some embodiments, nivolumab co-formulated with hyaluronidase is administered subcutaneously to the subject once every four weeks at a dose of or about 720 mg nivolumab and of or about 12,000 units hyaluronidase. In some embodiments, nivolumab co-formulated with hyaluronidase is administered subcutaneously to the subject once every four weeks at a dose of or about 960 mg nivolumab and of or about 12,000 units hyaluronidase. In some embodiments, nivolumab co-formulated with hyaluronidase is administered subcutaneously to the subject once every four weeks at a dose of or about 1200 mg nivolumab and of or about 12,000 units hyaluronidase.

In some embodiments, nivolumab co-formulated with hyaluronidase is administered subcutaneously to the subject once every four weeks at a dose of or about 1200 mg nivolumab and of or about 20,000 units hyaluronidase.

In some embodiments, the PD-1 therapy is pembrolizumab, and pembrolizumab is administered to the subject about once every three weeks at a dose of or about 200 mg or about once every six weeks at a dose of or about 400 mg. In some embodiments, pembrolizumab is administered to the subject about once every three weeks at a dose of or about 200 mg. In some embodiments, pembrolizumab is administered to the subject about once every six weeks at a dose of 400 mg.

In some embodiments, the PD-1 therapy is cemiplimab, and cemiplimab is administered to the subject about once every three weeks at a dose of or about 350 mg.

In some embodiments, the PD-1 therapy is dostarlimab, and dostarlimab is administered to the subject about once every three weeks at a dose of or about 500 mg for dose 1 through dose 4 and about once every six weeks at a dose of or about 1000 mg for dose 5 onwards.

In some embodiments, the PD-1 therapy is retifanlimab, and retifanlimab is administered to the subject about once every four weeks at a dose of or about 500 mg.

In some embodiments, the PD-1 therapy is toripalimab, and toripalimab is administered to the subject about once every two weeks at a dose of or about 3 mg/kg.

In some embodiments, the PD-1 therapy is atezolizumab, and atezolizumab is administered to the subject about once every two weeks at a dose of or about 840 mg, once every three weeks at a dose of or about 1200 mg, or once every four weeks at a dose of or about 1680 mg. In some embodiments, atezolizumab is administered to the subject about once every two weeks at a dose of or about 840 mg. In some embodiments, atezolizumab is administered to the subject about once every three weeks at a dose of or about 1200 mg. In some embodiments, atezolizumab is administered to the subject about once every four weeks at a dose of or about 1680 mg.

In some embodiments, the PD-1 therapy is avelumab, and avelumab is administered to the subject about once every two weeks at a dose of or about 800 mg.

In some embodiments, the PD-1 therapy is durvalumab, and durvalumab is administered to the subject about once every two weeks at a dose of or about 10 mg/kg.

In some instances, the engineered T cells are administered in combination with nivolumab according to a dose and dosing schedule described herein. In some embodiments, the engineered T cells are administered about once every two weeks at a dose of or about 30 mg, of or about 90 mg, of or about 250 mg, of or about 500 mg, or of or about 1000 mg in combination with nivolumab, wherein nivolumab is administered to the subject about once every two weeks at a dose of or about 240 mg, about once every three weeks at a dose of or about 360 mg, or about once every four weeks at a dose of or about 480 mg.

In some embodiments, the PD-1 therapy is administered to the patient for 4, 6, 8, 12, 15, 18, 20, 24, 30, 36, 40, 48, or 60 months. In some embodiments, administration of the PD-1 therapy is discontinued after 4, 6, 8, 12, 15, 18, 20, 24, 30, 36, 40, 48, or 60 months. In some embodiments, the PD-1 therapy is administered to the subject for 12 months or for up to 12 months. In some embodiments, administration of the PD-1 therapy is discontinued after 12 months. In some embodiments, the PD-1 therapy is administered to the subject for 24 months or for up to 24 months. In some embodiments, administration of the PD-1 therapy is discontinued after 24 months.

### 2. Tyrosine Kinase Inhibitors

In some embodiments, the at least one additional therapeutic agent or modality comprises at least one tyrosine kinase inhibitor (TKI). In some embodiments, the TKI is a small-molecule inhibitor of at least one of c-Met (HGFR), VEGFR1, VEGFR2, VEGFR3, AXL, RET, and FLT3. In some embodiments, the TKI is a small-molecule inhibitor of at least one of c-Met (HGFR), VEGFR2, AXL, RET, and FLT3. In some embodiments, the TKI is a small-molecule inhibitor of at least one of VEGFR1, VEGFR2, and VEGFR3. In some embodiments, the TKI is administered as an oral capsule or tablet.

In some embodiments, the at least one TKI is or comprises cabozantinib, tivozanib, lenvatinib, axitinib, pazopanib or sunitinib.

In some embodiments, the at least one TKI is or comprises cabozantinib. In some embodiments, cabozantinib is administered to the subject once daily. In some embodiments, cabozantinib is administered to the subject at a dose of or about 40 mg/day. In some embodiments, cabozantinib is administered to the subject at a dose of or about 60 mg/day. Cabozantinib can be taken without food.

In some embodiments, strong inducers or inhibitors of CYP3A4 are not administered with cabozantinib. In some embodiments, strong inducers or inhibitors of CYP3A4 are not administered within about 14 days prior to administration of cabozantinib. In some embodiments, strong inducers of CYP3A4 comprises carbamazepine, phenytoin, or rifampin. In some embodiments, strong inhibitors of CYP3A4 comprises clarithromycin, indinavir, itraconazole, ketoconazole, nefazodone, nelfinavir, ritonavir, saquinavir, or telithromycin.

In some embodiments, the at least one TKI is or comprises tivozanib. In some embodiments, tivozanib is administered to the subject once daily. In some embodiments, tivozanib is administered in a dosing cycle. For example, tivozanib can be administered to the subject in a 28-day cycle comprising taking one dose orally for 21 days followed by seven days off. In some embodiments, tivozanib is administered at a dose of or about 1.34 mg/day. In some embodiments, tivozanib is administered at a dose of or about 0.89 mg/day.

### VII. ARTICLES OF MANUFACTURE OR KITS

Also provided are articles of manufacture or kits containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., a cancer). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the CD70-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a CD70-associated disease, disorder or condition in an individual. The label or package insert may further indicate that the formulation is useful or intended for subcutaneous, intravenous, or other modes of administration for treating or preventing a CD70-expressing or CD70-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the CAR; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, the term “endogenous” refers to a referenced molecule, such as a polynucleotide (e.g. gene), or polypeptide, that is present in a native or unmodified cell, such as a T cell. For instance, the term when used in reference to expression of an endogenous gene refers to expression of a gene encoded by an endogenous nucleic acid contained within a T cell isolated from a subject.

As used herein, a “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

As used herein, the term “locus” or “loci” refers to a fixed position on a chromosome where a particular gene or genetic marker is located. Reference to a “gene loci” refers to a particular locus of a desired gene in which it is desired to target a genetic modification, such as a gene edit or integration of a transgene.

As used herein “genetic disruption” with reference to an endogenous gene loci refers to a loci that has been modified using gene editing for directed DNA cleavage so that no functional endogenous gene product is produced from the disrupted genetic loci. In certain embodiments, the disruption results in expression of non-functional protein products, including but not limited to truncations, deletions, point mutations and insertions. In other embodiments, the disruption results in no protein expression from the endogenous gene loci.

As used herein, the term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “transgene” with reference to a polynucleotide is intended to mean that the referenced molecule is introduced into the cell of interest such that it is transferred into the cell. Typically, a transgene sequence is not normally expressed by the cells. In some cases, the transgene sequence is thus heterologous or exogenous to the cell and introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values>0 and <2 if the variable is inherently continuous.

As used herein, a “subject” is a mammal, such as a human or other animal, and typically is human.

### IX. EXEMPLARY EMBODIMENTS

Among the provided embodiments are:

1. A chimeric antigen receptor (CAR) directed against CD70, wherein the CAR comprises a CD70-binding domain that binds to CD70 comprising a heavy chain only variable (VHH) region, wherein: (i) the VHH region comprises a CDR-1, CDR-2, and CDR-3 each comprising a sequence that is contained within SEQ ID NO: 1, wherein X is Q or pyroglutamate; or (ii) the VHH region comprises a CDR-1, CDR-2, and CDR-3 each comprising a sequence that is contained within SEQ ID NO:27, wherein X is Q or pyroglutamate. 2. The CAR of embodiment 1, further comprising a spacer domain, a transmembrane domain, and an intracellular signaling domain. 3. The CAR of embodiment 1 or embodiment 2, wherein the VHH region of the CD70-binding domain comprises CDR-1, CDR-2, and CDR-3 sequences set forth in SEQ ID NOs: 4, 5, and 6, respectively. 4. The CAR of embodiment 1 or embodiment 2, wherein the VHH region of the CD70-binding domain comprises CDR-1, CDR-2, and CDR-3 sequences set forth in SEQ ID NOs: 7, 8, and 9, respectively. 5. The CAR of embodiment 1 or embodiment 2, wherein the VHH region of the CD70-binding domain comprises CDR-1, CDR-2, and CDR-3 sequences set forth in SEQ ID NOs: 79, 80, and 81, respectively. 6. The CAR of embodiment 1 or embodiment 2, wherein the VHH region of the CD70-binding domain comprises CDR-1, CDR-2, and CDR-3 sequences set forth in SEQ ID NOs: 82, 83, and 84, respectively. 7. The CAR of any one of embodiments 1-6, wherein VHH region of the CD70-binding domain, wherein: (i) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or (ii) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate. 8. The CAR of any one of embodiments 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 2. 9. The CAR of any one of embodiments 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 3, wherein X is pyroglutamate. 10. The CAR of any one of embodiments 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO:28. 11. The CAR of any one of embodiments 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 29, wherein X is pyroglutamate. 12. The CAR of any one of embodiments 1-7, wherein the VHH region of the CD70-binding domain, wherein: (i) the VHH region comprises a sequence set forth in SEQ ID NO: 1, wherein X is Q or pyroglutamate; or (ii) the VHH region comprises a sequence set forth in SEQ ID NO: 27, wherein X is Q or pyroglutamate. 13. The CAR of any one of embodiments 1-12, wherein the VHH region comprises a sequence set forth in SEQ ID NO: 2. 14. The CAR of any one of embodiments 1-12, wherein the VHH region comprises a sequence set forth in SEQ ID NO: 3, wherein X is pyroglutamate. 15. The CAR of any one of embodiments 1-12, wherein the VHH region comprises a sequence set forth in SEQ ID NO: 28. 16. The CAR of any one of embodiments 1-12, wherein the VHH region comprises a sequence set forth in SEQ ID NO: 29, wherein X is pyroglutamate. 17. The CAR of any one of embodiments 2-16, wherein the spacer domain comprises a hinge domain from human CD28. 18. The CAR of any one of embodiments 2-17, wherein the spacer domain comprises an amino acid sequence having at least 85%, 86%, 87%, 88%, 89%, 90%, 91%, 92%, 93%, 94%, 95%, 96%, 97%, 98% or 99% identity to SEQ ID NO: 10. 19. The CAR of embodiment 18, wherein the spacer domain comprises the sequence set forth in SEQ ID NO: 10. 20. The CAR of any one of embodiments 2-19, wherein the transmembrane domain comprises a transmembrane domain from human CD28. 21. The CAR of any one of embodiments 2-20, wherein the transmembrane domain comprises an amino acid sequence having at least at or about 85%, 86%, 87%, 88%, 89%, 90%, 91%, 92%, 93%, 94%, 95%, 96%, 97%, 98% or 99% identity to SEQ ID NO: 11. 22. The CAR of any one of embodiments 2-13, wherein the transmembrane domain comprises the sequence set forth in SEQ ID NO: 11. 23. The CAR of any one of embodiments 2-21, wherein the intracellular signaling domain comprises a cytoplasmic signaling domain of a human CD3-zeta (CD3ζ) chain. 24. The CAR of any one of embodiments 2-23, wherein the intracellular signaling domain comprises an amino acid sequence having at least at or about 90%, 91%, 92%, 93%, 94%, 95%, 96%, 97%, 98% or 99% sequence identity to SEQ ID NO: 13. 25. The CAR of any one of embodiments 2-24, wherein the intracellular signaling domain comprises the amino acid sequence set forth in SEQ ID NO: 13. 26. The CAR of any one of embodiments 2-24, wherein the intracellular signaling region further comprises a costimulatory signaling domain. 27. The CAR of embodiment 26, wherein the costimulatory signaling region comprises an intracellular signaling domain of a T cell costimulatory molecule or a signaling portion thereof. 28. The CAR of embodiment 26 or embodiment 27, wherein the costimulatory signaling region comprises an intracellular signaling domain of human 4-1BB. 29. The CAR of any one of embodiments 26-28, wherein the costimulatory signaling region comprises an amino acid sequence having at least at or about 90%, 91%, 92%, 93%, 94%, 95%, 96%, 97%, 98% or 99% sequence identity to SEQ ID NO: 12. 30. The CAR of any one of embodiments 26-29, wherein the costimulatory signaling region comprises the amino acid sequence set forth in SEQ ID NO: 12.

31. The CAR of any one of embodiments 1-29, wherein the CAR comprises the amino acid sequence set forth in SEQ ID NO: 38 or SEQ ID NO: 41, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38 or SEQ ID NO: 41, wherein X is Q or pyroglutamate. 32. The CAR of any one of embodiments 1-31, wherein the CAR comprises the amino acid sequence set forth in SEQ ID NO: 38, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38, wherein X is Q or pyroglutamate. 33. The CAR of any one of embodiments 1-32, wherein the CAR comprises the amino acid sequence set forth in SEQ ID NO: 38. 34. The CAR of any one of embodiments 1-32, wherein the CAR comprises the amino acid sequence set forth in SEQ ID NO: 39, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 39. 35. The CAR of any one of embodiments 1-32, wherein the CAR comprises the amino acid sequence set forth in SEQ ID NO: 39. 36. The CAR of any one of embodiments 1-32, wherein the CAR comprises the amino acid sequence set forth in SEQ ID NO: 40, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 40, wherein X is pyroglutamate. 37. The CAR of any one of embodiments 1-33 and 36, wherein the CAR comprises the amino acid sequence set forth in SEQ ID NO: 40, wherein X is pyroglutamate. 38. The CAR of any one of embodiments 1-31, wherein the CAR comprises the amino acid sequence set forth in SEQ ID NO: 41, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 41, wherein X is Q or pyroglutamate. 39. The CAR of any one of embodiments 1-31 and 38, wherein the CAR comprises the amino acid sequence set forth in SEQ ID NO: 41, wherein X is Q or pyroglutamate. 40. The CAR of any one of embodiments 1-31 and 38, wherein the CAR comprises the amino acid sequence set forth in SEQ ID NO: 42, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 42. 41. The CAR of any one of embodiments 1-31 and 38-40, wherein the CAR comprises the amino acid sequence set forth in SEQ ID NO: 42. 42. The CAR of any one of embodiments 1-31 and 38, wherein the CAR comprises the amino acid sequence set forth in SEQ ID NO: 43, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 43, wherein X is pyroglutamate. 43. The CAR of any one of embodiments 1-31, 38, 39, and 42, wherein the CAR comprises the amino acid sequence set forth in SEQ ID NO: 43, wherein X is pyroglutamate. 44. The CAR of any one of embodiments 1-43, wherein the CAR comprises the amino acid sequence set forth in SEQ ID NO: 14 or SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14 or SEQ ID NO: 44, wherein X is Q or pyroglutamate. 45. The CAR of any one of embodiments 1-37 and 44, wherein the CAR comprises the amino acid sequence set forth in SEQ ID NO: 14, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14, wherein X is Q or pyroglutamate. 46. The CAR of any one of embodiments 1-37, 44, and 45, wherein the CAR comprises the amino acid sequence set forth in SEQ ID NO: 14. 47. The CAR of any one of embodiments 1-33, 44, and 45, wherein the CAR comprises the amino acid sequence set forth in SEQ ID NO: 15, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 15. 48. The CAR of any one of embodiments 1-33 and 44-47, wherein the CAR comprises the amino acid sequence set forth in SEQ ID NO: 15. 49. The CAR of any one of embodiments 1-33, 44, and 45, wherein the CAR comprises the amino acid sequence set forth in SEQ ID NO: 16, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 16, wherein X is pyroglutamate. 50. The CAR of any one of embodiments 33, 44-46, and 49, wherein the CAR comprises the amino acid sequence set forth in SEQ ID NO: 16, wherein X is pyroglutamate. 51. The CAR of any one of embodiments 1-31 and 38-44, wherein the CAR comprises the amino acid sequence set forth in SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 44, wherein X is Q or pyroglutamate. 52. The CAR of any one of embodiments 1-31, 38-44, and 51, wherein the CAR comprises the amino acid sequence set forth in SEQ ID NO: 44, wherein X is Q or pyroglutamate. 53. The CAR of any one of embodiments 1-31, 38, 44, and 51, wherein the CAR comprises the amino acid sequence set forth in SEQ ID NO: 45, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 45. 54. The CAR of any one of embodiments 1-31, 38-44, and 51-53, wherein the CAR comprises the amino acid sequence set forth in SEQ ID NO: 45. 55. The CAR of any one of embodiments 1-31, 38, 44, and 51, wherein the CAR comprises the amino acid sequence set forth in SEQ ID NO: 46, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 46, wherein X is pyroglutamate. 56. The CAR of any one of embodiments 1-31, 38-44, and 51, 52, and 55, wherein the CAR comprises the amino acid sequence set forth in SEQ ID NO: 46, wherein X is pyroglutamate. 57. A genetically engineered human T cell comprising the CAR of any one of embodiments 1-56. 58. A genetically engineered human T cell comprising (a) a first nucleotide sequence encoding the CAR of any one of embodiments 1-56. 59. The genetically engineered human T cell of embodiment 58, wherein the first nucleotide sequence encoding the CAR comprises the nucleic acid sequence set forth in SEQ ID NO: 17. 60. The genetically engineered human T cell of any one of embodiments 57-59, further comprising: (b) a first genetic disruption in an endogenous T Cell Receptor Alpha Constant (TRAC) gene; and (c) a second genetic disruption in an endogenous β2 microglobulin (B2M) gene. 61. A genetically engineered human T cell comprising: (a) a first nucleotide sequence encoding a chimeric antigen receptor (CAR); (b) a first genetic disruption in an endogenous TRAC gene; (c) a second genetic disruption in an endogenous B2 microglobulin (B2M) gene; (d) a second nucleotide sequence comprising a transgene encoding a single chain HLA-E fusion protein; and (e) a third nucleotide sequence comprising: (i) a first nucleic acid at least 15 nucleotides in length complementary to an mRNA encoding TGF-B Receptor 2 (TGFBR2); (ii) a second nucleic acid at least 15 nucleotides in length complementary to an mRNA encoding Protein Tyrosine Phosphatase Non-Receptor Type 2 (PTPN2); and (iii) a third nucleic acid at least 15 nucleotides in length complementary an mRNA encoding Fas Cell Surface Death Receptor (FAS). 62. The genetically engineered human T cell of embodiment 61, wherein first nucleotide sequence encoding a chimeric antigen receptor (CAR) encodes the CAR of any one of embodiments 1-56. 63. The genetically engineered human T cell of any one of embodiments 60-62, wherein the first genetic disruption and second genetic disruption are by a gene editing technique comprising a CRISPR-Cas system. 64. The genetically engineered human T cell of embodiment 63, wherein the CRISPR-Cas system is a ribonucleoprotein (RNP) complex comprising a Cas protein and a guide RNA (gRNA), optionally wherein the Cas protein is a Cas12a protein.

65. The genetically engineered human T cell of any one of embodiments 60-64, wherein the first genetic disruption in the endogenous TRAC gene is in a target site sequence in exon 1 of the endogenous TRAC gene. 66. The genetically engineered human T cell of embodiment 65, wherein the target site sequence in exon 1 of the endogenous TRAC gene is located at hg38 genomic coordinates chr14:22,547,528-22,547,548. 67. The genetically engineered human T cell of embodiment 65 or embodiment 66, wherein the target site sequence in exon 1 of the endogenous TRAC gene has the sequence set forth in SEQ ID NO: 59, a contiguous portion thereof of at least 14 nucleotides (nt), or a complementary sequence of the foregoing. 68. The genetically engineered human T cell of any one of embodiments 65-67, wherein the target site sequence in exon 1 of the endogenous TRAC gene has the sequence set forth in SEQ ID NO: 59. 69. The genetically engineered human T cell of any one of embodiments 60-68, wherein the first genetic disruption is by a CRISPR-Cas system that comprises a Cas12a protein and a guide RNA (gRNA) comprising a spacer sequence comprising the nucleic acid sequence of SEQ ID NO:58, or a contiguous portion thereof of at least 14 nt. 70. The genetically engineered human T cell of embodiment 69, wherein the CRISPR-Cas system is a ribonucleoprotein (RNP) complex comprising the Cas12a protein and the gRNA. 71. The genetically engineered human T cell of any one of embodiments 60-70, wherein the first genetic disruption disrupts one or more alleles of the endogenous TRAC gene. 72. The genetically engineered human T cell of any one of embodiments 60-71, wherein the first genetic disruption disrupts all alleles of the endogenous TRAC gene. 73. The genetically engineered human T cell of any one of embodiments 60-72, wherein the first genetic disruption reduces protein expression of a TCR alpha chain encoded from the endogenous TRAC gene. 74. The genetically engineered human T cell of embodiment 73, wherein the first genetic disruption reduces protein expression of the TCR alpha chain on the surface of the genetically engineered human T cell. 75. The genetically engineered human T cell of embodiment 73 or embodiment 74, wherein there is no detectable expression of the TCR alpha chain in the genetically engineered human T cell. 76. The genetically engineered human T cell of any one of embodiments 60-75, wherein the genetically engineered human T cell has reduced expression of CD3 on the cell surface of the genetically engineered human T cell. 77. The genetically engineered human T cell of any one of embodiments 60-76, wherein the genetically engineered cell does not express detectable CD3 on the cell surface of the genetically engineered human T cell.

78. The genetically engineered human T cell of any one of embodiments 60-77, wherein the second genetic disruption in the endogenous B2M gene is in a target site sequence in exon 2 of the endogenous B2M gene. 79. The genetically engineered human T cell of embodiment 78, wherein the target site sequence in exon 2 of the endogenous B2M gene is located at hg38 genomic coordinates chr15:44,715,614-44,715,634. 80. The genetically engineered human T cell of embodiment 78 or embodiment 79, wherein the target site sequence in exon 2 of the endogenous B2M gene has the sequence set forth in SEQ ID NO: 63, a contiguous portion thereof of at least 14 nucleotides (nt), or a complementary sequence of the foregoing. 81. The genetically engineered human T cell of any one of embodiments 78-80, wherein the target site sequence has the sequence set forth in SEQ ID NO: 63. 82. The genetically engineered human T cell of any one of embodiments 60-81, wherein the second genetic disruption is by a CRISPR-Cas system that comprises a Cas12a protein and a guide RNA (gRNA) comprising a spacer sequence comprising the nucleic acid sequence SEQ ID NO:62, or a contiguous portion thereof of at least 14 nt. 83. The genetically engineered human T cell of embodiment 82, wherein the CRISPR-Cas system is a ribonucleoprotein (RNP) complex comprising the Cas12a protein and the gRNA. 84. The genetically engineered human T cell of any one of embodiments 60-82, wherein the second genetic disruption disrupts one or more alleles of the endogenous B2M gene. 85. The genetically engineered human T cell of any one of embodiments 60-84, wherein the second genetic disruption disrupts all alleles of the endogenous B2M gene. 86. The genetically engineered human T cell of any one of embodiments 60-84, wherein the second genetic disruption reduces protein expression of B2M encoded from the endogenous B2M gene. 87. The genetically engineered human T cell of any one of embodiments 60-86, wherein there is no detectable expression of endogenous B2M in the genetically engineered human T cell. 88. The genetically engineered human T cell of any one of embodiments 60-87, wherein the genetically engineered human T cell has reduced expression of one or more HLA class I molecules. 89. The genetically engineered human T cell of any one of embodiments 60-88, wherein the genetically engineered human T cell has no detectable expression of one or more HLA class I molecules on the cell surface of the genetically engineered human T cell. 90. The genetically engineered human T cell of embodiment 88 or embodiment 89, wherein one or more HLA class I molecules is selected from HLA-A class I, HLA-B class I, and HLA-C class I.

91. The genetically engineered human T cell of any one of embodiments 57-60 and 63-90, further comprising: (d) a second nucleotide sequence comprising a transgene encoding a single chain HLA-E fusion protein. 92. The genetically engineered human T cell of any one of embodiments 61-91, wherein the single chain HLA-E fusion protein comprises: (1) at least a portion of the B2M protein, (2) at least a portion of an HLA-E class I chain, and (3) a peptide that is a portion of a signal sequence from an MHC class I molecule that is presented by the single chain HLA-E fusion protein when expressed on the cell surface of the genetically engineered human T cell. 93. The genetically engineered human T cell of embodiment 92, wherein: (1) the portion of the B2M protein comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:47; (2) the portion of an HLA-E class I chain comprises the sequence set forth in, or a sequence that is at least 85%, 86%, 87%, 88%, 89%, 90%, 91%, 92%, 93%, 94%, 95%, 96%, 97%, 98% or 99% identical to SEQ ID NO: 36; and (3) the peptide is VMAPRTLVL (SEQ ID NO:48), VMAPRTLLL (SEQ ID NO:49), VMAPRTVLL (SEQ ID NO:50), VMAPRTLFL (SEQ ID NO:51), or VMAPRTLIL (SEQ ID NO:52). 94. The genetically engineered human T cell of embodiment 93, wherein: (3) the peptide is VMAPRTLVL (SEQ ID NO:48). 95. The genetically engineered human T cell of embodiment 92, wherein: (1) the portion of the B2M protein comprises the sequence set forth in SEQ ID NO:47; (2) the portion of an HLA-E class I chain comprises the sequence set forth in SEQ ID NO: 36; and (3) the peptide is VMAPRTLVL (SEQ ID NO:48). 96. The genetically engineered human T cell of any one of embodiments 92-95, wherein the single chain HLA-E fusion protein further comprises: (4) a peptide linker that links (1) and (2). 97. The genetically engineered human T cell of any one of embodiments 96, wherein the peptide linker comprises a GS linker. 98. The genetically engineered human T cell of any one of embodiments 97, wherein the GS linker is a (G4S)x3 linker set forth in SEQ ID NO:53. 99. The genetically engineered human T cell of embodiment 97 or 98 wherein the GS linker is a (G4S)x4 linker set forth in SEQ ID NO: 54. 100. The genetically engineered human T cell of any one of embodiments 91-98, wherein the single chain HLA-E fusion protein comprises a sequence of amino acids that has at least about 85%, 86%, 87%, 88%, 89%, 90%, 91%, 92%, 93%, 94%, 95%, 96%, 97%, 98%, or 99% sequence identity to SEQ ID NO:34. 101. The genetically engineered human T cell of any one of embodiments 91-98, wherein the single chain HLA-E fusion protein comprises the sequence of amino acids set forth in SEQ ID NO:34. 102. The genetically engineered human T cell of any one of embodiments 91-101, wherein the single chain HLA-E fusion protein is capable of engaging an inhibitory receptor on the surface of an NK cell. 103. The genetically engineered human T cell of embodiment 102, wherein the inhibitory receptor on the surface of the NK cell is an NKG2A or NKG2B. 104. The genetically engineered human T cell of any one of embodiments 91-103, wherein the second nucleotide sequence encoding the single chain HLA-E fusion protein is present in the disrupted endogenous B2M gene in the T cell under the operable control of a promoter. 105. The genetically engineered human T cell of embodiment 104, wherein the promoter is an endogenous promoter of the endogenous B2M gene. 106. The genetically engineered human T cell of any one of embodiments 91-105, wherein the second nucleotide sequence encoding the single chain HLA-E fusion protein has been integrated in the disrupted endogenous B2M gene by homology directed repair (HDR).

107. The genetically engineered human T cell of any one of embodiments 57-60 and 63-106, further comprising: (e) a third nucleotide sequence comprising: (i) a first nucleic acid sequence at least 15 nucleotides in length complementary to an mRNA encoding TGF-B Receptor 2 (TGFBR2); (ii) a second nucleic acid sequence at least 15 nucleotides in length complementary to an mRNA encoding Protein Tyrosine Phosphatase Non-Receptor Type 2 (PTPN2); and (iii) a third nucleic acid sequence at least 15 nucleotides in length complementary an mRNA encoding Fas Cell Surface Death Receptor (FAS). 108. The genetically engineered human T cell of embodiment 61-107, wherein the third nucleotide sequence further comprises: (iv) a fourth nucleic acid at least 15 nucleotides in length complementary to an mRNA encoding human TGF-B Receptor 2 (TGFBR2). 109. The genetically engineered human T cell of embodiment 108, wherein the first and fourth nucleic acids are complementary to different nucleotides of the mRNA encoding human TGF-B Receptor 2 (TGFBR2). 110. The genetically engineered human T cell of any one of embodiments 61-109, wherein each nucleic acid of the third nucleotide sequence of (e) is a short hairpin RNA (shRNA), a small interfering RNA (siRNA), double stranded RNA (dsRNA), or an antisense oligonucleotide. 111. The genetically engineered human T cell of any one of embodiments 61-110, wherein each nucleic acid of the third nucleotide sequence of (e) is a short hairpin RNA (shRNA). 112. The genetically engineered human T cell of any one of embodiments 61-111, wherein: (i) the mRNA encoding TGFBR2 encodes human TGFBR2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85, (ii) the mRNA encoding PTPN2 encodes human PTPN2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:86, and (iii) the mRNA encoding FAS encodes human FAS and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:87. 113. The genetically engineered human T cell of any one of embodiments 61-112, wherein the mRNA encoding TGFBR2 comprises a sequence set forth in SEQ ID NO:85. 114. The genetically engineered human T cell of any one of embodiments 61-113, wherein the mRNA encoding PTPN2 comprises the sequence set forth in SEQ ID NO:86. 115. The genetically engineered human T cell of any one of embodiments 61-114, wherein the mRNA encoding FAS comprises the sequence set forth in SEQ ID NO:87. 116. The genetically engineered human T cell of any one of embodiments 61-115, wherein: (i) the first nucleic acid comprises a sequence complementary to nucleotides 2215 to 2236 of the mRNA encoding TGFBR2 comprising the sequence set forth in SEQ ID NO:85 (SEQ ID NO:74); the second nucleic acid comprises a sequence complementary to nucleotides 518 to 539 of the mRNA encoding PTPN2 comprising the sequence set forth in SEQ ID NO:86 (SEQ ID NO:73); and (iii) the third nucleic acid comprises a sequence complementary to nucleotides 1126 to 1147 of the mRNA encoding FAS comprising a sequence set forth in SEQ ID NO:87 (SEQ ID NO:72). 117. The genetically engineered human T cell of any one of embodiments 61-116, wherein the fourth nucleic acid comprises a sequence complementary to nucleotides 4430 to 4451 of the mRNA encoding TGFBR2 comprising the sequence set forth in SEQ ID NO:85 (SEQ ID NO:75). 118. The genetically engineered human T cell of any one of embodiments 61-117, wherein the third nucleotide sequence comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:71. 119. The genetically engineered human T cell of any one of embodiments 61-118, wherein the third nucleotide sequence comprises the sequence set forth in SEQ ID NO:71. 120. The genetically engineered human T cell of any one of embodiments 108-119, wherein the T cell is characterized by: reduced expression of TGFBR2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid and/or the fourth nucleic acid, reduced expression of PTPN2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid; and/or reduced expression of FAS by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the third nucleic acid.

121. The genetically engineered human T cell of any one of embodiments 61-120, wherein the first nucleotide sequence of (a) and the third nucleotide sequence of (e) are on a same polynucleotide. 122. The genetically engineered human T cell of embodiment 121, wherein the polynucleotide is present in the disrupted endogenous TRAC gene in the genetically engineered human T cell, and the first and third nucleotide sequences are under the operable control of a promoter. 123. The genetically engineered human T cell of embodiment 122, wherein the promoter is a heterologous promoter of the endogenous TRAC gene. 124. The genetically engineered human T cell of embodiment 122 or embodiment 123, wherein the heterologous promoter is a constitutive promoter. 125. The genetically engineered human T cell of embodiment 123 or embodiment 124, wherein the heterologous promoter is or comprises a human elongation factor 1 alpha (EF1α) promoter or a variant thereof 126. The genetically engineered human T cell of any one of embodiments 122-124, wherein the promoter is a synthetic promoter. 127. The genetically engineered human T cell of any one of embodiments 121-126, wherein the polynucleotide further comprises an intron between the first nucleotide of (a) and the second nucleotide of (c).

128. The genetically engineered human T cell of any one of embodiments 121-127, wherein the polynucleotide comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:67. 129. The genetically engineered human T cell of any one of embodiments 121-128, wherein the polynucleotide comprises the sequence set forth in SEQ ID NO:67. 130. The genetically engineered human T cell of any one of embodiments 121-129, wherein the polynucleotide has been integrated in the disrupted endogenous TRAC gene by homology directed repair (HDR). 131. The genetically engineered human T cell of any one of embodiments 60-130, wherein the genetically engineered human T cell further comprises: (f) a third genetic disruption in an endogenous CD70 gene. 132. The genetically engineered human T cell of embodiment 131, wherein the third genetic disruption in the endogenous CD70 gene is in a target site sequence in exon 2 of the endogenous CD70 gene. 133. The genetically engineered human T cell of embodiment 132, wherein the target site sequence in exon 2 of the endogenous CD70 gene is located at hg38 genomic coordinates chr19:6,590,122-6,590,142. 134. The genetically engineered human T cell of embodiment 132 or embodiment 133, wherein the target site sequence in exon 2 of the endogenous CD70 gene has the sequence set forth in SEQ ID NO: 66, a contiguous portion thereof of at least 14 nucleotides (nt), or a complementary sequence of the foregoing. 135. The genetically engineered human T cell of any one of embodiments 132-134, wherein the target site sequence in exon 2 of the endogenous CD70 gene has the sequence set forth in SEQ ID NO: 66. 136. The genetically engineered human T cell of any one of embodiments 131-135, wherein the third genetic disruption is by a CRISPR-Cas system that comprises a Cas12a protein and a guide RNA (gRNA) comprising a spacer sequence comprising the nucleic acid sequence of SEQ ID NO: 65, or a contiguous portion thereof of at least 14 nt. 137. The genetically engineered human T cell of embodiment 136, wherein the CRISPR-Cas system is a ribonucleoprotein (RNP) complex comprising the Cas12a protein and the gRNA. 138. The genetically engineered human T cell of any one of embodiments 131-137, wherein the third genetic disruption disrupts one or more alleles of the endogenous CD70 gene. 139. The genetically engineered human T cell of any one of embodiments 131-138, wherein the third genetic disruption disrupts all alleles of the endogenous CD70 gene. 140. The genetically engineered human T cell of any one of embodiments 131-139, wherein the third genetic disruption reduces protein expression of CD70 encoded from the endogenous CD70 gene. 141. The genetically engineered human T cell of any one of embodiments 131-140, wherein the third genetic disruption reduces protein expression of CD70 on the surface of the genetically engineered human T cell. 142. The genetically engineered human T cell of any one of embodiments 131-141, wherein there is no detectable expression of CD70 in the genetically engineered human T cell. 143. The genetically engineered human T cell of any one of embodiments 131-142, wherein the genetically engineered human T cell has reduced fratricide as compared to a control cell that does not comprise the third genetic disruption. 144. The genetically engineered human T cell of any one of embodiments 57-143, wherein the human T cell is a primary human T cell. 145. The genetically engineered human T cell of embodiment 144, wherein the primary human T cell is from a healthy human donor.

146. A genetically engineered human T cell comprising: (a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising: (i) a nucleotide sequence comprising the sequence set forth in SEQ ID NO:71 and (ii) a nucleotide sequence encoding a CAR comprising the amino acid sequence set forth in SEQ ID NO: 38 or SEQ ID NO: 41, wherein X is Q or pyroglutamate, is present in the disrupted endogenous TRAC gene in the genetically engineered human T cell, (b) a second genetic disruption in exon 2 of an endogenous B2M gene located at hg38 genomic coordinates chr15:44,715,614-44,715,634, wherein the second genetic disruption disrupts one or more alleles of the endogenous B2M gene, and wherein a nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and (c) a third genetic disruption in exon 2 of an endogenous CD70 gene located at hg38 genomic coordinates chr19:6,590,122-6,590,142, wherein the third genetic disruption disrupts all alleles of the endogenous CD70 gene. 147. A genetically engineered human T cell comprising: (a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising: (i) a nucleotide sequence comprising: (1) a first shRNA comprising the sequence set forth in SEQ ID NO:74, (2) a second shRNA comprising the sequence set forth in SEQ ID NO:73, (3) a third shRNA comprising the sequence set forth in SEQ ID NO:72, and (4) a fourth shRNA comprising the sequence set forth in SEQ ID NO:75; and (ii) a nucleotide sequence encoding a CAR targeting CD70 comprising the sequence set forth in SEQ ID NO: 38 or SEQ ID NO: 41, wherein X is Q or pyroglutamate, is present in the disrupted TRAC gene in the genetically engineered human T cell, (b) a second genetic disruption in exon 2 of an endogenous B2M gene located at hg38 genomic coordinates chr15:44,715,614-44,715,634, wherein the second genetic disruption disrupts one or more alleles of the endogenous B2M gene, and wherein the first nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and (c) a third genetic disruption in exon 2 of an endogenous CD70 gene located at hg38 genomic coordinates chr19:6,590,122-6,590,142, wherein the third genetic disruption disrupts all alleles of the endogenous CD70 gene.

148. A population of genetically engineered human T cells comprising the genetically engineered human T cell of any one of embodiments 57-147. 149. The population of genetically engineered human T cells of embodiment 148, wherein at least or at about 50%, at least or at about 60%, at least or at about 70%, at least or at about 80%, or at least or at about 90% of the cells in the population have a genetic modification selected from: (a) the first genetic disruption in the endogenous TRAC gene; (b) the second genetic disruption in the endogenous B2M gene; (c) a knock-in of the first nucleotide sequence transgene encoding the single chain HLA-E fusion protein; (d) a knock-in of the second nucleotide sequence; (e) a knock-in of the third nucleotide sequence encoding the CAR; and/or (f) the third genetic disruption in the endogenous CD70 gene. 150. The population of genetically engineered human T cells of embodiment 148 or embodiment 149, wherein at least or at about 50%, at least or at about 60%, at least or at about 70%, at least or at about 80%, or at least or at about 90% of the cells in the population is characterized by: (1) one or more of (a)-(f); (2) two or more of (a)-(f); (3) three or more of (a)-(f); (4) four or more of (a)-(f); (5) five or more of (a)-(f); or (6) all of (a)-(f). 151. A composition comprising the population of genetically engineered human T cells of any one of embodiments 148-150. 152. The composition of embodiment 151, wherein the composition is a pharmaceutical composition comprising a pharmaceutically acceptable excipient. 153. The composition of any one of embodiments 151 or embodiment 152, wherein the composition comprises a cryoprotectant, optionally wherein the cryoprotectant is DMSO.

154. A method of treatment comprising administering the genetically engineered human T cell of any one of embodiments 57-147, the population of genetically engineered human T cells of any one of embodiments 148-150, or the composition of any one of embodiments 151-153 to a subject having a disease or disorder. 155. The method of embodiment 154, wherein the disease or disorder is associated with CD70. 156. The method of embodiment 154 or embodiment 155, wherein the disease or disorder is a cancer. 157. The method of embodiment 156, wherein the cancer is a solid tumor. 158. The method of embodiment 157, wherein the solid tumor is a renal cell carcinoma. 159. The method of embodiment 158, wherein the renal cell carcinoma is a clear cell renal cell carcinoma.

160. The method of any one of embodiments 154-159, wherein the human subject is non-responsive or refractory to one or more standard therapies for cancer. 161. The method of any one of embodiments 154-160, wherein the human subject has developed drug resistance to one or more standard therapies for cancer. 162. The method of any one of embodiments 154-160, wherein the human subject has undergone surgery in an attempt to treat the disease or disorder. 163. The method of any one of embodiments 154-159, where in the human subject has not been previously treated for cancer. 164. The method of any one of embodiments 154-159 or 163, wherein the human subject has not undergone surgery in an attempt to treat the disease or disorder.

165. The method of any one of embodiments 154-164, wherein the method comprises allogeneic transfer, in which the cells are isolated and/or otherwise prepared from a donor other than the human subject. 166. The method of embodiment 165, wherein the donor is a healthy donor. 167. The method of embodiment 165 or embodiment 166, wherein the donor does not have the disease or disorder. 168. The method of any one of embodiments 165-167, wherein the donor and the human subject are genetically identical or genetically similar. 169. The method of any one of embodiments 165-168, wherein the human subject expresses a HLA class or supertype that is the same as the donor. 170. The method of any one of embodiments 165-168, wherein the human subject does not express a HLA class or supertype that is the same as the donor.

171. The method of any one of embodiments 154-170, comprising administering a dose comprising a therapeutically effective amount of the genetically engineered human T cell, the population of genetically engineered human T cells, or the composition. 172. The method of embodiment 171, wherein the dose comprises about 25×10⁶, about 50×10⁶, 100×10⁶, about 150×10⁶, about 200×10⁶, about 250×10⁶, about 300×10⁶, about 350×10⁶, about 400×10⁶, about 450×10⁶, about 500×10⁶, about 550×10⁶, about 650×10⁶, about 700×10⁶, about 750×10⁶, about 800×10⁶, about 850×10⁶, about 900×10⁶, about 950×10⁶, about 1×10⁹, about 1.1×10⁹, about 1.2×10⁹, about 1.3×10⁹, about 1.4×10⁹, or about 1.5×10⁹ engineered T cells. 173. The method of embodiment 172, wherein the dose comprises about 50×10⁶ engineered T cells. 174. The method of embodiment 172, wherein the dose comprises about 100×10⁶ engineered T cells. 175. The method of embodiment 172, wherein the dose comprises about 300×10⁶ engineered T cells. 176. The method of embodiment 172, wherein the dose comprises about 900×10⁶ engineered T cells.

177. The method of any one of embodiments 171-176, wherein the dose is administered via an intravenous (IV) infusion. 178. The method of embodiment 177, wherein the IV infusion is a single IV infusion. 179. The method of embodiment 177, wherein the subject receives more than one dose or more than one IV infusion. 180. The method of any one of embodiments 171-179, the dose is provided as a suspension for administration via IV infusion.

181. The method of any one of embodiments 154-180, wherein the human subject is pretreated with a lymphodepleting chemotherapy prior to administration of the genetically engineered human T cells. 182. The method of embodiment 181, wherein the lymphodepleting chemotherapy comprises treating the human subject with fludarabine IV (30 mg/m²/day) and cyclophosphamide IV (500 mg/m²/day) for three days prior to administration of the genetically engineered human T cells. 183. The method of embodiment 181, wherein the lymphodepleting chemotherapy comprises treating the human subject with fludarabine IV (30 mg/m²/day) and cyclophosphamide IV (500 mg/m²/day) for four days or five days prior to administration of the genetically engineered human T cells.

184. The method of any one of embodiments 154-183, further comprising at least one additional therapy, therapeutic agent, or modality. 185. The method of embodiment 184, wherein the at least one additional therapeutic agent or modality comprises at least one PD-1 therapy. 186. The method of embodiment 185, wherein the at least one PD-1 therapy is an antibody or antigen-binding fragment thereof that binds to PD-1. 187. The method of embodiment 185, wherein the at least one PD-1 therapy is an antibody or antigen-binding fragment thereof that binds to PD-L1. 188. The method of any one of embodiments 185-187, wherein the at least one PD-1 therapy is selected from the group consisting of nivolumab, pembrolizumab, cemiplimab, dostarlimab, retifanlimab, toripalimab, atezolizumab, avelumab, durvalumab, cosibelimab, camrelizumab, sintilimab, and tislelizumab.

189. The method of embodiment 188, wherein the at least one PD-1 therapy is nivolumab. 190. The method of embodiment 189, wherein the nivolumab is administered to the subject about once every two weeks at a dose of 240 mg. 191. The method of claim 189, wherein the nivolumab is administered to the human subject about once every two weeks at a dose of 600 mg. 192. The method of claim 189, wherein the nivolumab is administered to the human subject about once every two weeks at a dose of 720 mg. 193. The method of claim 189, wherein the nivolumab is administered to the human subject about once every two weeks at a dose of 960 mg. 194. The method of claim 189, wherein the nivolumab is administered to the human subject about once every two weeks at a dose of 1200 mg. 195. The method of embodiment 189, wherein the nivolumab is administered to the human subject about once every three weeks at a dose of 360 mg. 196. The method of claim 189, wherein the nivolumab is administered to the human subject about once every three weeks at a dose of 720 mg. 197. The method of claim 189, wherein the nivolumab is administered to the human subject about once every three weeks at a dose of 900 mg. 198. The method of claim 189, wherein the nivolumab is administered to the human subject about once every three weeks at a dose of 960 mg. 199. The method of claim 189, wherein the nivolumab is administered to the human subject about once every three weeks at a dose of 1200 mg. 200. The method of embodiment 189, wherein the nivolumab is administered to the human subject about once every four weeks at a dose of 480 mg. 201. The method of claim 189, wherein the nivolumab is administered to the human subject about once every four weeks at a dose of 720 mg. 202. The method of claim 189, wherein the nivolumab is administered to the human subject about once every four weeks at a dose of 960 mg. 203. The method of claim 189, wherein the nivolumab is administered to the human subject about once every four weeks at a dose of 1200 mg. 204. The method of embodiment 189, wherein the nivolumab is administered to the human subject about once every two weeks at a dose of between 3 mg/kg and 10 mg/kg. 205. The method of embodiment 189, wherein the nivolumab is administered to the human subject about once every two weeks at a dose of 3 mg/kg.

206. The method of any one of claims 189-205, wherein the nivolumab is administered intravenously. 207. The method of any one of claims 189-205, wherein the nivolumab is administered subcutaneously. 208. The method of any one of claims 189-205 and 207, wherein the nivolumab is co-formulated with hyaluronidase. 209. The method of claim 208, wherein the hyaluronidase is at a dose of about 10,000 units to about 20,000 units, inclusive. 210. The method of claim 208 or claim 209, wherein the hyaluronidase is at a dose of about 10,000 units; about 12,000 units; 15,000 units; about 16,000 units; or about 20,000 units. 211. The method of any one of claims 208-210, wherein the nivolumab co-formulated with hyaluronidase is administered to the human subject about once every two weeks at a dose of 600 mg nivolumab and a dose of about 10,000 units hyaluronidase. 212. The method of any one of claims 208-210, wherein the nivolumab co-formulated with hyaluronidase is administered to the human subject about once every three weeks at a dose of 900 mg nivolumab and a dose of about 15,000 units hyaluronidase. 213. The method of any one of claims 208-210, wherein the nivolumab co-formulated with hyaluronidase is administered to the human subject about once every four weeks at a dose of 1200 mg nivolumab and about 20,000 units hyaluronidase. 214. The method of any one of claims 208-210, wherein the nivolumab co-formulated with hyaluronidase is administered to the human subject about once every two to four weeks at a dose of 720 mg nivolumab and about 20,000 units hyaluronidase. 215. The method of any one of claims 208-210, wherein the nivolumab co-formulated with hyaluronidase is administered to the human subject about once every two to four weeks at a dose of 960 mg nivolumab and about 20,000 units hyaluronidase. 216. The method of any one of claims 208-210, wherein the nivolumab co-formulated with hyaluronidase is administered to the human subject about once every two to four weeks at a dose of 1200 mg nivolumab and about 20,000 units hyaluronidase. 217. The method of any one of claims 208-210, wherein the nivolumab co-formulated with hyaluronidase is administered to the human subject about once every two to four weeks at a dose of 720 mg nivolumab and about 12,000 units hyaluronidase. 218. The method of any one of claims 208-210, wherein the nivolumab co-formulated with hyaluronidase is administered to the human subject about once every two to four weeks at a dose of 960 mg nivolumab and about 16,000 units hyaluronidase. 219. The method of any one of claims 208-210, wherein the nivolumab co-formulated with hyaluronidase is administered to the human subject about once every two to four weeks at a dose of 1200 mg nivolumab and about 20,000 units hyaluronidase. 220. The method of any one of claims 214-219, wherein the once every two to four weeks is once every two weeks. 221. The method of any one of claims 214-219, wherein the once every two to four weeks is once every three weeks. 222. The method of any one of claims 214-219, wherein the once every two to four weeks is once every four weeks.

223. The method of any one of embodiments 185-222, wherein the at least one PD-1 therapy is administered to the human subject for 4, 6, 8, 12, 15, 18, 20, 24, 30, 36, 40, 48, or 60 months. 224. The method of any one of embodiments 185-222, wherein the at least one PD-1 therapy is discontinued after 4, 6, 8, 12, 15, 18, 20, 24, 30, 36, 40, 48, or 60 months. 225. The method of any one of embodiments 185-222, wherein the at least one PD-1 therapy is administered to the human subject for 12 months. 226. The method of any one of embodiments 185-222, wherein the at least one PD-1 therapy is administered to the human subject for 24 months. 227. The method of any one of embodiments 185-226, wherein the at least one PD-1 therapy is administered to the human subject until the disease or disorder progresses.

228. The method of any one of embodiments 185-227, wherein the at least one additional therapeutic agent or modality comprises at least one tyrosine kinase inhibitor (TKI). 229. The method of embodiment 228, wherein the at least one TKI is administered to the human subject as an oral capsule or tablet. 230. The method of embodiment 228 or 229, wherein the at least one TKI is a small-molecule inhibitor of at least one of c-Met (HGFR), VEGFR1, VEGFR2, VEGFR3, AXL, RET, and FLT3. 231. The method of embodiment 230, wherein the at least one TKI is a small-molecule inhibitor of at least one of c-Met (HGFR), VEGFR2, AXL, RET, and FLT3. 232. The method of any one of embodiments 228-231, wherein the at least one TKI is or comprises cabozantinib, tivozanib, lenvatinib, axitinib, pazopanib or sunitinib. 233. The method of any one of embodiments 228-232, wherein the at least one TKI is or comprises cabozantinib. 234. The method of embodiment 233, wherein the cabozantinib is administered to the human subject once daily. 235. The method of embodiment 233 or embodiment 234, wherein the cabozantinib is administered to the human subject until the disease or disorder progresses. 236. The method of any one of embodiments 233-235, wherein the cabozantinib is administered to the human subject at a dose of 40 mg/day. 237. The method of any one of embodiments 233-235, wherein the cabozantinib is administered to the human subject at a dose of 60 mg/day.

238. The method of any one of embodiments 228-232, wherein the at least one TKI is a small-molecule inhibitor of at least one of VEGFR1, VEGFR2, and VEGFR3. 239. The method of any one of embodiments 228-232 and embodiment 238, wherein the at least one TKI is or comprises tivozanib. 240. The method of embodiment 239, wherein the tivozanib is administered to the human subject once daily. 241. The method of embodiment 239 or embodiment 240, wherein the tivozanib is administered to the human subject until the disease or disorder progresses. 242. The method of any one of embodiments 239-241, wherein the tivozanib is administered to the human subject in a dosing cycle. 243. The method of embodiment 242, wherein the dosing cycle comprises a 28-day cycle comprising administering to the human subject one dose of tivozanib orally for 21 days followed by seven days without administration of tivozanib to the human subject. 244. The method of any one of embodiments 239-243, wherein the tivozanib is administered to the human subject at a dose of 1.34 mg/day. 245. The method of any one of embodiments 239-243, wherein the tivozanib is administered to the human subject at a dose of 0.89 mg/day.

246. A guide RNA (gRNA), wherein the gRNA targets an endogenous TRAC gene and comprises the sequence set forth in SEQ ID NO:55. 247. A guide RNA (gRNA), wherein the gRNA targets an endogenous CD70 gene and comprises the sequence set forth in SEQ ID NO:64.

248. A polynucleotide encoding the CAR of any one of embodiments 1-56. 249. A polynucleotide comprising the sequence set forth in, or a sequence that has at least about 85%, 86%, 87%, 88%, 89%, 90%, 91%, 92%, 93%, 94%, 95%, 96%, 97%, 98%, or 99% sequence identity to, SEQ ID NO:67. 250. The polynucleotide of embodiment 248 or embodiment 249, wherein the polynucleotide comprises the sequence set forth in SEQ ID NO:67.

251. A polynucleotide, wherein the polynucleotide comprises the sequence set forth in, or a sequence of amino acids that has at least about 85%, 86%, 87%, 88%, 89%, 90%, 91%, 92%, 93%, 94%, 95%, 96%, 97%, 98%, or 99% sequence identity to, SEQ ID NO:77. 252. The polynucleotide of embodiment 251, wherein the polynucleotide comprises the sequence set forth in SEQ ID NO:77.

253. A vector comprising the polynucleotide of any one of embodiments 258-252. 254. The vector of embodiment 253, wherein the vector is a viral vector. 255. The vector of embodiment 254, wherein the viral vector is an AAV.

256. A cell comprising the CAR of any one of embodiments 1-56, the gRNA of embodiment 246 or embodiment 247, the polynucleotide of any one of embodiments 248-252, or the vector of any one of embodiments 253-255. 257. The cell of embodiment 256, wherein the cell is a T cell. 258. The cell of embodiment 257, wherein the T cell is a primary T cell. 259. The cell of embodiment 258, wherein the primary T cell is from a human donor. 260. The cell of any one of embodiments 257-259, wherein the T cell is a CD4+ T cell, a CD8+ T cell, or a CD4+ and CD8+ T cell.

261. An antibody, or antigen-binding fragment thereof, comprising a CD70-binding domain that binds to CD70, wherein the CD70-binding domain comprises a heavy chain only variable region (VHH), wherein: (i) the VHH comprises a CDR-1, CDR-2, and CDR-3 each comprising a sequence that is contained within SEQ ID NO:1, wherein X is Q or pyroglutamate; or (ii) the VHH comprises a CDR-1, CDR-2, and CDR-3 each comprising a sequence that is contained within SEQ ID NO:27, wherein X is Q or pyroglutamate. 262. The antibody, or antigen-binding fragment thereof, of embodiment 261, wherein: (i) the VHH of the CD70-binding domain comprises CDR-1, CDR-2, and CDR-3 sequences set forth in SEQ ID NOs: 4, 5, and 6, respectively; (ii) the VHH of the CD70-binding domain comprises CDR-1, CDR-2, and CDR-3 sequences set forth in SEQ ID NOs: 7, 8, and 9, respectively; (iii) the VHH of the CD70-binding domain comprises CDR-1, CDR-2, and CDR-3 sequences set forth in SEQ ID NOs: 79, 80, and 81, respectively; or (iv) the VHH of the CD70-binding domain comprises CDR-1, CDR-2, and CDR-3 sequences set forth in SEQ ID NOs: 82, 83, and 84, respectively. 263. The antibody, or antigen-binding fragment thereof, of embodiment 261 or 262, wherein: (i) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or (ii) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate. 264. The antibody, or antigen-binding fragment thereof, of any one of embodiments 261-263, wherein: (i) the VHH comprises a sequence set forth in SEQ ID NO: 1, wherein X is Q or pyroglutamate; or (ii) the VHH comprises a sequence set forth in SEQ ID NO: 27, wherein X is Q or pyroglutamate.

### X. EXAMPLES

The following examples are included for illustrative purposes only and are not intended to limit the scope of the invention.

### Example 1—Generation and Assessment of Anti-CD70 Chimeric Antigen Receptor (CAR)

Anti-CD70 VHH binding domains were generated using alpacas that were immunized with recombinant extracellular binding domain (ECD) of human CD70. mRNA was harvested from extracted lymphocytes and about 1054 individual VHH were amplified and sequenced. These binders then underwent subsequent analysis when formatted as part of a chimeric antigen receptor and assessed for binding and activity.

The synthesized anti-CD70-encoding sequences were cloned into an exemplary polynucleotide construct to generate candidate polynucleotides encoding chimeric antigen receptors (CARs) containing the candidate CD70 VHH binders as the antigen-binding domains. Specifically, the polynucleotide CAR constructs contained nucleic acid sequences encoding a signal peptide; a candidate anti-CD70 VHH binder; a spacer containing a IgG-Long hinge (SEQ ID NO: 93), a IgG-Medium hinge (SEQ ID NO: 94), or a IgG-Short hinge (SEQ ID NO: 95); a human CD28 transmembrane domain (SEQ ID NO: 11); a human 4-1BB intracellular costimulatory domain (SEQ ID NO: 12); and a human CD3-zeta intracellular signaling region (SEQ ID NO: 13). Polynucleotides encoding the CARs were cloned into a lentiviral expression vector for transduction of T cells.

Viral preparations encoding the various candidate anti-CD70 CARs were individually introduced by transduction into two different Jurkat T cell lines containing a Nur77 knock-in reporter (see, e.g., WO 2019/089982). The Nur77 knock-in cell line contained nucleic acid sequences encoding a tandem dimer tomato (tdTomato) fluorescent protein reporter molecule knocked-in at the endogenous Nur77 locus, which is an immediate-early response gene induced by stimulation of signal from the T cell receptor and/or via molecules containing immunoreceptor tyrosine-based activation motif (ITAM). Further, the first of the two Jurkat T cell lines was genetically engineered to over-express CD70 while the second of the two Jurkat T cell lines was genetically engineered to contain a knock-out in the Jurkat T cell line. Both Jurkat reporter cell lines were assessed for cell surface expression of the CAR, and antigen-dependent and antigen-independent signaling.

After transduction, the Jurkat reporter cells were evaluated for the expression of red fluorescent protein after co-culture with THP-1, KG-1, and 786-O target cells expressing CD70 or parental K562 target cells. Jurkat reporter cells transduced with candidate anti-CD70 CARs exhibited varying levels of antigen-specific activity, and certain anti-CD70 CARs also exhibited tonic signaling as evidenced by detection of reporter cell activity when co-cultured in media with parental K562 cells lacking CD70. About 87 anti-CD70 CARs each incorporating a different anti-CD70 VHH or scFV binders were selected that exhibited high CD70 antigen-dependent activity and did not exhibit high tonic signaling. Of the 87 anti-CD70 CARs, 40 were VHH binders from alpacas. The VHH binders were humanized.

### Example 2—Assessment of CAR Expression and Function in Primary T Cells

The expression and function of the 87 anti-CD70 CARs selected in Example 1, including the humanized VHH binders, were assessed in primary human T cells.

CD4+ and CD8+ T cells were isolated by immunoaffinity-based enrichment from leukapheresis samples of one human donor. Isolated T cells were stimulated with anti-CD3/anti-CD28 antibody stimulation reagent in the presence of recombinant IL-2, IL-7, and IL-15, transduced with lentiviral preparations encoding a selected anti-CD70 CAR, and cultivated under conditions for expansion.

The primary T cells expressing one of the candidate CARs were co-cultured with target cells expressing CD70. The target cells also were labeled with NucLight Red (NLR) to monitor cytolytic cell killing via analysis of red fluorescent signal over time on an IncuCyte® instrument. To assess antigen-dependent CAR activity, co-cultures were assessed for activity of the target cells based on cytokine response by the CAR T cell by measurement of IFN-g, IL-2, and TNF-α in culture supernatant.

Based on the results of this screen, 27 anti-CD70 CARs were selected for further analysis. These CARs were then further tested using a similar experiment with readouts as previously described to determine the ability of the selected CARs to lessen tumor burden.

Primary human CD4+ and CD8+ T cells were isolated by immunoaffinity-based selection from human peripheral blood mononuclear cells (PBMCs) and were stimulated by culturing with an anti-CD3/anti-CD28 stimulatory reagent at 37° C. in media with recombinant cytokines. Stimulated cells were then electroporated with ribonucleoprotein (RNP) complexes containing (1) Cas12a and a Cas12a-based CD70 gRNA (SEQ ID NO:64) from two different donors to disrupt the CD70 locus, and (2) Cas9 and TRAC-targeting guide RNA (gRNA) containing the spacer sequence set forth in SEQ ID NO: 128 to disrupt the target site set forth in SEQ ID NO: 127 to knock-out the TRAC locus. After electroporation, an AAV preparation containing the DNA sequence encoding each tested CAR operably linked to an EF1α promoter with an HTLV non-coding element (SEQ ID NO: 20) and homology arms homologous to sequences surrounding the target integration site in the disrupted TRAC locus in order to facilitate homology-directed DNA repair was added to each well. After an overnight incubation for recovery, the T cells were cultured under conditions for expansion.

Two unique CD70 VHH binders were featured among the selected anti-CD70 CARs selected as a result of the AAV screen. The sequences of the CDRs for the exemplary CD70 VHH binders are presented in Table E1 below.

TABLE E1
Sequence identifiers (SEQ ID NO:) for CD70 Binders

| | | CDR-1, -2, -3 SEQ ID NO |
|---|---|---|
| Binder | VHH SEQ ID NO | (numbering scheme) |
| 16 | 2 | 4, 5, 6 (Kabat) |
| | | 7, 8, 9 (IMGT) |
| 18 | 28 | 79, 80, 81 (Kabat) |
| | | 82, 83, 84 (IMGT) |

### Example 3—Hinge Optimization of Anti-CD70 CAR

Cytolytic activity of CARs containing either of the two CD70 VHH binders described in Table E1 were further assessed when using one of two different hinge spacers: an IgGL hinge region (SEQ ID NO: 93) or a CD28 hinge region (SEQ ID NO: 10) to assess different spacers on the activity of the anti-CD70 CAR. The CAR sequences also contained a human CD28 transmembrane domain (SEQ ID NO: 11); a human 4-1BB intracellular costimulatory domain (SEQ ID NO: 12); and a human CD3-zeta intracellular signaling region (SEQ ID NO: 13), as described above.

CD4+ and CD8+ T cells isolated by immunoaffinity-based enrichment from leukapheresis samples from a human donor subject were stimulated and transduced with an anti-CD70 CAR containing one of the CD70 VHH binders described in Table E1 and either a IgGL or CD28 hinge region. Briefly, T cells were transduced with RNPs containing Cas12a and either a Cas12a-based CD70 gRNA (SEQ ID NO:64) or a Cas12a-based TRAC gRNA (SEQ ID NO:55) to knock out the CD70 and TRAC loci, respectively. Further, a donor template for the knock-in of each tested anti-CD70 CAR into the disrupted CD70 locus operably linked to an EF1α promoter with an HTLV non-coding element (SEQ ID NO: 20) was also transduced into the T cells. As a control, mock T cells also were engineered to similarly knock out the CD70 and TRAC loci, but the mock T cells were not engineered with a CAR by knock-in of the anti-CD70 CAR.

Engineered CAR T cells were then injected into NOD.Cg-Prkdc^{scid}IL-2rg^{tm1Wjl}/SzJ immunodeficient mice (NSG) mice that had been subcutaneously injected with renal cell carcinoma cell line ACHN (ATCC No. CRL-1611) xenografts. Each mouse was inoculated with 10 million ACHN tumor cells per mouse. Once the tumor volume within the mice reached an average of around 150 mm³, each mouse was injected with one million CAR T cells. The mock T cells containing knock-outs of the CD70 and TRAC loci and no CAR were injected as a control. Tumor volume was measured for 50 days following injection.

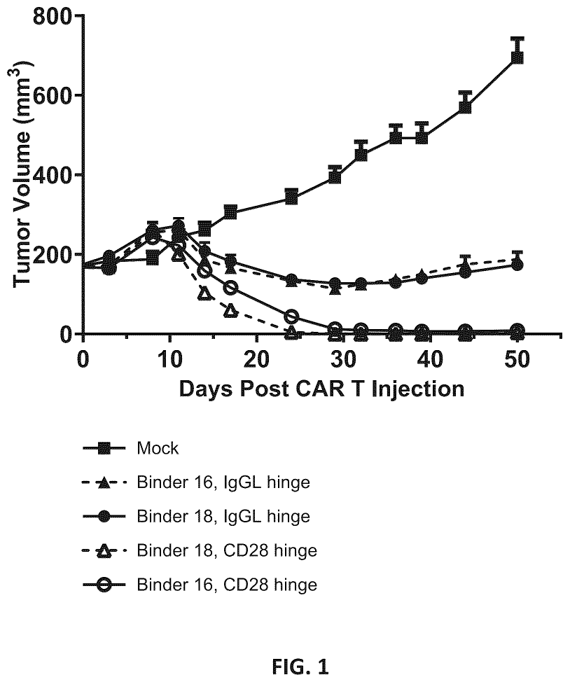

As shown in FIG. 1, use of CAR T cells containing either of the exemplary CD70 VHH binders resulted in decreased tumor volume as compared to the mock T cell control that contained a knock-out of CD70 and TRAC loci but no CAR. However, only CAR T cells containing the CD28 hinge region were able to completely clear tumors as compared to CARs containing the IgGL hinge region, which only resulted in partial clearing of tumors.

These results demonstrate that anti-CD70 CARs with a CD28 hinge domain result in strong cytolytic activity against target cells expressing CD70.

### Example 4—Function of Anti-CD70 CAR T Cells with Circulating CD27

The effect of circulating CD27 on the anti-CD70 CAR T cell described in Example 3 was assessed, since CD27 is the natural receptor for CD70 and it is known to be cleaved from the cell surface into a soluble form. The CAR tested in this example was the exemplary CAR that contained the anti-CD70 binding domain binder 16 (SEQ ID NO: 1), a CD28 hinge domain (SEQ ID NO: 10, a human CD28 transmembrane domain (SEQ ID NO: 11); a human 4-1BB intracellular costimulatory domain (SEQ ID NO: 12); and a human CD3-zeta intracellular signaling region (SEQ ID NO: 13). Renal cell carcinoma (RCC) patients often have circulating CD27 levels. Thus, the acute T cell function of exemplary anti-CD70 CARs were tested in the presence of a soluble CD27.

A498 or ACHN target cell lines were preincubated with a soluble anti-CD70 CAR-Fc (SEQ ID NO: 98 or SEQ ID NO: 129), dimeric soluble CD27 (Acro Bio), monomeric soluble CD27 (SEQ ID NO: 99), or no competitor. The concentration of the soluble CD27 was about 150 U/mL, corresponding to the average soluble CD27 concentration measured in circulation of RCC patients (Benhamouda et al., Clin Cancer Res, 2022). The concentration of the CAR-Fc was also equimolar to the concentration of soluble CD27 used. The anti-CD70 CAR-Fc was used to block binding of the anti-CD70 binder of the CAR to the cellular CD70 antigen.

CAR T cells from three different donors that were engineered with the exemplary CAR were co-cultured with either target cell line. Supernatant was collected and the production of IFNg was determined using an ELISA-based assay platform.

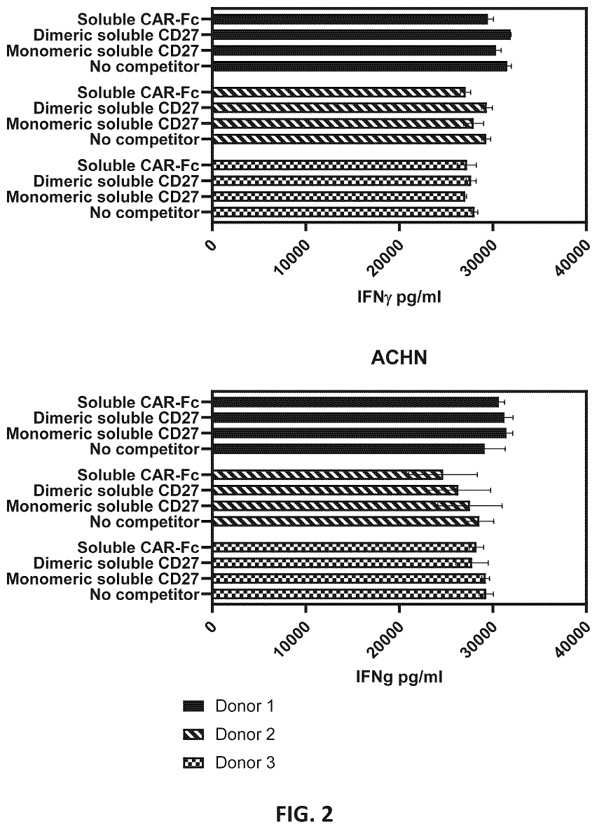

As shown in FIG. 2 below, the exemplary anti-CD70 CAR showed no strong decrease in IFN-g production when the cells were cultured with soluble CD27 or anti-CD70 CAR-Fc in the presence of either the A498 target cell line (top) or ACHN target cell line (bottom).

This result demonstrates that the anti-CD70 CAR activity was not blocked or reduced by soluble antigens, including antigens likely to be present in RCC patient populations.

### Example 5—Generation and Assessment of Engineered Allogeneic T Cells

Primary human T cells engineered with an exemplary CAR were engineered to produce an allogeneic T cell composition by a strategy to reduce graft vs. Host Disease (GvHD), host-versus-graft (HvG) rejections, and self-recognition resulting in NK cell-mediated rejection.

The strategy included engineering primary human T cells by gene editing with clustered regularly interspaced short palindromic repeats (CRISPR) technology to knock out (KO) the T-cell receptor alpha constant (TRAC) locus to abrogate T-cell receptor (TCR) expression to mitigate graft versus host interactions, β2 microglobulin (B2M) locus to abrogate human leukocyte antigen (HLA) class I expression and mitigate host versus graft interactions, and the cluster of differentiation 70 (CD70) locus to mitigate self-targeting of the CAR T cell and CAR T:CAR T fratricide; and introducing a histocompatibility antigen alpha chain E (HLA-E)-based fusion molecule by knock-in (KI) at the B2M locus to reduce natural killer (NK) cell mediated rejection. A DNA cassette set forth in SEQ ID NO: 67 encoding four shRNAs and an exemplary CAR, in this case the CAR set forth in SEQ ID NO: 38, targeting CD70, was knocked in (KI) at the TRAC locus. The four shRNAs included a first and second shRNA (SEQ ID NOs: 74 and 75) targeting the transforming growth factor, beta receptor II (TGFBR2) mRNA set forth in SEQ ID NO: 85, a third shRNA (SEQ ID NO: 73) targeting protein tyrosine phosphatase non-receptor 2 (PTPN2) mRNA set forth in SEQ ID NO: 86, and a fourth shRNA (SEQ ID NO: 72) targeting FS-7-associated surface antigen (FAS) loci set forth in SEQ ID NO: 87. Both the nucleotide sequence encoding the shRNAs and CAR (AAV vector #1; set forth in SEQ ID NO: 77), and the nucleotide sequence encoding the HLA-E fusion molecule (AAV vector #2; set forth in SEQ ID NO: 78) were delivered using recombinant adeno-associated viral vector serotype 6 (rAAV6 vector)-mediated homology directed recombination (HDR) to the respective CRISPR-edited loci.

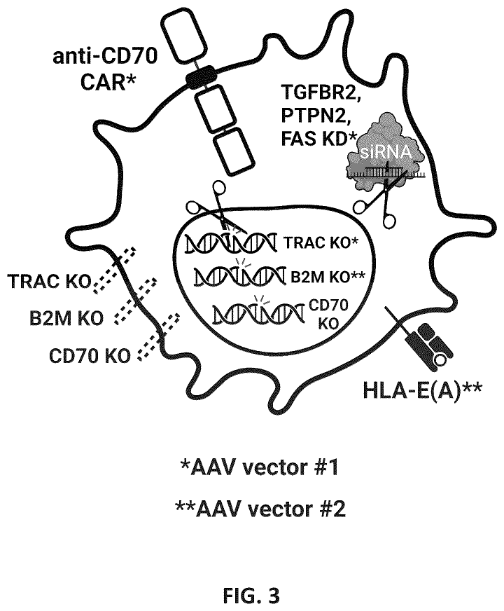

Table E2 and FIG. 3 provide a summary of the allogeneic engineering strategy.

TABLE E2
Allogeneic T Cell Functional Edits

| | | | Delivery |
|---|---|---|---|
| Locus Edit | Purpose | Function | Vehicle |
| KO TCR alpha | Knock-out TRAC locus | Prevent GvHD | RNP #1 |
| constant (TRAC) | disrupting endogenous TCR | | |
| | expression | | |
| KI anti-CD70 CAR | Express anti-CD70 CAR | Target CD70+ | AAV6 vector #1 |
| at TRAC locus | | tumors | |
| KD Transforming | Reduce TGFB-mediated | Enhance CAR T | AAV6 vector #1 |
| growth factor, beta | suppression of CAR | potency | |
| receptor II (TGFBR2) | | | |
| KD Protein | Reduce expression of a | Enhance T cell | AAV6 vector #1 |
| tyrosine phosphatase | negative regulator of T cell | fitness and | |
| non-receptor type 2 | signaling | persistence | |
| (PTPN2) | | | |
| KD FS-7- | Reduce expression of a | Enhance T cell | AAV6 vector #1 |
| associated surface | negative regulator of T cell | fitness and | |
| antigen (FAS) | signaling | persistence | |
| KO β2 | Knock-out B2M locus | Limit rejection | RNP #2 |
| microglobulin (B2M) | eliminating HLA Class I | of CAR T by host T | |
| | expression | cells (HvG reaction) | |
| KI HLA-E in B2M | Express HLA-E NK cell | Limit rejection | AAV6 vector #2 |
| locus | inhibitory ligand | of CAR T by host | |
| | | NK cells (HvG | |
| | | reaction) | |
| KO CD70 | Knock-out CD70 locus | Avoid CAR | RNP #3 |
| | disrupting endogenous CD70 | T:CAR T fratricide | |
| | expression | | |

Abbreviations: B2M, β2 microglobulin; CAR, chimeric antigen receptor; GvHD, graft versus host disease; HDR, homology-directed repair; HLA, human leukocyte antigen; HLA-E, HLA class I histocompatibility antigen, alpha chain E; HvG, host versus graft; KI, knock-in, KO, knock-out; KD, knock-down; NK, natural killer; TCR, T-cell receptor; TRAC, TCR alpha constant.

### A. Genomic Editing Strategy and Generation of Allogeneic Engineered T Cell Product

A systematic and comprehensive approach was used to select and characterize the specificity of the sgRNAs used to knock-out (KO) the TRAC, B2M, and CD70 loci, respectively. Candidate gRNA target sites were selected according to the protospacer adjacent motif (PAM) sequence for Cas12a. Each gRNA further comprised a scaffold sequence for the respective Cas and included base modifications.

CRISPR-associated protein (Cas)12a-based TRAC gRNA (SEQ ID NO:55) was selected for the disruption of the target site sequence GAGTCTCTCAGCTGGTACACG (SEQ ID NO:59) at the TRAC locus. The gRNA included a spacer sequence set forth in SEQ ID NO: 58 and a Cas12a binding scaffold sequence set forth in SEQ ID NO:57 added to the 5′-terminus of the spacer targeting domain, and was further modified with a DNA extension set forth in SEQ ID NO:56 and base modifications.

Cas12a-based B2M gRNA (SEQ ID NO:61) was selected for disruption of the target site sequence AGTGGGGGTGAATTCAGTGTA (SEQ ID NO:63) at the B2M locus. The gRNA included a spacer sequence set forth in SEQ ID NO:62 and a Cas12a binding scaffold sequence set forth in SEQ ID NO:57 added to the 5′-terminus of the spacer targeting domain, and was further modified with a DNA extension set forth in SEQ ID NO:56 and base modifications.

Cas12a-based CD70 gRNA (SEQ ID NO:64) was selected for disruption of the target site sequence TTCCAGTGGGACGTAGCTGAG (SEQ ID NO:66) at the CD70 locus. The gRNA included a spacer sequence set forth in SEQ ID NO:65 and a Cas12a binding sequence set forth in SEQ ID NO:57 added to the 5′-terminus of the spacer targeting domain, and was further modified with a DNA extension set forth in SEQ ID NO:56 and base modifications.

To engineer cells by the gene editing strategy, leukapheresis samples from healthy donors was collected, combined and cryopreserved. The cryopreserved leukapheresis samples were thawed and used to select primary human T cells by positive selection of CD4+ T cells and CD8+ T cells by immunoaffinity-based selection from thawed samples. The selected CD4+ and CD8+ T cells were stimulated by culturing the T cells with an anti-CD3/anti-CD28 antibody stimulatory reagent (Expamer) at 37° C. in media with recombinant cytokines.

For gene editing, the activated T cells were electroporated with ribonucleoprotein (RNP) complexes containing the respective nuclease and the gRNA. Specifically, for introducing a genetic disruption at the endogenous TRAC locus, the cells were electroporated with RNP complexes containing Cas12a and TRAC-targeting gRNA set forth in SEQ ID NO: 55 (RNP #1). For introducing a genetic disruption at the endogenous B2M locus, the cells were electroporated with RNP complexes containing Cas12a and B2M-targeting gRNA set forth in SEQ ID NO: 61 (RNP #2). For introducing a genetic disruption at the endogenous CD70 locus, the cells were electroporated with RNP complexes containing Cas12a and CD70-targeting gRNA set forth in SEQ ID NO: 64 (RNP #3).

After electroporation, a mixture of rAAV6 preparations each containing donor templates either for targeted integration (KI) of transgene sequence for the shRNA cassette and CAR at the TRAC locus (AAV vector #1) and/or for HLA-E at the B2M locus (AAV vector #2) were added to the cells in the presence of a DNA-PK inhibitor. The general structure of the donor templates were as follows: [5′ homology arm]-[transgene sequences]-[3′ homology arm].

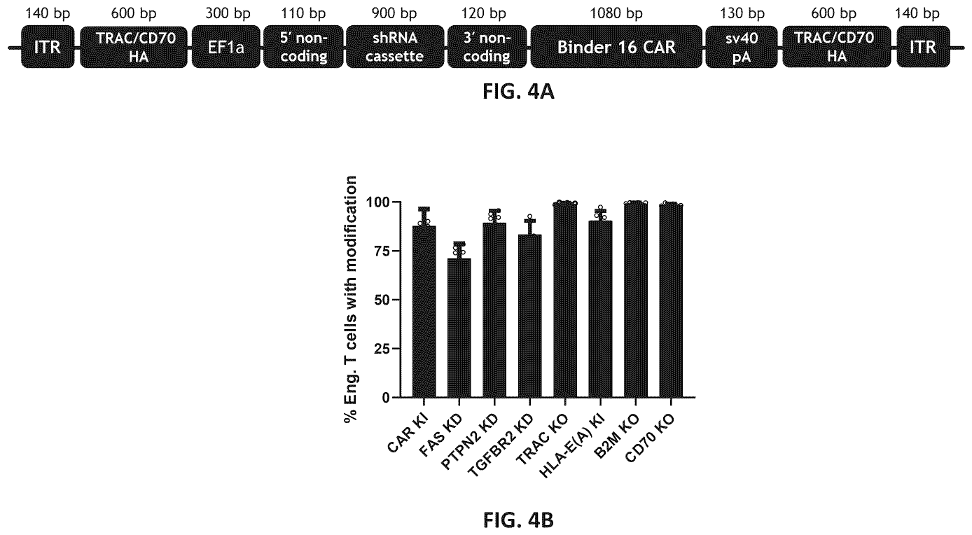

Specifically, AAV vector #1 contained the donor template for KI of the shRNA cassette and CAR into the TRAC locus included 600 base pair nucleic acid sequences homologous to sequences surrounding the target integration site in exon 1 of the human TRAC gene (5′ homology arm sequence set forth in SEQ ID NO:30; 3′ homology arm sequence set forth in SEQ ID NO:31), a shRNA cassette set forth in SEQ ID NO:71 and the CAR transgene nucleic acid sequence encoding the exemplary CAR (set forth in SEQ ID NO: 14 (where X is Q or pyroglutamate) with the signal peptide; SEQ ID NO:38 (where X is Q or pyroglutamate) without the signal peptide). The shRNA cassette encoded a first shRNA targeting TGFBR2 (guide sequence set forth in SEQ ID NO:74), a second shRNA also targeting TGFBR2 (shRNA guide sequence set forth in SEQ ID NO:75), a third shRNA targeting PTPN2 (guide sequence set forth in SEQ ID NO:73), and a fourth shRNA targeting FAS (guide sequence set forth in SEQ ID NO:72). The transgene sequence encoding the shRNA cassette and CAR also included a heterologous EF1α promoter (SEQ ID NO: 19) operably linked to the nucleotide sequence encoding both the shRNA cassette and the CAR to control expression of the shRNAs and CAR when expressed from a cell introduced with the donor template by HDR KI into the disrupted TRAC locus. Additionally, 5′ and 3′ non-coding element sequences (set forth in SEQ ID NO:69 and SEQ ID NO:70, respectively) surround the shRNA cassette. Finally, the template also included an SV4f polyA sequence (SEQ ID NO:76) following the C-terminus of the sequence encoding the shRNA cassette and CAR but prior to the 3′ homology arm sequence. The donor template without the homology arms is set forth in SEQ ID NO: 67 and with the homology arms is set forth in SEQ ID NO:77. A summary of each component of the donor template in AAV vector #1 is described in Table E3 below and FIG. 4A.

TABLE E3
Elements of AAV vector #1

| | Nucleotide Positions |
|---|---|
| | within the AAV |
| | vector #1 full sequence |
| Element | (SEQ ID NO: 77) |
| TRAC 5′ homology arm (SEQ ID | 1-600 |
| NO: 30) | |
| EF1α promoter (SEQ ID NO: 19) | 607-980 |
| 5′ non-coding element part 1 (SEQ ID | 981-1008 |
| NO: 69) | |
| shRNA cassette (SEQ ID NO: 71) | 1009-1907 |
| 5′ non-coding element part 2 | 1908-1953 |
| Polypurine tract | 1954-1971 |
| 5′ non-coding element part 3 | 1976-2019 |
| Anti-CD70 CAR (SEQ ID NO: 17) | 2032-3108 |
| SV40 polyA (SEQ ID NO: 76) | 3117-3250 |
| TRAC 3′ homology arm (SEQ ID | 3257-3856 |
| NO: 31) | |

Note:
The combination of a 5′ non-coding element part 2, polypurine tract, and 5′ non-coding element part 3 from nucleotide positions 1908-2019 is also referred to as a 3′ non-coding element set forth in SEQ ID NO: 70

AAV vector #2 contains the donor template for KI of an HLA-E fusion protein (set forth in SEQ ID NO: 78) into the B32M locus and included ˜800 base pair nucleic acid sequences homologous to sequences surrounding the target integration site of the human B32M gene (5′ homology arm sequence set forth in SEQ ID NO:32; 3′ homology arm sequence set forth in SEQ ID NO:33) and an HLA-E fusion transgene nucleic acid sequence set forth in SEQ ID NO:35 encoding the HLA-E fusion construct set forth in SEQ ID NO:34. The HLA-E fusion protein transgene sequence is inserted in the RNP site in exon 2 of B12M in frame with the endogenous amino acid sequence and driven by the endogenous B32M promoter. The HLA-E fusion protein contains a 5′ T2A cleavable sequence (SEQ ID NO:25) to remove endogenous B32M sequence amino acids. The remaining HLA-E fusion protein sequence encodes a single chain fusion protein with HLA-E specific binding peptide and B32M covalently attached to HLA-E through linkers. The encoded HLA-E fusion protein contained a B32M leader sequence (SEQ ID NO: 47; amino acids 1-20 of SEQ ID NO:34), a specific HLA-E binding peptide (SEQ ID NO: 48; amino acids 21-29 of SEQ ID NO:34), a (G4S)x3 linker (SEQ ID NO: 53; amino acids 30 to 44 of SEQ ID NO:34), B2M coding sequence without signal peptide (SEQ ID NO: 37; amino acids 45 to 143 of SEQ ID NO:34), (G4S)x4 linker (SEQ ID NO: 54; 144 to 163 of SEQ ID NO:34), and HLA-E coding sequence without signal peptide (SEQ ID NO: 36; amino acids 164 to 500 of SEQ ID NO:34). When expressed, the B2M leader is cleaved from the fusion protein. A summary of each component of the transgene in AAV vector #2 is described in Table E4 below.

TABLE E4
Elements of AAV vector #2

| | Nucleotide Positions |
|---|---|
| | within the AAV |
| | vector #2 full sequence |
| Element | (SEQ ID NO: 78) |
| B2M 5′ homology arm (SEQ ID NO: 32) | 152-950 |
| HLA-E fusion protein sequence (SEQ ID | 1020-2521 |
| NO: 35) | |
| B2M 3′ homology arm (SEQ ID NO: 33) | 2674-3473 |

Following electroporation and AAV transduction, cells were incubated for recovery under static conditions before expansion in serum-free media with recombinant cytokines. Expansion was carried out using a G-Rex. The cells were expanded for 7 days post transduction (between 5 to 7 cumulative fold doublings).

At 7 days post transduction, the harvested material was sampled, and cell counting was performed to calculate the total number of T cells followed by flow cytometry of the sample for T cell characterization at harvest. The product was then formulated with 10% DMSO by adding CryoStor® CS10 Cryopreservation Freeze Medium.

### B. Characterization of Allogeneic CAR-Engineered T Cells

Engineered allogenic cells generated using methods described in Example 5A above from six healthy human donors were assessed for expression of knock-in, knock-out, and knock-down efficiencies. HLA-E, CAR, TGFBR2, PTPN2, FAS, TRAC, and B2M perturbations were assessed by flow cytometry. Knock-out CD70 was assessed by Next Generation Sequencing (NGS).

Results are shown in FIG. 4B. The engineered cells exhibited efficient knock-out of CD70, TRAC (as determined from low percent CD3+ surface expression) and B2M (as determined low percent positive HLA-E expression) in the primary T cells, as well as high level expression of the recombinant CAR and HLA-E fusion protein. The results also demonstrated efficient knock-down of TGFBR2, PTPN2, and FAS. The results support the generation of T cells with (1) high efficiency expression of (i) an shRNA cassette that successfully knock-downs TGFBR2, PTPN2, and FAS, (ii) an exemplary recombinant CAR when integrated at the endogenous TRAC locus, and (iii) HLA-E when integrated at the endogenous B2M locus, and (2) containing a CD70 KO, the TRAC KO and the B2M KO. The results further support that the generation of the T cells with the indicated modifications are highly consistent donor-to-donor.

### Example 6—Validation of shRNA Cassette in Engineered Allogeneic T Cells

Experiments were performed to test the functionality of the AAV vector #1 transgene containing an shRNA cassette encoding different shRNAs targeting TGFBR2, PTPN2, and/or FAS that were knocked-in to the TRAC locus. Different promoters, KI sites, 5′ non-coding elements, and shRNAs were evaluated to determine how different parameters affected expression of CAR; knock-down of TGFBR2, PTPN2, and FAS; and the functional efficacy of the CAR T cells, such as cytolytic activity.

The experiments were carried out to assess the effect of the different shRNA cassettes to improve CAR T cell activity by reducing expression of TGFBR2, PTPN2 and FAS in the engineered T cells. Because TGFBR2 has the ability to suppress CAR T cell response, a purpose of decreasing TGFBR2 by KD is to improve the potency of CAR T cells. Because it is also believed that PTPN2 can antagonize cytokine signaling required for T-cell function, homeostasis, and differentiation, one rationale for decreasing PTPN2 by KD is to improve efficacy of CAR T cells. Because it is also believed that FAS can initiate T cell death after binding to FAS ligand, one rationale for decreasing FAS by KD is to decrease FAS ligand-mediated cell death of CAR T cells. Further, the use of shRNAs to KD these genes rather than by gene editing to knock out is believed to prevent introducing additional double-stranded breaks into the manufacture of engineered allogeneic T cells and minimize number of unique translocation type events; increasing number of double-stranded breaks may positively correlate with an increase in the number of translocation type events.

A. Assessment of Transgene Encoding shRNA Cassette and CAR KI into the TRAC Locus

Engineered CAR T cells were generated from two human donors following the collection and activation of T cells as previously described in Example 5.

For gene editing, the activated T cells were electroporated with RNP #1 and a donor template for KI of the shRNA cassette and CAR into the TRAC locus as previously described in Example 5, except that the non-coding element sequences flanking the shRNA cassette were replaced with a HTLV non-coding element sequences.

Following recovery and harvesting of the engineered T cells, expression of CAR, PTPN2, TGFBR2, and FAS was measured via flow cytometry in the engineered cells as compared to mock T cells that did not receive the shRNA cassette but contained a knock-out of the CD70 and TRAC loci. A polyclonal a-VHH antibody was used to measure expression CAR and monoclonal antibodies against PTPN2, TGFBR2, or FAS was used to measure knockdown of each of the respective mRNAs. In addition, engineered T cells were also cultured with a target cell line expressing CD70 as previously described in Example 2 to measure the cytolytic activity of the engineered T cells.

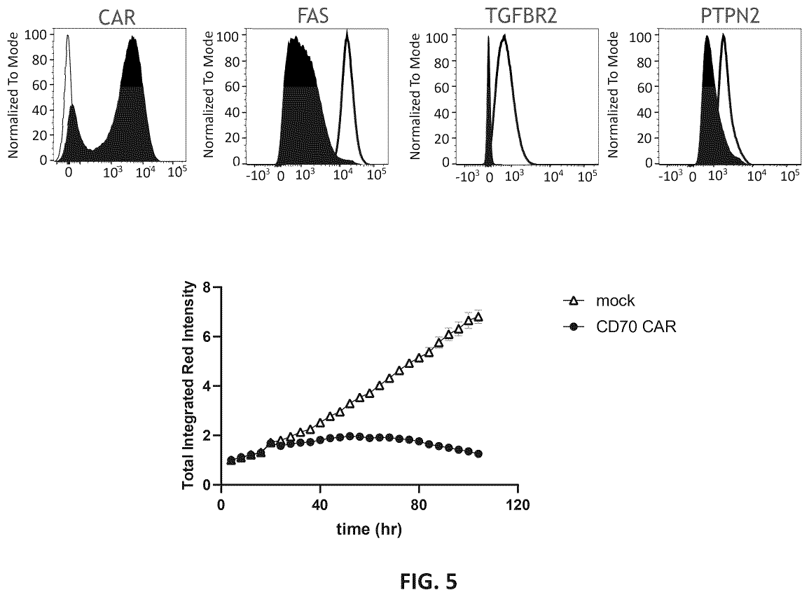

As shown in FIG. 5, cells containing the knock-in of the shRNA cassette and CAR had higher CAR expression (far left) as well as lower PTPN2 (far right), TGFBR2 (middle right), and FAS expression (middle left) (black) as compared to mock T cells that contained a knock-out of CD70 and TRAC loci but that did not contain a shRNA cassette and anti-CD70 CAR transgene (white with black border). Further, as shown in the bottom panel, there were fewer target cells, as measured by Incucyte red intensity, in conditions that were incubated with cells containing the knock-in of the shRNA cassette and CAR as compared to mock T cells that contained a knock-out of CD70 and TRAC loci but that did not contain a shRNA cassette and anti-CD70 CAR transgene.

These results demonstrate that the transgene sequence encoding the shRNA cassette and CAR upon integration into the TRAC locus is able to successfully express each of the different components encoded in the transgene. Further, knock-in of the transgene is able to enhance the ability of T cells to kill CD70-expressing target cells.

B. Assessment of Transgene Encoding shRNA Cassette and CAR KI into the TRAC Locus

Following validation of the transgene encoding the shRNA cassette and CAR, the design of the transgene was further optimized to enhance expression of CAR. Parameters to optimize included the specific promoter/non-coding element combination used (EF1α promoter-synthetic non-coding element (Table E5), EF1α promoter-HTLV non-coding element (Table E6), MND promoter-HTLV non-coding element (Table E7)) and the locus where the transgene was knocked-in (TRAC or CD70). The specific promoter/non-coding element combinations used are described in Table E5, Table E6, and Table E7 below.

TABLE E5
EF1α promoter-synthetic non-coding element

| | Nucleotide Position |
|---|---|
| Element | within SEQ ID NO: 77 |
| EF1α promoter (SEQ ID NO: 19) | 607-815 |
| 5′ non-coding element (SEQ ID NO: 69) | 816-1008 |
| shRNA cassette (SEQ ID NO: 71) | 1009-1907 |
| 3′ non-coding element (SEQ ID NO: 70) | 1908-2019 |

TABLE E6
EF1α promoter-HTLV non-coding element

| Element | SEQ ID NO: of Element |
|---|---|
| EF1α promoter | 19 |
| 5′ non-coding element | 124 |
| shRNA cassette | 71 |
| 3′ non-coding element | 125 |

TABLE E7
MND promoter-HTLV non-coding element

| Element | SEQ ID NO: of Element |
|---|---|
| MND promoter | 123 |
| 5′ non-coding element | 124 |
| shRNA cassette | 71 |
| 3′ non-coding element | 125 |

Engineered T cells were generated as described in Example 6A using either RNP #1 if the knock-in site was TRAC or RNP #3 if the knock-in site was CD70 as well as the different optimized transgene sequences in AAV vector #1. Mock T cells were also used as a control. Following recovery and harvesting of the engineered T cells, expression of CAR was measured for each of the conditions. In addition, engineered T cells containing either the EF1α promoter-HTVL non-coding element or EF1α promoter-synthetic non-coding element were injected into NOD.Cg-Prkdc^{scid}IL-2rg^{tm1Wjl}/SzJ immunodeficient mice (NSG) mice with subcutaneous renal cell carcinoma cell line 786-O xenografts.

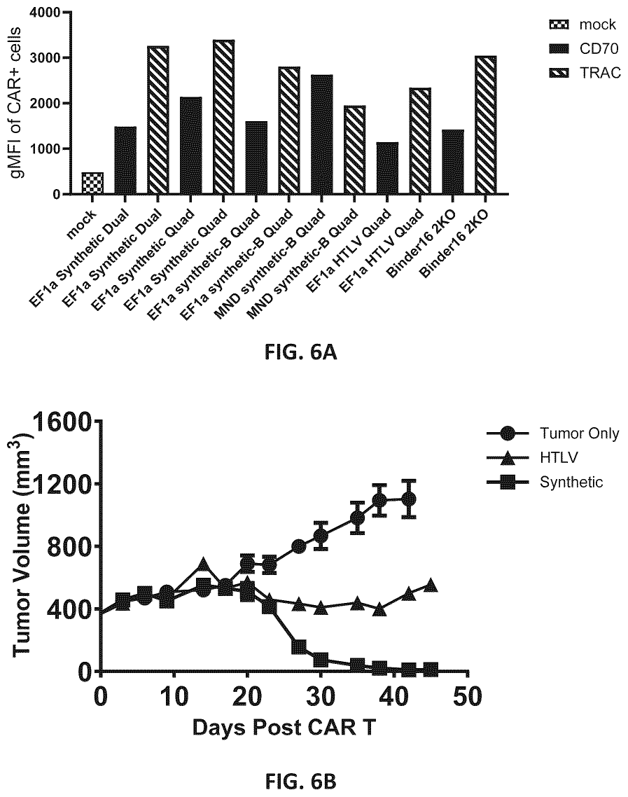

As shown in FIG. 6A, higher CAR expression occurred when the transgene was integrated into the TRAC locus as compared to the CD70 locus. In addition, while all tested promoter/non-coding element combinations resulted in CAR expression, the EF1α promoter-synthetic non-coding element (synthetic quad) combination resulted in the highest mean intensity of CAR expression as measured via flow cytometry. Further, as shown in FIG. 6B, use of the synthetic non-coding element resulted in greatest in vivo cytolytic activity, as shown by the greater decrease in tumor volume in mice that received engineered T cells containing the synthetic non-coding element as compared to the HTLV non-coding element.

These results demonstrate that knock-in of a transgene sequence encoding a shRNA cassette and CAR can lead to successful expression of CAR T cells with improved cytolytic potency against target cells.

C. Assessment of Optimized shRNA Cassette KI in Engineered T Cells

Experiments were performed to assess effect of genetic engineering to knock-in an optimized transgene encoding an shRNA cassette and the exemplary CAR set forth in SEQ ID NO: 14 that further contained the EF1α promoter set forth in SEQ ID NO: 19 and the synthetic non-coding element (full sequence set forth in SEQ ID NO:68; the 5′ non-coding element set forth in SEQ ID NO:69; and the 3′ non-coding element set forth in SEQ ID NO:70) on knock-down of the genes targeted by the shRNA cassette, expression of CAR, and cytolytic activity.

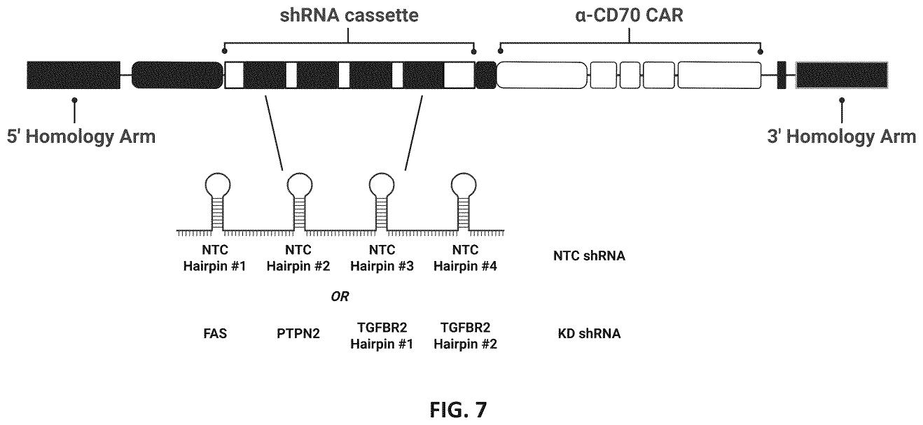

Engineered CAR T cells were generated from three human donors as previously described in Example 6A. As a control, engineered CAR T cells were also generated where the shRNA cassette was replaced with a non-targeting control (NTC) shRNA cassette that contained non-targeting hairpins that targeted luciferase and green fluorescent protein genes that are not present in the human genome. A schematic of the transgene flanked by TRAC homology arms and encoding: (a) either an exemplary shRNA cassette set forth in SEQ ID NO: 71 or a NTC shRNA cassette, and (b) an exemplary CD70 CAR is shown in FIG. 7.

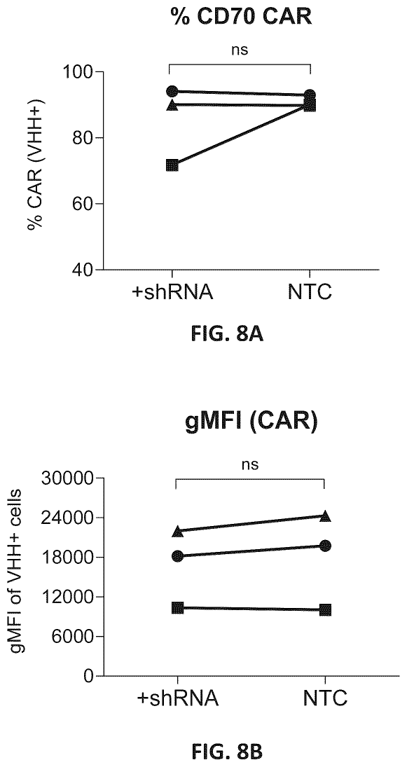

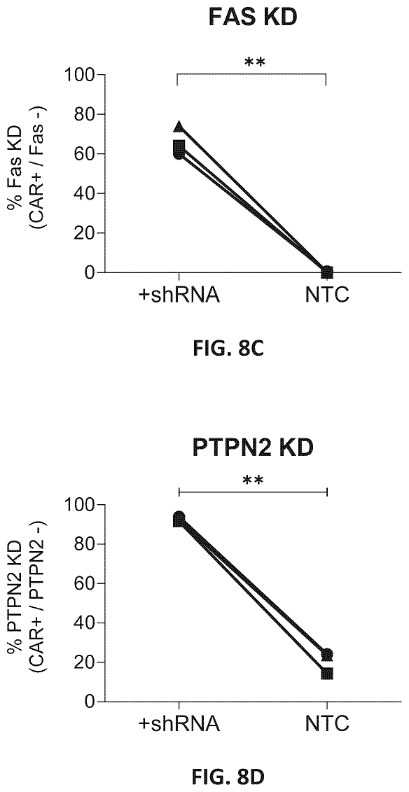

Following collection of the engineered CAR T cells, percentage of anti-CD70 CAR (FIG. 8A), gMFI of CAR T cells positive for the anti-CD70 VHH binder (FIG. 8B), percent of FAS knock-down (FIG. 8C), and percent of PTPN2 knock-down (FIG. 8D) was assessed in the three separate donors. As shown in FIG. 8A and FIG. 8B, expression of CAR occurred in the engineered CAR T cells regardless of the presence of the shRNA cassette. However, as shown in FIG. 8C and FIG. 8D, FAS and PTPN2 knock-down only occurred when a shRNA cassette, rather than a NTC shRNA cassette, was present.

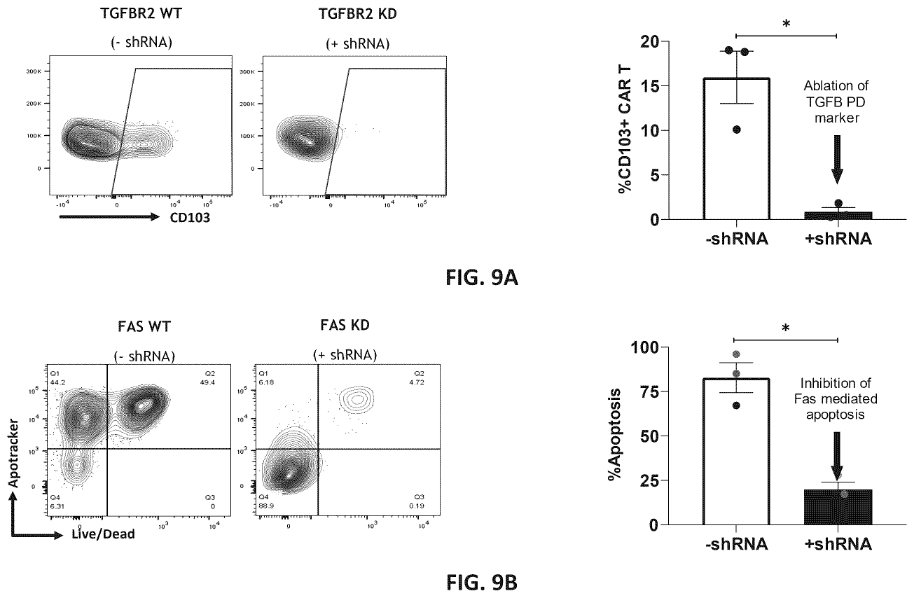

TGFBR2 and FAS knock-down were then further validated using additional pharmacological assays. CD103 is a TGFBR2 pharmacodynamic marker. Thus, levels of CD103 were measured using flow cytometry on engineered CAR T cells containing either a shRNA cassette (+shRNA) or NTC shRNA cassette (−shRNA). As shown in the representative flow cytometry plots (left) and summary plot (right) in FIG. 9A, engineered CAR T cells containing a shRNA cassette had reduced levels of CD103 as compared to engineered CAR T cells containing a NTC shRNA cassette.

Fas is part of a known pathway leading to apoptosis. Thus, following incubation of engineered CAR T cells with a Fas agonist, percentages of live or dead cells were measured using flow cytometry. As shown in the representative flow cytometry plots (left) and summary plot (right) in FIG. 9B, engineered CAR T cells containing a shRNA cassette had lower percentages of dead cells and lower levels of apoptosis as compared to engineered CAR T cells containing a NTC shRNA cassette.

The percentage of either CD103 positivity or apoptosis in engineered CART cells containing a CAR and either a shRNA cassette or NTC shRNA cassette is summarized in Table E8 below.

TABLE E8
Summary of Functional Outcomes
from Optimized shRNA Cassette

| Sample | % CD103+ | % Apoptosis |
|---|---|---|
| CAR + shRNA cassette | 0.9 ± 0.8 | 20.0 ± 7.2 |
| CAR + NTC shRNA cassette | 16.0 ± 5.1 | 83.0 ± 14.6 |

Error above represents standard deviation across three donors.

These results demonstrate that the optimized transgene sequence encoding the shRNA cassette and CAR that is driven by a EF1α promoter and incorporates synthetic non-coding elements is, upon integration into the TRAC locus, able to successfully express each of the different components encoded in the transgene, including lead to knock-down of PTPN2, TGFBR2, and FAS. Furthermore, the knock-down of PTPN2, TGFBR2, and FAS from the shRNA cassette is consistent across multiple donors.

### Example 7—Assessment of CD70 KO in Engineered T Cells Against Fratricide

Engineered T cells described in Example 2 were designed with knock-out of CD70 because a subset of activated T cells express CD70, thus leading to potential for fratricide or attack on CD70-expressing T cells by T cells that also express an anti-CD70 CAR. To assess effect of the knock-out (KO) of the endogenous CD70 locus in T cells expressing an anti-CD70 CAR on potential fratricide, engineered T cells were measured for harvest yield and viability, and were characterized for in vivo cytolytic activity.

The protective effect of a CD70 KO in an T cell against fratricide was first explored by developing a positive control cell expressing an anti-CD70 CAR where fratricide did not occur. To avoid fratricide in the positive control cell, a control “masking” anti-CD70 CAR (SEQ ID NO: 126) was used that was able to block binding of CD70 in cis. To validate that the “masking” anti-CD70 CAR was able to block binding of CD70, THP-1 cells expressing CD70 were engineered to express either the “masking” anti-CD70 CAR or, as a control, an anti-CD19 CAR that did not block detection binding of CD70. The engineered THP-1 cells were then co-cultured with T cells engineered to express the “masking” anti-CD70 CAR. As another control, some engineered THP-1 cells were also not exposed to T cells (THP-1 alone). THP-1 cells were labeled with NucLight Red (NLT) to monitor cytolytic cell killing via analysis of red fluorescence signal over time on an IncuCyte® instrument.

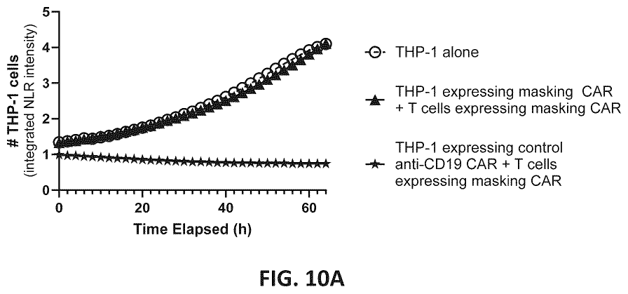

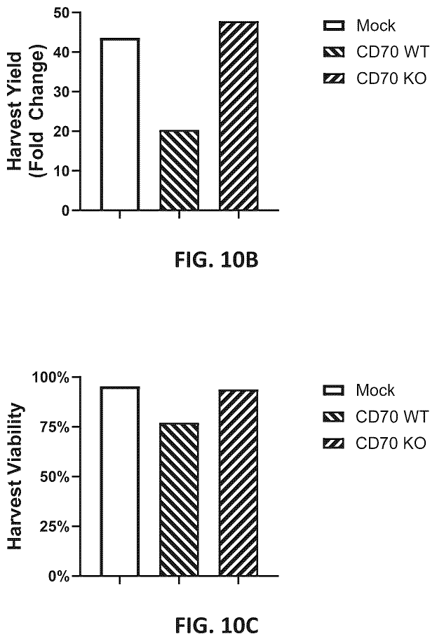

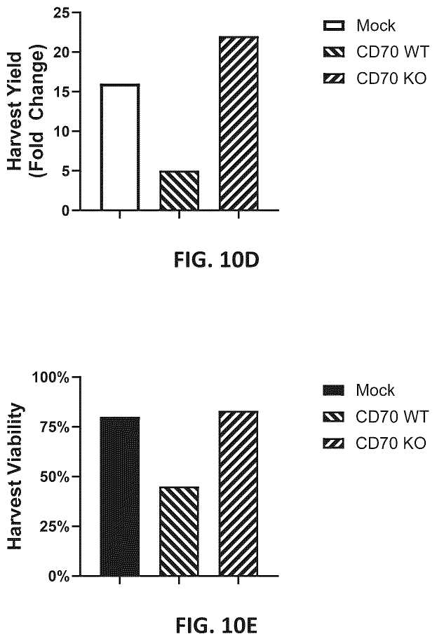

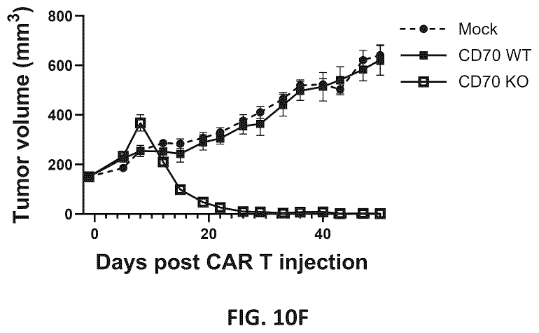

The results are shown in FIG. 10A. THP-1 cells expressing a “masking” anti-CD70 CAR sustained similar growth to THP-1 cells that were not exposed to T cells, indicating that the THP-1 cells expressing a “masking” anti-CD70 CAR were protected from T cell killing due to ability of the “masking” anti-CD70 CAR to mask the expression of CD70. In contrast, THP-1 cells that expressed a non-masking anti-CD19 CAR experienced more killing from T cells, indicating that these THP-1 cells were targeted by anti-CD70 CAR on T cells if no masking was present.

Following validation of the “masking” anti-CD70 CAR, engineered T cells were developed that expressed either the “masking” anti-CD70 CAR as a positive control or the exemplary anti-CD70 CAR set forth in SEQ ID NO:14. Specifically, CD4+ and CD8+ T cells from a human donor subject were introduced to an AAV into a TRAC locus by homology directed repair similar to methods described in Example 5, except the cells were engineered to integrate into the TRAC locus either the “masking” anti-CD70 CAR or the exemplary anti-CD70 CAR. In addition, some of the engineered T cells also had the CD70 locus knocked-out (CD70 KO) using the RNP #3 previously described in Example 5. As a control, mock T cells that contained a TRAC KO but no CAR transgene were also tested.

Following recovery and expansion of engineered T cells, harvested material was sampled and yield and viability was determined (see Table E9). As shown in FIG. 10B and FIG. 10C (engineered T cells expressing a “masking” anti-CD70 CAR) and FIG. 10D and FIG. 10E (engineered T cells expressing the exemplary anti-CD70 CAR), CD70 KO in T cells expressing an exemplary anti-CD70 CAR or a “masking” anti-CD70 CAR had higher yield (FIG. 10B and FIG. 10D) and viability (FIG. 10C and FIG. 10E) as compared to CD70 WT, validating that CD70 KO is a viable strategy to reduce fratricide even with a masking anti-CD70 CAR.

TABLE E9
Summary of Yield and Viability of
Engineered T cells with CD70 KO

| | CD70 | Harvest Yield | Harvest |
|---|---|---|---|
| CAR | genotype | (fold expansion) | Viability (%) |
| Mock | CD70 KO | 39.43 | 95.8 |
| ‘masking’ a-CD70 | CD70 WT | 20.3 | 77 |
| CAR | | | |
| ‘masking’ a-CD70 | CD70 KO | 47.8 | 93.8 |
| CAR | | | |
| Mock | CD70 KO | 16 | 80 |
| Exemplary a-CD70 | CD70 WT | 5 | 45 |
| CAR | | | |
| Exemplary a-CD70 | CD70 KO | 22 | 83 |
| CAR | | | |

Further, engineered T cells expressing the “masking” anti-CD70 CAR were further injected into NOD.Cg-Prkdc^{scid}IL-2rg^{tm1Wjl}/SzJ immunodeficient mice (NSG) mice with subcutaneous renal cell carcinoma cell line ACHN xenografts. As shown in FIG. 10F, tumors continued to grow in mice injected with either mock or CD70 WT cells; however, tumors were cleared in mice that received CD70 KO cells. These results show that CD70 KO can increase potency of engineered T cells expressing an anti-CD70 CAR, including in the case where the anti-CD70 CAR is a masking CAR.

Together, these results demonstrate that CD70 KO both protects against fratricide—and enhances potency—of anti-CD70 CAR T cells, as shown by increase harvest yield, harvest viability, and cytolytic activity.

### Example 8—Assessment of HLA-E Knock-In in Engineered Allogeneic T Cells Against NK-Mediated Host Vs. Graft Disease (HvG)

HvG (Host vs. Graft) effects can be triggered by both host T and NK cell recognition of a graft. Knocking out (KO) MHC class I and/or MHC class II from engineered T cells results in the loss of MHC class I expression, which in turn leads to a loss of B2M surface expression. B2M molecules, recognized by inhibitory receptors on the surface of NK cells, provide a “self” recognition signal that blocks NK cell activation and attack. The absence of B2M surface expression disrupts the delivery of these inhibitory “self” recognition signals, leading to NK cell activation and subsequent target cell (e.g., engineered T cell) killing. These effects can directly impact the persistence and potency of engineered T cells, including the engineered allogeneic T cells generated by methods described in Example 5.

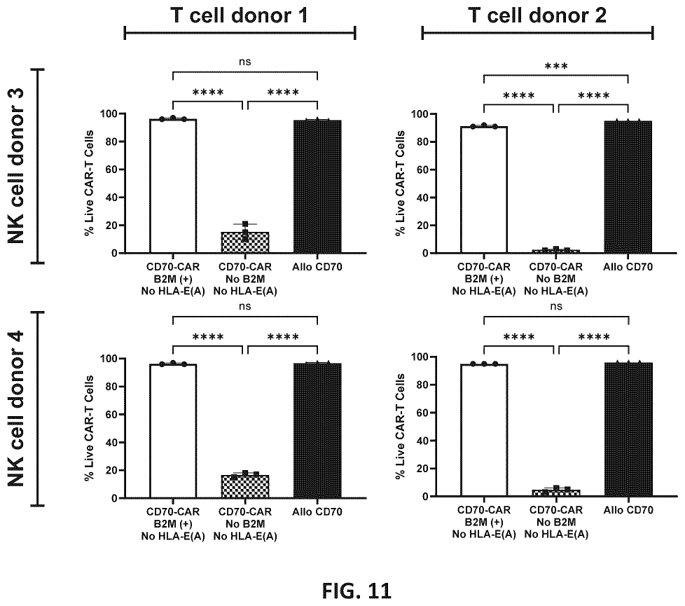

Reintroduction of engineered inhibitory signals can redirect NK-mediated responses. To reduce NK-mediated killing of the allogeneic engineered T cells, experiments were performed to determine if an engineered variant of HLA-E, a peptide-B2M-HLA-E fusion protein (HLA-E(A)), knocked into (KI) the B2M locus of the allogeneic T cells has the ability to counteract the NK cell activation. The HLA-E(A) molecule preferentially binds to the inhibitory receptor NKG2A on host NK cells while reducing binding to the activating NKG2C receptor. Engineered allogeneic T cells generated as described in Example 5 were generated from 2 healthy donors. Knock-in was performed using HDR driven integration of the transgene sequence delivered to T cells using rAAV6 vector during CRISPR mediated knockout of B2M. The resulting cells were plated at a 0.6:1 ratio with primary NK cells from 2 HLA-mismatched donors for 72 hours. As controls, T cells containing an exemplary anti-CD70 CAR (CAR set forth in SEQ ID NO:14), shRNA cassette, CD70 knock-out, and TRAC KO as previously described in Example 5A and additionally either (a) contained a wild-type B2M locus without HLA-E(A) addition, (b) contained a B2M KO but no knock-in of HLA-E(A), or (c) contained both the B2M KO and knock-in of the HLA-E fusion construct described in Example 5A (SEQ ID NO:78) by homology directed repair with the donor transgene template for insertion of a polynucleotide encoding the CAR and HLA-E fusion. After 72 hours, cells were analyzed by multicolor flow cytometry. NK cells were gated on CD56(+)CD5(−) cells, while T cells were gated on CD56(−)CD5(+) cells and further selected for CAR expression. The remaining % Live CD56(−)CD5(+) CAR(+) T cells are shown in FIG. 11. The control engineered T cells with endogenous expression of MHC-class I (no B2M KO; “CAR+, B2M WT control”) and without added HLA-E(A) maintained viability throughout the assay, as these cells provided inhibitory “self” signals to the NK cells. The controls with endogenous MHC-class I KO and no HLA-E(A) KI (B2M KO control) demonstrated a loss of viability after 72 hours and were rejected by NK cells. The inclusion of HLA-E(A) in the engineered T cells containing all modifications described in Table E2 (Allo CD70) were protected with a resulting viability greater than 85% live CAR-T cells after 72 hour incubation with NK cells.

These results demonstrate the protective capacity of the HLA-E knock in against NK-mediated HvG disease.

### Example 9—Functionality of shRNA Cassette KD in Engineered Allogeneic T Cells

The engineered allogeneic T cells generated in Example 5 were tested for cytolytic activity against target cells and proinflammatory cytokine production in response to co-culture with target cells when expressing both CAR and a shRNA cassette to knock-down FAS, PTPN2, and TGFBR2 or with both a CAR and a non-targeting control (NTC) shRNA cassette.

### A. Assessment of Cytolytic Activity in In Vitro 3D Tumor Spheroids

In this study, the efficacy of the engineered allogeneic T cells with or without the shRNA cassette was assessed in vitro using 3D tumor spheroids made of A498 tumor target cells. The A498 cells were labeled with NucLight Red (NLR) to monitor cytolytic cell killing via analysis of red fluorescent signal over time on an IncuCyte® instrument.

Allogeneic T cells with or without shRNA were initially incubated with 3D tumor spheroids at effector to target (E:T) cell ratios of 1:4, 1:8, or 1:16 on day 0 until day 8, when the cells were re-stimulated with a new batch of 3D at the same E:T cell ratios until day 16. To assess antigen-dependent CAR activity, co-cultures were assessed for cytolytic activity of the A498 target cells throughout the entire experiment and for cytokine production in the supernatant on the day 8 and day 16.

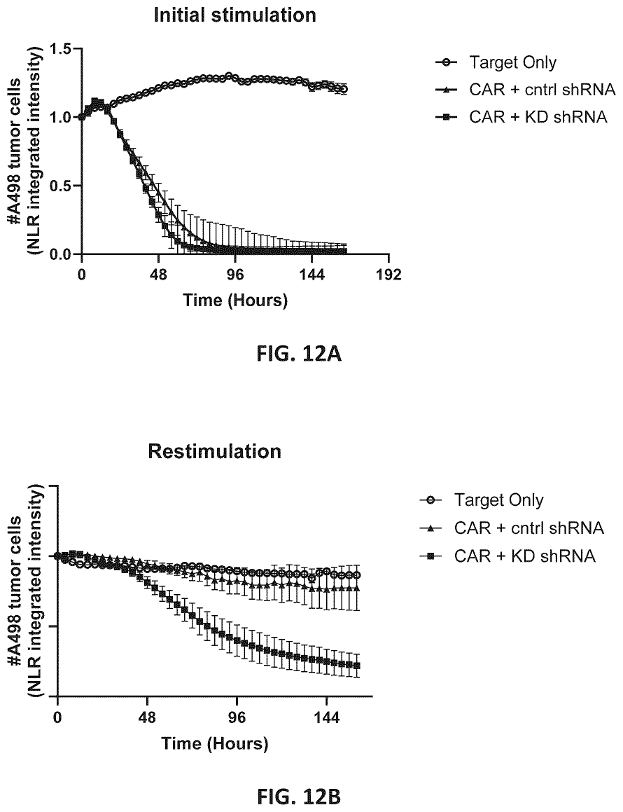

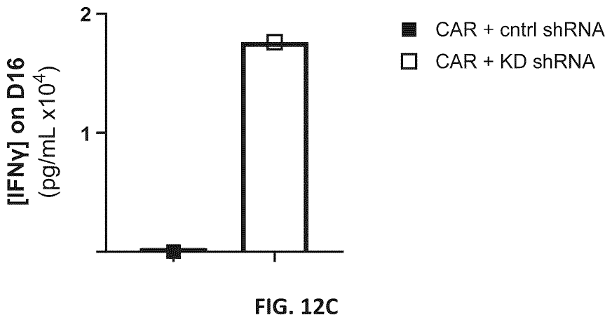

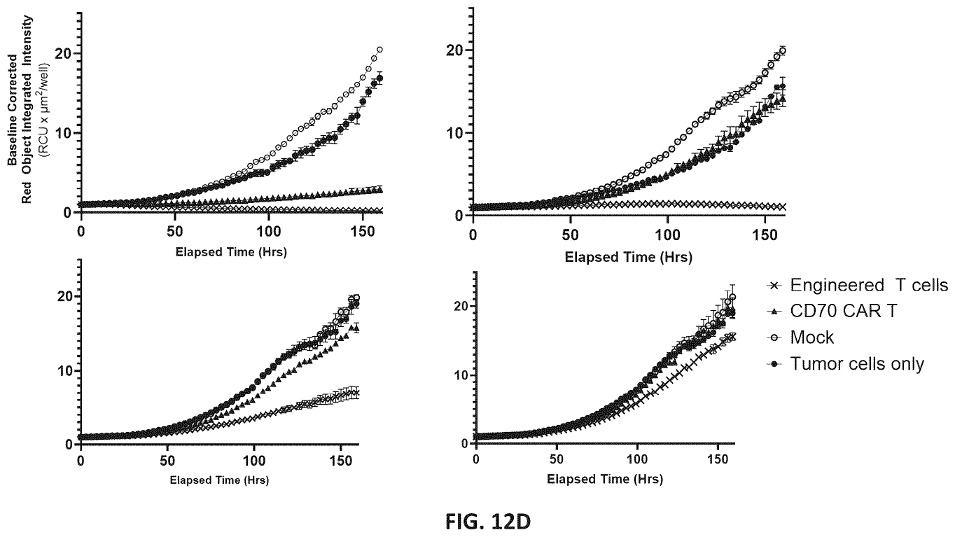

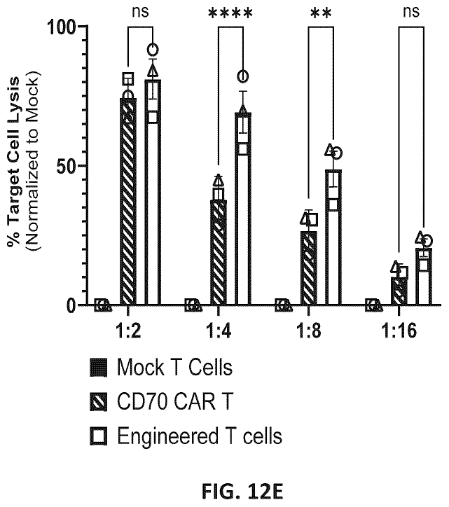

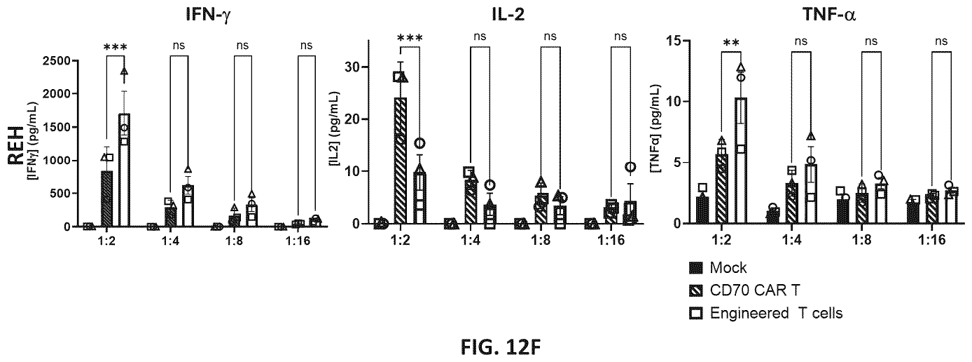

Cytolytic activity was determined by measuring total integrated fluorescence intensity in the 3D co-cultures by monitoring NucLight Red fluorescence for the entire time course using IncuCyte® S3 Live-Cell Analysis System. As shown in FIG. 12A, similar levels of cytolytic activity were observed for allogeneic T cells with or without the shRNA cassette, as compared to the target cells alone, during the initial stimulation. However, during the re-stimulation (FIG. 12B), cytolytic activity was only observed for allogeneic T cells with the shRNA cassette. In contrast, target cells experienced similar numbers when not incubated with T cell or when incubated with allogeneic T cells without the shRNA cassette.

From the same cultures as above, secreted IFN-γ was measured in supernatants collected on the final day of the experiment with engineered allogeneic T cells and the indicated tumor cell lines using an electrochemiluminescence cytokine immunoassay (FIG. 12C). Secreted IFN-γ levels were higher in engineered allogeneic T cells containing a shRNA cassette as compared to engineered allogeneic T cells without an shRNA cassette. Similar trends held for other measured cytokines, such as IL-2 and TNFa.

Together, these results demonstrate that the engineered allogeneic T cells have cytolytic activity against CD70 expressing tumor cells. Further, the potency and duration of the activity is increased when FAS, PTPN2, and TGFBR2 are knocked down.

B. Assessment of Cytolytic Activity and Cytokine Production with CD70+ REH ALL Tumor Cells

The functionality of the engineered allogeneic T cells described in Example 5 generated from three different donors was assessed when co-cultured with a 2D CD70+ REH acute lymphoblastic leukemia (ALL) cell line. The REH cells were labeled with NucLight Red (NLR) to monitor cytolytic cell killing via analysis of red fluorescent signal over time on an IncuCyte® instrument. To measure cytolytic activity, target cells were incubated with engineered CAR T cells with the exemplary KD shRNA cassette and target cell concentration was measured over 160 hours. Cytokine levels in the supernatant were also measured after 24 hours of co-culture. As controls, engineered CAR T cells with the exemplary KD shRNA were replaced with either mock T cells that did not contain a CAR knock-in transgene or engineered CAR T cells where the KD shRNA cassette was replaced with the NTC shRNA cassette.

Concentration of REH target cells following co-culture with T cells at different effector to target (E:T) ratios (1:2, top left; 1:4, top right; 1:8, bottom left; 1:16, bottom right) is shown in FIG. 12D. The results show that both the engineered CAR T cells with the exemplary KD shRNA cassette and the engineered CAR T cells with the NTC shRNA cassette both controlled the growth of the 2D REH tumor cells in a dose-dependent manner by approximately 50 to 100 hours after co-culture. However, the allogeneic CAR T cells with the KD shRNA cassette was more effective at killing REH tumor cells than the engineered CAR T cells with the NTC shRNA cassette at all E:T ratios tested.

Percent target cell lysis, normalized to the mock T cell control condition, at the indicated E:T ratios is shown in FIG. 12E. The results indicate that the engineered CAR T cells with the exemplary KD shRNA cassette demonstrated elevated percent lysis relative to the engineered CAR T cells with the NTC shRNA cassette after co-culture with the REH tumor cells.

FIG. 12F shows the production of proinflammatory cytokine IFNγ (left), IL-2 (middle), and TNFα (right) 24 hours after co-culture of T cells and REH cells at the indicated E:T ratio. Proinflammatory cytokines were readily detected following a 24 hour co-culture containing either the engineered CAR T cells with the exemplary KD shRNA cassette or the engineered CAR T cells with the NTC shRNA cassette. In contrast, proinflammatory cytokine levels for donor-matched mock process T cells controls were below the lower limit of detection except for TNFα levels that were above the lower limit of detection but lower in magnitude than the corresponding co-cultures with the engineered CAR T cells with the exemplary KD shRNA cassette and the engineered CAR T cells with the NTC shRNA cassette. Levels of IFNγ and TNFα detected following the 24 hour co-culture containing the engineered CAR T cells with the exemplary KD shRNA cassette were elevated relative to corresponding co-cultures containing the engineered T cells with the NTC shRNA cassette. Meanwhile, levels of IL-2 detected following 24 hour co-culture followed the opposite trend and were up to 40% lower in co-cultures containing the engineered CAR T cells with the exemplary KD shRNA cassette than the corresponding co-cultures cultures containing the CAR T cells with the NTC shRNA cassette.

Together, these results demonstrate that the engineered CAR T cells have dose-dependent cytolytic and cytokine activity against CD70 expressing tumor cells including REH ALL cells. Further, the potency of the activity is increased when FAS, PTPN2, and TGFBR2 are knocked down.

### B. Assessment of Anti-Tumor Efficacy in In Vivo RRC Xenograft Tumor Models

The efficacy of the engineered allogeneic T cells containing an exemplary anti-CD70 CAR transgene and knock-outs of CD70, TRAC, and B2M as described in Example 5A and with or without the shRNA cassette to kill target tumor cells was assessed in vivo using three different renal cell carcinoma (RCC) models. Characteristics of each model are summarized in Table E10 below.

TABLE E10
RCC tumor model characterization

| | | TGFB1 | TGFB1 | | |
|---|---|---|---|---|---|
| | | expression | expression | | |
| | | in vitro - | in vivo - | | |
| | CD70 | Mean | Mean | Growth | Growth |
| RCC | expression | (stdev) | (stdev) | rate in | rate in |
| Model | in vitro | pg/mL | pg/mL | vitro | vivo |
| 786-O | High | 2923 | 115.7 | Fast | Slow |
| | | (431) | (81.3) | | |
| A498 | High | 3234 | 228.9 | Moderate/ | Moderate |
| | | (377) | (80.5) | Fast | |
| ACHN | Medium | 3558 | 293.8 | Moderate | Slow |
| | | (426) | (503.6) | | |

Engineered allogeneic T cells containing either a shRNA cassette or NTC shRNA cassette were injected into NOD.Cg-Prkdc^{scid}IL-2rg^{tm1Wjl}/SzJ immunodeficient mice (NSG) mice with either a 786-O, A498, or ACHN xenograft. For experiments performed using the 786-O mice model, the injection per mouse contained either 50,000 or 150,000 cells from one of two donors.

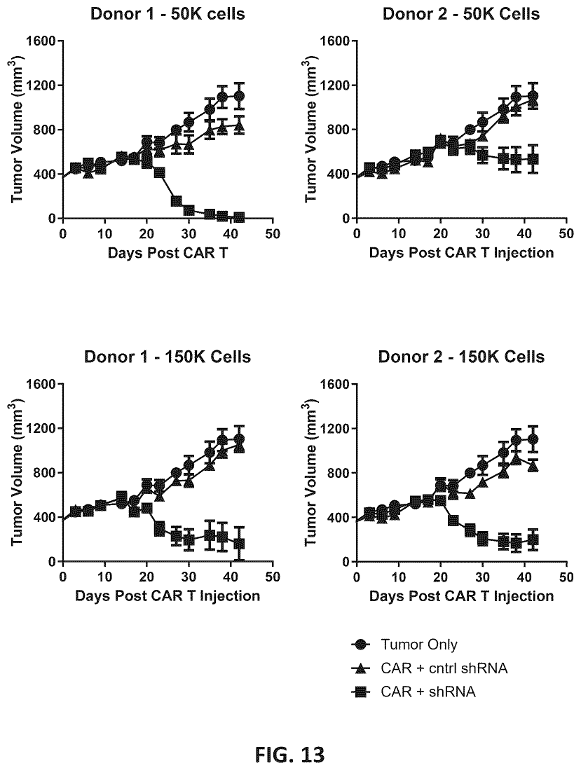

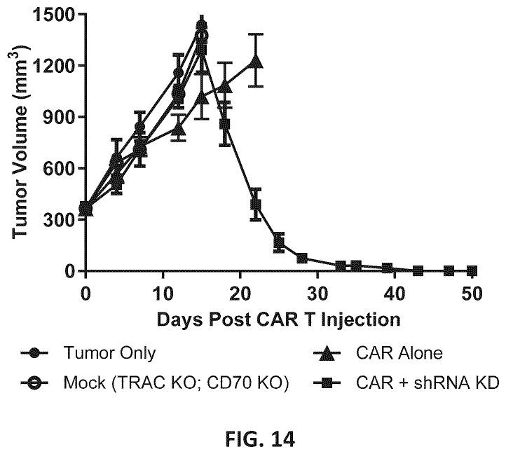

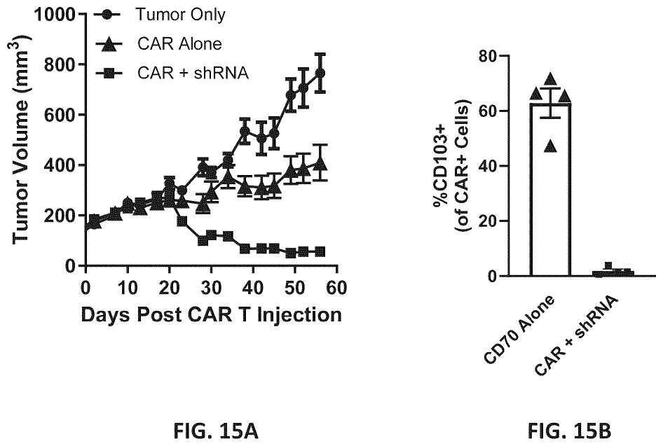

Results shown in FIG. 13 (786-O model), FIG. 14 (A498 model) and FIGS. 15A-15B (ACHN model) demonstrate that engineered allogeneic T cell containing a shRNA cassette were able to substantially reduce tumor volume size as compared to either engineered allogeneic T cells containing a NTC shRNA cassette (CAR alone) or when no T cells were injected (tumor only). Furthermore, as shown in FIG. 15B, use of the engineered allogeneic T cells containing the shRNA cassette reduced expression of the TGFBR2 pharmacodynamic marker CD103 as compared to use of the engineered allogeneic T cells containing the NTC shRNA cassette.

These results demonstrate the engineered allogeneic T cells exhibit strong cytolytic activity against CD70-expressing target cells in vitro and in vivo, including the ability to overcome TGFB suppression in vivo. The greater cytolytic activity shown by engineered allogeneic T cells containing the shRNA cassette may be caused by the knock-down of TGFBR2, which may then inhibit TGFB suppression in vivo. In addition, the engineered allogeneic T cells exhibited robust and dose-dependent proinflammatory cytokine production. Both the higher levels of cytolytic activity and cytokine production shown by engineered allogeneic T cells containing the shRNA cassette demonstrate that shRNA knockdown of FAS, PTPN2, and TGFBR2 promote the efficacy of the allogeneic T cells in their functional activity against tumor cells, including CD70-positive renal cell carcinoma (RCC).

### Example 10—Cytokine Production by Engineered Allogeneic T Cells Co-Cultured with CD70-Expressing Target Cells

The engineered CAR T cells generated in Example 5 were evaluated for functional activity, including cytokine production, in response to target cells expressing different levels of CD70 surface expression.

Engineered CAR T cells generated as described in Example 5 from healthy donors were co-cultured with K562-CD70-TetOn cells. K562-CD70-TetOn cells are a K562 human leukemia cell line derived from a patient with chronic myelogenous leukemia (CML) engineered to express surface CD70 in a dose-dependent manner in response to doxycycline hydrochloride. K562-CD70-TetOn or parental K562 target cells (2×10⁴ cells/well) were suspended in RPMI cell culture media and incubated with engineered CART cells or mock processed T cells at a 2:1 effector to target (E:T) ratio. Doxycycline hydrochloride was serially diluted 2-fold in dimethyl sulfoxide (DMSO) and final concentrations ranging from 0 ng/mL to 512 ng/mL were added to the plated CAR-T/Target cell mixtures in technical triplicates and plates were incubated at 37° C./5% CO₂ for 24 hours. Target cells were also incubated with equal volumes of media at the same conditions. Cytokine levels were measured using an electrochemiluminescence cytokine immunoassay (Meso Scale Discovery, Rockville, MD).

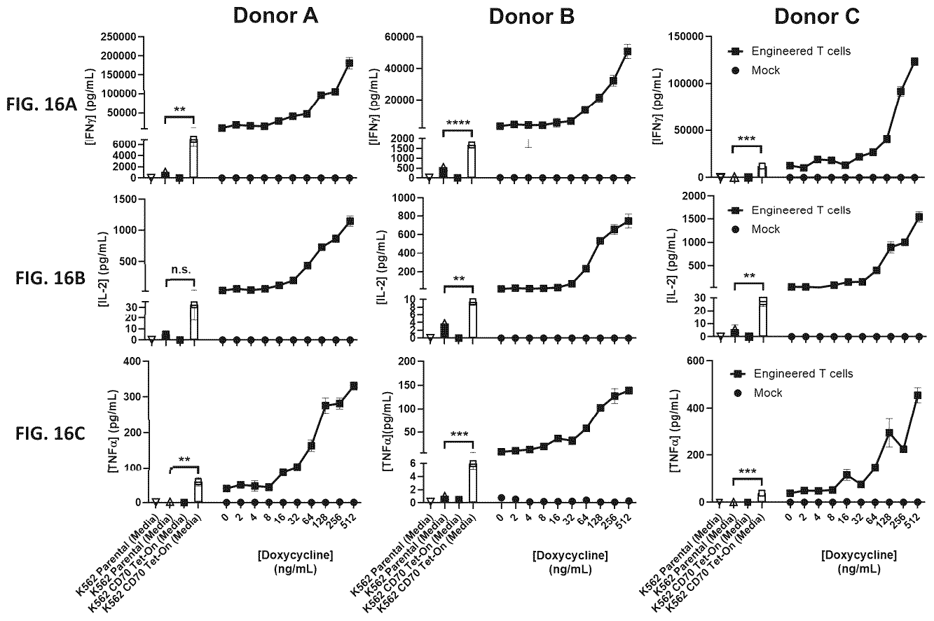

The results of electrochemiluminescence cytokine assays for IFNγ (FIG. 16A), IL-2 (FIG. 16B), and TNFα (FIG. 16C) are shown for mock processed T cells and the engineered allogeneic T cells from three different donors (Donor A, left; Donor B; middle; Donor C; right) following the 24 hour incubation. Data were plotted using GraphPad Prism software, vi1 (GraphPad Software Inc., La Jolla, CA). Statistical analysis was performed using 2-way ANOVA test.

The results demonstrate that the engineered allogeneic T cells generate inflammatory cytokine in response to CD70 expression levels in a dose-dependent manner. Further, the engineered allogeneic T cells respond to low levels of CD70 expression present on K562-CD70-TetOn cells in the absence of doxycycline treatment. In particular, proinflammatory cytokine production was observed in response to as low as 1% CD70-positive tumor cells.

### Example 11—Administration of Anti-CD70 CAR as a Monotherapy for Treatment of Clear Cell Renal Cell Carcinoma

Therapeutic compositions of allogeneic T cells containing an exemplary anti-CD70 CAR; knockout of TRAC, B2M, and CD70; and knock-in of a shRNA cassette as described and generated in Example 5 are administered to subjects with clear cell renal cell carcinoma (ccRCC). As part of the study, subjects receive different dosing of therapeutic composition of allogeneic T cells.

Subjects with ccRCC are treated. Eligibility criteria include being 18 or greater years of age; histologically confirmed diagnosis of a locally advanced and unresectable or metastatic RCC with clear cell component; ECOG performance status of 0 or 1; measurable disease using Response Evaluation Criteria in Solid Tumors version 1.1 (RECIST v1.1); and have previously received, been ineligible for, or declined all standard of care treatments. Exclusion criteria include a prior history of malignancies or lymphoproliferative disease, unless the subject has been free of the disease for two or greater years; and have received prior CD70-directed therapy.

Prior to administration of therapeutic compositions of allogeneic T cells, subjects receive lymphodepleting chemotherapy composed of intravenously delivered fludarabine 30 mg/m² and cyclophosphamide 500 mg/m² per day on Days −5, −4, and −3, where Day 1 is defined as the day of the infusion of compositions of allogeneic T cells. Reduced doses are available for subjects with decreased renal function.

On Day 1, subjects are pre-medicated 30 to 60 minutes prior to receiving an infusion of the composition of allogeneic T cells with 500 to 650 mg oral acetaminophen and 25 to 50 mg H1 antihistamine, such as diphenhydramine hydrochloride. Afterwards, subjects receive an intravenous infusion of the composition of allogeneic T cells with one of the following doses:

$$Dose level - 1 ( DL - 1 ) : 50 \times 1 {0}^{6} CAR + T cells \qquad ( 1 )$$

$$Dose level 1 ( DL 1 ) : 100 \times 1 {0}^{6} CAR + T cells \qquad ( 2 )$$

$$Dose level 2 ( DL 2 ) : 300 \times 1 {0}^{6} CAR + T cells \qquad ( 3 )$$

$$Dose level 3 ( DL 3 ) : 900 \times 1 {0}^{6} CAR + T cells \qquad ( 4 )$$

Dose level-1 (DL-1) is a de-escalating dose of allogeneic T cells that is pursued if dose-limiting toxicity (DLT) is observed at a higher dose level of allogeneic T cells, such as dose level 1 (DL1).

After infusion of the composition of allogeneic T cells on Day 1, subjects are hospitalized or monitored for at least seven days, with some patients hospitalized or monitored for up to 14 days. After release from the hospital, subjects return to the clinic twice a week for physical examination and laboratory tests until Day 29. Additional tests and screening for safety, disease status, research samples, and survival during post-treatment follow-up continue until 24 months after the infusion of the composition of allogeneic T cells on Day 1.

Primary and secondary endpoints related to safety assessment and tolerability of compositions of the allogeneic T cells are assessed, including incidence of adverse events (AEs), serious adverse events (SAEs), AEs leading to dose limiting toxicities (DLTs), AEs leading to discontinuation, and deaths. Additional secondary endpoints related to efficacy and pharmacokinetics (PK) are also assessed. PK parameters of the therapeutic compositions of allogeneic T cells include maximum drug concentration (Cmax), time to peak drug concentration (Tmax), area under the concentration-time curve (AUC), and persistence of the allogeneic T cells in peripheral blood from pre-dose to post-treatment follow-up. Measurements of efficacy include overall response rate (ORR), duration of response (DOR), progression-free survival (PFS), and overall survival (OS). OSS is defined as the proportion of subjects who achieve a best overall response (BOR) of complete response (CR) or partial response (PR) as assessed using RECIST v1.1. DOR is defined as the time between the date of the first documented response (CR or PR) to the date of first documented disease progression as assessed using RECIST v1.1, or death due to any cause, whichever occurs first.

### Example 12—Administration of Anti-CD70 CAR and Nivolumab for Treatment of Clear Cell Renal Cell Carcinoma

A combination therapy composed of therapeutic compositions of allogeneic T cells containing an exemplary anti-CD70 CAR; knockout of TRAC, B2M, and CD70; and knock-in of a shRNA cassette as described and generated in Example 5 and immuno-oncology therapy nivolumab are administered to subjects with clear cell renal cell carcinoma (ccRCC). As part of the study, subjects may receive different dosing of therapeutic composition of allogeneic T cells.

Subjects with ccRCC are treated. Eligibility criteria include being 18 or greater years of age; histologically confirmed diagnosis of a locally advanced and unresectable or metastatic RCC with clear cell component; ECOG performance status of 0 or 1; measurable disease using Response Evaluation Criteria in Solid Tumors version 1.1 (RECIST v1.1); and have previously received, been ineligible for, or declined all standard of care treatments. Exclusion criteria include a prior history of malignancies or lymphoproliferative disease, unless the subject has been free of the disease for two or greater years; have received prior CD70-directed therapy; and being relapsed or is refractory within 12 months of the most recent IO therapy.

Prior to administration of therapeutic compositions of allogeneic T cells, subjects receive lymphodepleting chemotherapy composed of intravenously delivered fludarabine 30 mg/m² and cyclophosphamide 500 mg/m² per day on Days −5, −4, and −3, where Day 1 is defined as the day of the infusion of compositions of allogeneic T cells. Reduced doses are available for subjects with decreased renal function.

On Day 1, subjects are pre-medicated 30 to 60 minutes prior to receiving an infusion of the composition of allogeneic T cells with 500 to 650 mg oral acetaminophen and 25 to 50 mg H1 antihistamine, such as diphenhydramine hydrochloride. Afterwards, subjects receive an intravenous infusion of the composition of allogeneic T cells at a dose of 100×10⁶ CAR+ T cells (DL1) or, if a dose-limiting toxicity is observed, at a dose of 50×10⁶ CAR+ T cells (DL-1).

Subjects are also treated with nivolumab. Specifically, starting on Day 21, subjects are administered nivolumab either intravenously or subcutaneously every four weeks. Alternatively, administration of nivolumab can start on Day 8. For subjects receiving intravenous nivolumab, 480 mg nivolumab is administered intravenously every four weeks (e.g., Q4W). Each infusion of nivolumab at a dose of 480 mg occurs over approximately 30 minutes until progression, unacceptable toxicity, withdrawal of consent, or completion of the 24 months of treatment following the infusion of the composition of allogeneic T cells. For subjects receiving subcutaneous nivolumab, 1200 mg nivolumab is administered subcutaneously every four weeks (e.g., Q4W). Each infusion of nivolumab at a dose of 1200 mg subcutaneously occurs over approximately 3-5 minutes and is co-formulated with hyaluronidase-nvhy (OPDIVO Qvantig™).

After infusion of the composition of allogeneic T cells on Day 1, subjects are hospitalized or monitored for at least seven days, with some patients hospitalized or monitored for up to 14 days. After release from the hospital, subjects return to the clinic twice a week for physical examination and laboratory tests until Day 29. Additional tests and screening for safety, disease status, research samples, and survival during post-treatment follow-up continue until 24 months after the infusion of the composition of allogeneic T cells on Day 1.

Primary and secondary endpoints related to safety assessment and tolerability of compositions of the allogeneic T cells are assessed, including incidence of adverse events (AEs), serious adverse events (SAEs), AEs leading to dose limiting toxicities (DLTs), AEs leading to discontinuation, and deaths. Additional secondary endpoints related to efficacy and pharmacokinetics (PK) are also assessed. PK parameters of the therapeutic compositions of allogeneic T cells include maximum drug concentration (Cmax), time to peak drug concentration (Tmax), area under the concentration-time curve (AUC), and persistence of the allogeneic T cells in peripheral blood from pre-dose to post-treatment follow-up. Measurements of efficacy include overall response rate (ORR), duration of response (DOR), progression-free survival (PFS), and overall survival (OS). OSS is defined as the proportion of subjects who achieve a best overall response (BOR of complete response (CR) or partial response (PR) as assessed using RECIST v1.1. DOR is defined as the time between the date of the first documented response (CR or PR) to the date of first documented disease progression as assessed using RECIST v1.1, or death due to any cause, whichever occurs first.

### Example 13—Administration of Anti-CD70 CAR and Cabozantinib for Treatment of Clear Cell Renal Cell Carcinoma

A combination therapy composed of therapeutic compositions of allogeneic T cells containing an exemplary anti-CD70 CAR; knockout of TRAC, B2M, and CD70; and knock-in of a shRNA cassette as described and generated in Example 5 and the tyrosine kinase inhibitor cabozantinib are administered to subjects with clear cell renal cell carcinoma (ccRCC). As part of the study, subjects may receive different dosing of therapeutic composition of allogeneic T cells.

Subjects with ccRCC are treated. Eligibility criteria include being 18 or greater years of age; histologically confirmed diagnosis of a locally advanced and unresectable or metastatic RCC with clear cell component; ECOG performance status of 0 or 1; measurable disease using Response Evaluation Criteria in Solid Tumors version 1.1 (RECIST v1.1); and have previously received, been ineligible for, or declined all standard of care treatments. Exclusion criteria include a prior history of malignancies or lymphoproliferative disease, unless the subject has been free of the disease for two or greater years; have received prior CD70-directed therapy; and inhibitors of P-gp efflux transporter within 14 days or 5 half-lives (whichever is longer) prior to start of treatment using the tyrosine kinase inhibitor.

Prior to administration of therapeutic compositions of allogeneic T cells, subjects receive lymphodepleting chemotherapy composed of intravenously delivered fludarabine 30 mg/m² and cyclophosphamide 500 mg/m² per day on Days −5, −4, and −3, where Day 1 is defined as the day of the infusion of compositions of allogeneic T cells. Reduced doses are available for subjects with decreased renal function.

Subjects receive an intravenous infusion of the composition of allogeneic T cells at a dose of 100×10⁶ CAR+ T cells (DL1) or, if a dose-limiting toxicity is observed, at a dose of 50×10⁶ CAR+ T cells (DL-1).

Subjects are also concurrently treated with cabozantinib. Specifically, starting on Day 60, subjects take 60 mg of cabozantinib orally once per day (e.g., QD) until 24 months after the administration of the composition of allogeneic T cells on Day 1.

After infusion of the composition of allogeneic T cells on Day 1, subjects are hospitalized or monitored for at least seven days, with some patients hospitalized or monitored for up to14 days. After release from the hospital, subjects return to the clinic twice a week for physical examination and laboratory tests until Day 29. Additional tests and screening for safety, disease status, research samples, and survival during post-treatment follow-up continue until 24 months after the infusion of the composition of allogeneic T cells on Day 1.

Primary and secondary endpoints related to safety assessment and tolerability of compositions of the allogeneic T cells are assessed, including incidence of adverse events (AEs), serious adverse events (SAEs), AEs leading to dose limiting toxicities (DLTs), AEs leading to discontinuation, and deaths. Additional secondary endpoints related to efficacy and pharmacokinetics (PK) are also assessed. PK parameters of the therapeutic compositions of allogeneic T cells include maximum drug concentration (Cmax), time to peak drug concentration (Tmax), area under the concentration-time curve (AUC), and persistence of the allogeneic T cells in peripheral blood from pre-dose to post-treatment follow-up. Measurements of efficacy include overall response rate (ORR), duration of response (DOR), progression-free survival (PFS), and overall survival (OS). OSS is defined as the proportion of subjects who achieve a best overall response (BOR of complete response (CR) or partial response (PR) as assessed using RECIST v1.1. DOR is defined as the time between the date of the first documented response (CR or PR) to the date of first documented disease progression as assessed using RECIST v1.1, or death due to any cause, whichever occurs first.

### Example 14—Administration of Anti-CD70 CAR and Tivozanib for Treatment of Clear Cell Renal Cell Carcinoma

A combination therapy composed of therapeutic compositions of allogeneic T cells containing an exemplary anti-CD70 CAR; knockout of TRAC, B2M, and CD70; and knock-in of a shRNA cassette as described and generated in Example 5 and the tyrosine kinase inhibitor tivozanib are administered to subjects with clear cell renal cell carcinoma (ccRCC). As part of the study, subjects may receive different dosing of therapeutic composition of allogeneic T cells.

Subjects with ccRCC are treated. Eligibility criteria include being 18 or greater years of age; histologically confirmed diagnosis of a locally advanced and unresectable or metastatic RCC with clear cell component; ECOG performance status of 0 or 1; measurable disease using Response Evaluation Criteria in Solid Tumors version 1.1 (RECIST v1.1); and have previously received, been ineligible for, or declined all standard of care treatments. Exclusion criteria include a prior history of malignancies or lymphoproliferative disease, unless the subject has been free of the disease for two or greater years; have received prior CD70-directed therapy; and inhibitors of P-gp efflux transporter within 14 days or 5 half-lives (whichever is longer) prior to start of treatment using the tyrosine kinase inhibitor.

Prior to administration of therapeutic compositions of allogeneic T cells, subjects receive lymphodepleting chemotherapy composed of intravenously delivered fludarabine 30 mg/m² and cyclophosphamide 500 mg/m² per day on Days −5, −4, and −3, where Day 1 is defined as the day of the infusion of compositions of allogeneic T cells. Reduced doses are available for subjects with decreased renal function.

Subjects receive an intravenous infusion of the composition of allogeneic T cells at a dose of 100×10⁶ CAR+ T cells (DL1) or, if a dose-limiting toxicity is observed, at a dose of 50×10⁶ CAR+ T cells (DL-1).

Subjects are also treated with tivozanib. Specifically, starting on Day 60, subjects take about 1.34 mg of tivozanib orally once per day (e.g., QD) for three weeks followed by one week off tivozanib, i.e., one week of rest. Accordingly, one cycle of treatment with tibozanib lasts four weeks total, with 3 weeks on treatment and 1 week off treatment. Cycles are repeated every 4 weeks until 24 months after the infusion of the composition of allogeneic T cells on Day 1.

After infusion of the composition of allogeneic T cells on Day 1, subjects are hospitalized or monitored for at least seven days, with some patients hospitalized or monitored for up to 14 days. After release from the hospital, subjects return to the clinic twice a week for physical examination and laboratory tests until Day 29. Additional tests and screening for safety, disease status, research samples, and survival during post-treatment follow-up continue until 24 months after the infusion of the composition of allogeneic T cells on Day 1.

Primary and secondary endpoints related to safety assessment and tolerability of compositions of the allogeneic T cells are assessed, including incidence of adverse events (AEs), serious adverse events (SAEs), AEs leading to dose limiting toxicities (DLTs), AEs leading to discontinuation, and deaths. Additional secondary endpoints related to efficacy and pharmacokinetics (PK) are also assessed. PK parameters of the therapeutic compositions of allogeneic T cells include maximum drug concentration (Cmax), time to peak drug concentration (Tmax), area under the concentration-time curve (AUC), and persistence of the allogeneic T cells in peripheral blood from pre-dose to post-treatment follow-up. Measurements of efficacy include overall response rate (ORR), duration of response (DOR), progression-free survival (PFS), and overall survival (OS). OSS is defined as the proportion of subjects who achieve a best overall response (BOR of complete response (CR) or partial response (PR) as assessed using RECIST v1.1. DOR is defined as the time between the date of the first documented response (CR or PR) to the date of first documented disease progression as assessed using RECIST v1.1, or death due to any cause, whichever occurs first.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCE TABLE

| SEQ | | |
|---|---|---|
| ID NO: | Sequence | Description |
| 1 | XVQLVESGGGLVQPGGSLRLSCAASGVTLEYSAIGWFRQAPGKEQEGVS | CD70 VHH |
| | CTSSSDGSIYYADSVKGRFTISRDNSKNTLYLQMNSLRAEDTAVYYCAI | (Binder 16) |
| | GVFHGGSCRGALDTWGQGTLVTVSS, wherein X is Q or | |
| | pyroglutamate | |
| 2 | QVQLVESGGGLVQPGGSLRLSCAASGVTLEYSAIGWFRQAPGKEQEGVS | CD70 VHH |
| | CTSSSDGSIYYADSVKGRFTISRDNSKNTLYLQMNSLRAEDTAVYYCAI | (Binder 16) |
| | GVFHGGSCRGALDTWGQGTLVTVSS | |
| 3 | XVQLVESGGGLVQPGGSLRLSCAASGVTLEYSAIGWFRQAPGKEQEGVS | CD70 VHH |
| | CTSSSDGSIYYADSVKGRFTISRDNSKNTLYLQMNSLRAEDTAVYYCAI | (Binder 16) |
| | GVFHGGSCRGALDTWGQGTLVTVSS, wherein X is | |
| | pyroglutamate | |
| 4 | YSAIG | CD70 CDR1 |
| | | Kabat (Binder |
| | | 16) |
| 5 | CTSSSDGSIYYADSVKG | CD70 CDR2 |
| | | Kabat (Binder |
| | | 16) |
| 6 | GVFHGGSCRGALDT | CD70 CDR3 |
| | | Kabat (Binder |
| | | 16) |
| 7 | GVTLEYSA | CD70 CDR1 |
| | | IMGT(Binder |
| | | 16) |
| 8 | TSSSDGSI | CD70 CDR2 |
| | | IMGT(Binder |
| | | 16) |
| 9 | AIGVFHGGSCRGALDT | CD70 CDR3 |
| | | IMGT(Binder |
| | | 16) |
| 10 | IEVMYPPPYLDNEKSNGTIIHVKGKHLCPSPLFPGPSKP | Human CD28 |
| | | Hinge |
| 11 | FWVLVVVGGVLACYSLLVTVAFIIFWV | Human CD28 |
| | | Transmembra |
| | | ne domain |
| 12 | KRGRKKLLYIFKQPFMRPVQTTQEEDGCSCRFPEEEEGGCEL | 4-1BB- |
| | | derived |
| | | intracellular |
| | | costimulatory |
| | | domain (aa) |
| 13 | RVKFSRSADAPAYQQGQNQLYNELNLGRREEYDVLDKRRGRDPEMGGKP | CD3-zeta |
| | RRKNPQEGLYNELQKDKMAEAYSEIGMKGERRRGKGHDGLYQGLSTATK | derived |
| | DTYDALHMQALPPR | intracellular |
| | | signaling |
| | | domain (aa) |
| 14 | MPLLLLLPLLWAGALAXVQLVESGGGLVQPGGSLRLSCAASGVTLEYSA | CD70 CAR |
| | IGWFRQAPGKEQEGVSCTSSSDGSIYYADSVKGRFTISRDNSKNTLYLQ | (Binder 16) |
| | MNSLRAEDTAVYYCAIGVFHGGSCRGALDTWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | (amino acid |
| | ELRVKFSRSADAPAYQQGQNQLYNELNLGRREEYDVLDKRRGRDPEMGG | sequence) |
| | KPRRKNPQEGLYNELQKDKMAEAYSEIGMKGERRRGKGHDGLYQGLSTA | |
| | TKDTYDALHMQALPPR, where X is Q or pyroglutamate | |
| 15 | MPLLLLLPLLWAGALAQVQLVESGGGLVQPGGSLRLSCAASGVTLEYSA | CD70 CAR |
| | IGWFRQAPGKEQEGVSCTSSSDGSIYYADSVKGRFTISRDNSKNTLYLQ | (Binder 16) |
| | MNSLRAEDTAVYYCAIGVFHGGSCRGALDTWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | (amino acid |
| | ELRVKFSRSADAPAYQQGQNQLYNELNLGRREEYDVLDKRRGRDPEMGG | sequence) |
| | KPRRKNPQEGLYNELQKDKMAEAYSEIGMKGERRRGKGHDGLYQGLSTA | |
| | TKDTYDALHMQALPPR | |
| 16 | MPLLLLLPLLWAGALAXVQLVESGGGLVQPGGSLRLSCAASGVTLEYSA | CD70 CAR |
| | IGWFRQAPGKEQEGVSCTSSSDGSIYYADSVKGRFTISRDNSKNTLYLQ | (Binder 16) |
| | MNSLRAEDTAVYYCAIGVFHGGSCRGALDTWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | (amino acid |
| | ELRVKFSRSADAPAYQQGQNQLYNELNLGRREEYDVLDKRRGRDPEMGG | sequence) |
| | KPRRKNPQEGLYNELQKDKMAEAYSEIGMKGERRRGKGHDGLYQGLSTA | |
| | TKDTYDALHMQALPPR, where X is pyroglutamate | |
| 17 | ATGCCCCTGCTGCTCCTGCTCCCTTTGCTGTGGGCGGGAGCCCTCGCGC | CD70 CAR |
| | AAGTGCAACTGGTAGAAAGCGGCGGCGGCCTGGTGCAGCCTGGCGGCTC | (Binder 16) |
| | TCTAAGACTGAGCTGCGCCGCAAGCGGCGTGACCCTGGAGTACAGCGCC | (nucleic acid |
| | ATCGGCTGGTTCAGACAAGCCCCCGGCAAGGAGCAAGAGGGCGTGAGCT | sequence) |
| | GCACAAGCAGCAGCGACGGCAGCATCTACTACGCCGACAGCGTGAAGGG | |
| | CAGATTCACCATCAGCAGAGACAACAGCAAGAACACCCTGTACCTGCAG | |
| | ATGAACAGCCTGAGAGCCGAGGACACCGCCGTGTACTACTGCGCCATCG | |
| | GCGTTTTCCATGGCGGTTCTTGCAGAGGCGCCCTTGATACCTGGGGCCA | |
| | AGGCACCCTGGTGACCGTGAGCAGCATTGAAGTTATGTATCCTCCTCCT | |
| | TACCTAGACAATGAGAAGAGCAATGGAACCATTATCCATGTGAAAGGGA | |
| | AACACCTTTGTCCAAGTCCCCTATTTCCCGGACCTTCTAAGCCCTTCTG | |
| | GGTGCTCGTGGTCGTTGGCGGAGTGCTGGCCTGTTACTCCCTGCTGGTT | |
| | ACCGTGGCCTTCATCATCTTTTGGGTCAAGCGGGGCAGAAAGAAGCTGC | |
| | TCTACATCTTCAAGCAGCCCTTCATGCGGCCCGTGCAGACCACACAAGA | |
| | GGAAGATGGCTGCTCCTGCCGATTCCCCGAGGAAGAAGAAGGCGGCTGC | |
| | GAGCTGAGAGTGAAGTTCAGCAGATCCGCCGACGCTCCAGCCTATCAGC | |
| | AGGGCCAAAACCAGCTGTACAACGAGCTGAACCTGGGGAGAAGAGAAGA | |
| | GTACGACGTGCTGGATAAGCGGAGAGGCAGAGATCCTGAAATGGGCGGC | |
| | AAGCCCAGACGGAAGAATCCTCAAGAGGGCCTGTATAATGAGCTGCAGA | |
| | AAGACAAGATGGCCGAGGCCTACAGCGAGATCGGAATGAAGGGCGAGCG | |
| | CAGAAGAGGCAAGGGACACGATGGACTGTACCAGGGCCTGAGCACCGCC | |
| | ACCAAGGATACCTATGACGCACTGCACATGCAGGCCCTGCCACCTAGA | |
| 18 | MPLLLLLPLLWAGALA | CD33 signal |
| | | peptide |
| 19 | GGATCTGCGATCGCTCCGGTGCCCGTCAGTGGGCAGAGCGCACATCGCC | EF1alpha |
| | CACAGTCCCCGAGAAGTTGGGGGGAGGGGTCGGCAATTGAACCGGTGCC | promoter |
| | TAGAGAAGGTGGCGCGGGGTAAACTGGGAAAGTGATGTCGTGTACTGGC | |
| | TCCGCCTTTTTCCCGAGGGTGGGGGAGAACCGTATATAAGTGCAGTAGT | |
| | CGCCGTGAACGTT | |
| 20 | ggatctgcgatcgctccggtgcccgtcagtgggcagagcgcacatcgcc | EF1a- |
| | cacagtccccgagaagttggggggaggggtcggcaattgaaccggtgcc | HTLV1R |
| | tagagaaggtggcgcggggtaaactgggaaagtgatgtcgtgtactggc | promoter |
| | tccgcctttttcccgagggtgggggagaaccgtatataagtgcagtagt | |
| | cgccgtgaacgttctttttcgcaacgggtttgccgccagaacacagctg | |
| | aagcttcgaggggctcgcatctctccttcacgcgcccgccgccctacct | |
| | gaggccgccatccacgccggttgagtcgcgttctgccgcctcccgcctg | |
| | tggtgcctcctgaactgcgtccgccgtctaggtaagtttaaagctcagg | |
| | tcgagaccgggcctttgtccggcgctcccttggagcctacctagactca | |
| | gccggctctccacgctttgcctgaccctgcttgctcaactctacgtctt | |
| | tgtttcgttttctgttctgcgccgttacagatccaagctgtgaccggcg | |
| | cctac | |
| 21 | VKQTLNFDLLKLAGDVESNPGP | F2A peptide |
| 22 | QCTNYALLKLAGDVESNPGP | E2A peptide |
| 23 | LEGGGEGRGSLLTCGDVEENPGPR | T2A peptide |
| 24 | EGRGSLLTCGDVEENPGP | T2A peptide |
| 25 | GSGEGRGSLLTCGDVEENPGP | T2A peptide |
| 26 | GSGATNFSLLKQAGDVEENPGP | P2A peptide |
| 27 | XVQLVESGGGLVQPGGSLRLSCAASGFTLDYHNIGWFRQAPGKELEGVL | VHH |
| | CISSSDGSTYYADSVKGRFTISRDNSKNTLYLQMNSLRAEDTAVYYCAT | (Binder 18) |
| | GLFYPGSCRGALDAWGQGTLVTVSS, wherein X is Q or | |
| | pyroglutamate | |
| 28 | QVQLVESGGGLVQPGGSLRLSCAASGFTLDYHNIGWFRQAPGKELEGVL | VHH |
| | CISSSDGSTYYADSVKGRFTISRDNSKNTLYLQMNSLRAEDTAVYYCAT | (Binder 18) |
| | GLFYPGSCRGALDAWGQGTLVTVSS | |
| 29 | XVQLVESGGGLVQPGGSLRLSCAASGFTLDYHNIGWFRQAPGKELEGVL | VHH |
| | CISSSDGSTYYADSVKGRFTISRDNSKNTLYLQMNSLRAEDTAVYYCAT | (Binder 18) |
| | GLFYPGSCRGALDAWGQGTLVTVSS, wherein X is | |
| | pyroglutamate | |
| 30 | GGCTTAGACGCAGGTGTTCTGATTTATAGTTCAAAACCTCTATCAATGA | TRAC 5′ |
| | GAGAGCAATCTCCTGGTAATGTGATAGATTTCCCAACTTAATGCCAACA | homology |
| | TACCATAAACCTCCCATTCTGCTAATGCCCAGCCTAAGTTGGGGAGACC | arm (nt) |
| | ACTCCAGATTCCAAGATGTACAGTTTGCTTTGCTGGGCCTTTTTCCCAT | |
| | GCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCA | |
| | GGTTTCCTTGAGTGGCAGGCCAGGCCTGGCCGTGAACGTTCACTGAAAT | |
| | CATGGCCTCTTGGCCAAGATTGATAGCTTGTGCCTGTCCCTGAGTCCCA | |
| | GTCCATCACGAGCAGCTGGTTTCTAAGATGCTATTTCCCGTATAAAGCA | |
| | TGAGACCGTGACTTGCCAGCCCCACAGAGCCCCGCCCTTGTCCATCACT | |
| | GGCATCTGGACTCCAGCCTGGGTTGGGGCAAAGAGGGAAATGAGATCAT | |
| | GTCCTAACCCTGATCCTCTTGTCCCACAGATATCCAGAACCCTGACCCT | |
| | GCCGTGTACCAG | |
| 31 | CTCTAAATCCAGTGACAAGTCTGTCTGCCTATTCACCGATTTTGATTCT | TRAC 3′ |
| | CAAACAAATGTGTCACAAAGTAAGGATTCTGATGTGTATATCACAGACA | homology |
| | AAACTGTGCTAGACATGAGGTCTATGGACTTCAAGAGCAACAGTGCTGT | arm (nt) |
| | GGCCTGGAGCAACAAATCTGACTTTGCATGTGCAAACGCCTTCAACAAC | |
| | AGCATTATTCCAGAAGACACCTTCTTCCCCAGCCCAGGTAAGGGCAGCT | |
| | TTGGTGCCTTCGCAGGCTGTTTCCTTGCTTCAGGAATGGCCAGGTTCTG | |
| | CCCAGAGCTCTGGTCAATGATGTCTAAAACTCCTCTGATTGGTGGTCTC | |
| | GGCCTTATCCATTGCCACCAAAACCCTCTTTTTACTAAGAAACAGTGAG | |
| | CCTTGTTCTGGCAGTCCAGAGAATGACACGGGAAAAAAGCAGATGAAGA | |
| | GAAGGTGGCAGGAGAGGGCACGTGGCCCAGCCTCAGTCTCTCCAACTGA | |
| | GTTCCTGCCTGCCTGCCTTTGCTCAGACTGTTTGCCCCTTACTGCTCTT | |
| | CTAGGCCTCATTCTAAGCCCCTTCTCCAAGTTGCCTCTCCTTATTTCTC | |
| | CCTGTCTGCCAA | |
| 32 | agcaattgctatgtcccaggcactctactagacacttcatacagtttag | B2M 5′ |
| | aaaatcagatgggtgtagatcaaggcaggagcaggaaccaaaaagaaag | homology |
| | gcataaacataagaaaaaaaatggaaggggtggaaacagagtacaataa | arm (nt) |
| | catgagtaatttgatgggggctattatgaactgagaaatgaactttgaa | |
| | aagtatcttggggccaaatcatgtagactcttgagtgatgtgttaagga | |
| | atgctatgagtgctgagagggcatcagaagtccttgagagcctccagag | |
| | aaaggctcttaaaaatgcagcgcaatctccagtgacagaagatactgct | |
| | agaaatctgctagaaaaaaaacaaaaaaggcatgtatagaggaattatg | |
| | agggaaagataccaagtcacggtttattcttcaaaatggaggtggcttg | |
| | ttgggaaggtggaagctcatttggccagagtggaaatggaattgggaga | |
| | aatcgatgaccaaatgtaaacacttggtgcctgatatagcttgacacca | |
| | agttagccccaagtgaaataccctggcaatattaatgtgtcttttcccg | |
| | atattcctcagGTACTCCAAAGATTCAGGTTTACTCACGTCATCCAGCA | |
| | GAGAATGGAAAGTCAAATTTCCTGAATTGCTATGTGTCTGGGTTTCATC | |
| | CATCCGACATTGAAGTTGACTTACTGAAGAATGGAGAGAGAATTGAAAA | |
| | AGTGGAGCATTCAGACTTGTCTTTCAGCAAGGACTGGTCTTTCTATCTC | |
| | TTGTACTACACTGAA | |
| 33 | ACTGAAAAAGATGAGTATGCCTGCCGTGTGAACCATGTGACTTTGTCAC | B2M 3′ |
| | AGCCCAAGATAGTTAAGTGGGgtaagtcttacattcttttgtaagctgc | homology |
| | tgaaagttgtgtatgagtagtcatatcataaagctgctttgatataaaa | arm (nt) |
| | aaggtctatggccatactaccctgaatgagtcccatcccatctgatata | |
| | aacaatctgcatattgggattgtcagggaatgttcttaaagatcagatt | |
| | agtggcacctgctgagatactgatgcacagcatggtttctgaaccagta | |
| | gtttccctgcagttgagcagggagcagcagcagcacttgcacaaataca | |
| | tatacactcttaacacttcttacctactggcttcctctagcttttgtgg | |
| | cagcttcaggtatatttagcactgaacgaacatctcaagaaggtatagg | |
| | cctttgtttgtaagtcctgctgtcctagcatcctataatcctggacttc | |
| | tccagtactttctggctggattggtatctgaggctagtaggaagggctt | |
| | gttcctgctgggtagctctaaacaatgtattcatgggtaggaacagcag | |
| | cctattctgccagccttatttctaaccattttagacatttgttagtaca | |
| | tggtattttaaaagtaaaacttaatgtcttccttttttttctccactgt | |
| | ctttttcatagATCGAGACATGTAAGCAGCATCATGGAGgtaagttttt | |
| | gaccttgagaaaatgtttttgtttcactgtcctgaggactatttataga | |
| | cagctctaacatgata | |
| 34 | MSRSVALAVLALLSLSGLEAVMAPRTLVLGGGGSGGGGSGGGGSIQRTP | HLA-E single |
| | KIQVYSRHPAENGKSNFLNCYVSGFHPSDIEVDLLKNGERIEKVEHSDL | chain fusion |
| | SFSKDWSFYLLYYTEFTPTEKDEYACRVNHVTLSQPKIVKWDRDMGGGG | (amino acid |
| | SGGGGSGGGGSGGGGSGSHSLKYFHTSVSRPGRGEPRFISVGYVDDTQF | sequence) |
| | VRFDNDAASPRMVPRAPWMEQEGSEYWDRETRSARDTAQIFRVNLRTLR | |
| | GYYNQSEAGSHTLQWMHGCELGPDGRFLRGYEQFAYDGKDYLTLNEDLR | |
| | SWTAVDTAAQISEQKSNDASEAEHQRAYLEDTCVEWLHKYLEKGKETLL | |
| | HLEPPKTHVTHHPISDHEATLRCWALGFYPAEITLTWQQDGEGHTQDTE | |
| | LVETRPAGDGTFQKWAAVVVPSGEEQRYTCHVQHEGLPEPVTLRWKPAS | |
| | QPTIPIVGIIAGLVLLGSVVSGAVVAAVIWRKKSSGGKGGSYYKAEWSD | |
| | SAQGSESHSL | |
| 35 | atgtcaagatctgtcgctctggctgtcctggccctgctgtctctgtctg | HLA-E single |
| | gacttgaagcagtgatggcccctagaacactggtgcttggaggcggagg | chain fusion |
| | atctggcggaggtggaagtggcggaggcggatctatccaacggacacct | (DNA) |
| | aagatccaagtgtacagcagacaccccgccgagaacggcaagagcaact | |
| | tcctgaactgctacgtgtccggcttccacccctctgacattgaggtgga | |
| | cctgctgaagaacggcgagcggatcgagaaggtggaacacagcgatctg | |
| | agcttctccaaggattggtccttctacctgctctactacaccgagttca | |
| | ctcctaccgagaaggacgagtacgcatgtagagtgaaccacgtgacact | |
| | gagccaacctaagatcgtgaaatgggacagagatatgggaggcggcggt | |
| | agtggcggcggaggaagcggaggcggaggttcaggtggtggtggatctg | |
| | gaagccacagcctgaagtactttcacacctccgtgtccagacctggcag | |
| | aggcgagcctagattcatcagcgtgggctacgtggacgacacccagttc | |
| | gtcagattcgacaacgacgccgcctctcctcggatggttcctagagcac | |
| | cctggatggaacaagagggcagcgagtactgggatcgcgagacaagaag | |
| | cgccagagacacagcccagatcttccgcgtgaacctgagaaccctgcgg | |
| | ggctactacaatcagtctgaggccggctctcacaccctgcagtggatgc | |
| | atggatgtgaactgggccccgacggacggttcctgagaggctatgagca | |
| | gttcgcctacgacggcaaggactacctgacactgaacgaggacctgaga | |
| | agctggaccgccgtggatacagccgctcagatcagcgagcagaagtcta | |
| | acgacgccagcgaggccgaacaccagagagcctatctggaagatacctg | |
| | cgtggaatggctgcacaagtacctggaaaagggcaaagagacactgctg | |
| | cacctggaaccacctaagacacatgtgacccaccatcctatcagcgacc | |
| | acgaggccacactgagatgttgggccctgggcttttaccctgccgagat | |
| | cacactgacatggcagcaggatggcgagggccacacacaggatacagag | |
| | ctggtggaaacaagacctgccggcgacggcaccttccagaaatgggctg | |
| | ctgtggtggttcccagcggcgaggaacagagatacacctgtcacgtgca | |
| | gcacgagggactgcctgaacctgtgactctgagatggaagcctgccagc | |
| | cagccaacaatccccatcgtgggaatcattgccggcctggtgctgctgg | |
| | gatctgtggtttctggtgcagtggtggccgccgtgatttggagaaagaa | |
| | gtcctctggcggcaaaggcggctcctactataaggccgagtggagcgat | |
| | tctgcccagggctctgaaagccactctctgtg | |
| 36 | GSHSLKYFHTSVSRPGRGEPRFISVGYVDDTQFVRFDNDAASPRMVPRA | mature HLA- |
| | PWMEQEGSEYWDRETRSARDTAQIFRVNLRTLRGYYNQSEAGSHTLQWM | E coding |
| | HGCELGPDGRFLRGYEQFAYDGKDYLTLNEDLRSWTAVDTAAQISEQKS | sequence |
| | NDASEAEHQRAYLEDTCVEWLHKYLEKGKETLLHLEPPKTHVTHHPISD | without signal |
| | HEATLRCWALGFYPAEITLTWQQDGEGHTQDTELVETRPAGDGTFQKWA | peptide |
| | AVVVPSGEEQRYTCHVQHEGLPEPVTLRWKPASQPTIPIVGIIAGLVLL | |
| | GSVVSGAVVAAVIWRKKSSGGKGGSYYKAEWSDSAQGSESHSL | |
| 37 | IQRTPKIQVYSRHPAENGKSNFLNCYVSGFHPSDIEVDLLKNGERIEKV | beta-2- |
| | EHSDLSFSKDWSFYLLYYTEFTPTEKDEYACRVNHVTLSQPKIVKWDRD | microglobulin |
| | M | mature |
| | | peptide |
| | | (NCBI |
| | | Reference |
| | | Sequence: |
| | | NP_004039.1) |
| | | (without |
| | | signal |
| | | peptide) |
| 38 | XVQLVESGGGLVQPGGSLRLSCAASGVTLEYSAIGWFRQAPGKEQEGVS | CD70 CAR |
| | CTSSSDGSIYYADSVKGRFTISRDNSKNTLYLQMNSLRAEDTAVYYCAI | (Binder 16) |
| | GVFHGGSCRGALDTWGQGTLVTVSSIEVMYPPPYLDNEKSNGTIIHVKG | without signal |
| | KHLCPSPLFPGPSKPFWVLVVVGGVLACYSLLVTVAFIIFWVKRGRKKL | peptide |
| | LYIFKQPFMRPVQTTQEEDGCSCRFPEEEEGGCELRVKFSRSADAPAYQ | (amino acid |
| | QGQNQLYNELNLGRREEYDVLDKRRGRDPEMGGKPRRKNPQEGLYNELQ | sequence) |
| | KDKMAEAYSEIGMKGERRRGKGHDGLYQGLSTATKDTYDALHMQALPPR, | |
| | wherein X is Q or pyroglutamate | |
| 39 | QVQLVESGGGLVQPGGSLRLSCAASGVTLEYSAIGWFRQAPGKEQEGVS | CD70 CAR |
| | CTSSSDGSIYYADSVKGRFTISRDNSKNTLYLQMNSLRAEDTAVYYCAI | (Binder 16) |
| | GVFHGGSCRGALDTWGQGTLVTVSSIEVMYPPPYLDNEKSNGTIIHVKG | without signal |
| | KHLCPSPLFPGPSKPFWVLVVVGGVLACYSLLVTVAFIIFWVKRGRKKL | peptide |
| | LYIFKQPFMRPVQTTQEEDGCSCRFPEEEEGGCELRVKFSRSADAPAYQ | (amino acid |
| | QGQNQLYNELNLGRREEYDVLDKRRGRDPEMGGKPRRKNPQEGLYNELQ | sequence) |
| | KDKMAEAYSEIGMKGERRRGKGHDGLYQGLSTATKDTYDALHMQALPPR | |
| 40 | XVQLVESGGGLVQPGGSLRLSCAASGVTLEYSAIGWFRQAPGKEQEGVS | CD70 CAR |
| | CTSSSDGSIYYADSVKGRFTISRDNSKNTLYLQMNSLRAEDTAVYYCAI | without signal |
| | GVFHGGSCRGALDTWGQGTLVTVSSIEVMYPPPYLDNEKSNGTIIHVKG | peptide |
| | KHLCPSPLFPGPSKPFWVLVVVGGVLACYSLLVTVAFIIFWVKRGRKKL | (amino acid |
| | LYIFKQPFMRPVQTTQEEDGCSCRFPEEEEGGCELRVKFSRSADAPAYQ | sequence) |
| | QGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR, | |
| | wherein X is pyroglutamate | |
| 41 | VQLVESGGGLVQPGGSLRLSCAASGFTLDYHNIGWFRQAPGKELEGVLC | CD70 CAR |
| | ISSSDGSTYYADSVKGRFTISRDNSKNTLYLQMNSLRAEDTAVYYCATG | (Binder 18) |
| | LFYPGSCRGALDAWGQGTLVTVSSIEVMYPPPYLDNEKSNGTIIHVKGK | without signal |
| | HLCPSPLFPGPSKPFWVLVVVGGVLACYSLLVTVAFIIFWVKRGRKKLL | peptide |
| | YIFKQPFMRPVQTTQEEDGCSCRFPEEEEGGCELRVKFSRSADAPAYQQ | (amino acid |
| | GQNQLYNELNLGRREEYDVLDKRRGRDPEMGGKPRRKNPQEGLYNELQK | sequence) |
| | DKMAEAYSEIGMKGERRRGKGHDGLYQGLSTATKDTYDALHMQALPPR, | |
| | wherein X is Q or pyroglutamate | |
| 42 | QVQLVESGGGLVQPGGSLRLSCAASGFTLDYHNIGWFRQAPGKELEGVL | CD70 CAR |
| | CISSSDGSTYYADSVKGRFTISRDNSKNTLYLQMNSLRAEDTAVYYCAT | (Binder 18) |
| | GLFYPGSCRGALDAWGQGTLVTVSSIEVMYPPPYLDNEKSNGTIIHVKG | without signal |
| | KHLCPSPLFPGPSKPFWVLVVVGGVLACYSLLVTVAFIIFWVKRGRKKL | peptide |
| | LYIFKQPFMRPVQTTQEEDGCSCRFPEEEEGGCELRVKFSRSADAPAYQ | (amino acid |
| | QGQNQLYNELNLGRREEYDVLDKRRGRDPEMGGKPRRKNPQEGLYNELQ | sequence) |
| | KDKMAEAYSEIGMKGERRRGKGHDGLYQGLSTATKDTYDALHMQALPPR | |
| 43 | XVQLVESGGGLVQPGGSLRLSCAASGFTLDYHNIGWFRQAPGKELEGVL | CD70 CAR |
| | CISSSDGSTYYADSVKGRFTISRDNSKNTLYLQMNSLRAEDTAVYYCAT | (Binder 18) |
| | GLFYPGSCRGALDAWGQGTLVTVSSIEVMYPPPYLDNEKSNGTIIHVKG | without signal |
| | KHLCPSPLFPGPSKPFWVLVVVGGVLACYSLLVTVAFIIFWVKRGRKKL | peptide |
| | LYIFKQPFMRPVQTTQEEDGCSCRFPEEEEGGCELRVKFSRSADAPAYQ | (amino acid |
| | QGQNQLYNELNLGRREEYDVLDKRRGRDPEMGGKPRRKNPQEGLYNELQ | sequence) |
| | KDKMAEAYSEIGMKGERRRGKGHDGLYQGLSTATKDTYDALHMQALPPR, | |
| | wherein X is pyroglutamate | |
| 44 | MPLLLLLPLLWAGALAXVQLVESGGGLVQPGGSLRLSCAASGFTLDYHN | CD70 CAR |
| | IGWFRQAPGKELEGVLCISSSDGSTYYADSVKGRFTISRDNSKNTLYLQ | (Binder 18) |
| | MNSLRAEDTAVYYCATGLFYPGSCRGALDAWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTA | |
| | TKDTYDALHMQALPPR, wherein X is Q or pyroglutamate | |
| 45 | MPLLLLLPLLWAGALAQVQLVESGGGLVQPGGSLRLSCAASGFTLDYHN | CD70 CAR |
| | IGWFRQAPGKELEGVLCISSSDGSTYYADSVKGRFTISRDNSKNTLYLQ | (Binder 18) |
| | MNSLRAEDTAVYYCATGLFYPGSCRGALDAWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTA | |
| | TKDTYDALHMQALPPR | |
| 46 | MPLLLLLPLLWAGALAXVQLVESGGGLVQPGGSLRLSCAASGFTLDYHN | CD70 CAR |
| | IGWFRQAPGKELEGVLCISSSDGSTYYADSVKGRFTISRDNSKNTLYLQ | (Binder 18) |
| | MNSLRAEDTAVYYCATGLFYPGSCRGALDAWGQGTLVTVSSIEVMYPPP | with signal |
| | YLDNEKSNGTIIHVKGKHLCPSPLFPGPSKPFWVLVVVGGVLACYSLLV | peptide |
| | TVAFIIFWVKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTA | |
| | TKDTYDALHMQALPPR, wherein X is pyroglutamate | |
| 47 | MSRSVALAVLALLSLSGLEA | B2m leader |
| 48 | VMAPRTLVL | HLA-E fusion |
| | | protein signal |
| | | peptide |
| 49 | VMAPRTLLL | HLA-E fusion |
| | | protein |
| | | peptide |
| 50 | VMAPRTVL | HLA-E fusion |
| | | protein |
| | | peptide |
| 51 | VMAPRTLEL | HLA-E fusion |
| | | protein |
| | | peptide |
| 52 | VMAPRTLIL | HLA-E fusion |
| | | protein |
| | | peptide |
| 53 | GGGGSGGGGSGGGGS | (G4S)x3 |
| | | linker |
| 54 | GGGGSGGGGSGGGGSGGGGS | (G4S)x4 |
| | | linker |
| 55 | ATGTGTTTTTGTCAAAAGACCTTTTrUrArArUrUrUrCrUrArCrUrC | TRAC gRNA |
| | rUrUrGrUrArGrArUrGrArGrUrCrUrCrUrCrArGrCrUrGrGrUr | |
| | ArCrArCrG | |
| 56 | ATGTGTTTTTGTCAAAAGACCTTTT | DNA |
| | | extension |
| 57 | rUrArArUrUrUrCrUrArCrUrCrUrUrGrUrArGrArU | Scaffold |
| | | (RNA) |
| 58 | rGrArGrUrCrUrCrUrCrArGrCrUrGrGrUrArCrArCrG | TRAC guide |
| | | sequence |
| | | (RNA) |
| 59 | GAGTCTCTCAGCTGGTACACG | TRAC target |
| | | site |
| 60 | aatataagtggaggcgtcgcgctggcgggcattcctgaagctgacagca | Homo sapiens |
| | ttcgggccgagatgtctcgctccgtggccttagctgtgctcgcgctact | beta-2- |
| | ctctctttctggcctggaggctatccagcgtactccaaagattcaggtt | microglobulin |
| | tactcacgtcatccagcagagaatggaaagtcaaatttcctgaattgct | (B2M), |
| | atgtgtctgggtttcatccatccgacattgaagttgacttactgaagaa | nucleic acid |
| | tggagagagaattgaaaaagtggagcattcagacttgtctttcagcaag | NCBI |
| | gactggtctttctatctcttgtactacactgaattcacccccactgaaa | Reference |
| | aagatgagtatgcctgccgtgtgaaccatgtgactttgtcacagcccaa | Sequence: |
| | gatagttaagtgggatcgagacatgtaagcagcatcatggaggtttgaa | NM_004048.2 |
| | gatgccgcatttggattggatgaattccaaattctgcttgcttgctttt | |
| | taatattgatatgcttatacacttacactttatgcacaaaatgtagggt | |
| | tataataatgttaacatggacatgatcttctttataattctactttgag | |
| | tgctgtctccatgtttgatgtatctgagcaggttgctccacaggtagct | |
| | ctaggagggctggcaacttagaggtggggagcagagaattctcttatcc | |
| | aacatcaacatcttggtcagatttgaactcttcaatctcttgcactcaa | |
| | agcttgttaagatagttaagcgtgcataagttaacttccaatttacata | |
| | ctctgcttagaatttgggggaaaatttagaaatataattgacaggatta | |
| | ttggaaatttgttataatgaatgaaacattttgtcatataagattcata | |
| | tttacttcttatacatttgataaagtaaggcatggttgtggttaatctg | |
| | gtttatttttgttccacaagttaaataaatcataaaacttgatgtgtta | |
| | tctctta | |
| 61 | ATGTGTTTTTGTCAAAAGACCTTTTrUrArArUrUrUrCrUrArCrUrC | B2M gRNA |
| | rUrUrGrUrArGrArUrArGrUrGrGrGrGrGrUrGrArArUrUrCrAr | |
| | GrUrGrUrA | |
| 62 | rArGrUrGrGrGrGrGrUrGrArArUrUrCrArGrUrGrUrA | B2M guide |
| | | sequence |
| | | (RNA) |
| 63 | AGTGGGGGTGAATTCAGTGTA | B2M target |
| | | site |
| 64 | ATGTGTTTTTGTCAAAAGACCTTTTrUrArArUrUrUrCrUrArCrUrC | CD70 gRNA |
| | rUrUrGrUrArGrArUrUrUrCrCrArGrUrGrGrGrArCrGrUrArGr | |
| | CrUrGrArG | |
| 65 | rUrUrCrCrArGrUrGrGrGrArCrGrUrArGrCrUrGrArG | CD70 guide |
| | | sequence |
| | | (RNA) |
| 66 | TTCCAGTGGGACGTAGCTGAG | CD70 target |
| | | site |
| 67 | GGATCCGGATCTGCGATCGCTCCGGTGCCCGTCAGTGGGCAGAGCGCAC | CD70 CAR |
| | ATCGCCCACAGTCCCCGAGAAGTTGGGGGGAGGGGTCGGCAATTGAACC | TRAC KI |
| | GGTGCCTAGAGAAGGTGGCGCGGGGTAAACTGGGAAAGTGATGTCGTGT | template |
| | ACTGGCTCCGCCTTTTTCCCGAGGGTGGGGGAGAACCGTATATAAGTGC | (DNA) |
| | AGTAGTCGCCGTGAACGTTCTTTTTCGCAACGGGTTTGCCGCCAGAACA | |
| | CAGCTGAAGCTTCGAGGGGCTCGCATCTCTCCTTCACGCGCCCGCCGCC | |
| | CTACCTGAGGCCGCCATCCACGCCGGTTGAGTCGCGTTCTGCCGCCTCC | |
| | CGCCTGTGGTGCCTCCTGAACTGCGTCCGCCGTCTAGGTAAGTCGACTC | |
| | GTTGGATCCCCACTACCCGGATCAACGCCCTAGGTTTATGTTTGGATGA | |
| | ACTGACATACGCGTATCCGTCTTAAGAATCTTTTCAAACACTAGTAGTG | |
| | AAATATATATTAAACTAGTGTTTGAAAAGATTCTTATTACGGTAACGCG | |
| | GAATTCGCAACTATTTTATCAATTTTTTGCGTCGACACTTCAAGGGGCT | |
| | TGCGGCCGCAACCATCTCCATGGCTGTTTGAATGAGGCTTCAGTACTTT | |
| | ACAGAATCGTTGCCTGCACATCTTGGAAACACTTGCTGGGATTACTTCG | |
| | ACTTCTTAACCCAACAGAAGGCTCGAGAAGGTATATTGCTGTTGACAGT | |
| | GAGCGCCAGTGTGAAGCTCTTGTCAGATAGTGAAGCCACAGATGTATCT | |
| | GACAAGAGCTTCACACTGATGCCTACTGCCTCGGACTTCAAGGGGCTAG | |
| | AATTCGAGCAATTATCTTGTTTACTAAAACTGAATACCTTGCTATCTCT | |
| | TTGATACATTTTTACAAAGCTGAATTAAAATGGTATAAATTAAATCACT | |
| | TTGACGGTGACTTAGGAGTATGCCGGATCAACGCCCTAGGTTTATGTTT | |
| | GGATGAACTGACATACGCGTATCCGTCTAAAAATCAATCTCATTTCCTG | |
| | GTAGTGAAATATATATTAAACCAGGAAATGAGATTGATTTTTTTACGGT | |
| | AACGCGGAATTCGCAACTATTTTATCAATTTTTTGCGTCGACGACTGTG | |
| | ACAGCAGAGTATGCCGGATCAACGCCCTAGGTTTATGTTTGGATGAACT | |
| | GACATACGCGTATCCGTCTTATGTAAAAGACAAACAATGCGTAGTGAAA | |
| | TATATATTAAACGCATTGTTTGTCTTTTACATATTACGGTAACGCGGAA | |
| | TTCGCAACTATTTTATCAATTTTTTGCGTCGACCGGAACTATCTTGAAG | |
| | AGTAGTAGTGGACTAGTGTGACGCTGCTGACCCCTTTCTTTCCCTTCTA | |
| | CAGATCCAAGCTGTGACCGGCGCCTACACCTGCAGCCCAAGCTTACCGC | |
| | TAGCGCCACCATGCCCCTGCTGCTCCTGCTCCCTTTGCTGTGGGCGGGA | |
| | GCCCTCGCGCAAGTGCAACTGGTAGAAAGCGGCGGCGGCCTGGTGCAGC | |
| | CTGGCGGCTCTCTAAGACTGAGCTGCGCCGCAAGCGGCGTGACCCTGGA | |
| | GTACAGCGCCATCGGCTGGTTCAGACAAGCCCCCGGCAAGGAGCAAGAG | |
| | GGCGTGAGCTGCACAAGCAGCAGCGACGGCAGCATCTACTACGCCGACA | |
| | GCGTGAAGGGCAGATTCACCATCAGCAGAGACAACAGCAAGAACACCCT | |
| | GTACCTGCAGATGAACAGCCTGAGAGCCGAGGACACCGCCGTGTACTAC | |
| | TGCGCCATCGGCGTTTTCCATGGCGGTTCTTGCAGAGGCGCCCTTGATA | |
| | CCTGGGGCCAAGGCACCCTGGTGACCGTGAGCAGCATTGAAGTTATGTA | |
| | TCCTCCTCCTTACCTAGACAATGAGAAGAGCAATGGAACCATTATCCAT | |
| | GTGAAAGGGAAACACCTTTGTCCAAGTCCCCTATTTCCCGGACCTTCTA | |
| | AGCCCTTCTGGGTGCTCGTGGTCGTTGGCGGAGTGCTGGCCTGTTACTC | |
| | CCTGCTGGTTACCGTGGCCTTCATCATCTTTTGGGTCAAGCGGGGCAGA | |
| | AAGAAGCTGCTCTACATCTTCAAGCAGCCCTTCATGCGGCCCGTGCAGA | |
| | CCACACAAGAGGAAGATGGCTGCTCCTGCCGATTCCCCGAGGAAGAAGA | |
| | AGGCGGCTGCGAGCTGAGAGTGAAGTTCAGCAGATCCGCCGACGCTCCA | |
| | GCCTATCAGCAGGGCCAAAACCAGCTGTACAACGAGCTGAACCTGGGGA | |
| | GAAGAGAAGAGTACGACGTGCTGGATAAGCGGAGAGGCAGAGATCCTGA | |
| | AATGGGCGGCAAGCCCAGACGGAAGAATCCTCAAGAGGGCCTGTATAAT | |
| | GAGCTGCAGAAAGACAAGATGGCCGAGGCCTACAGCGAGATCGGAATGA | |
| | AGGGCGAGCGCAGAAGAGGCAAGGGACACGATGGACTGTACCAGGGCCT | |
| | GAGCACCGCCACCAAGGATACCTATGACGCACTGCACATGCAGGCCCTG | |
| | CCACCTAGATGAGTCGACTGCTTTATTTGTGAAATTTGTGATGCTATTG | |
| | CTTTATTTGTAACCATTATAAGCTGCAATAAACAAGTTAACAACAACAA | |
| | TTGCATTCATTTTATGTTTCAGGTTCAGGGGGAGGTGTGGGAGGTTTTT | |
| | TAAAGGGCCC | |
| 68 | CTTTTTCGCAACGGGTTTGCCGCCAGAACACAGCTGAAGCTTCGAGGGG | Non-coding |
| | CTCGCATCTCTCCTTCACGCGCCCGCCGCCCTACCTGAGGCCGCCATCC | element (full |
| | ACGCCGGTTGAGTCGCGTTCTGCCGCCTCCCGCCTGTGGTGCCTCCTGA | sequence) |
| | ACTGCGTCCGCCGTCTAGGTAAGTCGACTCGTTGGATCCCCACTACCGG | |
| | AACTATCTTGAAGAGTAGTAGTGGACTAGTGTGACGCTGCTGACCCCTT | |
| | TCTTTCCCTTCTACAGATCCAAGCTGTGACCGGCGCCTACACCTGCAGC | |
| | CCAAGCTTACC | |
| 69 | CTTTTTCGCAACGGGTTTGCCGCCAGAACACAGCTGAAGCTTCGAGGGG | 5′ non-coding |
| | CTCGCATCTCTCCTTCACGCGCCCGCCGCCCTACCTGAGGCCGCCATCC | element (193 |
| | ACGCCGGTTGAGTCGCGTTCTGCCGCCTCCCGCCTGTGGTGCCTCCTGA | bp) |
| | ACTGCGTCCGCCGTCTAGGTAAGTCGACTCGTTGGATCCCCACTAC | |
| 70 | CGGAACTATCTTGAAGAGTAGTAGTGGACTAGTGTGACGCTGCTGACCC | 3′ non-coding |
| | CTTTCTTTCCCTTCTACAGATCCAAGCTGTGACCGGCGCCTACACCTGC | element (112 |
| | AGCCCAAGCTTACC | bp) |
| 71 | CCGGATCAACGCCCTAGGTTTATGTTTGGATGAACTGACATACGCGTAT | shRNA |
| | CCGTCTTAAGAATCTTTTCAAACACTAGTAGTGAAATATATATTAAACT | cassette |
| | AGTGTTTGAAAAGATTCTTATTACGGTAACGCGGAATTCGCAACTATTT | |
| | TATCAATTTTTTGCGTCGACACTTCAAGGGGCTTGCGGCCGCAACCATC | |
| | TCCATGGCTGTTTGAATGAGGCTTCAGTACTTTACAGAATCGTTGCCTG | |
| | CACATCTTGGAAACACTTGCTGGGATTACTTCGACTTCTTAACCCAACA | |
| | GAAGGCTCGAGAAGGTATATTGCTGTTGACAGTGAGCGCCAGTGTGAAG | |
| | CTCTTGTCAGATAGTGAAGCCACAGATGTATCTGACAAGAGCTTCACAC | |
| | TGATGCCTACTGCCTCGGACTTCAAGGGGCTAGAATTCGAGCAATTATC | |
| | TTGTTTACTAAAACTGAATACCTTGCTATCTCTTTGATACATTTTTACA | |
| | AAGCTGAATTAAAATGGTATAAATTAAATCACTTTGACGGTGACTTAGG | |
| | AGTATGCCGGATCAACGCCCTAGGTTTATGTTTGGATGAACTGACATAC | |
| | GCGTATCCGTCTAAAAATCAATCTCATTTCCTGGTAGTGAAATATATAT | |
| | TAAACCAGGAAATGAGATTGATTTTTTTACGGTAACGCGGAATTCGCAA | |
| | CTATTTTATCAATTTTTTGCGTCGACGACTGTGACAGCAGAGTATGCCG | |
| | GATCAACGCCCTAGGTTTATGTTTGGATGAACTGACATACGCGTATCCG | |
| | TCTTATGTAAAAGACAAACAATGCGTAGTGAAATATATATTAAACGCAT | |
| | TGTTTGTCTTTTACATATTACGGTAACGCGGAATTCGCAACTATTTTAT | |
| | CAATTTTTTGCGTCGAC | |
| 72 | TTAAGAATCTTTTCAAACACTA | FAS shRNA |
| | | guide |
| 73 | TCTGACAAGAGCTTCACACTGA | PTPN2 |
| | | shRNA guide |
| 74 | TAAAAATCAATCTCATTTCCTG | TGFBR2 |
| | | shRNA 1 |
| | | guide |
| 75 | TTATGTAAAAGACAAACAATGC | TGFBR2 |
| | | shRNA 2 |
| | | guide |
| 76 | TGCTTTATTTGTGAAATTTGTGATGCTATTGCTTTATTTGTAACCATTA | Sv40 pA |
| | TAAGCTGCAATAAACAAGTTAACAACAACAATTGCATTCATTTTATGTT | |
| | TCAGGTTCAGGGGGAGGTGTGGGAGGTTTTTTAAA | |
| 77 | GGCTTAGACGCAGGTGTTCTGATTTATAGTTCAAAACCTCTATCAATGA | AAV6 vector |
| | GAGAGCAATCTCCTGGTAATGTGATAGATTTCCCAACTTAATGCCAACA | #1 full insert |
| | TACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCAT | |
| | GCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCA | |
| | GGTTTCCTTGAGTGGCAGGCCAGGCCTGGCCGTGAACGTTCACTGAAAT | |
| | CATGGCCTCTTGGCCAAGATTGATAGCTTGTGCCTGTCCCTGAGTCCCA | |
| | GTCCATCACGAGCAGCTGGTTTCTAAGATGCTATTTCCCGTATAAAGCA | |
| | TGAGACCGTGACTTGCCAGCCCCACAGAGCCCCGCCCTTGTCCATCACT | |
| | GGCATCTGGACTCCAGCCTGGGTTGGGGCAAAGAGGGAAATGAGATCAT | |
| | GTCCTAACCCTGATCCTCTTGTCCCACAGATATCCAGAACCCTGACCCT | |
| | GCCGTGTACCAGGGATCCGGATCTGCGATCGCTCCGGTGCCCGTCAGTG | |
| | GGCAGAGCGCACATCGCCCACAGTCCCCGAGAAGTTGGGGGGAGGGGTC | |
| | GGCAATTGAACCGGTGCCTAGAGAAGGTGGCGCGGGGTAAACTGGGAAA | |
| | GTGATGTCGTGTACTGGCTCCGCCTTTTTCCCGAGGGTGGGGGAGAACC | |
| | GTATATAAGTGCAGTAGTCGCCGTGAACGTTCTTTTTCGCAACGGGTTT | |
| | GCCGCCAGAACACAGCTGAAGCTTCGAGGGGCTCGCATCTCTCCTTCAC | |
| | GCGCCCGCCGCCCTACCTGAGGCCGCCATCCACGCCGGTTGAGTCGCGT | |
| | TCTGCCGCCTCCCGCCTGTGGTGCCTCCTGAACTGCGTCCGCCGTCTAG | |
| | GTAAGTCGACTCGTTGGATCCCCACTACCCGGATCAACGCCCTAGGTTT | |
| | ATGTTTGGATGAACTGACATACGCGTATCCGTCTTAAGAATCTTTTCAA | |
| | ACACTAGTAGTGAAATATATATTAAACTAGTGTTTGAAAAGATTCTTAT | |
| | TACGGTAACGCGGAATTCGCAACTATTTTATCAATTTTTTGCGTCGACA | |
| | CTTCAAGGGGCTTGCGGCCGCAACCATCTCCATGGCTGTTTGAATGAGG | |
| | CTTCAGTACTTTACAGAATCGTTGCCTGCACATCTTGGAAACACTTGCT | |
| | GGGATTACTTCGACTTCTTAACCCAACAGAAGGCTCGAGAAGGTATATT | |
| | GCTGTTGACAGTGAGCGCCAGTGTGAAGCTCTTGTCAGATAGTGAAGCC | |
| | ACAGATGTATCTGACAAGAGCTTCACACTGATGCCTACTGCCTCGGACT | |
| | TCAAGGGGCTAGAATTCGAGCAATTATCTTGTTTACTAAAACTGAATAC | |
| | CTTGCTATCTCTTTGATACATTTTTACAAAGCTGAATTAAAATGGTATA | |
| | AATTAAATCACTTTGACGGTGACTTAGGAGTATGCCGGATCAACGCCCT | |
| | AGGTTTATGTTTGGATGAACTGACATACGCGTATCCGTCTAAAAATCAA | |
| | TCTCATTTCCTGGTAGTGAAATATATATTAAACCAGGAAATGAGATTGA | |
| | TTTTTTTACGGTAACGCGGAATTCGCAACTATTTTATCAATTTTTTGCG | |
| | TCGACGACTGTGACAGCAGAGTATGCCGGATCAACGCCCTAGGTTTATG | |
| | TTTGGATGAACTGACATACGCGTATCCGTCTTATGTAAAAGACAAACAA | |
| | TGCGTAGTGAAATATATATTAAACGCATTGTTTGTCTTTTACATATTAC | |
| | GGTAACGCGGAATTCGCAACTATTTTATCAATTTTTTGCGTCGACCGGA | |
| | ACTATCTTGAAGAGTAGTAGTGGACTAGTGTGACGCTGCTGACCCCTTT | |
| | CTTTCCCTTCTACAGATCCAAGCTGTGACCGGCGCCTACACCTGCAGCC | |
| | CAAGCTTACCGCTAGCGCCACCATGCCCCTGCTGCTCCTGCTCCCTTTG | |
| | CTGTGGGCGGGAGCCCTCGCGCAAGTGCAACTGGTAGAAAGCGGCGGCG | |
| | GCCTGGTGCAGCCTGGCGGCTCTCTAAGACTGAGCTGCGCCGCAAGCGG | |
| | CGTGACCCTGGAGTACAGCGCCATCGGCTGGTTCAGACAAGCCCCCGGC | |
| | AAGGAGCAAGAGGGCGTGAGCTGCACAAGCAGCAGCGACGGCAGCATCT | |
| | ACTACGCCGACAGCGTGAAGGGCAGATTCACCATCAGCAGAGACAACAG | |
| | CAAGAACACCCTGTACCTGCAGATGAACAGCCTGAGAGCCGAGGACACC | |
| | GCCGTGTACTACTGCGCCATCGGCGTTTTCCATGGCGGTTCTTGCAGAG | |
| | GCGCCCTTGATACCTGGGGCCAAGGCACCCTGGTGACCGTGAGCAGCAT | |
| | TGAAGTTATGTATCCTCCTCCTTACCTAGACAATGAGAAGAGCAATGGA | |
| | ACCATTATCCATGTGAAAGGGAAACACCTTTGTCCAAGTCCCCTATTTC | |
| | CCGGACCTTCTAAGCCCTTCTGGGTGCTCGTGGTCGTTGGCGGAGTGCT | |
| | GGCCTGTTACTCCCTGCTGGTTACCGTGGCCTTCATCATCTTTTGGGTC | |
| | AAGCGGGGCAGAAAGAAGCTGCTCTACATCTTCAAGCAGCCCTTCATGC | |
| | GGCCCGTGCAGACCACACAAGAGGAAGATGGCTGCTCCTGCCGATTCCC | |
| | CGAGGAAGAAGAAGGCGGCTGCGAGCTGAGAGTGAAGTTCAGCAGATCC | |
| | GCCGACGCTCCAGCCTATCAGCAGGGCCAAAACCAGCTGTACAACGAGC | |
| | TGAACCTGGGGAGAAGAGAAGAGTACGACGTGCTGGATAAGCGGAGAGG | |
| | CAGAGATCCTGAAATGGGCGGCAAGCCCAGACGGAAGAATCCTCAAGAG | |
| | GGCCTGTATAATGAGCTGCAGAAAGACAAGATGGCCGAGGCCTACAGCG | |
| | AGATCGGAATGAAGGGCGAGCGCAGAAGAGGCAAGGGACACGATGGACT | |
| | GTACCAGGGCCTGAGCACCGCCACCAAGGATACCTATGACGCACTGCAC | |
| | ATGCAGGCCCTGCCACCTAGATGAGTCGACTGCTTTATTTGTGAAATTT | |
| | GTGATGCTATTGCTTTATTTGTAACCATTATAAGCTGCAATAAACAAGT | |
| | TAACAACAACAATTGCATTCATTTTATGTTTCAGGTTCAGGGGGAGGTG | |
| | TGGGAGGTTTTTTAAAGGGCCCCTCTAAATCCAGTGACAAGTCTGTCTG | |
| | CCTATTCACCGATTTTGATTCTCAAACAAATGTGTCACAAAGTAAGGAT | |
| | TCTGATGTGTATATCACAGACAAAACTGTGCTAGACATGAGGTCTATGG | |
| | ACTTCAAGAGCAACAGTGCTGTGGCCTGGAGCAACAAATCTGACTTTGC | |
| | ATGTGCAAACGCCTTCAACAACAGCATTATTCCAGAAGACACCTTCTTC | |
| | CCCAGCCCAGGTAAGGGCAGCTTTGGTGCCTTCGCAGGCTGTTTCCTTG | |
| | CTTCAGGAATGGCCAGGTTCTGCCCAGAGCTCTGGTCAATGATGTCTAA | |
| | AACTCCTCTGATTGGTGGTCTCGGCCTTATCCATTGCCACCAAAACCCT | |
| | CTTTTTACTAAGAAACAGTGAGCCTTGTTCTGGCAGTCCAGAGAATGAC | |
| | ACGGGAAAAAAGCAGATGAAGAGAAGGTGGCAGGAGAGGGCACGTGGCC | |
| | CAGCCTCAGTCTCTCCAACTGAGTTCCTGCCTGCCTGCCTTTGCTCAGA | |
| | CTGTTTGCCCCTTACTGCTCTTCTAGGCCTCATTCTAAGCCCCTTCTCC | |
| | AAGTTGCCTCTCCTTATTTCTCCCTGTCTGCCAA | |
| 78 | tgcaggcagctgcgcgctcgctcgctcactgaggccgcccgggcaaagc | AAV6 vector |
| | ccgggcgtcgggcgacctttggtcgcccggcctcagtgagcgagcgagc | #2 full insert |
| | gcgcagagagggagtggccaactccatcactaggggttcctgatatcac | |
| | gcgtagcaattgctatgtcccaggcactctactagacacttcatacagt | |
| | ttagaaaatcagatgggtgtagatcaaggcaggagcaggaaccaaaaag | |
| | aaaggcataaacataagaaaaaaaatggaaggggtggaaacagagtaca | |
| | ataacatgagtaatttgatgggggctattatgaactgagaaatgaactt | |
| | tgaaaagtatcttggggccaaatcatgtagactcttgagtgatgtgtta | |
| | aggaatgctatgagtgctgagagggcatcagaagtccttgagagcctcc | |
| | agagaaaggctcttaaaaatgcagcgcaatctccagtgacagaagatac | |
| | tgctagaaatctgctagaaaaaaaacaaaaaaggcatgtatagaggaat | |
| | tatgagggaaagataccaagtcacggtttattcttcaaaatggaggtgg | |
| | cttgttgggaaggtggaagctcatttggccagagtggaaatggaattgg | |
| | gagaaatcgatgaccaaatgtaaacacttggtgcctgatatagcttgac | |
| | accaagttagccccaagtgaaataccctggcaatattaatgtgtctttt | |
| | cccgatattcctcagGTACTCCAAAGATTCAGGTTTACTCACGTCATCC | |
| | AGCAGAGAATGGAAAGTCAAATTTCCTGAATTGCTATGTGTCTGGGTTT | |
| | CATCCATCCGACATTGAAGTTGACTTACTGAAGAATGGAGAGAGAATTG | |
| | AAAAAGTGGAGCATTCAGACTTGTCTTTCAGCAAGGACTGGTCTTTCTA | |
| | TCTCTTGTACTACACTGAAggatccGGCAGCGGCGAGGGCAGAGGCAGC | |
| | CTGCTGACCTGCGGCGACGTGGAGGAGAACCCCGGCCCCATGTCAAGAT | |
| | CTGTCGCTCTGGCTGTCCTGGCCCTGCTGTCTCTGTCTGGACTTGAAGC | |
| | AGTGATGGCCCCTAGAACACTGGTGCTTGGAGGCGGAGGATCTGGCGGA | |
| | GGTGGAAGTGGCGGAGGCGGATCTATCCAACGGACACCTAAGATCCAAG | |
| | TGTACAGCAGACACCCCGCCGAGAACGGCAAGAGCAACTTCCTGAACTG | |
| | CTACGTGTCCGGCTTCCACCCCTCTGACATTGAGGTGGACCTGCTGAAG | |
| | AACGGCGAGCGGATCGAGAAGGTGGAACACAGCGATCTGAGCTTCTCCA | |
| | AGGATTGGTCCTTCTACCTGCTCTACTACACCGAGTTCACTCCTACCGA | |
| | GAAGGACGAGTACGCATGTAGAGTGAACCACGTGACACTGAGCCAACCT | |
| | AAGATCGTGAAATGGGACAGAGATATGGGAGGCGGCGGTAGTGGCGGCG | |
| | GAGGAAGCGGAGGCGGAGGTTCAGGTGGTGGTGGATCTGGAAGCCACAG | |
| | CCTGAAGTACTTTCACACCTCCGTGTCCAGACCTGGCAGAGGCGAGCCT | |
| | AGATTCATCAGCGTGGGCTACGTGGACGACACCCAGTTCGTCAGATTCG | |
| | ACAACGACGCCGCCTCTCCTCGGATGGTTCCTAGAGCACCCTGGATGGA | |
| | ACAAGAGGGCAGCGAGTACTGGGATCGCGAGACAAGAAGCGCCAGAGAC | |
| | ACAGCCCAGATCTTCCGCGTGAACCTGAGAACCCTGCGGGGCTACTACA | |
| | ATCAGTCTGAGGCCGGCTCTCACACCCTGCAGTGGATGCATGGATGTGA | |
| | ACTGGGCCCCGACGGACGGTTCCTGAGAGGCTATGAGCAGTTCGCCTAC | |
| | GACGGCAAGGACTACCTGACACTGAACGAGGACCTGAGAAGCTGGACCG | |
| | CCGTGGATACAGCCGCTCAGATCAGCGAGCAGAAGTCTAACGACGCCAG | |
| | CGAGGCCGAACACCAGAGAGCCTATCTGGAAGATACCTGCGTGGAATGG | |
| | CTGCACAAGTACCTGGAAAAGGGCAAAGAGACACTGCTGCACCTGGAAC | |
| | CACCTAAGACACATGTGACCCACCATCCTATCAGCGACCACGAGGCCAC | |
| | ACTGAGATGTTGGGCCCTGGGCTTTTACCCTGCCGAGATCACACTGACA | |
| | TGGCAGCAGGATGGCGAGGGCCACACACAGGATACAGAGCTGGTGGAAA | |
| | CAAGACCTGCCGGCGACGGCACCTTCCAGAAATGGGCTGCTGTGGTGGT | |
| | TCCCAGCGGCGAGGAACAGAGATACACCTGTCACGTGCAGCACGAGGGA | |
| | CTGCCTGAACCTGTGACTCTGAGATGGAAGCCTGCCAGCCAGCCAACAA | |
| | TCCCCATCGTGGGAATCATTGCCGGCCTGGTGCTGCTGGGATCTGTGGT | |
| | TTCTGGTGCAGTGGTGGCCGCCGTGATTTGGAGAAAGAAGTCCTCTGGC | |
| | GGCAAAGGCGGCTCCTACTATAAGGCCGAGTGGAGCGATTCTGCCCAGG | |
| | GCTCTGAAAGCCACTCTCTGTGAactagtgtcgactgctttatttgtga | |
| | aatttgtgatgctattgctttatttgtaaccattataagctgcaataaa | |
| | caagttaacaacaacaattgcattcattttatgtttcaggttcaggggg | |
| | aggtgtgggaggttttttaaagggcccACTGAAAAAGATGAGTATGCCT | |
| | GCCGTGTGAACCATGTGACTTTGTCACAGCCCAAGATAGTTAAGTGGGg | |
| | taagtcttacattcttttgtaagctgctgaaagttgtgtatgagtagtc | |
| | atatcataaagctgctttgatataaaaaaggtctatggccatactaccc | |
| | tgaatgagtcccatcccatctgatataaacaatctgcatattgggattg | |
| | tcagggaatgttcttaaagatcagattagtggcacctgctgagatactg | |
| | atgcacagcatggtttctgaaccagtagtttccctgcagttgagcaggg | |
| | agcagcagcagcacttgcacaaatacatatacactcttaacacttctta | |
| | cctactggcttcctctagcttttgtggcagcttcaggtatatttagcac | |
| | tgaacgaacatctcaagaaggtataggcctttgtttgtaagtcctgctg | |
| | tcctagcatcctataatcctggacttctccagtactttctggctggatt | |
| | ggtatctgaggctagtaggaagggcttgttcctgctgggtagctctaaa | |
| | caatgtattcatgggtaggaacagcagcctattctgccagccttatttc | |
| | taaccattttagacatttgttagtacatggtattttaaaagtaaaactt | |
| | aatgtcttccttttttttctccactgtctttttcatagATCGAGACATG | |
| | TAAGCAGCATCATGGAGgtaagtttttgaccttgagaaaatgtttttgt | |
| | ttcactgtcctgaggactatttatagacagctctaacatgatatcgcga | |
| | ctgcaggcgatctctaggaacccctagtgatggagttggccactccctc | |
| | tctgcgcgctcgctcgctcactgaggccgggcgaccaaaggtcgcccga | |
| | cgcccgggctttgcccgggcggcctcagtgagcgagcgagcgcgcagct | |
| | gcctgcatatctctcgatccttgggctatcgaaacttaattaaccagtc | |
| | aagtcagctacttggcgagatcgacttgtctgggtttcgactacgctca | |
| | gaattgcgtcagtcaagttcgatctggtccttgctattgcacccgttct | |
| | ccgattacgagtttcatttaaatcatgtgagcaaaaggccagcaaaagg | |
| | ccaggaaccgtaaaaaggccgcgttgctggcgtttttccataggctccg | |
| | cccccctgacgagcatcacaaaaatcgacgctcaagtcagaggtggcga | |
| | aacccgacaggactataaagataccaggcgtttccccctggaagctccc | |
| | tcgtgcgctctcctgttccgaccctgccgcttaccggatacctgtccgc | |
| | ctttctcccttcgggaagcgtggcgctttctcatagctcacgctgtagg | |
| | tatctcagttcggtgtaggtcgttcgctccaagctgggctgtgtgcacg | |
| | aaccccccgttcagcccgaccgctgcgccttatccggtaactatcgtct | |
| | tgagtccaacccggtaagacacgacttatcgccactggcagcagccact | |
| | ggtaacaggattagcagagcgaggtatgtaggcggtgctacagagttct | |
| | tgaagtggtggcctaactacggctacactagaagaacagtatttggtat | |
| | ctgcgctctgctgaagccagttaccttcggaaaaagagttggtagctct | |
| | tgatccggcaaacaaaccaccgctggtagcggtggtttttttgtttgca | |
| | agcagcagattacgcgcagaaaaaaaggatctcaagaagatcctttgat | |
| | cttttctacggggtctgacgctcagtggaacgaaaactcacgttaaggg | |
| | attttggtcatgagattatcaaaaaggatcttcacctagatccttttaa | |
| | attaaaaatgaagttttaaatcaatctaaagtatatatgagtaaacttg | |
| | gtctgacagttaccaatgcttaatcagtgaggcacctatctcagcgatc | |
| | tgtctatttcgttcatccatagttgcatttaaatttccgaactctccaa | |
| | ggccctcgtcggaaaatcttcaaacctttcgtccgatccatcttgcagg | |
| | ctacctctcgaacgaactatcgcaagtctcttggccggccttgcgcctt | |
| | ggctattgcttggcagcgcctatcgccaggtattactccaatcccgaat | |
| | atccgagatcgggatcacccgagagaagttcaacctacatcctcaatcc | |
| | cgatctatccgagatccgaggaatatcgaaatcggggcgcgcctggtgt | |
| | accgagaacgatcctctcagtgcgagtctcgacgatccatatcgttgct | |
| | tggcagtcagccagtcggaatccagcttgggacccaggaagtccaatcg | |
| | tcagatattgtactcaagcctggtcacggcagcgtaccgatctgtttaa | |
| | acctagatattgatagtctgatcggtcaacgtataatcgagtcctagct | |
| | tttgcaaacatctatcaagagacaggatcagcaggaggctttcgcatga | |
| | ttgaacaagatggattgcacgcaggttctccggcggcttgggtggagag | |
| | gctattcggctatgactgggcacaacagacaatcggctgctctgatgcc | |
| | gccgtgttccggctgtcagcgcaggggcgtccggttctttttgtcaaga | |
| | ccgacctgtccggtgccctgaatgaactgcaagacgaggcagcgcggct | |
| | atcgtggctggcgacgacgggcgttccttgcgcggctgtgctcgacgtt | |
| | gtcactgaagcgggaagggactggctgctattgggcgaagtgccggggc | |
| | aggatctcctgtcatctcaccttgctcctgccgagaaagtatccatcat | |
| | ggctgatgcaatgcggcggctgcatacgcttgatccggctacctgccca | |
| | ttcgaccaccaagcgaaacatcgcatcgagcgagcacgtactcggatgg | |
| | aagccggtcttgtcgatcaggatgatctggacgaagagcatcaggggct | |
| | cgcgccagccgaactgttcgccaggctcaaggcgtctatgcccgacggc | |
| | gaggatctcgtcgtgacccacggcgatgcctgcttgccgaatatcatgg | |
| | tggaaaatggccgcttttctggattcatcgactgtggccgtctgggtgt | |
| | ggcggaccgctatcaggacatagcgttggctacccgtgatattgctgaa | |
| | gagcttggcggcgaatgggctgaccgcttccttgtgctttacggtatcg | |
| | ccgcgcccgattcgcagcgcatcgccttctatcgccttcttgacgagtt | |
| | cttctgaccgattctaggtgcattggcgcagaaaaaaatgcctgatgcg | |
| | acgctgcgcgtcttatactcccacatatgccagattcagcaacggatac | |
| | ggcttccccaacttgcccacttccatacgtgtcctccttaccagaaatt | |
| | tatccttaaggtcgtttaaactcgactctggctctatcgaatctccgtc | |
| | gtttcgagcttacgcgaacagccgtggcgctcatttgctcgtcgggcat | |
| | cgaatctcgtcagctatcgtcagcttacctttttggcagcgatcgctta | |
| | accctagaaagatagtctgcgtaaaattgacgca | |
| 79 | YHNIG | CD70 CDR1 |
| | | CAR (Binder |
| | | 18) Kabat |
| 80 | CISSSDGSTYYADSVKG | CD70 CDR2 |
| | | CAR (Binder |
| | | 18) Kabat |
| 81 | GLFYPGSCRGALDA | CD70 CDR3 |
| | | CAR (Binder |
| | | 18) Kabat |
| 82 | GFTLDYHN | CD70 CDR1 |
| | | CAR (Binder |
| | | 18) IMGT |
| 83 | ISSSDGST | CD70 CDR2 |
| | | CAR (Binder |
| | | 18) IMGT |
| 84 | ATGLFYPGSCRGALDA | CD70 CDR3 |
| | | CAR (Binder |
| | | 18) IMGT |
| 85 | actcgcgcgcacggagcgacgacacccccgcgcgtgcacccgctcggga | TGFBR2 |
| | caggagccggactcctgtgcagcttccctcggccgccgggggcctcccc | mRNA |
| | gcgcctcgccggcctccaggccccctcctggctggcgagcgggcgccac | |
| | atctggcccgcacatctgcgctgccggcccggcgcggggtccggagagg | |
| | gcgcggcgcggaggcgcagccaggggtccgggaaggcgccgtccgctgc | |
| | gctgggggctcggtctatgacgagcagcggggtctgccatgggtcgggg | |
| | gctgctcaggggcctgtggccgctgcacatcgtcctgtggacgcgtatc | |
| | gccagcacgatcccaccgcacgttcagaagtcggttaataacgacatga | |
| | tagtcactgacaacaacggtgcagtcaagtttccacaactgtgtaaatt | |
| | ttgtgatgtgagattttccacctgtgacaaccagaaatcctgcatgagc | |
| | aactgcagcatcacctccatctgtgagaagccacaggaagtctgtgtgg | |
| | ctgtatggagaaagaatgacgagaacataacactagagacagtttgcca | |
| | tgaccccaagctcccctaccatgactttattctggaagatgctgcttct | |
| | ccaaagtgcattatgaaggaaaaaaaaaagcctggtgagactttcttca | |
| | tgtgttcctgtagctctgatgagtgcaatgacaacatcatcttctcaga | |
| | agaatataacaccagcaatcctgacttgttgctagtcatatttcaagtg | |
| | acaggcatcagcctcctgccaccactgggagttgccatatctgtcatca | |
| | tcatcttctactgctaccgcgttaaccggcagcagaagctgagttcaac | |
| | ctgggaaaccggcaagacgcggaagctcatggagttcagcgagcactgt | |
| | gccatcatcctggaagatgaccgctctgacatcagctccacgtgtgcca | |
| | acaacatcaaccacaacacagagctgctgcccattgagctggacaccct | |
| | ggtggggaaaggtcgctttgctgaggtctataaggccaagctgaagcag | |
| | aacacttcagagcagtttgagacagtggcagtcaagatctttccctatg | |
| | aggagtatgcctcttggaagacagagaaggacatcttctcagacatcaa | |
| | tctgaagcatgagaacatactccagttcctgacggctgaggagcggaag | |
| | acggagttggggaaacaatactggctgatcaccgccttccacgccaagg | |
| | gcaacctacaggagtacctgacgcggcatgtcatcagctgggaggacct | |
| | gcgcaagctgggcagctccctcgcccgggggattgctcacctccacagt | |
| | gatcacactccatgtgggaggcccaagatgcccatcgtgcacagggacc | |
| | tcaagagctccaatatcctcgtgaagaacgacctaacctgctgcctgtg | |
| | tgactttgggctttccctgcgtctggaccctactctgtctgtggatgac | |
| | ctggctaacagtgggcaggtgggaactgcaagatacatggctccagaag | |
| | tcctagaatccaggatgaatttggagaatgttgagtccttcaagcagac | |
| | cgatgtctactccatggctctggtgctctgggaaatgacatctcgctgt | |
| | aatgcagtgggagaagtaaaagattatgagcctccatttggttccaagg | |
| | tgcgggagcacccctgtgtcgaaagcatgaaggacaacgtgttgagaga | |
| | tcgagggcgaccagaaattcccagcttctggctcaaccaccagggcatc | |
| | cagatggtgtgtgagacgttgactgagtgctgggaccacgacccagagg | |
| | cccgtctcacagcccagtgtgtggcagaacgcttcagtgagctggagca | |
| | tctggacaggctctcggggaggagctgctcggaggagaagattcctgaa | |
| | gacggctccctaaacactaccaaatagctcttctggggcaggctgggcc | |
| | atgtccaaagaggctgcccctctcaccaaagaacagaggcagcaggaag | |
| | ctgcccctgaactgatgcttcctggaaaaccaagggggtcactcccctc | |
| | cctgtaagctgtggggataagcagaaacaacagcagcagggagtgggtg | |
| | acatagagcattctatgcctttgacattgtcataggataagctgtgtta | |
| | gcacttcctcaggaaatgagattgatttttacaatagccaataacattt | |
| | gcactttattaatgcctgtatataaatatgaatagctatgttttatata | |
| | tatatatatatatctatatatgtctatagctctatat | |
| | atatagccataccttgaaaagagacaaggaaaaacatcaaatattccca | |
| | ggaaattggttttattggagaactccagaaccaagcagagaaggaaggg | |
| | acccatgacagcattagcatttgacaatcacacatgcagtggttctctg | |
| | actgtaaaacagtgaactttgcatgaggaaagaggctccatgtctcaca | |
| | gccagctatgaccacattgcacttgcttttgcaaaataatcattccctg | |
| | cctagcacttctcttctggccatggaactaagtacagtggcactgtttg | |
| | aggaccagtgttcccggggttcctgtgtgcccttatttctcctggactt | |
| | ttcatttaagctccaagccccaaatctggggggctagtttagaaactct | |
| | ccctcaacctagtttagaaactctaccccatctttaataccttgaatgt | |
| | tttgaaccccactttttaccttcatgggttgcagaaaaatcagaacaga | |
| | tgtccccatccatgcgattgccccaccatctactaatgaaaaattgttc | |
| | tttttttcatctttcccctgcacttatgttactattctctgctcccagc | |
| | cttcatccttttctaaaaaggagcaaattctcactctaggctttatcgt | |
| | gtttactttttcattacacttgacttgattttctagttttctatacaaa | |
| | caccaatgggttccatctttctgggctcctgattgctcaagcacagttt | |
| | ggcctgatgaagaggatttcaactacacaatactatcattgtcaggact | |
| | atgacctcaggcactctaaacatatgttttgtttggtcagcacagcgtt | |
| | tcaaaaagtgaagccactttataaatatttggagattttgcaggaaaat | |
| | ctggatccccaggtaaggatagcagatggttttcagttatctccagtcc | |
| | acgttcacaaaatgtgaaggtgtggagacacttacaaagctgcctcact | |
| | tctcactgtaaacattagctctttccactgcctacctggaccccagtct | |
| | aggaattaaatctgcacctaaccaaggtcccttgtaagaaatgtccatt | |
| | caagcagtcattctctgggtatataatatgattttgactaccttatctg | |
| | gtgttaagatttgaagttggccttttattggactaaaggggaactcctt | |
| | taagggtctcagttagcccaagtttcttttgcttatatgttaatagttt | |
| | taccctctgcattggagagaggagtgctttactccaagaagctttcctc | |
| | atggttaccgttctctccatcatgccagccttctcaacctttgcagaaa | |
| | ttactagagaggatttgaatgtgggacacaaaggtcccatttgcagtta | |
| | gaaaatttgtgtccacaaggacaagaacaaagtatgagctttaaaactc | |
| | cataggaaacttgttaatcaacaaagaagtgttaatgctgcaagtaatc | |
| | tcttttttaaaactttttgaagctacttattttcagccaaataggaata | |
| | ttagagagggactggtagtgagaatatcagctctgtttgga | |
| | tggtggaaggtctcattttattgagatttttaagatacatgcaaaggtt | |
| | tggaaatagaacctctaggcaccctcctcagtgtggggggctgagagt | |
| | taaagacagtgtggctgcagtagcatagaggcgcctagaaattccactt | |
| | gcaccgtagggcatgctgataccatcccaatagctgttgcccattgacc | |
| | tctagtggtgagtttctagaatactggtccattcatgagatattcaaga | |
| | ttcaagagtattctcacttctgggttatcagcataaactggaatgtagt | |
| | gtcagaggatactgtggcttgttttgtttatgtttttttttcttattca | |
| | agaaaaaagaccaaggaataacattctgtagttcctaaaaatactgact | |
| | tttttcactactatacataaagggaaagttttattcttttatggaacac | |
| | ttcagctgtactcatgtattaaaataggaatgtgaatgctatatactct | |
| | ttttatatcaaaagtctcaagcacttatttttattctatgcattgtttg | |
| | tcttttacataaataaaatgtttattagattgaataaagcaaaatactc | |
| | aggtgagcatcctgcctcctgttcccattcctagtagctaaa | |
| 86 | gcatgcgccgcagcgccagcgctctccccggatcgtgcggggcctgagc | PTPN2 |
| | ctctccgccggcgcaggctctgctcgcgccagctcgctcccgcagccat | mRNA |
| | gcccaccaccatcgagcgggagttcgaagagttggatactcagcgtcgc | |
| | tggcagccgctgtacttggaaattcgaaatgagtcccatgactatcctc | |
| | atagagtggccaagtttccagaaaacagaaatcgaaacagatacagaga | |
| | tgtaagcccatatgatcacagtcgtgttaaactgcaaaatgctgagaat | |
| | gattatattaatgccagtttagttgacatagaagaggcacaaaggagtt | |
| | acatcttaacacagggtccacttcctaacacatgctgccatttctggct | |
| | tatggtttggcagcagaagaccaaagcagttgtcatgctgaaccgcatt | |
| | gtggagaaagaatcggttaaatgtgcacagtactggccaacagatgacc | |
| | aagagatgctgtttaaagaaacaggattcagtgtgaagctcttgtcaga | |
| | agatgtgaagtcgtattatacagtacatctactacaattagaaaatatc | |
| | aatagtggtgaaaccagaacaatatctcactttcattatactacctggc | |
| | cagattttggagtccctgaatcaccagcttcatttctcaatttcttgtt | |
| | taaagtgagagaatctggctccttgaaccctgaccatgggcctgcggtg | |
| | atccactgtagtgcaggcattgggcgctctggcaccttctctctggtag | |
| | acacttgtcttgttttgatggaaaaaggagatgatattaacataaaaca | |
| | agtgttactgaacatgagaaaataccgaatgggtcttattcagacccca | |
| | gatcaactgagattctcatacatggctataatagaaggagcaaaatgta | |
| | taaagggagattctagtatacagaaacgatggaaagaactttctaagga | |
| | agacttatctcctgcctttgatcattcaccaaacaaaataatgactgaa | |
| | aaatacaatgggaacagaataggtctagaagaagaaaaactgacaggtg | |
| | accgatgtacaggactttcctctaaaatgcaagatacaatggaggagaa | |
| | cagtgagagtgctctacggaaacgtattcgagaggacagaaaggccacc | |
| | acagctcagaaggtgcagcagatgaaacagaggctaaatgagaatgaac | |
| | gaaaaagaaaaaggtggttatattggcaacctattctcactaagatggg | |
| | gtttatgtcagtcattttggttggcgcttttgttggctggacactgttt | |
| | tttcagcaaaatgccctataaacaattaattttgcccagcaagcttctg | |
| | cactagtaactgacagtgctacattaatcataggggtttgtctgcagca | |
| | aacgcctcatatcccaaaaacggtgcagtagaatagacatcaaccagat | |
| | aagtgatatttacagtcacaagcccaacatctcaggactcttgactgca | |
| | ggttcctctgaaccccaaactgtaaatggctgtctaaaataaagacatt | |
| | catgtttgttaaaaactggtaaattttgcaactgtattcatacatgtca | |
| | aacacagtatttcacctgaccaacattgagatatcctttatcacaggat | |
| | ttgtttttggaggctatctggattttaacctgcacttgatataagcaat | |
| | aaatattgtggttttatctacgttattggaaagaaaatgacatttaaat | |
| | aatgtgtgtaatgtataatgtactattgacatgggcatcaacactttta | |
| | ttcttaagcatttcagggtaaatatattttataagtatctatttaatct | |
| | tttgtagttaactgtactttttaagagctcaatttgaaaaatctgttac | |
| | taaaaaaataaattgtatgtcgattgaattgtactggatacattttcca | |
| | tttttctaaagagaagtttgatatgagcagttagaagttggaataagca | |
| | atttctactatatattgcatttcttttatgttttacagttttccccatt | |
| | ttaaaaagaaaagcaaacaaagaaacaaaagtttttcctaaaaatatct | |
| | ttgaaggaaaattctccttactgggatagtcaggtaaacagttggtcaa | |
| | gactttgtaaagaaattggtttctgtaaatcccattattgatatgttta | |
| | tttttcatgaaaatttcaatgtagttggggtagattatgatttaggaag | |
| | caaaagtaagaagcagcattttatgattcataatttcagtttactagac | |
| | tgaagttttgaagtaaacacttttcagtttctttcta | |
| | cttcaataaatagtatgattatatgcaaaccttacattgtcattttaac | |
| | ttaatgaatattttttaaagcaaactgtttaatgaatttaactgctcat | |
| | ttgaatgctagctttcctcagatttcaacattccattcagtgtttaatt | |
| | tgtcttacttaaacttgaaattgttgttacaaatttaattgctaggagg | |
| | catggatagcatacattattatggatagcataccttatttcagtggttt | |
| | tcaaactatgctcattggatgtccaggtgggtcaagaggttactttcaa | |
| | ccacagcatctctgccttgtctctttatatgccacataagatttctgca | |
| | taaggcttaagtattttaaagggggcagttatcatttaaaaacagtttg | |
| | gtcgggcgcggtggctcatgcctgtaatcccagcactttgggaggctga | |
| | agtgggcagatcacctgaggtcaggagttcaagaccagcctggccaacg | |
| | tggtgaaacaccatctctactaaaaatgcaaaaattagctgggcatggt | |
| | ggagggcacctgtaatctcagctactcaggaggctgaggtaggagaatt | |
| | gcttgaacccaggagatggaggttgcagtgagctgagatcacgtcactg | |
| | cactccagccagggcgacagagcgagactccatctcaaaagaaacaaac | |
| | aaaaaaaacagtttgggccgggtgtggtggctcacgcttgtaatcccag | |
| | cacttcggaaggccaagggggcggatcacgaggtcaagagatgg | |
| | agactgtcctggccaacatggtgaaatcccttctttactaaaaatacaa | |
| | aaattatctgggcgtggtggtgcatgcctgtagtcccagctccttggga | |
| | ggctaaggcaggagaatcacttgaacccgggaggcagaggttgcagtga | |
| | gccgagattgcaccactgcactccagcctggcaacagagcaagacttcg | |
| | tctcaaaaaaaaaaaaaaaaaaagtttgaaaaccattggtatagataga | |
| | tattttgaattgatttgcatagtctccttgaatgtgttaaattatgttg | |
| | aaagtatgaaagcaggatgtaggtggtactacatattaaataagattta | |
| | tataaca | |
| 87 | ctcttctcccgcgggttggtggacccgctcagtacggagttggggaagc | FAS mRNA |
| | tctttcacttcggaggattgctcaacaaccatgctgggcatctggaccc | |
| | tcctacctctggttcttacgtctgttgctagattatcgtccaaaagtgt | |
| | taatgcccaagtgactgacatcaactccaagggattggaattgaggaag | |
| | actgttactacagttgagactcagaacttggaaggcctgcatcatgatg | |
| | gccaattctgccataagccctgtcctccaggtgaaaggaaagctaggga | |
| | ctgcacagtcaatggggatgaaccagactgcgtgccctgccaagaaggg | |
| | aaggagtacacagacaaagcccatttttcttccaaatgcagaagatgta | |
| | gattgtgtgatgaaggacatggcttagaagtggaaataaactgcacccg | |
| | gacccagaataccaagtgcagatgtaaaccaaactttttttgtaactct | |
| | actgtatgtgaacactgtgacccttgcaccaaatgtgaacatggaatca | |
| | tcaaggaatgcacactcaccagcaacaccaagtgcaaagaggaaggatc | |
| | cagatctaacttggggtggctttgtcttcttcttttgccaattccacta | |
| | attgtttgggtgaagagaaaggaagtacagaaaacatgcagaaagcaca | |
| | gaaaggaaaaccaaggttctcatgaatctccaactttaaatcctgaaac | |
| | agtggcaataaatttatctgatgttgacttgagtaaatatatcac | |
| | cactattgctggagtcatgacactaagtcaagttaaaggctttgttcga | |
| | aagaatggtgtcaatgaagccaaaatagatgagatcaagaatgacaatg | |
| | tccaagacacagcagaacagaaagttcaactgcttcgtaattggcatca | |
| | acttcatggaaagaaagaagcgtatgacacattgattaaagatctcaaa | |
| | aaagccaatctttgtactcttgcagagaaaattcagactatcatcctca | |
| | aggacattactagtgactcagaaaattcaaacttcagaaatgaaatcca | |
| | aagcttggtctagagtgaaaaacaacaaattcagttctgagtatatgca | |
| | attagtgtttgaaaagattcttaatagctggctgtaaatactgcttggt | |
| | tttttactgggtacattttatcatttattagcgctgaagagccaacata | |
| | tttgtagatttttaatatctcatgattctgcctccaaggatgtttaaaa | |
| | tctagttgggaaaacaaacttcatcaagagtaaatgcagtggcatgcta | |
| | agtacccaaataggagtgtatgcagaggatgaaagattaagattatgct | |
| | ctggcatctaacatatgattctgtagtatgaatgtaatcagtgtatgtt | |
| | agtacaaatgtctatccacaggctaaccccactctatgaatcaatagaa | |
| | gaagctatgaccttttgctgaaatatcagttactgaacaggcaggccac | |
| | tttgcctctaaattacctctgataattctagagattttaccatatttct | |
| | aaactttgtttataactctgagaagatcatatttatgtaaagtatatgt | |
| | atttgagtgcagaatttaaataaggctctacctcaaagacctttgcaca | |
| | gtttattggtgtcatattatacaatatttcaattgtgaattcacataga | |
| | aaacattaaattataatgtttgactattatatatgtgtatgcattttac | |
| | tggctcaaaactacctacttctttctcaggcatcaaaagcattttgagc | |
| | aggagagtattactagagctttgccacctctccatttttgccttggtgc | |
| | tcatcttaatggcctaatgcacccccaaacatggaaatatcaccaaaaa | |
| | atacttaatagtccaccaaaaggcaagactgcccttagaaattctagcc | |
| | tggtttggagatactaactgctctcagagaaagtagctttgtgacatgt | |
| | catgaacccatgtttgcaatcaaagatgataaaatagattcttattttt | |
| | cccccacccccgaaaatgttcaataatgtcccatgtaaaacctgctaca | |
| | aatggcagcttatacatagcaatggtaaaatcatcatctggatttagga | |
| | attgctcttgtcatacccccaagtttctaagatttaagattctccttac | |
| | tactatcctacgtttaaatatctttgaaagtttgtattaaatgtgaatt | |
| | ttaagaaataatatttatatttctgtaaatgtaaactgtgaagatagtt | |
| | ataaactgaagcagatacctggaaccacctaaagaacttccatttatgg | |
| | aggatttttttgccccttgtgtttggaattataaaatataggtaaaagt | |
| | acgtaattaaataatgtttttggtatttctggttttctcttttttggta | |
| | ggggcttgctttttggttttgtcttccttttctctaactgatgctaaat | |
| | ataacttgtctttaatgcttcttggatcccttagaaggtacttcctttt | |
| | taaccttaacccttttagtagttaaataattatttccataggttgctat | |
| | tgccaagaagacctcttccaaacagcacatgattattcgtcaaacagtt | |
| | tcgtattccagatactggaatgtggataagaaagtatacatttcaaggg | |
| | gtaggttttattattaagaaagccaaatgaggattttgaaatattcttt | |
| | cctgcatattatccattctagctacatgctggccagtgggccacctttc | |
| | ttttctgcaatttaatgctagtaatatattctatttaacccatgagtcc | |
| | caaagtattagcatttcaacatgtaagcatgtcggtaagatagttgtgc | |
| | tttgcttagggttccctcctgtgttatggtctggaaagtgtctttaggc | |
| | agaaagtctgagtgatcacagggttcactcattaatttctcttttctga | |
| | gccatcatagtctgtgctgtctgctctccagttttctatttctagacag | |
| | aagtagggcaagttaggtactagttattcttcatggccagaagtgcaag | |
| | ttctactttgcaagacaagattaagttagagaacaccctattccacttt | |
| | ggtgaactcagagcaagaactttgagttcctttgggaggaagacagtgg | |
| | agaagtctttgtacttggtgatgtggtttttttcctcatggcttcacct | |
| | agtggccccaagcatgacttctcccatgtcaatgagcacagccacattc | |
| | ccgagttgaggtgaccccacggtccagaatcatcctcattctggtgaac | |
| | ctggttctctttgtggtgggcatactgggtaggagaatcacccaaaggt | |
| | cacccatgagctgcagaaaaaaaggctatttgcagaaggagctcacaga | |
| | tcacattgaaagcattgcatattcaaacatcttggtcttctttattggc | |
| | atgcccacagggtcttctgacctctgattagatcagacactttttagat | |
| | attgaatcatcagtttctgtacaactatctgaataaggtatataatcaa | |
| | tgaaatttagaatttttttctatgcttactcctgattggtaatttgttt | |
| | gggtttagaattctatacaaggccatttgtaattttcctcagcacttta | |
| | aaaatattaaaccatgttttcttaa | |
| 88 | gagtctaaatacggaccgccttgtcctccttgtcccgctcctcctgttg | IgG-Long |
| | ccggaccttccgtgttcctgtttcctccaaaacctaaggacaccctgat | hinge |
| | gatcagcaggacccctgaagtgacctgcgtggtggtggatgtgtcccaa | |
| | gaggatcccgaggtgcagttcaactggtatgtggacggcgtggaagtgc | |
| | acaacgccaagaccaagcctagagaggaacagttccagagcacctacag | |
| | agtggtgtccgtgctgacagtgctgcaccaggattggctgaacggcaaa | |
| | gagtacaagtgcaaggtgtccaacaagggcctgcctagcagcatcgaga | |
| | aaaccatctccaaggccaagggccagccaagagagccccaggtttacac | |
| | actgcctccaagccaagaggaaatgaccaagaatcaggtgtccctgaca | |
| | tgcctggtcaagggcttctacccctccgatatcgccgtggaatgggaga | |
| | gcaatggccagcctgagaacaactacaagaccacacctcctgtgctgga | |
| | cagcgacggcagtttcttcctgtattctcggctcaccgtggataaatca | |
| | agatggcaagagggcaacgtgttcagctgcagcgtgatgcacgaggccc | |
| | tgcacaaccactacacccagaaaagcctgagcctgtctctgggcaag | |
| 89 | GAGTCTAAATACGGACCGCCTTGTCCTTCTTGTCCCGGCCAGCCAAGAG | IgG-Medium |
| | AGCCCCAGGTTTACACACTGCCTCCAAGCCAAGAGGAAATGACCAAGAA | hinge |
| | TCAGGTGTCCCTGACATGCCTGGTCAAGGGCTTCTACCCCTCCGATATC | |
| | GCCGTGGAATGGGAGAGCAATGGCCAGCCTGAGAACAACTACAAGACCA | |
| | CACCTCCTGTGCTGGACAGCGACGGCAGTTTCTTCCTGTATAGTAAACT | |
| | CACCGTGGATAAATCAAGATGGCAAGAGGGCAACGTGTTCAGCTGCAGC | |
| | GTGATGCACGAGGCCCTGCACAACCACTACACCCAGAAAAGCCTGAGCC | |
| | TGTCTCTGGGCAAG | |
| 90 | GAGTCTAAATACGGACCGCCTTGTCCTCCTTGTCCCATG | IgG-Short |
| | | hinge |
| 91 | accagcaccaccacaccagctcctagacctccaactcctgctcctacaa | CD8a hinge |
| | tcgccagccagcctctgtctctgaggcccgaagcttgtagaccagctgc | |
| | tggcggagccgtgcatacaagaggactggatttcgcctgcgac | |
| 92 | atctacatctgggccccactggctggaacatgtggcgttctgctgctga | CD8a |
| | gcctggtcatcaccctgtactgc | transmembrane |
| | | domain |
| 93 | ESKYGPPCPPCPAPPVAGPSVFLFPPKPKDTLMISRTPEVTCVVVDVSQ | IgG-Long |
| | EDPEVQFNWYVDGVEVHNAKTKPREEQFQSTYRVVSVLTVLHQDWLNGK | hinge |
| | EYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKS | |
| | RWQEGNVFSCSVMHEALHNHYTQKSLSLSLGK | |
| 94 | ESKYGPPCPSCPGQPREPQVYTLPPSQEEMTKNQVSLTCLVKGFYPSDI | IgG-Medium |
| | AVEWESNGQPENNYKTTPPVLDSDGSFFLYSKLTVDKSRWQEGNVFSCS | hinge |
| | VMHEALHNHYTQKSLSLSLGK | |
| 95 | ESKYGPPCPPCPM | IgG-Short |
| | | hinge |
| 96 | TSTTTPAPRPPTPAPTIASQPLSLRPEACRPAAGGAVHTRGLDFACD | CD8a hinge |
| 97 | IYIWAPLAGTCGVLLLSLVITLYC | CD8a |
| | | transmembrane |
| | | domain |
| 98 | QVQLVESGGGLVQPGGSLRLSCAASGVTLEYSAIGWFRQAPGKEQEGVS | anti-CD70 |
| | CTSSSDGSIYYADSVKGRFTISRDNSKNTLYLQMNSLRAEDTAVYYCAI | CAR-Fc |
| | GVFHGGSCRGALDTWGQGTLVTVSSGSAAAEPKSSDKTHTSPPSPAPEA | (Binder 16) |
| | EGAPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPI | |
| | EKTISKAKGQPREPQVYTLPPSREEMTKNQVSLRCHVKGFYPSDIAVEW | |
| | ESNGQPENNYKTTKPVLDSDGSFRLASTLTVDKSRWQQGNVFSCSVLHE | |
| | ALHNHYTQKSLSLSPG | |
| 99 | ATPAPKSCPERHYWAQGKLCCQMCEPGTFLVKDCDQHRKAAQCDPCIPG | Monomeric |
| | VSFSPDHHTRPHCESCRHCNSGLLVRNCTITANAECACRNGWQCRDKEC | soluble CD27 |
| | TECDPLPNPSLTARSSQALSPHPQPTHLPYVSEMLEARTAGHMQTLADF | |
| | RQLPARTLSTHWPPQRSLSSSDFIRGSAAAEPKSSDKTHTSPPSPAPEA | |
| | EGAPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPI | |
| | EKTISKAKGQPREPQVYTLPPSREEMTKNQVSLRCHVKGFYPSDIAVEW | |
| | ESNGQPENNYKTTKPVLDSDGSFRLASTLTVDKSRWQQGNVFSCSVLHE | |
| | ALHNHYTQKSLSLSPG | |
| 100 | GPSVFLFPPKPKDTLMISRTPEVTCVVVDVSQEDPEVQFNWYVDGVEVH | IgGL hinge |
| | NAKTKPREEQFQSTYRVVSVLTVLHQDWLNGKEYKCKVSNKGLPSSIEK | region |
| | TISKAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMHEAL | |
| | HNHYTQKSLSLSLG | |
| 101 | X1PPX2P, X1 is glycine, cysteine or arginine and | Hinge |
| | X2 is cysteine or threonine | consensus |
| 102 | TTTPAPRPPTPAPTIASQPLSLRPEACRPAAGGAVHTRGLDFACD | CD8a hinge |
| | | domain |
| 103 | IEVMYPPPYLDNEKSNGTIIHVKGKHLCPSPLFPGPSKP | CD28 hinge |
| | | domain |
| 104 | ESKYGPPCPPCP | Short Spacer |
| | | (IgG4 hinge) |
| | | (aa) |
| 105 | ESKYGPPCPPCPGQPREPQVYTLPPSQEEMTKNQVSLTCLVKGFYPSDI | Hinge-CH3 |
| | AVEWESNGQPENNYKTTPPVLDSDGSFFLYSRLTVDKSRWQEGNVFSCS | spacer |
| | VMHEALHNHYTQKSLSLSLGK | Homo sapiens |
| 106 | RWPESPKAQASSVPTAQPQAEGSLAKATTAPATTRNTGRGGEEKKKEKE | IgD-hinge-Fc |
| | KEEQEERETKTPECPSHTQPLGVYLLTPAVQDLWLRDKATFTCFVVGSD | Homo sapiens |
| | LKDAHLTWEVAGKVPTGGVEEGLLERHSNGSQSQHSRLTLPRSLWNAGT | |
| | SVTCTLNHPSLPPQRLMALREPAAQAPVKLSLNLLASSDPPEAASWLLC | |
| | EVSGFSPPNILLMWLEDQREVNTSGFAPARPPPQPGSTTFWAWSVLRVP | |
| | APPSPQPATYTCVVSHEDSRTLLNASRSLEVSYVTDH | |
| 107 | EPKSCDKTHTCPPCP | Hinge |
| 108 | ERKCCVECPPCP | Hinge |
| 109 | ELKTPLGDTHTCPRCPEPKSCDTPPPCPRCPEPKSCDTPPPCPRCPEPK | Hinge |
| | SCDTPPPCPRCP | |
| 110 | ESKYGPPCPSCP | Hinge |
| 111 | YGPPCPPCP | Hinge |
| 112 | KYGPPCPPCP | Hinge |
| 113 | EVVVKYGPPCPPCP | Hinge |
| 114 | ASTKGPSVFPLAPCSRSTSESTAALGCLVKDYFPEPVTVSWNSGALTSG | Human IgG2 |
| | VHTFPAVLQSSGLYSLSSVVTVPSSNFGTQTYTCNVDHKPSNTKVDKTV | Fc (Uniprot |
| | ERKCCVECPPCPAPPVAGPSVFLFPPKPKDTLMISRTPEVTCVVVDVSH | P01859) |
| | EDPEVQFNWYVDGVEVHNAKTKPREEQFNSTFRVVSVLTVVHQDWLNGK | |
| | EYKCKVSNKGLPAPIEKTISKTKGQPREPQVYTLPPSREEMTKNQVSLT | |
| | CLVKGFYPSDISVEWESNGQPENNYKTTPPMLDSDGSFFLYSKLTVDKS | |
| | RWQQGNVFSCSVMHEALHNHYTQKSLSLSPGK | |
| 115 | ASTKGPSVFPLAPCSRSTSESTAALGCLVKDYFPEPVTVSWNSGALTSG | Human IgG4 |
| | VHTFPAVLQSSGLYSLSSVVTVPSSSLGTKTYTCNVDHKPSNTKVDKRV | Fc (Uniprot |
| | ESKYGPPCPSCPAPEFLGGPSVFLFPPKPKDTLMISRTPEVTCVVVDVS | P01861) |
| | QEDPEVQFNWYVDGVEVHNAKTKPREEQFNSTYRVVSVLTVLHQDWLNG | |
| | KEYKCKVSNKGLPSSIEKTISKAKGQPREPQVYTLPPSQEEMTKNQVSL | |
| | TCLVKGFYPSDIAVEWESNGQPENNYKTTPPVLDSDGSFFLYSRLTVDK | |
| | SRWQEGNVFSCSVMHEALHNHYTQKSLSLSLGK | |
| 116 | IEVMYPPPYLDNEKSNGTIIHVKGKHLCPSPLFPGPSKPFWVLVVVGGV | CD28 (amino |
| | LACYSLLVTVAFIIFWV | acids 114- |
| | | 179 of |
| | | Accession |
| | | No. P10747) |
| | | Homo sapiens |
| 117 | RSKRSRLLHSDYMNMTPRRPGPTRKHYQPYAPPRDFAAYRS | CD28 (amino |
| | | acids 180- |
| | | 220 of |
| | | P10747) |
| | | Homo sapiens |
| 118 | RSKRSRGGHSDYMNMTPRRPGPTRKHYQPYAPPRDFAAYRS | CD28 (LL to |
| | | GG) |
| | | Homo sapiens |
| 119 | (G₄S)₃₋₄ | Linker, |
| | | repeated 3-4 |
| | | times |
| 120 | (G₄S)₂₋₃ | Linker, |
| | | repeated 2-3 |
| | | times |
| 121 | GGGAS(G₄S)₂ | Linker |
| 122 | GCGASGGGGSGGGGS | Linker |
| 123 | gaacagagaaacaggagaatatgggccaaacaggatatctgtggtaagc | MND |
| | agttcctgccccggctcagggccaagaacagttggaacagcagaatatg | promoter |
| | ggccaaacaggatatctgtggtaagcagttcctgccccggctcagggcc | |
| | aagaacagatggtccccagatgcggtcccgccctcagcagtttctagag | |
| | aaccatcagatgtttccagggtgccccaaggacctgaaatgaccctgtg | |
| | ccttatttgaactaaccaatcagttcgcttctcgcttctgttcgcgcgc | |
| | ttctgctccccgagctctatataagcagagctcgtttagtgaaccgtca | |
| | gatc | |
| 124 | tattgcggtagtttatcacagttaaattgctaacgcagtcagtgcttct | 5′ non-coding |
| | gacacaacagtctcgaacttaagctgcagaagttggtcgtgaggcactg | element |
| | ggcaggtaagtatcaaggttacaagacaggtttaaggagaccaatagaa | |
| | actgggcttgtcgagacagagaagactcttgcgttt | |
| 125 | aactgataggcacctattggtcttactgacatccactttgcctttctct | 3′ non-coding |
| | ccacaggtgtccactcccagttcaattacagctcttaaggctagagtac | element |
| | tcaattc | |
| 126 | atgcccctgctgctcctgctccctttgctgtgggcgggagccctcgcgc | Masking CAR |
| | aaattcagctggtgcagtccggccccgaagttaaaaagcccggcgaaac | |
| | cgtgaagattagttgcaaggctagcgggtatacctttacaaactacggc | |
| | atgaactgggtcaagcaggctcctggaaaagggctgaagtggatgggtt | |
| | ggatcaacacatacactggggaaccaacgtacgccgatgcctttaaggg | |
| | gcgattcgccttctctctcgaaacctcagcaagtacagcatacttgcag | |
| | attaacaaccttaagaacgaagatactgctacttatttctgcgccaggg | |
| | actacggggattacggaatggattattggggacagggaactagtgtgac | |
| | tgtttcatccgggggcggcgggtccggtggtgggggatccggaggcggg | |
| | ggatcagacatcgtgctgacccagagccccgctagcctggccgtgagcc | |
| | ttggacagcgcgccaccatcagttgtcgagcgtctaagtctgtatccac | |
| | tagcggatactcatttatgcactggtaccagcagaagcctggccagccg | |
| | cccaagttgcttatctatttggcatccaacctggaatccggggttcctg | |
| | cgcgcttctccggtagtggatccggcaccgattttaccctcaatatcca | |
| | cccggttgaggaggaggacgccgctacatactattgtcagcattcacgc | |
| | gaggtgccatggacctttggaggcgggaccaaactcgagattaagaggg | |
| | agtctaaatacggaccgccttgtcctccttgtcccgctcctcctgttgc | |
| | cggaccttccgtgttcctgtttcctccaaaacctaaggacaccctgatg | |
| | atcagcaggacccctgaagtgacctgcgtggtggtggatgtgtcccaag | |
| | aggatcccgaggtgcagttcaactggtatgtggacggcgtggaagtgca | |
| | caacgccaagaccaagcctagagaggaacagttccagagcacctacaga | |
| | gtggtgtccgtgctgacagtgctgcaccaggattggctgaacggcaaag | |
| | agtacaagtgcaaggtgtccaacaagggcctgcctagcagcatcgagaa | |
| | aaccatctccaaggccaagggccagccaagagagccccaggtttacaca | |
| | ctgcctccaagccaagaggaaatgaccaagaatcaggtgtccctgacat | |
| | gcctggtcaagggcttctacccctccgatatcgccgtggaatgggagag | |
| | caatggccagcctgagaacaactacaagaccacacctcctgtgctggac | |
| | agcgacggcagtttcttcctgtattctcggctcaccgtggataaatcaa | |
| | gatggcaagagggcaacgtgttcagctgcagcgtgatgcacgaggccct | |
| | gcacaaccactacacccagaaaagcctgagcctgtctctgggcaagatg | |
| | ttctgggtgctcgtggtcgttggcggagtgctggcctgttactccctgc | |
| | tggttaccgtggccttcatcatcttttgggtcaagcggggcagaaagaa | |
| | gctgctctacatcttcaagcagcccttcatgcggcccgtgcagaccaca | |
| | caagaggaagatggctgctcctgccgattccccgaggaagaagaaggcg | |
| | gctgcgagctgagagtgaagttcagcagatccgccgacgctccagccta | |
| | tcagcagggccaaaaccagctgtacaacgagctgaacctggggagaaga | |
| | gaagagtacgacgtgctggataagcggagaggcagagatcctgaaatgg | |
| | gcggcaagcccagacggaagaatcctcaagagggcctgtataatgagct | |
| | gcagaaagacaagatggccgaggcctacagcgagatcggaatgaagggc | |
| | gagcgcagaagaggcaagggacacgatggactgtaccagggcctgagca | |
| | ccgccaccaaggatacctatgacgcactgcacatgcaggccctgccacc | |
| | taga | |
| 127 | GAGAATCAAAATCGGTGAAT | Cas9 TRAC |
| | | gRNA target |
| | | site |
| 128 | GAGAAUCAAAAUCGGUGAAU | Cas9 TRAC |
| | | gRNA spacer |
| 129 | QVQLVESGGGLVQPGGSLRLSCAASGFTLDYHNIGWFRQAPGKELEGVL | anti-CD70 |
| | CISSSDGSTYYADSVKGRFTISRDNSKNTLYLQMNSLRAEDTAVYYCAT | CAR-Fc |
| | GLFYPGSCRGALDAWGQGTLVTVSSGSAAAEPKSSDKTHTSPPSPAPEA | (Binder 18) |
| | EGAPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPI | |
| | EKTISKAKGQPREPQVYTLPPSREEMTKNQVSLRCHVKGFYPSDIAVEW | |
| | ESNGQPENNYKTTKPVLDSDGSFRLASTLTVDKSRWQQGNVFSCSVLHE | |
| | ALHNHYTQKSLSLSPG | |

What is claimed:

1. A chimeric antigen receptor (CAR) directed against CD70, wherein the CAR comprises a CD70-binding domain that binds to CD70 comprising a heavy chain only variable (VHH) region, wherein:
(i) the VHH region comprises a CDR-1, CDR-2, and CDR-3 each comprising a sequence that is contained within SEQ ID NO: 1, wherein X is Q or pyroglutamate; or
(ii) the VHH region comprises a CDR-1, CDR-2, and CDR-3 each comprising a sequence that is contained within SEQ ID NO:27, wherein X is Q or pyroglutamate.

2. The CAR of claim 1, further comprising a spacer domain, a transmembrane domain, and an intracellular signaling domain.

3. The CAR of claim 1 or claim 2, wherein the VHH region of the CD70-binding domain comprises CDR-1, CDR-2, and CDR-3 sequences set forth in SEQ ID NOs: 4, 5, and 6, respectively.

4. The CAR of claim 1 or claim 2, wherein the VHH region of the CD70-binding domain comprises CDR-1, CDR-2, and CDR-3 sequences set forth in SEQ ID NOs: 7, 8, and 9, respectively.

5. The CAR of claim 1 or claim 2, wherein the VHH region of the CD70-binding domain comprises CDR-1, CDR-2, and CDR-3 sequences set forth in SEQ ID NOs: 79, 80, and 81, respectively.

6. The CAR of claim 1 or claim 2, wherein the VHH region of the CD70-binding domain comprises CDR-1, CDR-2, and CDR-3 sequences set forth in SEQ ID NOs: 82, 83, and 84, respectively.

7. The CAR of any one of claims 1-6, wherein VHH region of the CD70-binding domain, wherein:
(i) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or
(ii) the VHH region comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate.

8. The CAR of any one of claims 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 2.

9. The CAR of any one of claims 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 3, wherein X is pyroglutamate.

10. The CAR of any one of claims 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO:28.

11. The CAR of any one of claims 1-7, wherein the VHH region comprises a sequence set forth in, or a sequence that is at 85%, 86%, 87%, 88%, 89%, 90%, 91%, 92%, 93%, 94%, 95%, 96%, 97%, 98% or 99% identical to, SEQ ID NO: 29, wherein X is pyroglutamate.

12. The CAR of any one of claims 1-7, wherein the VHH region of the CD70-binding domain, wherein:
(i) the VHH region comprises a sequence set forth in SEQ ID NO: 1, wherein X is Q or pyroglutamate; or
(ii) the VHH region comprises a sequence set forth in SEQ ID NO: 27, wherein X is Q or pyroglutamate.

13. The CAR of any one of claims 1-12, wherein the VHH region comprises a sequence set forth in SEQ ID NO: 2.

14. The CAR of any one of claims 1-12, wherein the VHH region comprises a sequence set forth in SEQ ID NO: 3, wherein X is pyroglutamate.

15. The CAR of any one of claims 1-12, wherein the VHH region comprises a sequence set forth in SEQ ID NO: 28.

16. The CAR of any one of claims 1-12, wherein the VHH region comprises a sequence set forth in SEQ ID NO: 29, wherein X is pyroglutamate.

17. The CAR of any one of claims 2-16, wherein the spacer domain comprises a hinge domain from human CD28.

18. The CAR of any one of claims 2-17, wherein the spacer domain comprises an amino acid sequence having at least 85%, 86%, 87%, 88%, 89%, 90%, 91%, 92%, 93%, 94%, 95%, 96%, 97%, 98% or 99% identity to SEQ ID NO: 10.

19. The CAR of claim 18, wherein the spacer domain comprises the sequence set forth in SEQ ID NO: 10.

20. The CAR of any one of claims 2-19, wherein the transmembrane domain comprises a transmembrane domain from human CD28.

21. The CAR of any one of claims 2-20, wherein the transmembrane domain comprises an amino acid sequence having at least at or about 85%, 86%, 87%, 88%, 89%, 90%, 91%, 92%, 93%, 94%, 95%, 96%, 97%, 98% or 99% identity to SEQ ID NO:11.

22. The CAR of any one of claims 2-13, wherein the transmembrane domain comprises the sequence set forth in SEQ ID NO: 11.

23. The CAR of any one of claims 2-21, wherein the intracellular signaling domain comprises a cytoplasmic signaling domain of a human CD3-zeta (CD3ζ) chain.

24. The CAR of any one of claims 2-23, wherein the intracellular signaling domain comprises an amino acid sequence having at least at or about 90%, 91%, 92%, 93%, 94%, 95%, 96%, 97%, 98% or 99% sequence identity to SEQ ID NO: 13.

25. The CAR of any one of claims 2-24, wherein the intracellular signaling domain comprises the amino acid sequence set forth in SEQ ID NO: 13.

26. The CAR of any one of claims 2-24, wherein the intracellular signaling domain further comprises a costimulatory signaling region.

27. The CAR of claim 26, wherein the costimulatory signaling region comprises an intracellular signaling domain of a T cell costimulatory molecule or a signaling portion thereof.

28. The CAR of claim 26 or claim 27, wherein the costimulatory signaling region comprises an intracellular signaling domain of human 4-1BB.

29. The CAR of any one of claims 26-28, wherein the costimulatory signaling region comprises an amino acid sequence having at least at or about 90%, 91%, 92%, 93%, 94%, 95%, 96%, 97%, 98% or 99% sequence identity to SEQ ID NO: 12.

30. The CAR of any one of claims 26-29, wherein the costimulatory signaling region comprises the amino acid sequence set forth in SEQ ID NO: 12.

31. The CAR of any one of claims 1-29, wherein the CAR comprises the amino acid sequence set forth in SEQ ID NO: 38 or SEQ ID NO: 41, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38 or SEQ ID NO: 41, wherein X is Q or pyroglutamate.

32. The CAR of any one of claims 1-31, wherein the CAR comprises the amino acid sequence set forth in SEQ ID NO: 38, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 38, wherein X is Q or pyroglutamate.

33. The CAR of any one of claims 1-32, wherein the CAR comprises the amino acid sequence set forth in SEQ ID NO: 38, wherein X is Q or pyroglutamate.

34. The CAR of any one of claims 1-32, wherein the CAR comprises the amino acid sequence set forth in SEQ ID NO: 39, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 39.

35. The CAR of any one of claims 1-32, wherein the CAR comprises the amino acid sequence set forth in SEQ ID NO: 39.

36. The CAR of any one of claims 1-32, wherein the CAR comprises the amino acid sequence set forth in SEQ ID NO: 40, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 40, wherein X is pyroglutamate.

37. The CAR of any one of claims 1-33 and 36, wherein the CAR comprises the amino acid sequence set forth in SEQ ID NO: 40, wherein X is pyroglutamate.

38. The CAR of any one of claims 1-31, wherein the CAR comprises the amino acid sequence set forth in SEQ ID NO: 41, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 41, wherein X is Q or pyroglutamate.

39. The CAR of any one of claims 1-31 and 38, wherein the CAR comprises the amino acid sequence set forth in SEQ ID NO: 41, wherein X is Q or pyroglutamate.

40. The CAR of any one of claims 1-31 and 38, wherein the CAR comprises the amino acid sequence set forth in SEQ ID NO: 42, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 42.

41. The CAR of any one of claims 1-31 and 38-40, wherein the CAR comprises the amino acid sequence set forth in SEQ ID NO: 42.

42. The CAR of any one of claims 1-31 and 38, wherein the CAR comprises the amino acid sequence set forth in SEQ ID NO: 43, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 43, wherein X is pyroglutamate.

43. The CAR of any one of claims 1-31, 38, 39, and 42, wherein the CAR comprises the amino acid sequence set forth in SEQ ID NO: 43, wherein X is pyroglutamate.

44. The CAR of any one of claims 1-43, wherein the CAR comprises the amino acid sequence set forth in SEQ ID NO: 14 or SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14 or SEQ ID NO: 44, wherein X is Q or pyroglutamate.

45. The CAR of any one of claims 1-37 and 44, wherein the CAR comprises the amino acid sequence set forth in SEQ ID NO: 14, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 14, wherein X is Q or pyroglutamate.

46. The CAR of any one of claims 1-37, 44, and 45, wherein the CAR comprises the amino acid sequence set forth in SEQ ID NO: 14, wherein X is Q or pyroglutamate.

47. The CAR of any one of claims 1-33, 44, and 45, wherein the CAR comprises the amino acid sequence set forth in SEQ ID NO: 15, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 15.

48. The CAR of any one of claims 1-33 and 44-47, wherein the CAR comprises the amino acid sequence set forth in SEQ ID NO: 15.

49. The CAR of any one of claims 1-33, 44, and 45, wherein the CAR comprises the amino acid sequence set forth in SEQ ID NO: 16, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 16, wherein X is pyroglutamate.

50. The CAR of any one of claims 33, 44-46, and 49, wherein the CAR comprises the amino acid sequence set forth in SEQ ID NO: 16, wherein X is pyroglutamate.

51. The CAR of any one of claims 1-31 and 38-44, wherein the CAR comprises the amino acid sequence set forth in SEQ ID NO: 44, wherein X is Q or pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 44, wherein X is Q or pyroglutamate.

52. The CAR of any one of claims 1-31, 38-44, and 51, wherein the CAR comprises the amino acid sequence set forth in SEQ ID NO: 44, wherein X is Q or pyroglutamate.

53. The CAR of any one of claims 1-31, 38, 44, and 51, wherein the CAR comprises the amino acid sequence set forth in SEQ ID NO: 45, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 45.

54. The CAR of any one of claims 1-31, 38-44, and 51-53, wherein the CAR comprises the amino acid sequence set forth in SEQ ID NO: 45.

55. The CAR of any one of claims 1-31, 38, 44, and 51, wherein the CAR comprises the amino acid sequence set forth in SEQ ID NO: 46, wherein X is pyroglutamate, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 46, wherein X is pyroglutamate.

56. The CAR of any one of claims 1-31, 38-44, and 51, 52, and 55, wherein the CAR comprises the amino acid sequence set forth in SEQ ID NO: 46, wherein X is pyroglutamate.

57. A genetically engineered human T cell comprising the CAR of any one of claims 1-56.

58. A genetically engineered human T cell comprising (a) a first nucleotide sequence encoding the CAR of any one of claims 1-56.

59. The genetically engineered human T cell of claim 58, wherein the first nucleotide sequence encoding the CAR comprises the nucleic acid sequence set forth in SEQ ID NO: 17.

60. The genetically engineered human T cell of any one of claims 57-59, further comprising:
(b) a first genetic disruption in an endogenous T Cell Receptor Alpha Constant (TRAC) gene; and
(c) a second genetic disruption in an endogenous β₂ microglobulin (B2M) gene.

61. A genetically engineered human T cell comprising:
(a) a first nucleotide sequence encoding a chimeric antigen receptor (CAR);
(b) a first genetic disruption in an endogenous TRAC gene;
(c) a second genetic disruption in an endogenous B₂ microglobulin (B2M) gene;
(d) a second nucleotide sequence comprising a transgene encoding a single chain HLA-E fusion protein; and
(e) a third nucleotide sequence comprising: (i) a first nucleic acid at least 15 nucleotides in length complementary to an mRNA encoding TGF-B Receptor 2 (TGFBR2); (ii) a second nucleic acid at least 15 nucleotides in length complementary to an mRNA encoding Protein Tyrosine Phosphatase Non-Receptor Type 2 (PTPN2); and (iii) a third nucleic acid at least 15 nucleotides in length complementary an mRNA encoding Fas Cell Surface Death Receptor (FAS).

62. The genetically engineered human T cell of claim 61, wherein first nucleotide sequence encoding a chimeric antigen receptor (CAR) encodes the CAR of any one of claims 1-56.

63. The genetically engineered human T cell of any one of claims 60-62, wherein the first genetic disruption and second genetic disruption are by a gene editing technique comprising a CRISPR-Cas system.

64. The genetically engineered human T cell of claim 63, wherein the CRISPR-Cas system is a ribonucleoprotein (RNP) complex comprising a Cas protein and a guide RNA (gRNA), optionally wherein the Cas protein is a Cas12a protein.

65. The genetically engineered human T cell of any one of claims 60-64, wherein the first genetic disruption in the endogenous TRAC gene is in a target site sequence in exon 1 of the endogenous TRAC gene.

66. The genetically engineered human T cell of claim 65, wherein the target site sequence in exon 1 of the endogenous TRAC gene is located at hg38 genomic coordinates chr14:22,547,528-22,547,548.

67. The genetically engineered human T cell of claim 65 or claim 66, wherein the target site sequence in exon 1 of the endogenous TRAC gene has the sequence set forth in SEQ ID NO: 59, a contiguous portion thereof of at least 14 nucleotides (nt), or a complementary sequence of the foregoing.

68. The genetically engineered human T cell of any one of claims 65-67, wherein the target site sequence in exon 1 of the endogenous TRAC gene has the sequence set forth in SEQ ID NO: 59.

69. The genetically engineered human T cell of any one of claims 60-68, wherein the first genetic disruption is by a CRISPR-Cas system that comprises a Cas12a protein and a guide RNA (gRNA) comprising a spacer sequence comprising the nucleic acid sequence of SEQ ID NO:58, or a contiguous portion thereof of at least 14 nt.

70. The genetically engineered human T cell of claim 69, wherein the CRISPR-Cas system is a ribonucleoprotein (RNP) complex comprising the Cas12a protein and the gRNA.

71. The genetically engineered human T cell of any one of claims 60-70, wherein the first genetic disruption disrupts one or more alleles of the endogenous TRAC gene.

72. The genetically engineered human T cell of any one of claims 60-71, wherein the first genetic disruption disrupts all alleles of the endogenous TRAC gene.

73. The genetically engineered human T cell of any one of claims 60-72, wherein the first genetic disruption reduces protein expression of a TCR alpha chain encoded from the endogenous TRAC gene.

74. The genetically engineered human T cell of claim 73, wherein the first genetic disruption reduces protein expression of the TCR alpha chain on the surface of the genetically engineered human T cell.

75. The genetically engineered human T cell of claim 73 or claim 74, wherein there is no detectable expression of the TCR alpha chain in the genetically engineered human T cell.

76. The genetically engineered human T cell of any one of claims 60-75, wherein the genetically engineered human T cell has reduced expression of CD3 on the cell surface of the genetically engineered human T cell.

77. The genetically engineered human T cell of any one of claims 60-76, wherein the genetically engineered human T cell does not express detectable CD3 on the cell surface of the genetically engineered human T cell.

78. The genetically engineered human T cell of any one of claims 60-77, wherein the second genetic disruption in the endogenous B2M gene is in a target site sequence in exon 2 of the endogenous B2M gene.

79. The genetically engineered human T cell of claim 78, wherein the target site sequence in exon 2 of the endogenous B2M gene is located at hg38 genomic coordinates chr15:44,715,614-44,715,634.

80. The genetically engineered human T cell of claim 78 or claim 79, wherein the target site sequence in exon 2 of the endogenous B2M gene has the sequence set forth in SEQ ID NO: 63, a contiguous portion thereof of at least 14 nucleotides (nt), or a complementary sequence of the foregoing.

81. The genetically engineered human T cell of any one of claims 78-80, wherein the target site sequence has the sequence set forth in SEQ ID NO: 63.

82. The genetically engineered human T cell of any one of claims 60-81, wherein the second genetic disruption is by a CRISPR-Cas system that comprises a Cas12a protein and a guide RNA (gRNA) comprising a spacer sequence comprising the nucleic acid sequence SEQ ID NO:62, or a contiguous portion thereof of at least 14 nt.

83. The genetically engineered human T cell of claim 82, wherein the CRISPR-Cas system is a ribonucleoprotein (RNP) complex comprising the Cas12a protein and the gRNA.

84. The genetically engineered human T cell of any one of claims 60-82, wherein the second genetic disruption disrupts one or more alleles of the endogenous B2M gene.

85. The genetically engineered human T cell of any one of claims 60-84, wherein the second genetic disruption disrupts all alleles of the endogenous B2M gene.

86. The genetically engineered human T cell of any one of claims 60-84, wherein the second genetic disruption reduces protein expression of B2M encoded from the endogenous B2M gene.

87. The genetically engineered human T cell of any one of claims 60-86, wherein there is no detectable expression of endogenous B2M in the genetically engineered human T cell.

88. The genetically engineered human T cell of any one of claims 60-87, wherein the genetically engineered human T cell has reduced expression of one or more HLA class I molecules.

89. The genetically engineered human T cell of any one of claims 60-88, wherein the genetically engineered human T cell has no detectable expression of one or more HLA class I molecules on the cell surface of the genetically engineered human T cell.

90. The genetically engineered human T cell of claim 88 or claim 89, wherein one or more HLA class I molecules is selected from HLA-A class I, HLA-B class I, and HLA-C class I.

91. The genetically engineered human T cell of any one of claims 57-60 and 63-90, further comprising:
(d) a second nucleotide sequence comprising a transgene encoding a single chain HLA-E fusion protein.

92. The genetically engineered human T cell of any one of claims 61-91, wherein the single chain HLA-E fusion protein comprises:
(1) at least a portion of the B2M protein,
(2) at least a portion of an HLA-E class I chain, and
(3) a peptide that is a portion of a signal sequence from an MHC class I molecule that is presented by the single chain HLA-E fusion protein when expressed on the cell surface of the genetically engineered human T cell.

93. The genetically engineered human T cell of claim 92, wherein: (1) the portion of the B2M protein comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:47; (2) the portion of an HLA-E class I chain comprises the sequence set forth in, or a sequence that is at least 85%, 86%, 87%, 88%, 89%, 90%, 91%, 92%, 93%, 94%, 95%, 96%, 97%, 98% or 99% identical to SEQ ID NO: 36; and (3) the peptide is VMAPRTLVL (SEQ ID NO:48), VMAPRTLLL (SEQ ID NO:49), VMAPRTVLL (SEQ ID NO:50), VMAPRTLFL (SEQ ID NO:51), or VMAPRTLIL (SEQ ID NO:52).

94. The genetically engineered human T cell of claim 93, wherein: (3) the peptide is VMAPRTLVL (SEQ ID NO:48).

95. The genetically engineered human T cell of claim 92, wherein: (1) the portion of the B2M protein comprises the sequence set forth in SEQ ID NO:47; (2) the portion of an HLA-E class I chain comprises the sequence set forth in SEQ ID NO: 36; and (3) the peptide is VMAPRTLVL (SEQ ID NO:48).

96. The genetically engineered human T cell of any one of claims 92-95, wherein the single chain HLA-E fusion protein further comprises: (4) a peptide linker that links (1) and (2).

97. The genetically engineered human T cell of any one of claim 96, wherein the peptide linker comprises a GS linker.

98. The genetically engineered human T cell of any one of claim 97, wherein the GS linker is a (G4S)x3 linker set forth in SEQ ID NO:53.

99. The genetically engineered human T cell of claim 97 or 98 wherein the GS linker is a (G4S)x4 linker set forth in SEQ ID NO: 54.

100. The genetically engineered human T cell of any one of claims 91-98, wherein the single chain HLA-E fusion protein comprises a sequence of amino acids that has at least about 85%, 86%, 87%, 88%, 89%, 90%, 91%, 92%, 93%, 94%, 95%, 96%, 97%, 98%, or 99% sequence identity to SEQ ID NO:34.

101. The genetically engineered human T cell of any one of claims 91-98, wherein the single chain HLA-E fusion protein comprises the sequence of amino acids set forth in SEQ ID NO:34.

102. The genetically engineered human T cell of any one of claims 91-101, wherein the single chain HLA-E fusion protein is capable of engaging an inhibitory receptor on the surface of an NK cell.

103. The genetically engineered human T cell of claim 102, wherein the inhibitory receptor on the surface of the NK cell is an NKG2A or NKG2B.

104. The genetically engineered human T cell of any one of claims 91-103, wherein the second nucleotide sequence encoding the single chain HLA-E fusion protein is present in the disrupted endogenous B2M gene in the T cell under the operable control of a promoter.

105. The genetically engineered human T cell of claim 104, wherein the promoter is an endogenous promoter of the endogenous B2M gene.

106. The genetically engineered human T cell of any one of claims 91-105, wherein the second nucleotide sequence encoding the single chain HLA-E fusion protein has been integrated in the disrupted endogenous B2M gene by homology directed repair (HDR).

107. The genetically engineered human T cell of any one of claims 57-60 and 63-106, further comprising (e) a third nucleotide sequence comprising:
(i) a first nucleic acid sequence at least 15 nucleotides in length complementary to an mRNA encoding TGF-B Receptor 2 (TGFBR2);
(ii) a second nucleic acid sequence at least 15 nucleotides in length complementary to an mRNA encoding Protein Tyrosine Phosphatase Non-Receptor Type 2 (PTPN2); and
(iii) a third nucleic acid sequence at least 15 nucleotides in length complementary an mRNA encoding Fas Cell Surface Death Receptor (FAS).

108. The genetically engineered human T cell of claim 61-107, wherein the third nucleotide sequence further comprises: (iv) a fourth nucleic acid at least 15 nucleotides in length complementary to an mRNA encoding human TGF-B Receptor 2 (TGFBR2).

109. The genetically engineered human T cell of claim 108, wherein the first and fourth nucleic acids are complementary to different nucleotides of the mRNA encoding human TGF-B Receptor 2 (TGFBR2).

110. The genetically engineered human T cell of any one of claims 61-109, wherein each nucleic acid of the third nucleotide sequence of (e) is a short hairpin RNA (shRNA), a small interfering RNA (siRNA), double stranded RNA (dsRNA), or an antisense oligonucleotide.

111. The genetically engineered human T cell of any one of claims 61-110, wherein each nucleic acid of the third nucleotide sequence of (e) is a short hairpin RNA (shRNA).

112. The genetically engineered human T cell of any one of claims 61-111, wherein: (i) the mRNA encoding TGFBR2 encodes human TGFBR2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:85, (ii) the mRNA encoding PTPN2 encodes human PTPN2 and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:86, and (iii) the mRNA encoding FAS encodes human FAS and comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:87.

113. The genetically engineered human T cell of any one of claims 61-112, wherein the mRNA encoding TGFBR2 comprises a sequence set forth in SEQ ID NO:85.

114. The genetically engineered human T cell of any one of claims 61-113, wherein the mRNA encoding PTPN2 comprises the sequence set forth in SEQ ID NO:86.

115. The genetically engineered human T cell of any one of claims 61-114, wherein the mRNA encoding FAS comprises the sequence set forth in SEQ ID NO:87.

116. The genetically engineered human T cell of any one of claims 61-115, wherein: (i) the first nucleic acid comprises a sequence complementary to nucleotides 2215 to 2236 of the mRNA encoding TGFBR2 comprising the sequence set forth in SEQ ID NO:85 (SEQ ID NO:74); the second nucleic acid comprises a sequence complementary to nucleotides 518 to 539 of the mRNA encoding PTPN2 comprising the sequence set forth in SEQ ID NO:86 (SEQ ID NO:73); and (iii) the third nucleic acid comprises a sequence complementary to nucleotides 1126 to 1147 of the mRNA encoding FAS comprising a sequence set forth in SEQ ID NO:87 (SEQ ID NO:72).

117. The genetically engineered human T cell of any one of claims 61-116, wherein the fourth nucleic acid comprises a sequence complementary to nucleotides 4430 to 4451 of the mRNA encoding TGFBR2 comprising the sequence set forth in SEQ ID NO:85 (SEQ ID NO:75).

118. The genetically engineered human T cell of any one of claims 61-117, wherein the third nucleotide sequence comprises the sequence set forth in, or a sequence that is at least 85%, 86%, 87%, 88%, 89%, 90%, 91%, 92%, 93%, 94%, 95%, 96%, 97%, 98% or 99% identical to, SEQ ID NO:71.

119. The genetically engineered human T cell of any one of claims 61-118, wherein the third nucleotide sequence comprises the sequence set forth in SEQ ID NO:71.

120. The genetically engineered human T cell of any one of claims 108-119, wherein the T cell is characterized by:
reduced expression of TGFBR2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid and/or the fourth nucleic acid,
reduced expression of PTPN2 by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid; and/or
reduced expression of FAS by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the third nucleic acid.

121. The genetically engineered human T cell of any one of claims 61-120, wherein the first nucleotide sequence of (a) and the third nucleotide sequence of (e) are on a same polynucleotide.

122. The genetically engineered human T cell of claim 121, wherein the polynucleotide is present in the disrupted endogenous TRAC gene in the genetically engineered human T cell, and the first and third nucleotide sequences are under the operable control of a promoter.

123. The genetically engineered human T cell of claim 122, wherein the promoter is a heterologous promoter of the endogenous TRAC gene.

124. The genetically engineered human T cell of claim 122 or claim 123, wherein the heterologous promoter is a constitutive promoter.

125. The genetically engineered human T cell of claim 123 or claim 124, wherein the heterologous promoter is or comprises a human elongation factor 1 alpha (EF1α) promoter or a variant thereof.

126. The genetically engineered human T cell of any one of claims 122-124, wherein the promoter is a synthetic promoter.

127. The genetically engineered human T cell of any one of claims 121-126, wherein the polynucleotide further comprises an intron between the first nucleotide of (a) and the second nucleotide of (c).

128. The genetically engineered human T cell of any one of claims 121-127, wherein the polynucleotide comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO:67.

129. The genetically engineered human T cell of any one of claims 121-128, wherein the polynucleotide comprises the sequence set forth in SEQ ID NO:67.

130. The genetically engineered human T cell of any one of claims 121-129, wherein the polynucleotide has been integrated in the disrupted endogenous TRAC gene by homology directed repair (HDR).

131. The genetically engineered human T cell of any one of claims 60-130, wherein the genetically engineered human T cell further comprises: (f) a third genetic disruption in an endogenous CD70 gene.

132. The genetically engineered human T cell of claim 131, wherein the third genetic disruption in the endogenous CD70 gene is in a target site sequence in exon 2 of the endogenous CD70 gene.

133. The genetically engineered human T cell of claim 132, wherein the target site sequence in exon 2 of the endogenous CD70 gene is located at hg38 genomic coordinates chr19:6,590,122-6,590,142.

134. The genetically engineered human T cell of claim 132 or claim 133, wherein the target site sequence in exon 2 of the endogenous CD70 gene has the sequence set forth in SEQ ID NO: 66, a contiguous portion thereof of at least 14 nucleotides (nt), or a complementary sequence of the foregoing.

135. The genetically engineered human T cell of any one of claims 132-134, wherein the target site sequence in exon 2 of the endogenous CD70 gene has the sequence set forth in SEQ ID NO: 66.

136. The genetically engineered human T cell of any one of claims 131-135, wherein the third genetic disruption is by a CRISPR-Cas system that comprises a Cas12a protein and a guide RNA (gRNA) comprising a spacer sequence comprising the nucleic acid sequence of SEQ ID NO: 65, or a contiguous portion thereof of at least 14 nt.

137. The genetically engineered human T cell of claim 136, wherein the CRISPR-Cas system is a ribonucleoprotein (RNP) complex comprising the Cas12a protein and the gRNA.

138. The genetically engineered human T cell of any one of claims 131-137, wherein the third genetic disruption disrupts one or more alleles of the endogenous CD70 gene.

139. The genetically engineered human T cell of any one of claims 131-138, wherein the third genetic disruption disrupts all alleles of the endogenous CD70 gene.

140. The genetically engineered human T cell of any one of claims 131-139, wherein the third genetic disruption reduces protein expression of CD70 encoded from the endogenous CD70 gene.

141. The genetically engineered human T cell of any one of claims 131-140, wherein the third genetic disruption reduces protein expression of CD70 on the surface of the genetically engineered human T cell.

142. The genetically engineered human T cell of any one of claims 131-141, wherein there is no detectable expression of CD70 in the genetically engineered human T cell.

143. The genetically engineered human T cell of any one of claims 131-142, wherein the genetically engineered human T cell has reduced fratricide as compared to a control cell that does not comprise the third genetic disruption.

144. The genetically engineered human T cell of any one of claims 57-143, wherein the human T cell is a primary human T cell.

145. The genetically engineered human T cell of claim 144, wherein the primary human T cell is from a healthy human donor.

146. A genetically engineered human T cell comprising:
(a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising: (i) a nucleotide sequence comprising the sequence set forth in SEQ ID NO:71 and (ii) a nucleotide sequence encoding a CAR comprising the amino acid sequence set forth in SEQ ID NO: 38 or SEQ ID NO: 41, wherein X is Q or pyroglutamate, is present in the disrupted endogenous TRAC gene in the genetically engineered human T cell,
(b) a second genetic disruption in exon 2 of an endogenous B2M gene located at hg38 genomic coordinates chr15:44,715,614-44,715,634, wherein the second genetic disruption disrupts one or more alleles of the endogenous B2M gene, and wherein a nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and
(c) a third genetic disruption in exon 2 of an endogenous CD70 gene located at hg38 genomic coordinates chr19:6,590,122-6,590,142, wherein the third genetic disruption disrupts all alleles of the endogenous CD70 gene.

147. A genetically engineered human T cell comprising:
(a) a first genetic disruption in exon 1 of an endogenous TRAC gene located at hg38 genomic coordinates chr14:22,547,528-22,547,548, wherein the first genetic disruption disrupts one or more alleles of the endogenous TRAC gene, and wherein a polynucleotide comprising:
(i) a nucleotide sequence comprising: (1) a first shRNA comprising the sequence set forth in SEQ ID NO:74, (2) a second shRNA comprising the sequence set forth in SEQ ID NO:73, (3) a third shRNA comprising the sequence set forth in SEQ ID NO:72, and (4) a fourth shRNA comprising the sequence set forth in SEQ ID NO:75; and
(ii) a nucleotide sequence encoding a CAR targeting CD70 comprising the sequence set forth in SEQ ID NO: 38 or SEQ ID NO: 41, wherein X is Q or pyroglutamate, is present in the disrupted TRAC gene in the genetically engineered human T cell,
(b) a second genetic disruption in exon 2 of an endogenous B2M gene located at hg38 genomic coordinates chr15:44,715,614-44,715,634, wherein the second genetic disruption disrupts one or more alleles of the endogenous B2M gene, and wherein the first nucleotide sequence encoding a single chain HLA-E fusion protein comprising the sequence set forth in SEQ ID NO:34 is present in the disrupted endogenous B2M gene in the genetically engineered human T cell under the operable control of an endogenous promoter of the endogenous B2M gene; and
(c) a third genetic disruption in exon 2 of an endogenous CD70 gene located at hg38 genomic coordinates chr19:6,590,122-6,590,142, wherein the third genetic disruption disrupts all alleles of the endogenous CD70 gene.

148. A population of genetically engineered human T cells comprising the genetically engineered human T cell of any one of claims 57-147.

149. The population of genetically engineered human T cells of claim 148, wherein at least or at about 50%, at least or at about 60%, at least or at about 70%, at least or at about 80%, or at least or at about 90% of the cells in the population have a genetic modification selected from:
(a) the first genetic disruption in the endogenous TRAC gene;
(b) the second genetic disruption in the endogenous B2M gene;
(c) a knock-in of the first nucleotide sequence transgene encoding the single chain HLA-E fusion protein;
(d) a knock-in of the second nucleotide sequence;
(e) a knock-in of the third nucleotide sequence encoding the CAR; and/or
(f) the third genetic disruption in the endogenous CD70 gene.

150. The population of genetically engineered human T cells of claim 148 or claim 149, wherein at least or at about 50%, at least or at about 60%, at least or at about 70%, at least or at about 80%, or at least or at about 90% of the cells in the population is characterized by:
(1) one or more of (a)-(f);
(2) two or more of (a)-(f);
(3) three or more of (a)-(f);
(4) four or more of (a)-(f);
(5) five or more of (a)-(f); or
(6) all of (a)-(f).

151. A composition comprising the population of genetically engineered human T cells of any one of claims 148-150.

152. The composition of claim 151, wherein the composition is a pharmaceutical composition comprising a pharmaceutically acceptable excipient.

153. The composition of claim 151 or claim 152, wherein the composition comprises a cryoprotectant, optionally wherein the cryoprotectant is DMSO.

154. A method of treatment comprising administering the genetically engineered human T cell of any one of claims 57-147, the population of genetically engineered human T cells of any one of claims 148-150, or the composition of any one of claims 151-153 to a human subject having a disease or disorder.

155. The method of claim 154, wherein the disease or disorder is associated with CD70.

156. The method of claim 154 or claim 155, wherein the disease or disorder is a cancer.

157. The method of claim 156, wherein the cancer is a solid tumor.

158. The method of claim 157, wherein the solid tumor is a renal cell carcinoma.

159. The method of claim 158, wherein the renal cell carcinoma is a clear cell renal cell carcinoma.

160. The method of any one of claims 154-159, wherein the human subject is non-responsive or refractory to one or more standard therapies for cancer.

161. The method of any one of claims 154-160, wherein the human subject has developed drug resistance to one or more standard therapies for cancer.

162. The method of any one of claims 154-160, wherein the human subject has undergone surgery in an attempt to treat the disease or disorder.

163. The method of any one of claims 154-159, wherein the human subject has not been previously treated for cancer.

164. The method of any one of claims 154-159 or 163, wherein the human subject has not undergone surgery in an attempt to treat the disease or disorder.

165. The method of any one of claims 154-164, wherein the method comprises allogeneic transfer, in which the cells are isolated and/or otherwise prepared from a donor other than the human subject.

166. The method of claim 165, wherein the donor is a healthy donor.

167. The method of claim 165 or claim 166, wherein the donor does not have the disease or disorder.

168. The method of any one of claims 165-167, wherein the donor and the human subject are genetically identical or genetically similar.

169. The method of any one of claims 165-168, wherein the human subject expresses a HLA class or supertype that is the same as the donor.

170. The method of any one of claims 165-168, wherein the human subject does not express a HLA class or supertype that is the same as the donor.

171. The method of any one of claims 154-170, comprising administering a dose comprising a therapeutically effective amount of the genetically engineered human T cell, the population of genetically engineered human T cells, or the composition.

172. The method of claim 171, wherein the dose comprises about 25×10⁶, about 50×10⁶, 100×10⁶, about 150×10⁶, about 200×10⁶, about 250×10⁶, about 300×10⁶, about 350×10⁶, about 400×10⁶, about 450×10⁶, about 500×10⁶, about 550×10⁶, about 650×10⁶, about 700×10⁶, about 750×10⁶, about 800×10⁶, about 850×10⁶, about 900×10⁶, about 950×10⁶, about 1×10⁹, about 1.1×10⁹, about 1.2×10⁹, about 1.3×10⁹, about 1.4×10⁹, or about 1.5×10⁹ CAR+ engineered T cells.

173. The method of claim 172, wherein the dose comprises about 50×10⁶ CAR+ engineered T cells.

174. The method of claim 172, wherein the dose comprises about 100×10⁶ CAR+ engineered T cells.

175. The method of claim 172, wherein the dose comprises about 300×10⁶ CAR+ engineered T cells.

176. The method of claim 172, wherein the dose comprises about 900×10⁶ CAR+ engineered T cells.

177. The method of any one of claims 171-176, wherein the dose is administered via an intravenous (IV) infusion.

178. The method of claim 177, wherein the IV infusion is a single IV infusion.

179. The method of claim 177, wherein the human subject receives more than one dose or more than one IV infusion.

180. The method of any one of claims 171-179, the dose is provided as a suspension for administration via IV infusion.

181. The method of any one of claims 154-180, wherein the human subject is pretreated with a lymphodepleting chemotherapy prior to administration of the genetically engineered human T cells.

182. The method of claim 181, wherein the lymphodepleting chemotherapy comprises treating the human subject with fludarabine IV (30 mg/m²/day) and cyclophosphamide IV (500 mg/m²/day) for three days prior to administration of the genetically engineered human T cells.

183. The method of claim 181, wherein the lymphodepleting chemotherapy comprises treating the human subject with fludarabine IV (30 mg/m²/day) and cyclophosphamide IV (500 mg/m²/day) for four days or five days prior to administration of the genetically engineered human T cells.

184. The method of any one of claims 154-183, further comprising at least one additional therapy, therapeutic agent, or modality.

185. The method of claim 184, wherein the at least one additional therapeutic agent or modality comprises at least one PD-1 therapy.

186. The method of claim 185, wherein the at least one PD-1 therapy is an antibody or antigen-binding fragment thereof that binds to PD-1.

187. The method of claim 185, wherein the at least one PD-1 therapy is an antibody or antigen-binding fragment thereof that binds to PD-L1.

188. The method of any one of claims 185-187, wherein the at least one PD-1 therapy is selected from the group consisting of nivolumab, pembrolizumab, cemiplimab, dostarlimab, retifanlimab, toripalimab, atezolizumab, avelumab, durvalumab, cosibelimab, camrelizumab, sintilimab, and tislelizumab.

189. The method of claim 188, wherein the at least one PD-1 therapy is nivolumab.

190. The method of claim 189, wherein the nivolumab is administered to the human subject about once every two weeks at a dose of 240 mg.

191. The method of claim 189, wherein the nivolumab is administered to the human subject about once every two weeks at a dose of 600 mg.

192. The method of claim 189, wherein the nivolumab is administered to the human subject about once every two weeks at a dose of 720 mg.

193. The method of claim 189, wherein the nivolumab is administered to the human subject about once every two weeks at a dose of 960 mg.

194. The method of claim 189, wherein the nivolumab is administered to the human subject about once every two weeks at a dose of 1200 mg.

195. The method of claim 189, wherein the nivolumab is administered to the human subject about once every three weeks at a dose of 360 mg.

196. The method of claim 189, wherein the nivolumab is administered to the human subject about once every three weeks at a dose of 720 mg.

197. The method of claim 189, wherein the nivolumab is administered to the human subject about once every three weeks at a dose of 900 mg.

198. The method of claim 189, wherein the nivolumab is administered to the human subject about once every three weeks at a dose of 960 mg.

199. The method of claim 189, wherein the nivolumab is administered to the human subject about once every three weeks at a dose of 1200 mg.

200. The method of claim 189, wherein the nivolumab is administered to the human subject about once every four weeks at a dose of 480 mg.

201. The method of claim 189, wherein the nivolumab is administered to the human subject about once every four weeks at a dose of 720 mg.

202. The method of claim 189, wherein the nivolumab is administered to the human subject about once every four weeks at a dose of 960 mg.

203. The method of claim 189, wherein the nivolumab is administered to the human subject about once every four weeks at a dose of 1200 mg.

204. The method of claim 189, wherein the nivolumab is administered to the human subject about once every two weeks at a dose of between 3 mg/kg and 10 mg/kg.

205. The method of claim 189, wherein the nivolumab is administered to the human subject about once every two weeks at a dose of 3 mg/kg.

206. The method of any one of claims 189-205, wherein the nivolumab is administered intravenously.

207. The method of any one of claims 189-205, wherein the nivolumab is administered subcutaneously.

208. The method of any one of claims 189-205 and 207, wherein the nivolumab is co-formulated with hyaluronidase.

209. The method of claim 208, wherein the hyaluronidase is at a dose of about 10,000 units to about 20,000 units, inclusive.

210. The method of claim 208 or claim 209, wherein the hyaluronidase is at a dose of about 10,000 units; about 12,000 units; 15,000 units; about 16,000 units; or about 20,000 units.

211. The method of any one of claims 208-210, wherein the nivolumab co-formulated with hyaluronidase is administered to the human subject about once every two weeks at a dose of 600 mg nivolumab and a dose of about 10,000 units hyaluronidase.

212. The method of any one of claims 208-210, wherein the nivolumab co-formulated with hyaluronidase is administered to the human subject about once every three weeks at a dose of 900 mg nivolumab and a dose of about 15,000 units hyaluronidase.

213. The method of any one of claims 208-210, wherein the nivolumab co-formulated with hyaluronidase is administered to the human subject about once every four weeks at a dose of 1200 mg nivolumab and about 20,000 units hyaluronidase.

214. The method of any one of claims 208-210, wherein the nivolumab co-formulated with hyaluronidase is administered to the human subject about once every two to four weeks at a dose of 720 mg nivolumab and about 20,000 units hyaluronidase.

215. The method of any one of claims 208-210, wherein the nivolumab co-formulated with hyaluronidase is administered to the human subject about once every two to four weeks at a dose of 960 mg nivolumab and about 20,000 units hyaluronidase.

216. The method of any one of claims 208-210, wherein the nivolumab co-formulated with hyaluronidase is administered to the human subject about once every two to four weeks at a dose of 1200 mg nivolumab and about 20,000 units hyaluronidase.

217. The method of any one of claims 208-210, wherein the nivolumab co-formulated with hyaluronidase is administered to the human subject about once every two to four weeks at a dose of 720 mg nivolumab and about 12,000 units hyaluronidase.

218. The method of any one of claims 208-210, wherein the nivolumab co-formulated with hyaluronidase is administered to the human subject about once every two to four weeks at a dose of 960 mg nivolumab and about 16,000 units hyaluronidase.

219. The method of any one of claims 208-210, wherein the nivolumab co-formulated with hyaluronidase is administered to the human subject about once every two to four weeks at a dose of 1200 mg nivolumab and about 20,000 units hyaluronidase.

220. The method of any one of claims 214-219, wherein the once every two to four weeks is once every two weeks.

221. The method of any one of claims 214-219, wherein the once every two to four weeks is once every three weeks.

222. The method of any one of claims 214-219, wherein the once every two to four weeks is once every four weeks.

223. The method of any one of claims 185-222, wherein the at least one PD-1 therapy is administered to the human subject for 4, 6, 8, 12, 15, 18, 20, 24, 30, 36, 40, 48, or 60 months.

224. The method of any one of claims 185-222, wherein the at least one PD-1 therapy is discontinued after 4, 6, 8, 12, 15, 18, 20, 24, 30, 36, 40, 48, or 60 months.

225. The method of any one of claims 185-222, wherein the at least one PD-1 therapy is administered to the human subject for 12 months.

226. The method of any one of claims 185-222, wherein the at least one PD-1 therapy is administered to the human subject for 24 months.

227. The method of any one of claims 185-226, wherein the at least one PD-1 therapy is administered to the human subject until the disease or disorder progresses.

228. The method of any one of claims 185-227, wherein the at least one additional therapeutic agent or modality comprises at least one tyrosine kinase inhibitor (TKI).

229. The method of claim 228, wherein the at least one TKI is administered to the human subject as an oral capsule or tablet.

230. The method of claim 228 or claim 229, wherein the at least one TKI is a small-molecule inhibitor of at least one of c-Met (HGFR), VEGFR1, VEGFR2, VEGFR3, AXL, RET, and FLT3.

231. The method of claim 230, wherein the at least one TKI is a small-molecule inhibitor of at least one of c-Met (HGFR), VEGFR2, AXL, RET, and FLT3.

232. The method of any one of claims 228-231, wherein the at least one TKI is or comprises cabozantinib, tivozanib, lenvatinib, axitinib, pazopanib or sunitinib.

233. The method of any one of claims 228-232, wherein the at least one TKI is or comprises cabozantinib.

234. The method of claim 233, wherein the cabozantinib is administered to the human subject once daily.

235. The method of claim 233 or claim 234, wherein the cabozantinib is administered to the human subject until the disease or disorder progresses.

236. The method of any one of claims 233-235, wherein the cabozantinib is administered to the human subject at a dose of 40 mg/day.

237. The method of any one of claims 233-235, wherein the cabozantinib is administered to the human subject at a dose of 60 mg/day.

238. The method of any one of claims 228-232, wherein the at least one TKI is a small-molecule inhibitor of at least one of VEGFR1, VEGFR2, and VEGFR3.

239. The method of any one of claims 228-232 and claim 238, wherein the at least one TKI is or comprises tivozanib.

240. The method of claim 239, wherein the tivozanib is administered to the human subject once daily.

241. The method of claim 239 or claim 240, wherein the tivozanib is administered to the human subject until the disease or disorder progresses.

242. The method of any one of claims 239-241, wherein the tivozanib is administered to the human subject in a dosing cycle.

243. The method of claim 242, wherein the dosing cycle comprises a 28-day cycle comprising administering to the human subject one dose of tivozanib orally for 21 days followed by seven days without administration of tivozanib to the human subject.

244. The method of any one of claims 239-243, wherein the tivozanib is administered to the human subject at a dose of 1.34 mg/day.

245. The method of any one of claims 239-243, wherein the tivozanib is administered to the human subject at a dose of 0.89 mg/day.

246. A guide RNA (gRNA), wherein the gRNA targets an endogenous TRAC gene and comprises the sequence set forth in SEQ ID NO:55.

247. A guide RNA (gRNA), wherein the gRNA targets an endogenous CD70 gene and comprises the sequence set forth in SEQ ID NO:64.

248. A polynucleotide encoding the CAR of any one of claims 1-56.

249. A polynucleotide comprising the sequence set forth in, or a sequence that has at least about 85%, 86%, 87%, 88%, 89%, 90%, 91%, 92%, 93%, 94%, 95%, 96%, 97%, 98%, or 99% sequence identity to, SEQ ID NO:67.

250. The polynucleotide of claim 248 or claim 249, wherein the polynucleotide comprises the sequence set forth in SEQ ID NO:67.

251. A polynucleotide, wherein the polynucleotide comprises the sequence set forth in, or a sequence of amino acids that has at least about 85%, 86%, 87%, 88%, 89%, 90%, 91%, 92%, 93%, 94%, 95%, 96%, 97%, 98%, or 99% sequence identity to, SEQ ID NO:77.

252. The polynucleotide of claim 251, wherein the polynucleotide comprises the sequence set forth in SEQ ID NO:77.

253. A vector comprising the polynucleotide of any one of claims 258-252.

254. The vector of claim 253, wherein the vector is a viral vector.

255. The vector of claim 254, wherein the viral vector is an AAV.

256. A cell comprising the CAR of any one of claims 1-56, the gRNA of claim 246 or claim 247, the polynucleotide of any one of claims 248-252, or the vector of any one of claims 253-255.

257. The cell of claim 256, wherein the cell is a T cell.

258. The cell of claim 257, wherein the T cell is a primary T cell.

259. The cell of claim 258, wherein the primary T cell is from a human donor.

260. The cell of any one of claims 257-259, wherein the T cell is a CD4+ T cell, a CD8+ T cell, or a CD4+ and CD8+ T cell.

261. An antibody, or antigen-binding fragment thereof, comprising a CD70-binding domain that binds to CD70, wherein the CD70-binding domain comprises a heavy chain only variable region (VHH), wherein:
(i) the VHH comprises a CDR-1, CDR-2, and CDR-3 each comprising a sequence that is contained within SEQ ID NO: 1, wherein X is Q or pyroglutamate; or
(ii) the VHH comprises a CDR-1, CDR-2, and CDR-3 each comprising a sequence that is contained within SEQ ID NO:27, wherein X is Q or pyroglutamate.

262. The antibody, or antigen-binding fragment thereof, of claim 261, wherein:
(i) the VHH of the CD70-binding domain comprises CDR-1, CDR-2, and CDR-3 sequences set forth in SEQ ID NOs: 4, 5, and 6, respectively;
(ii) the VHH of the CD70-binding domain comprises CDR-1, CDR-2, and CDR-3 sequences set forth in SEQ ID NOs: 7, 8, and 9, respectively;
(iii) the VHH of the CD70-binding domain comprises CDR-1, CDR-2, and CDR-3 sequences set forth in SEQ ID NOs: 79, 80, and 81, respectively; or
(iv) the VHH of the CD70-binding domain comprises CDR-1, CDR-2, and CDR-3 sequences set forth in SEQ ID NOs: 82, 83, and 84, respectively.

263. The antibody, or antigen-binding fragment thereof, of claim 261 or claim 262, wherein:
(i) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 1, wherein X is Q or pyroglutamate; or
(ii) the VHH comprises a sequence set forth in, or a sequence that is at least 85%, 86%, 87%, 88%, 89%, 90%, 91%, 92%, 93%, 94%, 95%, 96%, 97%, 98% or 99% identical to, SEQ ID NO: 27, wherein X is Q or pyroglutamate.

264. The antibody, or antigen-binding fragment thereof, of any one of claims 261-263, wherein:
(i) the VHH comprises a sequence set forth in SEQ ID NO: 1, wherein X is Q or pyroglutamate; or
(ii) the VHH comprises a sequence set forth in SEQ ID NO: 27, wherein X is Q or pyroglutamate.
